(19) United States
(12) Patent Application Publication
(10) Pub. No.: US 2025/0002563 A1
(43) Pub. Date: Jan. 2, 2025

(54) BROADLY NEUTRALIZING NANOBODIES FOR CORONAVIRUS AND USES THEREOF

(71) Applicant: UNIVERSITY OF PITTSBURGH-OF THE COMMONWEALTH SYSTEM OF HIGHER EDUCATION, Pittsburgh, PA (US)

(72) Inventors: Yi SHI, Wexford, PA (US); Yufei XIANG, Pittsburgh, PA (US)

(21) Appl. No.: 18/690,481

(22) PCT Filed: Sep. 12, 2022
(86) PCT No.: PCT/US2022/076293

§ 371 Date: Mar. 8, 2024

Related U.S. Application Data
(60) Provisional application No. 63/242,886, filed on Sep. 10, 2021.
(60) Provisional application No. 63/291,921, filed on Dec. 20, 2021.

(51) Int. Cl. C07K 16/10; A61K 47/64; A61P 31/14

(52) U.S. Cl. CPC C07K 16/1003; A61K 47/643; A61P 31/14; C07K 2317/569

(57) ABSTRACT

Disclosed herein are coronavirus neutralizing antibodies and uses thereof for treating and/or preventing a coronavirus infection in a subject.

### CROSS-REFERENCE TO RELATED APPLICATIONS

This application claims the benefit of U.S. Provisional Application No. 63/242,886, filed Sep. 10, 2021, and U.S. Provisional Application No. 63/291,921, filed Dec. 20, 2021, which are expressly incorporated herein by reference in their entireties.

### STATEMENT REGARDING FEDERALLY SPONSORED RESEARCH OR DEVELOPMENT

This invention was made with government support under grant number GM137905 awarded by the National Institutes of Health. The government has certain rights in the invention.

### BACKGROUND

Nanobodies (Nbs) are natural antigen-binding fragments derived from the VHH domain of camelid heavy-chain only antibodies (HcAbs). They are characterized by their small size and outstanding structural robustness, excellent solubility and stability, ease of bioengineering and manufacturing, low immunogenicity in humans and fast tissue penetration. For these reasons, Nbs have emerged as promising agents for cutting-edge biomedical, diagnostic and therapeutic applications.

A novel, highly transmissible coronavirus SARS-COV-2 (severe acute respiratory syndrome coronavirus 2) {Zhu, 2020; Zhou, 2020} has infected more than 20 million people and has claimed over 700,000 lives, with the numbers still on the rise. Despite preventive measures, such as quarantines and lock-downs that help curb viral transmission, the virus often rebounds following the lifts on social restrictions. Safe and effective therapeutics and vaccines remain in dire need.

Like other zoonotic coronaviruses, SARS-COV-2 produces the surface spike glycoprotein (S), which is then cleaved into S1 and S2 subunits forming the homotrimeric viral spike to interact with host cells. The interaction is mediated by the S1 receptor-binding domain (RBD), which binds the peptidase domain (PD) of angiotensin-converting enzyme-2 (hACE2) as a host receptor {Wrapp, 2020}. Structural studies have revealed different stages of the spike trimer {Walls, 2020; Cai, 2020}. In the prefusion stage, the RBD switches between an inactive, closed conformation, and an active open structure necessary for interacting with hACE2. In the post-fusion stage, S1 dissociates from the trimer, and S2 undergoes a dramatic conformational change to trigger host membrane fusion. Most recently, investigations into COVID-19 convalescence individuals' sera have led to the identifications of highly potent neutralizing IgG antibodies (NAbs) primarily targeting the RBD but also the N-terminal domain (NTD) of the spike trimer {Cao, 2020; Robbiani, 2020; Hansen, 2020; Liu, 2020; Brouwer, 2020; Chi, 2020}. High-quality NAbs may overcome the risks of the Fc-associated antibody-dependent enhancement (ADE) and are promising therapeutic and prophylactic candidates {Zohar, 2020; Eroshenko, 2020}.

The VHH antibodies or nanobodies (Nbs) are minimal, monomeric antigen-binding fragments derived from camelid single-chain antibodies {Muyldermans, 2013}. Unlike IgG antibodies, Nbs are characterized by small sizes (˜15 kDa), high solubility and stability, ease of bioengineering into bi/multivalent forms, and low-cost microbial productions. Because of the robust physicochemical properties, Nbs are flexible for drug administration such as aerosolization, making their use against the respiratory, viral targets appealing {Vanlandschoot, 2011; Detalle, 2016}. Previous efforts have yielded broadly neutralizing Nbs for different challenging viruses, including Dengue, RSV, and HIV {Vanlandschoot, 2011}. However, highly potent camelid Nbs comparable to the human NAbs remain unavailable {Huo, 2020; Wrapp, 2020; Konwarh, 2020}. The development of highly effective anti-SARS-COV-2 Nbs can provide a novel means for efficient and economic strategies for therapeutics and point-of-care diagnosis.

### SUMMARY

Provided herein are coronavirus (e.g., SARS-CoV-2, SARS-CoV, or a sarbecovirus) neutralizing nanobodies and uses thereof for preventing or treating coronavirus infection. The nanobodies disclosed herein are surprisingly effective at reducing coronavirus viral load and preventing and treating a coronavirus infection. In some embodiments, the coronavirus neutralizing nanobody comprises one or more complementarity determining regions 3 (CDR3), wherein the CDR3 comprises an amino acid sequence selected from the group consisting of SEQ ID NO: 1 through SEQ ID NO: 1057. In some embodiments, the nanobody comprises a multimer of one or more CDR3s, wherein the CDR3 comprises a sequence selected from the group consisting of SEQ ID NO: 1 through SEQ ID NO: 1057. In some embodiments, the nanobody comprises a multimer (including, for example, a homodimer, a heterodimer, a homotrimer, or a heterotrimer) of one or more amino acid sequences comprising a sequence selected from the group consisting of SEQ ID NO: 1058 through SEQ ID NO: 2114 and SEQ ID NO: 2117 through SEQ ID NO: 2119. In some embodiments, the nanobody comprises a multimer (including, for example, a homodimer, a heterodimer, a homotrimer, or a heterotrimer) of one or more amino acid sequences comprising a sequence selected from the group consisting of SEQ ID NO: 1058-1072, 1074, 1075, 1077, 1078, 1079, 1080, 1082, 1083, 1084, 1085, 1086, 1087, 1089, 1090, 1091, 1092, 1093, 1096, 1097, 1098, 1099, 1100, 1103, 1105, 1106, 1111, 1118, 1122, 1127, 1128, 1129, 1130, 1132, 1133, 1134, 1138, 1146, 1147, 1148, 1149, 1150, 1153, 1154, 1155, 1156, 1157, 1161, 1185, 1191, 1193, 1194, 1196, 1197, 1198, 1200, 1203, 1204, 1205, 1208, 1209, 1211, 1220, 1221, 1222, 1229, 1234, 1237, 1240, 1241, 1249, 1260, 1261, 1262, 1271, 1274, 1276, 1278, 1286, 1287, 1288, 1291, 1294, 2117, 2118, and 2119. In some embodiments, the coronavirus neutralizing nanobody is conjugated or linked to a human serum albumin binding nanobody or nanobody fragment. In some examples, the nanobody described herein exhibits high potency, with an IC50 of less than about 1 ng/1 ml.

The method provided herein comprises uses of the nanobodies described herein for treating or preventing a coronavirus infection (e.g., a sarbecovirus, SARS-CoV-2 or SARS-CoV). In some examples, the method comprises administering the nanobody at a dose of about 0.2 mg/kg of body weight. The nanobody can be administered to a subject intratracheally, intranasally, or through an inhalation route. The nanobody has an increased serum half-life or in vivo stability as compared to a control.

### DESCRIPTION OF DRAWINGS

FIG. 1(A-F) shows production and characterizations of high-affinity RBD Nbs for SARS-CoV-2 neutralization. FIG. 1a shows the binding affinities of 71 Nbs towards RBD by ELISA. The pie chart shows the number of Nbs according to affinity and solubility. FIG. 1B shows screening of 49 soluble, high-affinity Nbs by SARS-CoV-2-GFP pseudovirus neutralization assay. n=1 for Nbs with neutralization potency IC50<=50 nM, n=2 for Nbs with neutralization potency IC50>50 nM. FIG. 1C shows that the neutralization potency of 18 highly potent Nbs was calculated based on the pseudotyped SARS-CoV-2 neutralization assay (luciferase). Purple, red, and yellow lines denote Nbs 20, 21, and 89 with IC50<0.2 nM. Two different purifications of the pseudovirus were used. The average neutralization percentage was shown for each data point (n=5 for Nbs 20, 21; n=2 for all other Nbs). FIG. 1D shows the neutralization potency of the top 14 neutralizing Nbs by SARS-CoV-2 plaque reduction neutralization test (PRNT). The average neutralization percentage was shown for each data point (n=4 for Nbs 20, 21, and 89; n=2 for other Nbs). FIG. 1E shows a table summary of pseudotyped and SARS-CoV-2 neutralization potency for 18 Nbs. N/A: not tested. FIG. 1F shows the SPR binding kinetics measurement of Nb21.

FIG. 2(A-G) shows Nb epitope mapping by integrative structural proteomics. FIG. 2A shows a summary of Nb epitopes based on size exclusion chromatography (SEC) analysis. Light salmon color: Nbs that bind the same RBD epitope. Sea green: Nbs of different epitopes. FIG. 2B shows a representation of SEC profiling of RBD, RBD-Nb21 complex, and RBD-Nb21-Nb105 complex. The y-axis represents UV 280 nm absorbance units (mAu). FIG. 2C shows a cartoon model showing the localization of five Nbs that bind different epitopes: Nb20 (medium purple), Nb34 (light sea green), Nb93 (salmon), Nb105 (pale goldenrod) and Nb95 (light pink) in complex with the RBD (gray). Blue and red lines represent DSS cross-links shorter or longer than 28 Å, respectively. FIG. 2D shows top 10 scoring cross-linking based models for each Nb (cartoons) on top of the RBD surface. FIG. 2E shows the surface display of different Nb neutralization epitopes on the RBD in complex with hACE2 (cartoon model in blue). FIG. 2e: Schematics of five unique RBD epitopes for Nb binding. The residue numbers of the RBD were shown (from aa 333 to aa 533). FIG. 2F shows schematics of five unique RBD epitopes for Nb binding. The residue numbers of the RBD were shown (from aa 333 to aa 533). FIG. 2G shows an overview of the Nb neutralization epitopes revealed by cross-linking.

FIG. 3(A-F) shows crystal structure analysis of an ultrahigh affinity Nb in complex with the RBD. FIG. 3A shows cartoon presentation of Nb20 in complex with the RBD. CDR1, 2, and 3 are in red, green, and orange, respectively. FIG. 3B shows zoomed-in view of an extensive polar interaction network that centers on R35 of Nb20. FIG. 3C shows zoomed-in view of hydrophobic interactions. FIG. 3D shows surface presentation of the Nb20-RBD and hACE2-RBD complex (PDB: 6M0J). FIG. 3E shows surface presentation of RBD with hACE2 binding epitope colored in steel blue and Nb20 epitope colored in medium purple. FIG. 3F shows the CDR1 and CDR3 residues (medium violet pink and light goldenrod in spheres, respectively) of Nb20 overlap with hACE2 binding site (light blue) on the RBD (gray).

FIG. 4(A-F) shows mechanisms of SARS-CoV-2 neutralization by Nbs. FIG. 4A shows hACE2 (grey) binding to spike trimer conformation (wheat, plum, and light blue colors) with one RBD up (PDBs 6VSB, 6LZG). FIG. 4B shows that Nb20 (Epitope I, medium purple) partially overlaps with the hACE2 binding site and can bind the closed spike conformation with all RBDs down (PDB 6VXX). FIG. 4C shows a summary of spike conformations accessible (+) to the Nbs of different epitopes. FIG. 4D shows that Nb93 (Epitope II, salmon) partially overlaps with the hACE2 binding site and can bind to spike conformations with at least one RBD up (PDB 6VSB). FIG. 4(E-F) shows that Nb34 (Epitope III, light sea blue) and Nb95 (Epitope IV, light pink) do not overlap with the hACE2 binding site and bind to spike conformations with at least two open RBDs (PDB 6XCN).

FIG. 5(A-E) shows development of multivalent Nb cocktails for highly efficient SARS-CoV-2 neutralization. FIG. 5A shows schematics of the cocktail design. FIG. 5B show pseudotyped SARS-CoV-2 neutralization assay of multivalent Nbs. The average neutralization percentage of each data point was shown (n=2). FIG. 5C shows SARS-CoV-2 PRNT of monomeric and trimeric forms of Nbs 20 and 21. The average neutralization percentage of each data point was shown (n=2 for the trimers, n=4 for the monomers). FIG. 5D shows a summary table of the neutralization potency measurements of the multivalent Nbs. N/A: not tested. FIG. 5E shows mapping mutations to localization of Nb epitopes on the RBD. The x-axis corresponds to the RBD residue numbers (333 to 533). Rows in different colors represent different epitope residues. Epitope I: 351, 449-450, 452-453, 455-456, 470, 472, 483-486, 488-496; Epitope II: 403, 405-406, 408,409, 413-417, 419-421, 424, 427, 455-461, 473-478, 487, 489, 505; Epitope III: 53, 355, 379-383, 392-393, 396, 412-413, 424-431, 460-466, 514-520; Epitope IV: 333-349, 351-359, 361, 394, 396-399, 464-466, 468, 510-511, 516; Epitope V: 353, 355-383, 387, 392-394, 396, 420, 426-431, 457, 459-468, 514, 520.

FIG. 6(A-C) shows development of RBD-specific Nbs for potent SARS-COV-2 neutralization. FIG. 6A shows detection of strong and specific serologic activities after immunization of SARS-CoV-2 RBD. FIG. 6B shows neutralization potency of the immunized camelid's serum against pseudotyped SARS-CoV-2-Luciferase. FIG. 6C shows neutralization potency of Nbs against pseudotyped SARS-CoV-2-Luciferase.

FIG. 7(A-C) shows identification of a large repertoire of high-affinity Nbs by proteomics. FIG. 7A shows the schematic of high-affinity RBD-Nb identification by camelid immunization and quantitative Nb proteomics. Briefly, a camelid was immunized by the RBD. High-affinity, RBD-specific single-chain VHH antibodies were affinity isolated from the immunized serum, and analyzed by quantitative proteomics to identify the high-affinity, RBD-specific Nbs (see Methods). A VHH (Nb) cDNA library from the plasma B cells of the immunized camelid was created to facilitate proteomic analysis. FIG. 7B shows sequence logo and sequence logo of 120 high-affinity RBD Nbs. The amino acid occurrence at each position is shown. CDR: complementarity determining region. FR: framework. FIG. 7C shows the phylogenetic tree of the Nbs constructed by the maximum likelihood model.

FIG. 8 shows correlation analysis of 18 highly potent SARS-CoV-2 neutralizing Nbs. A plot showing a linear correlation of Nb neutralization IC50s between the pseudotyped virus neutralization assay and the SARS-CoV-2 PRNT.

FIG. 9(A-D) shows biophysical analysis of the outstanding neutralizing Nbs. FIG. 9(A-B) shows binding kinetics of Nbs 20 and 89 by surface plasmon resonance (SPR). FIG. 9C shows thermostability analysis of Nbs 20, 21, and 89. The values represent the average thermostability (Tm, ° C.) based on three replicates. The standard deviations (SD) of the measurements are 0.17, 0.93, and 0.8° C. for Nbs 20, 21, and 89. FIG. 9D shows stability analysis of Nb21 by SEC. Purified recombinant Nb21 was stored at room temperature for ˜6 weeks before subject to SEC analysis. The dominant peak represents Nb 21 monomer.

FIG. 10 shows SEC analysis of RBD-Nb complexes.

FIG. 11(A-I) shows SEC analysis of RBD-Nb complexes. The SEC profiles of RBD-Nb complexes showing 9 Nbs that have overlapping epitopes with Nb21. FIG. 11(A-H) shows the SEC profiles of RBD-Nb complexes showing five Nbs that have unique and non-overlapping epitopes with Nb21. FIG. 11I shows sequence alignment of the CDR3s of 18 highly potent neutralizing Nbs and CDR3 lengths comparing Nbs from epitope I and others.

FIG. 12(A-C) shows competitive ELISA analysis of hACE2 and Nbs for RBD binding and conversation analysis of RBD across different coronaviruses. FIG. 12A shows competitive ELISA of hACE2 and Nbs (20, 21, 93, and 95) for RBD binding. Y-axis: percentage of the normalized ACE2 signal. X-axis: Nb concentration (nM). FIG. 12B shows the surface display of different Nb neutralization epitopes on RBD in complex with hACE2 (cartoon model in blue).

FIG. 12C shows the conservation analysis of the spike protein RBD using the ConSurf web server, oriented as in FIG. 12B. The conservation is based on 150 sequences automatically extracted by the ConSurf server.

FIG. 13 shows structural comparisons of Nb20 with published RBD Nb structures. Overlays of Nb20 (purple ribbon) and three other RBD-Nbs (PDBs 6YZ5, 7C8V, and 7C8 W) in complex with RBD (yellow/grey ribbon).

FIG. 14(A-D) shows structural modeling of Nb 21-RBD interaction based on the Nb20-RBD crystal structure. In FIG. 14A, alignment of Nb21 with Nb20. The four residue differences between the two Nbs were shown. FIG. 14B shows zoom-in views showing the addition of new polar interaction between N52 (Nb21) and N450 (RBD). The model of Nb21 is superimposed based on the crystal structure of Nb20. FIG. 14C shows surface presentation of RBD. The hACE2 binding epitope is in steel blue and the Nb20 epitope is in medium purple. FIG. 14D shows structural alignment of Nb20-RBD complex with hACE2-RBD complex. The CDR1 and CDR3 residues (medium violet pink and goldenrod in spheres, respectively) of Nb20 overlap with the hACE2 binding site (steel blue) on RBD (grey ribbon).

FIG. 15(A-D) shows the biophysical properties of multivalent Nbs. FIG. 15A shows the expression levels of multivalent Nbs from E. coli whole cell lysates. FIG. 15B shows SDS-PAGE analysis of the purified multivalent Nbs. FIG. 15C shows thermostability analysis of ANTE-CoV2-Nab21T_{EK}, ANTE-CoV2-Nab20T_{EK}, ANTE-CoV2-Nab21T_{GS}, and ANTE-CoV2-Nab20T_{GS}. The values represent the average thermostability (Tm, ° C.) based on three replicates. The standard deviations of the measurements are 0.6, 0.27, 0.169, and 0.72° C., respectively. FIG. 15D shows high stability of the multivalent Nbs under the pseudovirus neutralization condition. Different Nb constructs were incubated under the pseudovirus neutralization assay condition without the virus for 72 hours. An anti-His6 mouse monoclonal antibody (Genscript) was used to detect the Nb constructs (His6 tag at the C terminus) by western blot.

FIG. 16(A-G) shows stability test of the multivalent Nbs. FIG. 16A shows SARS-CoV-2 PRNT of the homo-trimeric forms of Nbs 20 and 21 with an EK linker. The average neutralization percentage and the standard deviation of each data point were shown (n=2). FIG. 16(B-C) shows the SEC analysis of ANTE-CoV2-Nab20T_{GS} and ANTE-CoV2-Nab21TEK before and after lyophilization or aerosolization. FIG. 16(D-E) shows pseudotyped SARS-CoV-2 neutralization assay using ANTE-CoV2-Nab20T_{GS} and ANTE-CoV2-Nab21T_{EK} before and after lyophilization or aerosolization (n=2). FIG. 16F shows a summary table of the neutralization potency measurements of the homo-trimeric Nbs. FIG. 16G shows a portable mesh nebulizer (producing ≤5 μm aerosol particles) used in the study.

FIG. 17 shows the neutralization epitopes and virus mutations mapped on the RBD crystal structure. The dashed line (upper panel) indicates epitope V that partially overlaps with epitopes III and IV. The mutations (lower panel) are colored in gradient blue (0-100 mutation count from the GISAID), where darker blue indicates more frequent mutations.

FIG. 18(A-D) shows composite 2Fc-Fo electron density maps of the representative areas of RBD-Nb20 complex contoured at 1.0σ. FIG. 18A shows map of the whole complex shown as purple mesh. FIG. 18B shows map of the three CDRs of Nb20 as purple mesh. FIG. 18C shows map of the extended external loop region of RBD shown as purple mesh. FIG. 18D shows map of residues involved in the interactions between RBD and Nb20 shown as red mesh. RBD is colored in gray and Nb20 is colored in blue.

FIG. 19(A-C) shows correlation analysis of 18 highly potent SARS-CoV-2 neutralizing Nbs. FIG. 19A depicts a plot showing a linear correlation of Nb neutralization potency (IC50s) between two different SARS-COV-2 viral assays (pseudotype virus vs. authentic virus). FIG. 19B shows a heat map showing the correlation between SHM and Nb pseaudovirus neutralization potency, Pearson r=−0.408. FIG. 19C shows a heatmap showing the correlation between ELISA affinity and Nb pseudovirus neutralization potency, Pearson r=−0.639.

FIG. 20(A-B) shows structural modeling of Nb 21 based on the Nb 20-RBD crystal structure. FIG. 20A shows a structural model of Nb 21 in complex with RbD. FIG. 20B shows zoom-in views showing the addition of new polar interaction between N52 (Nb21) and N450 (RBD). The model of Nb21 is superimposed based on the crystal structure of Nb20.

FIG. 21(A-B) shows structural comparisons of Nb 20 with published RBD Nb structures. FIG. 21A shows an zoom-in view showing the cacodylate ion that is embedded in the interaction of Nb20-RBD. FIG. 21B shows structural overlays of Nb 20 and other RBD-Nbs.

FIG. 22 depicts an example of a computing system that executes methods and procedures described in certain embodiments of the present disclosure.

FIG. 23(A-D) shows identification and characterization of psNbs. (FIG. 23A) Phylogenetic tree of 19 RBDs from all 4 clades of sarbeoviruses, constructed by the maximum likelihood. (FIG. 23B) The neutralization of polyclonal VHHs from two immunization bleeds against pseudovirus of SARS-CoV-2, its variants and SARS-CoV. Their ELISA IC₅₀s against 4 RBD clades were also shown. (FIG. 23C) Schematics for proteomic identification of psNbs from immunized sera. (FIG. 23D) A map summarizing RBD binding and neutralization for 100 high-affinity psNbs. Nbs are represented by dots of various sizes and colors. Two Nbs are connected if their CDR sequence identity is >70%. Their neutralization potencies against pseudotyped SARS-CoV-2 (D614G) are represented by the size of dots. Breadth of sarbecovirus RBD binding is indicated by the filled gradient color (Table 2) and the SEC epitope groups are colored on the outer circle (FIG. 31). The highest Nb concentration used for binding and neutralization was 8 μM and 2.5 μM, respectively.

FIG. 24(A-G) shows binding and neutralization of 17 psNbs and development of a bispecific and inhalable psNb (PiN-118). (FIG. 24A) Heatmap summary of psNbs for binding to different RBDs by ELISA. White with cross mark: no binding. Silver: binding signal was detected at 8 μM yet the IC₅₀ cannot be determined. Gradient blue to gray: IC₅₀ between 0.8 nM to 8 μM. (FIG. 24B) The neutralization potency of psNbs against pseudotyped SARS-CoV-2, its variants and SARS-CoV. N/A: no neutralization detected at the highest Nb concentration (2.5 μM). (FIG. 24C) The neutralization potency against SARS-CoV-2 Munich strain and the Delta VOC by the PRNT assay. (FIG. 24D) Binding kinetic measurements of 5p-118 for RBD_{SARS-CoV-2} and RBD_{SARS-CoV} by surface plasmon resonance (SPR). (FIG. 24E) SPR measurements of 5p-132 for RBD_{SARS-CoV-2} and RBD_{BM-4831-CoV}. (FIG. 24F) SEC analysis of 5p-118 before and after aerosolization with soft mist inhaler. (FIG. 24G) Neutralization potency of a bispecific psNb construct (PiN-118) by the PRNT assay.

FIG. 25(A-C) shows a summary of broadly neutralizing RBD epitopes and spike conformations. (FIG. 25A) Clustering analysis of psNbs epitopes. RBS residues are in yellow. RBD glycosylation sites (N331 and 343) are denoted. SarbeCoVirus sequence conservation is shown in green gradient. The number of psNb: RBD interaction atoms per epitope residue is shown in blue gradient. (FIG. 25B) Structural representations of five classes of psNbs in complex with RBD and the corresponding epitopes. Light green: class I (5p-182), gold: class II (5p-118), pink: class III (5p-93), light blue: IV (Nb113) and purple: V (5p-64). VOC residues are in red. RBD glycosylation (N343) is in cornflower blue. (FIG. 25C) Structural diversity of psNbs in complex with the SARS-CoV-2 pre-fusion spike glycoprotein (by cryoEM).

FIG. 26(A-G) shows the structural diversity, convergence and evolution of class II psNbs. (FIG. 26A) Phylogenetic analysis of psNbs from SEC group B by the maximum likelihood. Structurally determined psNbs are in red. Scale of the evolutionary distance: 0-0.35. (FIG. 26B) Superposition of class II psNbs reveals two subclasses II(A) and II(B). (FIG. 26C) The overlap between II(A) and II(B) epitopes. Thistle: shared epitope; blue: non-overlapping epitope of II(A); salmon: non-overlapping epitope of II(B); light yellow: ACE2 with modeled glycans (N90 and N322). (FIG. 26D) Structural overview of class II psNbs in complex with RBD and epitopes. Light blue: Nb95 and Nb105; cornflower blue: 5p-60; navajo white: Nb117; dark salmon: 5p-38; gold: 5p-35; brown: 5p-118. FIG. 26(E-G) Comparison of CDR bindings between three pairs of related psNbs. FIG. 26E) Nb117 and 5p-38, FIG. 26F) 5p-35 and 5p-118, FIG. 26G) 5p-60 and Nb105.

FIG. 27(A-I) shows the structural insights of four classes of psNbs. (FIG. 27A) Superposition of 5p-182: RBD with ACE2 showing steric effects. (FIG. 27B) Comparison of buried epitope surface area between 5p-182 and Nb21. (FIG. 27C) Heatpmap showing interactions between RBS Nbs and mutations from VOCs (Alpha, Beta, Gamma, Delta and Omicron). (FIG. 27D) Surface representation showing all CDRs form extensive interaction with RBD. Purple: CDR1; light yellow: CDR2; Salmon: CDR3; Gray: FR2. (FIG. 27E) Structure superimposition of Class III Nbs (5p-93 and Nb17). (FIG. 27F) Surface representation showing epitopes of Nb17 and 5p-93. Conserved residues with RBD sequence identity >0.85 are highlighted. (FIG. 27G) Structures showing CDR3 of class IV psNbs inserting into the cavity on RBD and different utilities of CDRs. Shared conserved epitopes of class IV psNbs were labeled on the RBD using surface representation. (FIG. 27H) Structure showing CDR3 of Class V 5p-64 binding to RBD. (FIG. 27I) Superposition of 5p-64: RBD on all-RBD-up Spike showing the clash between 5p-64 and spike.

FIG. 28(A-H) shows the mechanisms of broad neutralization. (FIG. 28A-28B) The correlation between relative cross reactive binding and epitope conservation for FIG. 28A) all Nbs and FIG. 28B) non-RBS Nbs. (FIG. 28C) Surface representation showing geometry features of Nb:RBD interface of 5 classes of Nbs: flat epitope for class II, III and V epitope; convex epitope for class I and IV epitope. (FIG. 28D) Comparison of average root-mean-square-fluctuation (RMSF) between psNb and non psNb. (FIG. 28E) Comparison of viral fitness of epitope between psNb and non psNb. The viral fitness is obtained from evaluating the mutational effects on expression level. More negative values correspond to higher loss of viral fitness. (FIG. 28F) Comparison of utilities of framework residues for binding to RBD between psNbs and non-psNbs. (FIG. 28G) Mechanisms of viral neutralization. Class I Nbs directly clash with ACE2; class II Nbs clash with N322 glycan on ACE2; class III, IV and V do not compete with ACE2 binding. (FIG. 28H) Correlation between neutralization potency and the distance between Nb epitope and receptor binding sites. The distance is calculated based on centroids Nb epitope and ACE2 epitope.

FIG. 29(A-B) shows an analysis of the total VHHs isolated from serum after RBD immunization. (FIG. 29A) ELISA binding of total VHHs from the initial and booster bleeds against four representative RBDs. (FIG. 29B) Pseudovirus neutralization assay of total VHHs of the initial and booster bleeds against SARS-CoV-2, SARS-CoV-2 variants and SARS-CoV.

FIG. 30(A-B) shows an analysis of experimentally verified psNbs. (FIG. 30A) Phylogenetic analysis of the psNb CDR sequences and their physicochemical properties including isoelectric point (pI), hydropathy. (FIG. 30B) The individual psNb isolated from either 1st and 2nd bleed was synthesized and evaluated for RBD_{SARS-CoV-2} and RBD_{SARS-CoV} binding by ELISA. The percentage was calculated as the number of psNbs in a certain affinity range divided by the total number of the psNbs.

FIG. 31(A-C) shows size exclusion chromatography (SEC) and surface plasmon resonance analysis of representative psNbs for RBD binding. (FIG. 31A) SEC profiles of the reconstituted nanobodies of non-overlapping epitopes in complex with RBD_{SARS-CoV-2}. Left panel shows the structures that help illustrate competitive SEC experiments. (FIG. 31B) Competitive SEC of psNbs. Group A psNbs compete with Nb21. Group B psNbs compete with Nb105. Group C psNbs compete with Nb36. Group D psNbs do not compete with any of these three Nbs. Group E psNbs dissociate from RBD. (FIG. 31C) A representative SEC unclassified psNb (5P-156) was covalently coated to the Fc2 of a CM5 sensorchip. Fc1 of the sensorchip was non-coated as control. 1 μM deglycosylated RBD_{SARS-CoV-2} in the HBS-EP buffer was injected to the surface at 20 μl/min for 3 mins with dissociation for 3 mins, followed by a sequential injection of 1 μM glycosylated RBD_{SARS-CoV-2}. The responses were recorded as the difference between the signals from Fc2 and Fc1.

FIG. 32 shows ELISA of 16 psNbs against the RBDs from the WT SARS-CoV-2, the concerning variants and 18 different sarbeoviruses that span all four clades.

FIG. 33 shows the neutralization assays of 17 psNbs against the pseudotyped SARS-CoV-2 (Wuhan-Hu-1, D614G), the VOCs and SARS-CoV.

FIG. 34 shows a PRNT assay of 17 presentative psNbs against a clinical isolate of SARS-CoV-2 (Munich strain) and the Delta variant.

FIG. 35(A-F) shows biophysical characterizations of highly broad psNbs (5P-118 and 5P-132). (FIG. 35(A-B)) SPR kinetic measurement of 5p-118 against RBD_{RmYN02} and RBD_{BM-4831}. (FIG. 35(C-D)) SPR kinetic measurement of 5p-132 against RBD_{SARS-CoV} and RBD_{BM-4831} (FIG. 35E) SEC analysis of 5p-132 pre and post aerosolization. (FIG. 35F) Pseudovirus neutralization of 5p-118 and 5p-132 pre and post aerosolization.

FIG. 36 shows structural analysis of Nb binding to VOC mutations.

FIG. 37(A-F) shows Cryo-EM Structure determination workflow illustrated by 5p-60. (FIG. 37A) Representative micrograph of spike with 5p-60. (FIG. 37B) Representative 2D class averages of spike with 5p-60. (FIG. 37C) Gold-standard Fourier shell correlation (FSC) curves calculated from two independently refined half-maps with different masks for spike with 5p-60. (FIG. 37D) Local resolution map shown at two density thresholds (left: 0.5 and right: 0.1). (FIG. 37E) Masking of the RBD:psNb density for focused classification. (FIG. 37F) Results of focused classification. Classes with strong RBD:psNb densities (blue rectangle boxes) are selected for further local refinements.

FIG. 38(A-E) shows interactions of epitope II psNbs in complex with the spike or RBD. (FIG. 38A) Interactions between RBD and Nb117. (FIG. 38B) Interactions between RBD and 5p-38. (FIG. 38C) Interactions between RBD and 5p-35. (FIG. 38D) Interactions between RBD and 5p-118. (FIG. 38E) Interactions between RBD and 5p-60.

FIG. 39 shows structural comparison of Nb113 and an IgG antibody (S2H97).

FIG. 40 shows hACE2 competition assay with the SARS-CoV-2 spike by ELISA.

FIG. 41(A-B) shows RBD epitope prediction by ScanNet. (FIG. 41A) The RBD surface is colored by epitope propensity (from blue=0.0 to red=0.35 and higher) using ChimeraX. (FIG. 41B) RBD epitope propensity profile.

FIG. 42 shows scatter plots of antibody hit rates against conservation scores for surface residues for 11 antigens. A negative trend is found for most antigens. Notable outliers include the envelope protein of HIV and the genome polyprotein of Hepatitis C; this can be due to (i) over-representation of broadly neutralizing antibodies in the PDB and/or (ii) extensive glycosylation.

FIG. 43 shows scatter plots of ScanNet epitope propensity scores against conservation scores of surface residues for 11 antigens. A negative trend is observed between the conservation score and the ScanNet epitope propensity score which was calculated from antigen structure only. This suggests that the association between conservation and antibody hit rate arises solely from structural properties.

FIG. 44(A-C) shows comparison of overall structures of 5p-118 and Nb117 bound to SARS-CoV-2 RBD. Structures are shown as ribbon representation. Glycans in SARS-CoV-2 RBD and ACE2 are shown in sticks and labeled with their corresponding asparagine(N) position number. CC12.1 was used to complex with 5p-118 and SARS-CoV-2 to obtain diffraction-quality crystals for structure determination. 5p-118 is shown in dark red, Nb117 in beige, RBD in light gray, ACE2 in light yellow, and CC12.1 in white. Structures of 5p-118, Nb117, and ACE2 bound to SARS-CoV-2 are superimposed for comparison in c.

FIG. 45(A-C). Representative cryo-EM densities of psNbs in complex with the spike or RBD. (FIG. 45A) High-resolution cryo-EM maps allow the modeling of residue 621 to 640 for the spike protein, which are absent in the starting structure (PDB: 7CAK). The map was contoured at level 0.15. (FIG. 45B) High-resolution cryoEM maps enable the delineation of complex glycan composition at Asn1074 with a Fucose (FUC) attached to the first N-acetylglucosamine (NAG). The map was contoured at level 0.15. (FIG. 45C) High-resolution cryoEM maps facilitated modeling of the RBD (blue): 5p-60 (yellow) interface. The map was contoured at level 0.64.

FIG. 46(A-D). Cryo-EM Structure determination of RBD with 5p-182, 5p-93, 5p-64 and a class II psNb (1-25) and focused refinement. (FIG. 46A) Representative micrograph of RBD with four psNbs. (FIG. 46B) Representative 2D class averages of RBD and psNbs complex. (FIG. 46C) Gold-standard Fourier shell correlation (FSC) curves calculated from two independently refined half-maps with different masks for RBD and psNbs complex. (FIG. 46D) Local resolution map contoured at level 0.36.

FIG. 47(A-B). SEC analysis of 5p-182 (2-67) with RBD and benchmark Nbs, and quantitative MS evaluation of Nbs 20 and 21. (FIG. 47A) Competitive SEC analysis of 5p-182 (2-67) with Nb21 and other high-affinity benchmark Nbs for RBD binding. (FIG. 47B) MS quantification of a CDR3 tryptic peptide specific to Nbs 20 and 21. The Y axis represents the total MS1 ion abundance (relative abundance) of the peptide. Signals below 104-5 are generally considered chemical backgrounds. The sequence in FIG. 47B is SEQ ID NO: 2130 (YCAARDIETAEY).

FIG. 48(A-F). Interactions of class I, III, IV and V psNbs in complex with SARS-CoV-2 spike or RBD. (FIG. 48A) Interactions between RBD and 5p-182. (FIG. 48B) Interactions between RBD and 5p-93. (FIG. 48C) Interactions between RBD and Nb113. (FIG. 48D) Interactions between RBD and 4P-56. (FIG. 48E) Interactions between RBD and 5p-179. (FIG. 48F) Interactions between RBD and 5p-64.

FIG. 49. Comparison of the utility of framework residues of psNbs and non-psNbs for binding to RBD.

### DETAILED DESCRIPTION

Provided herein are coronavirus (e.g., a sarbecovirus and/or SARS-CoV-2) neutralizing nanobodies and uses thereof for preventing or treating infection of one or more coronaviruses. These nanobodies were elucidated through an application of our integrative proteomic platform for in-depth discovery, classification, and high-throughput structural characterization of antigen-engaged Nb repertoires. A collection of diverse, soluble, stable, and high-affinity camelid Nbs that target the SARS-CoV-2 spike protein receptor-binding domain (RBD) were developed and characterized. The majority of the high-affinity RBD Nbs efficiently neutralize SARS-CoV-2, and in some embodiments, efficiently neutralize another coronavirus. A subset has exceptional neutralization potency, comparable with, or better than the most potent human NAbs.

### Terminology

As used in the specification and claims, the singular form “a,” “an,” and “the” include plural references unless the context clearly dictates otherwise. For example, the term “a cell” includes a plurality of cells, including mixtures thereof.

The term “about” as used herein when referring to a measurable value such as an amount, a percentage, and the like, is meant to encompass variations of ±20%, ±10%, ±5%, or ±1% from the measurable value.

“Administration” to a subject or “administering” includes any route of introducing or delivering to a subject an agent. Administration can be carried out by any suitable route, including oral, intravenous, intraperitoneal, intranasal, inhalation and the like. Administration includes self-administration and the administration by another.

The terms “antibody” and “antibodies” are used herein in a broad sense and include polyclonal antibodies, monoclonal antibodies, and bi-specific antibodies. In addition to intact immunoglobulin molecules, also included in the term “antibodies” are fragments or polymers of those immunoglobulin molecules, and human or humanized versions of immunoglobulin molecules or fragments thereof. Antibodies are usually heterotetrameric glycoproteins of about 150,000 daltons, composed of two identical light (L) chains and two identical heavy (H) chains. Each heavy chain has at one end a variable domain (V_{H}) followed by a number of constant domains. Each light chain has a variable domain at one end (V_{L}) and a constant domain at its other end.

The antibodies can be tested for their desired activity using the in vitro assays described herein, or by analogous methods, after which, their in vivo therapeutic and/or prophylactic activities are tested according to known clinical testing methods. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse. The heavy chain constant domains that correspond to the different classes of immunoglobulins are called alpha, delta, epsilon, gamma, and mu, respectively.

The terms “antigenic determinant” and “epitope” may also be used interchangeably herein, referring to the location on the antigen or target recognized by the antigen-binding molecule (such as the nanobodies of the invention). Epitopes can be formed both from contiguous amino acids (a “linear epitope”) or noncontiguous amino acids juxtaposed by tertiary folding of a protein. The latter epitope, one created by at least some noncontiguous amino acids, is described herein as a “conformational epitope.” An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed (1996).

The terms “antigen binding site”, “binding site” and “binding domain” refer to the specific elements, parts or amino acid residues of a polypeptide, such as a nanobody, that bind the antigenic determinant or epitope.

The terms “CDR” and “complementarity determining region” are used interchangeably and refer to a part of the variable chain of an antibody that participates in binding to an antigen. Accordingly, a CDR is a part of, or is, an “antigen binding site.” Generally, native antibodies comprise six CDRs; three in the heavy chain (heavy chain complementarity determining region (CDRH)1, CDRH2, and CDRH3), and three in the heavy chain (light chain complementarity determining region (CDRL)1, CDRL2, and CDRL3). In some embodiments, the nanobody comprises three CDR (CDR1, CDR2, and CDR3) that collectively form an antigen binding site.

The term “hypervariable region” or “HVR”, as used herein, refers to each of the regions of an antibody variable domain which are hypervariable in sequence and/or form structurally defined loops (“hypervariable loops”). Generally, native four-chain antibodies comprise six HVRs; three in the VH (H1, H2, H3), and three in the VL (L1, L2, L3). HVRs generally comprise amino acid residues from the hypervariable loops and/or from the complementarity determining regions (CDRs), the latter being of highest sequence variability and/or involved in antigen recognition. CDRs generally comprise the amino acid residues that form the hypervariable loops. Hypervariable regions (HVRs) are also referred to as “complementarity determining regions” (CDRs), and these terms are used herein interchangeably in reference to portions of the variable region that form the antigen-binding regions. The amino acid sequence boundaries of a CDR can be determined by one of skill in the art using any of a number of known numbering schemes, including those described by Kabat et al., supra (“Kabat” numbering scheme): Al-Lazikani et al., 1997. J. Mol. Biol., 273:927-948 (“Chothia” numbering scheme); MacCallum et al., 1996, J. Mol. Biol, 262:732-745 (“Contact” numbering scheme); Lefranc et al., Dev. Comp. Immunol., 2003, 27:55-77 (“IMGT” numbering scheme); and Honegge and Pluckthun, J. Mol. Biol., 2001, 309:657-70 (“AHo” numbering scheme); each of which is incorporated by reference in its entirety.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed.

“Composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, a bacterium, a vector, polynucleotide, cells, salts, esters, amides, proagents, active metabolites, isomers, fragments, analogs, and the like. When the terms “composition” is used, then, or when a particular composition is specifically identified, it is to be understood that the term includes the composition per se as well as pharmaceutically acceptable, pharmacologically active vector, polynucleotide, salts, esters, amides, proagents, conjugates, active metabolites, isomers, fragments, analogs, etc. In some aspects, the composition disclosed herein comprises the nanobody disclosed herein.

“Effective amount” encompasses, without limitation, an amount that can ameliorate, reverse, mitigate, prevent, or diagnose a symptom or sign of a medical condition or disorder (e.g., an infection). Unless dictated otherwise, explicitly or by context, an “effective amount” is not limited to a minimal amount sufficient to ameliorate a condition. The severity of a disease or disorder, as well as the ability of a treatment to prevent, treat, or mitigate, the disease or disorder can be measured, without implying any limitation, by a biomarker or by a clinical parameter. In some embodiments, the term “effective amount of a recombinant nanobody” refers to an amount of a recombinant nanobody sufficient to prevent, treat, or mitigate an infection.

The “fragments” or “functional fragments,” whether attached to other sequences or not, can include insertions, deletions, substitutions, or other selected modifications of particular regions or specific amino acids residues, provided the activity of the fragment is not significantly altered or impaired compared to the nonmodified peptide or protein. These modifications can provide for some additional property, such as to remove or add amino acids capable of disulfide bonding, to increase its bio-longevity, to alter its secretory characteristics, etc. In any case, the functional fragment must possess a bioactive property, such as binding to a coronavirus and/or ameliorating coronavirus infection.

As used herein, a “functional selection step” is a method by which nanobodies are divided into different fractions or groups based upon a functional characteristic. In some embodiments, the functional characteristic is nanobody or CD3 region antigen affinity. In other embodiments, the functional characteristic is nanobody thermostability. In other embodiments, the functional characteristic is nanobody intracellular penetration. Accordingly, the present invention includes a method of identifying a group of complementarity determining region (CDR)3 region nanobody amino acid sequences (CDR3 sequences) wherein a reduced number of the CDR3 sequences are false positives as compared to a control, the method comprising: obtaining a blood sample from a camelid immunized with the antigen; using the blood sample to obtain a nanobody cDNA library; identifying the sequence of each cDNA in the library; isolating nanobodies from the same or a second blood sample from the camelid immunized with the antigen; performing a functional selection step; digesting the nanobodies with trypsin or chymotrypsin to create a group of digestion products; performing a mass spectrometry analysis of the digestion products to obtain mass spectrometry data; selecting sequences identified in step c. that correlate with the mass spectrometry data; identifying sequences of CDR3 regions in the sequences from step g.; and excluding from the CDR3 region sequences from step h. those sequences having less than a calculated fragmentation coverage percentage; wherein the non-excluded sequences comprise a group having the reduced number of false positive CDR3 sequences. It should be understood that the method steps following the functional selection step can be performed separately on each different fraction or group created by the functional selection.

The “half-life” of an amino acid sequence, compound or polypeptide of the invention can generally be defined as the time taken for the serum concentration of the amino acid sequence, compound or polypeptide to be reduced by 50%, in vivo, for example due to degradation of the sequence or compound and/or clearance or sequestration of the sequence or compound by natural mechanisms. The in vivo half-life of a nanobody, amino acid sequence, compound or polypeptide of the invention can be determined in any manner known, such as by pharmacokinetic analysis. these, for example, Kenneth, A et al., Chemical Stability of Pharmaceuticals: A Handbook for Pharmacists; Peters et al., Pharmacokinete analysis: A Practical Approach (1996); “Pharmacokinetics”, M Gibaldi & D Perron, published by Marcel Dekker, 2nd Rev. edition (1982).

The term “identity” or “homology” shall be construed to mean the percentage of nucleotide bases or amino acid residues in the candidate sequence that are identical with the bases or residues of a corresponding sequence to which it is compared, after aligning the sequences and introducing gaps, if necessary to achieve the maximum percent identity for the entire sequence, and not considering any conservative substitutions as part of the sequence identity. A polynucleotide or polynucleotide region (or a polypeptide or polypeptide region) that has a certain percentage (for example, 80%, 85%, 90%, or 95%) of “sequence identity” to another sequence means that, when aligned, that percentage of bases (or amino acids) are the same in comparing the two sequences. In some embodiments, the identity or homology is determined over the entirety of the compared sequences, or in other words, the full length of the sequences are compared. This alignment and the percent homology or sequence identity can be determined using software programs known in the art. Such alignment can be provided using, for instance, the method of Needleman et al. (1970) J. Mol. Biol. 48: 443-453, implemented conveniently by computer programs such as the Align program (DNAstar, Inc.).

As used herein, the terms “nanobody”, “VHH”, “VHH antibody fragment” and “single domain antibody” are used indifferently and designate a variable domain of a single heavy chain of an antibody of the type found in Camelidae, which are without any light chains, such as those derived from Camelids as described in PCT Publication No. WO1994004678, which is incorporated by reference in its entirety.

As used herein, “operatively linked” refers to the arrangement of polypeptide segments within a single polypeptide chain, where the individual polypeptide segments can be, without limitation, a protein, fragments thereof, linking peptides, and/or signal peptides. The term operatively linked can refer to direct fusion of different individual polypeptides within the single polypeptides or fragments thereof where there are no intervening amino acids between the different segments as well as when the individual polypeptides are connected to one another via a “linker” that comprises one or more intervening amino acids. In some embodiments, the linker is between about 10 and about 40 amino acids. In some embodiments, the linker is between about 15 and about 35 amino acids. In some embodiments, the linker is about 25 amino acids. In some embodiments, the linker is about 31 amino acids. In some embodiments, the linker comprises SEQ ID NO:2115 (EGKSSGSGSESKSTGGGGSEGKSSGSGSESKST).

The term “neutralize” refers to a nanbody's ability to reduce infectivity of SARS CoV-2 or another coronavirus. It should be understood that “neutralizing” does not require a 100% neutralization and only requires a partial neutralization. In some embodiments, an about 30%, about 40%, about 50%, about 60%, about 70%, about 80%, or about 90% neutralization is obtained. In some embodiments, infectivity is reduced about 100%, about 90%, about 80%, about 70% or about 60%. “Infectivity” refers to the ability of a virus to bind to and enter a cell. As an example, a nanobody that reduces infectivity by a virus by 100% reduces the virus' entry into a cell by 100% as compared to a control. Accordingly, included herein are embodiments where the nanobody reduces infectivity of SARS CoV-2 or a coronavirus by about 100%, about 99%, about 98%, about 97%, about 96%, about 95%, about 94%, about 93%, about 92%, about 91%, about 90%, about 85%, about 80%, about 75%, about 70%, about 65%, about 60%, about 55%, or about 50% as compared to a control. In some embodiments, the concentration of nanobody required to achieve neutralization of SARS CoV-2 or a coronavirus by about 50% (e.g., an IC50) is less than 1 ng/1 ml.

The term “nucleic acid” as used herein means a polymer composed of nucleotides, e.g., deoxyribonucleotides (DNA) or ribonucleotides (RNA). The terms “ribonucleic acid” and “RNA” as used herein mean a polymer composed of ribonucleotides. The terms “deoxyribonucleic acid” and “DNA” as used herein mean a polymer composed of deoxyribonucleotides.

The terms “pharmaceutically effective amount”, “therapeutically effective amount” or “therapeutically effective dose” refer to the amount of a compound such as a SARS-CoV-2 or coronavirus neutralizing nanobody that will elicit the biological or medical response of a tissue, system, animal, or human that is being sought by the researcher, veterinarian, medical doctor or other clinician. In some embodiments, a desired response is a clinical improvement of, or reduction of an undesired symptom associated with, a SARS-CoV-2 or coronavirus infection. In some embodiments, a desired response is a prevention of a SARS-CoV-2 or coronavirus infection. In some instances, a desired biological or medical response is achieved following administration of multiple dosages of the composition to the subject over a period of days, weeks, or years. The terms “pharmaceutically effective amount”, “therapeutically effective amount” or “therapeutically effective dose” include that amount of a compound such as a SARS-CoV-2 or coronavirus neutralizing nanobody that, when administered, is sufficient to prevent development of, or alleviate to some extent, one or more of the symptoms of the condition or disorder being treated. The therapeutically effective amount will vary depending on the compound such as a selective bacterial β-glucuronidase inhibitor, the disorder or conditions and its severity, the route of administration, time of administration, rate of excretion, drug combination, judgment of the treating physician, dosage form, and the age, weight, general health, sex and/or diet of the subject to be treated. In the context of the present method, a pharmaceutically or therapeutically effective amount or dose of a SARS-CoV-2 or coronavirus neutralizing nanobody includes an amount that is sufficient to reduce one or more of shortness of breath, pneumonia, cough, fatigue, muscle or body aches, headache, loss of taste or smell, sore throat, nausea, vomiting, diarrhea, persistent pain or pressure in the chest, trouble breathing, and death caused by a SARS-CoV-2 or coronavirus infection.

The terms “polynucleotide” and “oligonucleotide” are used interchangeably, and refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: a gene or gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. The term also refers to both double- and single-stranded molecules. Unless otherwise specified or required, any embodiment of this invention that is a polynucleotide encompasses both the double-stranded form and each of two complementary single-stranded forms known or predicted to make up the double-stranded form.

The term “polypeptide” is used in its broadest sense to refer to a compound of two or more subunit amino acids, amino acid analogs, or peptidomimetics. The subunits may be linked by peptide bonds. In another embodiment, the subunit may be linked by other bonds, e.g., ester, ether, etc. As used herein the term “amino acid” refers to either natural and/or unnatural or synthetic amino acids, including glycine and both the D or L optical isomers, and amino acid analogs and peptidomimetics. A peptide of three or more amino acids is commonly called an oligopeptide if the peptide chain is short. If the peptide chain is long, the peptide is commonly called a polypeptide or a protein.

“Recombinant” used in reference to a polypeptide refers herein to a combination of two or more polypeptides, which combination is not naturally occurring.

The term “required fragmentation coverage percentage” refers to a percentage obtained using the following formula:
f(x,Enzyme) is the function to calculate fragmentation coverage (%) of peptides digested by Enzyme
x is the length of CDR3 that the peptide mapped

$$\begin{matrix}f ( x , chymotrypsin ) = 0.0023 {x}^{2} - 0.0497 x + 0.7723 , x [ 5 , 30 ] \\ f ( x , t rypsin ) = 0.00006 {x}^{2} - 0.00444 x + 0.9194 , x [ 5 , 30 ]\end{matrix}$$

The terms “specific binding,” “specifically binds,” “selective binding,” and “selectively binds” mean that a nanobody exhibits appreciable affinity for a particular antigen or epitope and, generally, does not exhibit significant cross-reactivity with other non-spike protein receptor binding domain antigens and epitopes. Appreciable binding affinity includes binding with an affinity of at least 10⁶ M⁻¹, specifically at least 10⁷ M⁻¹, more specifically at least 10⁸ M⁻¹, yet more specifically at least 10⁹ M⁻¹, or even yet more specifically at least 10¹⁰ M⁻¹. A binding affinity can also be indicated as a range of affinities, for example, 10⁶ M⁻¹ to 10¹⁰ M⁻¹, specifically 10⁷ M⁻¹ to 10¹⁰ M⁻¹, more specifically 10⁸ M⁻¹ to 10¹⁰ M⁻¹. A nanobody that “does not exhibit significant cross reactivity” is one that will not appreciably bind to an undesirable entity (e.g., an undesirable proteinaceous entity such as a non-spike protein receptor binding domain). A nanobody specific for a particular epitope will, for example, not significantly cross react with other epitopes on the same protein or peptide. Specific binding can be determined according to any art-recognized means for determining Such binding. In some embodiments, specific binding is determined according to Scatchard analysis and/or competitive binding assays.

The term “subject” is defined herein to include animals such as mammals, including, but not limited to, primates (e.g., humans), cows, sheep, goats, horses, dogs, cats, rabbits, rats, mice and the like. In some embodiments, the subject is a human.

The terms “treat,” “treating,” “treatment,” and grammatical variations thereof as used herein, include partially or completely delaying, alleviating, mitigating or reducing the intensity of one or more attendant symptoms of a SARS-CoV-2, sarbecovirus or coronavirus infection and/or alleviating, mitigating or impeding one or more causes of a SARS-CoV-2, sarbecovirus or coronavirus infection. In some instances, the terms “treat”, “treating”, “treatment” and grammatical variations thereof, refer to reducing detectable SARS-CoV-2, sarbecovirus or coronavirus in the subject. In some instances, the terms “treat”, “treating”, “treatment” and grammatical variations thereof, refer to achieving a negative test result for SARS-CoV-2, sarbecovirus or coronavirus in the subject. In some instances, the terms “treat”, “treating”, “treatment” and grammatical variations thereof, refer to reducing SARS-CoV-2, sarbecovirus or coronavirus viral load in the subject. In some instances, the terms “treat”, “treating”, “treatment” and grammatical variations thereof, refer to reducing one or more of shortness of breath, pneumonia, cough, fatigue, muscle or body aches, headache, loss of taste or smell, sore throat, nausea, vomiting, diarrhea, persistent pain or pressure in the chest, trouble breathing, and death caused by a SARS-CoV-2, sarbecovirus or coronavirus infection.

### Compositions and Methods

Provided herein is the development and characterization of a large collection of diverse, high-affinity camelid Nbs. In some examples, the Nbs disclosed herein can target the S1-receptor binding domain (RBD) of a coronavirus spike protein or a coronavirus spike protein. In particular, the coronavirus Nbs (e.g., SARS-CoV-2 Nbs) described herein are capable of neutralizing coronavirus (e.g., SARS-CoV-2) infectivity in a cell culture system. Therefore, the coronavirus Nbs (e.g., SARS-CoV-2 Nbs) of the present invention are useful for treating or preventing a coronavirus infection (e.g., SARS-CoV-2, sarbecovirus or coronavirus infection), and included herein are methods of treating a coronavirus infection (e.g., SARS-CoV-2, sarbecovirus or coronavirus infection) in a subject comprising administering to the subject a therapeutically effective amount of a neutralizing nanobody described herein. Also included herein are methods of preventing a coronavirus infection (e.g., SARS-CoV-2, sarbecovirus or coronavirus infection) in a subject comprising administering to the subject a therapeutically effective amount of a neutralizing nanobody described herein.

The term “sarbecovirus” refers to a subgenus of genus betacoronavirus, family Coronaviridae. In some embodiments, the sarbecovirus is SARS-CoV. In some embodiments, the sarbecovirus is SARS-CoV-2. As used herein, a “sarbecovirus neutralizing nanobody” is a nanobody that can neutralize more than one member of the sarbecovirus subgenus.

The present invention includes nanobodies that comprise one or more complementarity determining regions 3 (CDR3), wherein the CDR3 comprises an amino acid sequence selected from the group consisting of SEQ ID NO: 1 through SEQ ID NO: 1057. In some embodiments, the nanobodies disclosed herein are sarbecovirus, SARS-CoV, and/or SARS-CoV-2 neutralizing nanobodies. In some embodiments, the coronavirus nanobody comprises a sequence that has about 80%, about 85%, about 90%, about 95%, about 96%, about 97%, about 98%, or about 99% homology with an amino acid sequence selected from the group consisting of SEQ ID NO: 1 through SEQ ID NO: 1057. In some embodiments, the nanobody comprises a sequence that has about 80%, about 85%, about 90%, about 95%, about 96%, about 97%, about 98%, or about 99% homology with an amino acid sequence selected from the group consisting of SEQ ID NO: 1058 through SEQ ID NO: 2114 and SEQ ID NO: 2117 through SEQ ID NO: 2119. In some embodiments, the nanobody comprises the sequence selected from the group consisting of SEQ ID NO: 1058 through SEQ ID NO: 2114 and SEQ ID NO: 2117 through SEQ ID NO: 2119.

In some embodiments, the nanobody comprises a sequence that has about 80%, about 85%, about 90%, about 95%, about 96%, about 97%, about 98%, or about 99% homology with an amino acid sequence selected from the group consisting of SEQ ID NO: 1058-1072, 1074, 1075, 1077, 1078, 1079, 1080, 1082, 1083, 1084, 1085, 1086, 1087, 1089, 1090, 1091, 1092, 1093, 1096, 1097, 1098, 1099, 1100, 1103, 1105, 1106, 1111, 1118, 1122, 1127, 1128, 1129, 1130, 1132, 1133, 1134, 1138, 1146, 1147, 1148, 1149, 1150, 1153, 1154, 1155, 1156, 1157, 1161, 1185, 1191, 1193, 1194, 1196, 1197, 1198, 1200, 1203, 1204, 1205, 1208, 1209, 1211, 1220, 1221, 1222, 1229, 1234, 1237, 1240, 1241, 1249, 1260, 1261, 1262, 1271, 1274, 1276, 1278, 1286, 1287, 1288, 1291, 1294, 2117, 2118, and 2119.

In some embodiments, the nanobody comprises a multimer (e.g., homodimer or homotrimer) of CDR3s, wherein the CDR3 comprises an amino acid sequence that has about 80%, about 85%, about 90%, about 95%, about 96%, about 97%, about 98%, or about 99% homology with an amino acid sequence selected from the group consisting of SEQ ID NO: 1 through SEQ ID NO: 1057. In some embodiments, the nanobody comprises a multimer (e.g., homodimer or homotrimer) of CDR3s, wherein the CDR3 comprises an amino acid sequence that has about 80%, about 85%, about 90%, about 95%, about 96%, about 97%, about 98%, or about 99% homology with an amino acid sequence selected from the group consisting of SEQ ID NO:1 through SEQ ID NO: 1057.

In some embodiments, the nanobody comprises a multimer (e.g., homodimer or homotrimer) of CDR3s, wherein the CDR3 comprises the amino acid sequence set forth in SEQ ID NO: 1 through SEQ ID NO: 1057. In some embodiments, the nanobody is a homotrimer comprising three copies of a CDR3, wherein the CDR3 comprises the one amino acid sequence comprises a sequence selected from the group consisting of SEQ ID NO: 1 through SEQ ID NO: 1057. In some embodiments, the nanobody is a homodimer comprising two copies of one CDR3, wherein the CDR3 comprises the one amino acid sequence comprises a sequence selected from the group consisting of SEQ ID NO: 1 through SEQ ID NO: 1057.

In some embodiments, the nanobody comprises a multimer (e.g., homodimer or homotrimer) of the amino acid sequence set forth in SEQ ID NO: 1058 through SEQ ID NO: 2114 and SEQ ID NO: 2117 through SEQ ID NO: 2119. In some embodiments, the nanobody is a homotrimer comprising three copies of one amino acid sequence, wherein the one amino acid sequence comprises a sequence selected from the group consisting of SEQ ID NO: 1058 through SEQ ID NO: 2114 and SEQ ID NO: 2117 through SEQ ID NO: 2119. In some embodiments, the nanobody is a homodimer comprising two copies of one amino acid sequence, wherein the one amino acid sequence comprises a sequence selected from the group consisting of SEQ ID NO: 1058 through SEQ ID NO: 2114 and SEQ ID NO: 2117 through SEQ ID NO: 2119.

In some embodiments, the nanobody comprises a multimer (e.g., homodimer or homotrimer) of the amino acid sequence set forth in SEQ ID NO: 1058-1072, 1074, 1075, 1077, 1078, 1079, 1080, 1082, 1083, 1084, 1085, 1086, 1087, 1089, 1090, 1091, 1092, 1093, 1096, 1097, 1098, 1099, 1100, 1103, 1105, 1106, 1111, 1118, 1122, 1127, 1128, 1129, 1130, 1132, 1133, 1134, 1138, 1146, 1147, 1148, 1149, 1150, 1153, 1154, 1155, 1156, 1157, 1161, 1185, 1191, 1193, 1194, 1196, 1197, 1198, 1200, 1203, 1204, 1205, 1208, 1209, 1211, 1220, 1221, 1222, 1229, 1234, 1237, 1240, 1241, 1249, 1260, 1261, 1262, 1271, 1274, 1276, 1278, 1286, 1287, 1288, 1291, 1294, 2117, 2118, and 2119.

In some embodiments, the nanobody is a homotrimer comprising three copies of one amino acid sequence, wherein the one amino acid sequence comprises a sequence selected from the group consisting of SEQ ID NO: 1058-1072, 1074, 1075, 1077, 1078, 1079, 1080, 1082, 1083, 1084, 1085, 1086, 1087, 1089, 1090, 1091, 1092, 1093, 1096, 1097, 1098, 1099, 1100, 1103, 1105, 1106, 1111, 1118, 1122, 1127, 1128, 1129, 1130, 1132, 1133, 1134, 1138, 1146, 1147, 1148, 1149, 1150, 1153, 1154, 1155, 1156, 1157, 1161, 1185, 1191, 1193, 1194, 1196, 1197, 1198, 1200, 1203, 1204, 1205, 1208, 1209, 1211, 1220, 1221, 1222, 1229, 1234, 1237, 1240, 1241, 1249, 1260, 1261, 1262, 1271, 1274, 1276, 1278, 1286, 1287, 1288, 1291, 1294, 2117, 2118, and 2119. In some embodiments, the nanobody is a homotrimer comprising three copies of one amino acid sequence, wherein the one amino acid sequence comprises the sequence of SEQ ID NO: 1147 or 1161.

In some embodiments, the nanobody is a homodimer comprising two copies of one amino acid sequence, wherein the one amino acid sequence comprises a sequence selected from the group consisting of SEQ ID NO: 1058-1072, 1074, 1075, 1077, 1078, 1079, 1080, 1082, 1083, 1084, 1085, 1086, 1087, 1089, 1090, 1091, 1092, 1093, 1096, 1097, 1098, 1099, 1100, 1103, 1105, 1106, 1111, 1118, 1122, 1127, 1128, 1129, 1130, 1132, 1133, 1134, 1138, 1146, 1147, 1148, 1149, 1150, 1153, 1154, 1155, 1156, 1157, 1161, 1185, 1191, 1193, 1194, 1196, 1197, 1198, 1200, 1203, 1204, 1205, 1208, 1209, 1211, 1220, 1221, 1222, 1229, 1234, 1237, 1240, 1241, 1249, 1260, 1261, 1262, 1271, 1274, 1276, 1278, 1286, 1287, 1288, 1291, 1294, 2117, 2118, and 2119.

In some embodiments, the nanobody is a heterotrimer comprising three different amino acid sequences, wherein each different amino acid sequence comprises a sequence selected from the group consisting of SEQ ID NO: 1 through SEQ ID NO: 1057. In some embodiments, the nanobody is a heterotrimer comprising three different amino acid sequences, wherein each different amino acid sequence comprises a sequence selected from the group consisting of SEQ ID NO: 1058 through SEQ ID NO: 2114 and SEQ ID NO: 2117 through SEQ ID NO: 2119.

In some embodiments, the nanobody is a heterotrimer comprising three different amino acid sequences, wherein each different amino acid sequence comprises a sequence selected from the group consisting of SEQ ID NO: 1058-1072, 1074, 1075, 1077, 1078, 1079, 1080, 1082, 1083, 1084, 1085, 1086, 1087, 1089, 1090, 1091, 1092, 1093, 1096, 1097, 1098, 1099, 1100, 1103, 1105, 1106, 1111, 1118, 1122, 1127, 1128, 1129, 1130, 1132, 1133, 1134, 1138, 1146, 1147, 1148, 1149, 1150, 1153, 1154, 1155, 1156, 1157, 1161, 1185, 1191, 1193, 1194, 1196, 1197, 1198, 1200, 1203, 1204, 1205, 1208, 1209, 1211, 1220, 1221, 1222, 1229, 1234, 1237, 1240, 1241, 1249, 1260, 1261, 1262, 1271, 1274, 1276, 1278, 1286, 1287, 1288, 1291, 1294, 2117, 2118, and 2119.

In some embodiments, the nanobody is a heterodimer comprising two different amino acid sequences, wherein each different amino acid sequence comprises a sequence selected from the group consisting of SEQ ID NO: 1 through SEQ ID NO: 1057. In some embodiments, the nanobody is a heterodimer comprising two different amino acid sequences, wherein each different amino acid sequence comprises a sequence selected from the group consisting of SEQ ID NO: 1058 through SEQ ID NO: 2114 and SEQ ID NO: 2117 through SEQ ID NO: 2119. In some embodiments, the nanobody is a heterodimer comprising two different amino acid sequences, wherein each different amino acid sequence comprises a sequence selected from the group consisting of SEQ ID NO: 1058-1072, 1074, 1075, 1077, 1078, 1079, 1080, 1082, 1083, 1084, 1085, 1086, 1087, 1089, 1090, 1091, 1092, 1093, 1096, 1097, 1098, 1099, 1100, 1103, 1105, 1106, 1111, 1118, 1122, 1127, 1128, 1129, 1130, 1132, 1133, 1134, 1138, 1146, 1147, 1148, 1149, 1150, 1153, 1154, 1155, 1156, 1157, 1161, 1185, 1191, 1193, 1194, 1196, 1197, 1198, 1200, 1203, 1204, 1205, 1208, 1209, 1211, 1220, 1221, 1222, 1229, 1234, 1237, 1240, 1241, 1249, 1260, 1261, 1262, 1271, 1274, 1276, 1278, 1286, 1287, 1288, 1291, 1294, 2117, 2118, and 2119. In some embodiments, the heterodimer comprises SEQ ID NO: 1147 and SEQ ID NO: 1161.

In some embodiments, the nanobody disclosed herein comprises a sequence of SEQ ID NO: 1075, SEQ ID NO: 1078, SEQ ID NO: 1084, SEQ ID NO: 1086, SEQ ID NO: 1122, SEQ ID NO: 1147, SEQ ID NO: 1161, SEQ ID NO: 1208, SEQ ID NO: 1211, SEQ ID NO: 1249, SEQ ID NO: 1276, SEQ ID NO: 2117 or SEQ ID NO: 2118. In some embodiments, the linker is between about 10 and about 40 amino acids. In some embodiments, the linker is between about 15 and about 35 amino acids. In some embodiments, the linker is about 25 amino acids. In some embodiments, the linker is about 31 amino acids. In some embodiments, the linker comprises SEQ ID NO: 2115 (EGKSSGSGSESKSTGGGGSEGKSSGSGSESKST) or a fragment thereof.

Accordingly, included herein is a homotrimer nanobody comprising three copies of one amino acid sequence, wherein the one amino acid sequence comprises a sequence selected from the group consisting of SEQ ID NO: 1 through SEQ ID NO: 1057 or a fragment thereof, and wherein the three copies are separated by linker sequences. Also included is a heterotrimer nanobody comprising three different amino acid sequences, wherein each different amino acid sequence comprises a sequence selected from the group consisting of SEQ ID NO: 1 through SEQ ID NO: 1057 or a fragment thereof, and wherein the different amino acid sequences are separated by linker sequences. Also included is a homodimer nanobody comprising two copies of one amino acid sequence, wherein the one amino acid sequence comprises a sequence selected from the group consisting of SEQ ID NO: 1 through SEQ ID NO: 1057 or a fragment thereof, and wherein the two copies are separated by linker sequences. Further included is a heterodimer nanobody comprising two different amino acid sequences, wherein each different amino acid sequence comprises a sequence selected from the group consisting of SEQ ID NO: 1 through SEQ ID NO: 1057 or a fragment thereof, and wherein the different amino acid sequences are separated by linker sequences.

In some embodiments, the sarbecovirus, SARS-CoV, and/or SARS-CoV-2 neutralizing nanobody disclosed herein is conjugated or linked to a nanobody or a nanobody fragment, that specifically binds to human serum albumin for the purpose of increasing the half-life of the sarbecovirus, SARS-CoV, and/or SARS-CoV-2 neutralizing nanobody.

With regard to the human serum albumin portion of the nanobody, “serum albumin” is a type of globular protein in vertebrate blood. Serum albumin is produced by the liver. “Human serum albumin” or “HSA” refers herein to a polypeptide that synthesizes and hydrolyzes cyclic adenosine 5′-diphosphate-ribose, and is encoded by the ALB gene. In some embodiments, the HSA polypeptide is that identified in one or more publicly available databases as follows: HGNC: 399, Entrez Gene: 213, Ensembl: ENSG00000163631, OMIM: 103600, UniProtKB: P02768. In some embodiments, the HSA polypeptide comprises the sequence of SEQ ID NO: 2124, or a polypeptide sequence having at or greater than about 80%, about 85%, about 90%, about 95%, or about 98% homology with SEQ ID NO: 2124, or a polypeptide comprising a portion of SEQ ID NO: 2124. The HSA polypeptide of SEQ ID NO: 2124 may represent an immature or pre-processed form of mature HSA, and accordingly, included herein are mature or processed portions of the HSA polypeptide in SEQ ID NO: 2124.

In some embodiments, the human serum albumin-binding nanobody comprises a sequence selected from SEQ ID NO: 2125-2127. In some embodiments, the human serum albumin-binding nanobody used herein are those as described in PCT Publication No. WO2021/178804, which is incorporated by reference in its entirety.

In some embodiments, the sarbecovirus, SARS-CoV, and/or SARS-CoV-2 neutralizing nanobody reduces infectivity of a sarbecovirus, SARS-CoV, and/or SARS-CoV-2 by about 100%, about 90%, about 80%, about 70%, about 60%, or about 50%. In some embodiments the sarbecovirus, SARS-CoV, and/or SARS-CoV-2 Nb reduces infectivity of a sarbecovirus, SARS-CoV, and/or SARS-CoV-2 by about 100%, about 99%, about 98%, about 97%, about 96%, about 95%, about 94%, about 93%, about 92%, about 91%, about 90%, about 85%, about 80%, about 75%, about 70%, about 65%, about 60%, about 55%, or about 50%. Reduced infectivity can be determined using any method, including a cell culture system used to determine viral neutralization. In some embodiments, the concentration of nanobody required to achieve a reduced infectivity of a sarbecovirus, SARS-CoV, and/or SARS-CoV-2 by about 50% (e.g., an IC50) is less than 1 ng/1 ml.

In some embodiments, the sarbecovirus, SARS-CoV, and/or SARS-CoV-2 neutralizing nanobody binds specifically to the concave, hACE2 binding sites. In some embodiments, the binding affinity is a femtomolar binding.

As noted above, also included herein are methods of treating and/or preventing a sarbecovirus, SARS-CoV, and/or SARS-CoV-2 infection in a subject comprising administering to the subject a therapeutically effective amount of a sarbecovirus, SARS-CoV, and/or SARS-CoV-2 neutralizing nanobody disclosed herein. In some embodiments of the methods, the sarbecovirus, SARS-CoV, and/or SARS-CoV-2 neutralizing nanobody comprises an amino acid sequence selected from the group consisting of SEQ ID NO: 1 through SEQ ID NO: 1057. In some embodiments of the methods, the sarbecovirus, SARS-CoV, and/or SARS-CoV-2 neutralizing nanobody comprises an amino acid sequence selected from the group consisting of SEQ ID NO: 1058 through SEQ ID NO: 2114 and SEQ ID NO: 2117 through SEQ ID NO: 2119.

In some embodiments, the nanobodies disclosed herein comprise paratopes that specifically bind to epitopes on a viral protein (e.g., a SARS-CoV-2 spike protein). In some embodiments, the nanobodies specifically bind to a SARS-CoV-2 spike protein (SEQ ID NO: 2116). In some embodiments, the nanobodies specifically bind to the receptor binding domain (RBD) of a SARS-CoV-2 spike protein, wherein the RBD comprises an amino acid sequence of residue numbers 334-527 of SEQ ID NO: 2116. The term “epitope”, also known as antigenic determinant, refers to the part of an antigen that is recognized by the immune system (e.g., antibodies). The part of an antibody that binds to the epitope is referred herein as a “paratope”. Antibody-antigen interactions occur between the sequence regions on the antibody (paratope) and the antigen (epitope) at the binding interface. Consequently, paratopes and epitopes can manifest in two ways: (1) as a continuous stretch of interacting residues or (2) discontinuously, separated by one or more non-interacting residues (gaps) due to protein folding.

In some embodiments, the nanobody disclosed herein comprises an amino acid sequence having the same amino acids residues at positions 30, 31, 32, 33, 47, 48, 49, 50, 51, 52, 53, 54, 55, 56, 57, 58, 59, 60, 61, 62, 63, 105, 106, 107, 108, 109, 110 and 114 relative to SEQ ID NO: 1211, and wherein the nanobody specifically binds to amino acids at positions 453, 455, 456, 472, 473, 475, 476, 477, 483, 484, 485, 486, 487, 488, 489, 490, 491, 492 and 493 of SEQ ID NO: 2120.

In some embodiments, the nanobody disclosed herein comprises an amino acid sequence having the same amino acids residues at positions 1, 2, 3, 25, 26, 27, 28, 29, 30, 31, 32, 33, 52, 53, 54, 74, 100, 101, 102, 103, 104, 105, 106, 111, 113 and 114 relative to SEQ ID NO: 1276, and wherein the nanobody specifically binds to amino acids at positions 364, 365, 366, 367, 369, 370, 371, 372, 374, 375, 376, 377, 378, 379, 380, 381, 382, 383, 384, 385, 386, 387, 388, 389, 407, 408, 409, 410, 411, 412, 413, 414, 429, 431, 432, 433, 435 and 526 of SEQ ID NO: 2120.

In some embodiments, the nanobody disclosed herein an amino acid sequence having the same amino acids residues at positions 1, 2, 28, 29, 30, 31, 32, 33, 57, 59, 98, 99, 100, 101, 102, 103, 104, 108, 109, 110, and 111 relative to SEQ ID NO: 2118, and wherein the nanobody specifically binds to amino acids at positions 368, 369, 370, 371, 374, 376, 377, 378, 379, 380, 381, 382, 383, 384, 385, 408, 410, 411, 412, 413, 414, 415, 426, 427, 428, 429 and 430 of SEQ ID NO: 2120.

In some embodiments, the nanobody disclosed herein comprises an amino acid sequence having the same amino acids residues at positions 1, 2, 26, 30, 31, 32, 52, 56, 98, 99, 100, 101, 102, 103, 104, 107, 108, 109 and 110 relative to SEQ ID NO: 1078, and wherein the nanobody specifically binds to amino acids at positions 369, 372, 377, 378, 379, 380, 381, 382, 383, 384, 385, 386, 411, 412, 413, 414, 415, 427, 428, and 429 of SEQ ID NO: 2120.

In some embodiments, the nanobody disclosed herein comprises an amino acid sequence having the same amino acids residues at positions 47, 52, 53, 54, 56, 57, 58, 59, 60, 61, 62, 65, 99, 100, 101, 102, 103, 104, 105, 106, 107, 108, 109, 110, 111, 112, 113 and 114 relative to SEQ ID NO: 1084, and wherein the nanobody specifically binds to amino acids at positions 368, 369, 370, 371, 372, 373, 374, 375, 376, 377, 378, 379, 380, 381, 382, 383, 384, 385, 386, 404, 405, 407, 408, 411, 412, 432, 433, 434, 435, 436, 437, 503, 504, 508 and 510 of SEQ ID NO: 2120. In some embodiments, the nanobody disclosed herein comprises an amino acid sequence having the same amino acids residues at positions 1, 2, 28, 31, 32, 99, 100, 101, 102, 103, 104, 106, 107, 110, 111, 112, 113, 114, 115, 116 and 117 relative to SEQ ID NO: 1075, and wherein the nanobody specifically binds to amino acids at positions 375, 376, 377, 378, 379, 380, 381, 382, 383, 384, 385, 386, 387, 408, 410, 411, 412, 413, 414, 415, 416, 426, 427, 428, 429, 430, 431 and 432 of SEQ ID NO: 2120.

In some embodiments, the nanobody disclosed herein comprises an amino acid sequence having the same amino acids residues at positions 2, 26, 28, 30, 31, 32, 52, 53, 54, 99, 100, 101, 102, 103, 104, 107, 112, 113, 114, 115, 116 and 117 relative to SEQ ID NO: 1147, and wherein the nanobody specifically binds to amino acids at positions 369, 370, 374, 375, 376, 377, 378, 379, 380, 381, 382, 383, 384, 385, 390, 408, 410, 411, 412, 413, 414, 426, 427, 428, 429 and 430 of SEQ ID NO: 2120.

In some embodiments, the nanobody disclosed herein comprises an amino acid sequence having the same amino acids residues at positions 31, 52, 56, 57, 100, 101, 102, 103, 104, 105, 106, 107, 108, 109, 110, 111, 112, 113, 114 and 115 relative to SEQ ID NO: 1122, and wherein the nanobody specifically binds to amino acids at positions 337, 340, 346, 348, 349, 351, 352, 353, 354, 355, 356, 357, 358, 359, 394, 396, 454, 464, 465, 466, 467, 468, 469 and 516 of SEQ ID NO: 2120.

In some embodiments, the nanobody disclosed herein comprises an amino acid sequence having the same amino acids residues at positions 33, 38, 43, 44, 46, 47, 50, 56, 57, 58, 59, 60, 61, 62, 63, 64, 65, 101, 102, 103, 104, 105 and 106 relative to SEQ ID NO: 1249, and wherein the nanobody specifically binds to amino acids at positions 357, 380, 381, 382, 393, 394, 396, 426, 427, 428, 429, 430, 462, 463, 516, 517, 518, 519, 520, 521, 522 and 523 of SEQ ID NO: 2120.

In some embodiments, the nanobody disclosed herein comprises an amino acid sequence having the same amino acids residues at positions 1, 2, 26, 27, 28, 30, 31, 32, 52, 53, 54, 97, 98, 99, 100, 101, 102, 103, 104, 105, 107, 108, 109, 110 and 111 relative to SEQ ID NO: 1161 and wherein the nanobody specifically binds to amino acids at positions 380, 381, 382, 386, 387, 389, 390, 391, 392, 393, 426, 428, 429, 430, 431, 462, 464, 513, 514, 515, 516, 517, 518, 519, 520, 521, 522, 52 and 525 of SEQ ID NO: 2120.

In some embodiments, the nanobody disclosed herein comprises an amino acid sequence having the same amino acids residues at positions 27, 28, 29, 30, 31, 32, 33, 50, 52, 53, 54, 55, 56, 58, 60, 100, 101, 102, 103, 104, 105, 106, 107, 110 and 111 relative to SEQ ID NO: 2117 and wherein the nanobody specifically binds to amino acids at positions 355, 380, 381, 382, 383, 385, 386, 387, 388, 389, 390, 391, 392, 393, 394, 396, 426, 427, 428, 429, 430, 462, 463, 464, 465, 515, 516, 517, 518, 519, 520, 521 and 522 of SEQ ID NO: 2120.

In some embodiments, the nanobody disclosed herein comprises an amino acid sequence having the same amino acids residues at positions 46, 48, 115, 116, 117, 118, 119, 120, 121, 122, 123, 124 and 125 relative to SEQ ID NO: 1208 and wherein the nanobody specifically binds to amino acids at positions 355, 396, 426, 428, 429, 430, 431, 463, 464, 514, 515, 516, 517, 518, 519 and 520 of SEQ ID NO: 2120.

In some embodiments, the nanobody disclosed herein comprises an amino acid sequence having the same amino acids residues at positions 45, 47, 49, 57, 58, 59, 60, 61, 65, 99, 101, 103, 104, 105, 106, 107, 108, 109, 110, 111, 112, 113 and 115 relative to SEQ ID NO: 1086 and wherein the nanobody specifically binds to amino acids at positions 333, 334, 336, 359, 360, 361, 362, 363, 364, 365, 382, 384, 386, 387, 388, 389, 390, 391, 392, 393, 517, 518, 519, 520, 521, 522, 523, 524, 525 and 526 of SEQ ID NO: 2120.

It should be understood that in some embodiments, the SARS-CoV-2 nanobodies disclosed herein can cross-react with other coronavirus spike proteins. Accordingly, the invention includes treatment of coronaviruses other than SARS-CoV-2 with the nanobodies disclosed herein. Coronaviruses constitute the subfamily Orthocoronavirinae, in the family Coronaviridae, order Nidovirales, and realm Riboviria. They are enveloped viruses with a positive-sense single-stranded RNA genome and a nucleocapsid of helical symmetry. The genome size of coronaviruses ranges from approximately 27 to 34 kilobases. The structure of coronavirus generally consists of the following: spike protein, hemagglutinin-esterease dimer (HE), a membrane glycoprotein (M), an envelope protein (E) a nucleoclapid protein (N) and RNA. The coronavirus family comprises genera including, for example, alphacoronavius (e.g., Human coronavirus 229E, Human coronavirus NL63, Miniopterus bat coronavirus 1, Miniopterus bat coronavirus HKU8, Porcine epidemic diarrhea virus, Rhinolophus bat coronavirus HKU2, Scotophilus bat coronavirus 512), betacoronavirus (e.g., SARS-CoV-2, Betacoronavirus 1, Human coronavirus HKU1, Murine coronavirus, Pipistrellus bat coronavirus HKU5, Rousettus bat coronavirus HKU9, Severe acute respiratory syndrome-related coronavirus, Tylonycteris bat coronavirus HKU4, Middle East respiratory syndrome-related coronavirus (MERS), Human coronavirus OC43, Hedgehog coronavirus 1 (EriCoV)), gammacoronavirus (e.g., Beluga whale coronavirus SW1, Infectious bronchitis virus), and deltacoronavirus (e.g., Bulbul coronavirus HKU11, Porcine coronavirus HKU15). In some embodiments, the nanobodies disclosed herein can cross-react with other coronaviruses or other coronavirus spike proteins. In some embodiments, the nanobody cross reacts with Ratq13, pang17, SARS-CoV, WIVI, SHC014, Rs4081, RmYNo2, RF1, Yun11, BtKy72, BM4831. Accordingly, in some aspects, disclosed herein are nanobodies and uses thereof for treating and/or preventing an infection with a coronavirus. In some embodiments, the coronavirus is SARS-CoV. In some embodiments, the coronavirus is MERS-CoV.

In some embodiments, the nanobody is administered at a dose of about 0.01 mg/kg of body weight, about 0.05 mg/kg of body weight, about 0.1 mg/kg of body weight, about 0.15 mg/kg of body weight, about 0.2 mg/kg of body weight, about 0.25 mg/kg of body weight, about 0.3 mg/kg of body weight, about 0.35 mg/kg of body weight, about 0.4 mg/kg of body weight, about 0.45 mg/kg of body weight, about 0.5 mg/kg of body weight, about 0.55 mg/kg of body weight, about 0.6 mg/kg of body weight, about 0.65 mg/kg of body weight, about 0.7 mg/kg of body weight, about 0.75 mg/kg of body weight, about 0.8 mg/kg of body weight, about 0.85 mg/kg of body weight, about 0.9 mg/kg of body weight, about 0.95 mg/kg of body weight, about 1 mg/kg of body weight, about 2 mg/kg of body weight, about 3 mg/kg of body weight, about 4 mg/kg of body weight, about 5 mg/kg of body weight, about 6 mg/kg of body weight, about 7 mg/kg of body weight, about 8 mg/kg of body weight, about 9 mg/kg of body weight, about 10 mg/kg of body weight, or about 20 mg/kg of body weight. In some embodiments, the nanobody is administered at a dose of at least about 0.01 mg/kg of body weight (e.g., at least about 0.1 mg/kg of body weight, at least about 0.2 mg/kg of body weight, at least about 0.3 mg/kg of body weight, at least about 0.5 mg/kg of body weight, at least about 1.0 mg/kg of body weight, at least about 2 mg/kg of body weight, at least about 5 mg/kg of body weight, at least about 10 mg/kg of body weight, at least about 50 mg/kg of body weight, or at least about 100 mg/kg of body weight).

The disclosed methods can be employed 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 years; 12, 11, 10, 9, 8, 7, 6, 5, 4, 3, 2, or 1 months; 30, 29, 28, 27, 26, 25, 24, 23, 22, 21, 20, 19, 18, 17, 16, 15, 14, 13, 12, 11, 10, 9, 8, 7, 6, 5, 4, or 3 days; 60, 48, 36, 30, 24, 18, 15, 12, 10, 9, 8, 7, 6, 5, 4, 3, or 2 hours prior to the onset of a COVID-19 symptom; or 1, 2, 3, 4, 5, 6, 7, 8, 9, 10, 15, 20, 25, 30, 35, 40, 45, 50, 55, 60, 75, 90, 105, 120 minutes; 3, 4, 5, 6, 7, 8, 9, 10, 11, 12, 15, 18, 24, 30, 36, 48, 60 hours; 3, 4, 5, 6, 7, 8, 9, 10, 11, 12, 13, 14, 15, 16, 17, 18, 19, 20, 21, 22, 23, 24, 25, 26, 27, 28, 29, 30, 45, 60, 90 or more days; 4, 5, 6, 7, 8, 9, 10, 11, 12 or more months; 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1 years after the onset of symptoms of sarbecovirus, SARS-CoV, and/or SARS-CoV-2 infection (e.g., COVID-19 symptom). In some embodiments, the disclosed methods can be employed prior to or following the administering of another anti-SARS-CoV-2 agent.

A coronavirus or SARS-CoV-2 neutralizing nanobody described herein can be administered to the subject via any route including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation or via an implanted reservoir. In some embodiments, the nanobody is administered intratracheally, intranasally, or through an inhalation route. In some embodiments, the coronavirus or SARS-CO-V2 neutralizing nanobody is in an aerosol form. The term “parenteral” includes subcutaneous, intravenous, intramuscular, intra-articular, intra-synovial, intrasternal, intrathecal, intrahepatic, intralesional, and intracranial injections or infusion techniques.

Dosing frequency for a coronavirus or SARS-CoV-2 neutralizing nanobody of any preceding aspects, includes, but is not limited to, at least once every year, once every two years, once every three years, once every four years, once every five years, once every six years, once every seven years, once every eight years, once every nine years, once every ten year, at least once every two months, once every three months, once every four months, once every five months, once every six months, once every seven months, once every eight months, once every nine months, once every ten months, once every eleven months, at least once every month, once every three weeks, once every two weeks, once a week, twice a week, three times a week, four times a week, five times a week, six times a week, daily, twice a day, three times a day, four times a day, or five times a day. Administration can also be continuous and adjusted to maintaining a level of the compound within any desired and specified range.

### EXAMPLES

### Example 1. Versatile, Multivalent Nanobody Cocktails Efficiently Neutralize SARS-CoV-2

A collection of diverse, soluble, stable, and high-affinity camelid Nbs that target the RBD were developed and characterized. The majority of the high-affinity RBD Nbs efficiently neutralize SARS-CoV-2. A subset has exceptional neutralization potency, comparable with, or better than the most potent human NAbs. Integrative structural approach was used and multiple epitopes of neutralizing Nbs were mapped. An atomic structure of an elite Nb of sub-ng/ml neutralization potency was determined in complex with the RBD. The results revealed that while highly potent neutralizing Nbs predominantly recognize the concave, hACE2 binding site, efficient neutralization can also be accomplished through other RBD epitopes. Finally, structural characterizations facilitated the bioengineering of multivalent Nbs into multi-epitope cocktails that achieved remarkable neutralization potencies of as low as 0.058 ng/ml (1.3 μM), which can be sufficient to prevent the generation of escape mutants

### Development of Highly Potent SARS-COV-2 Neutralizing Nbs

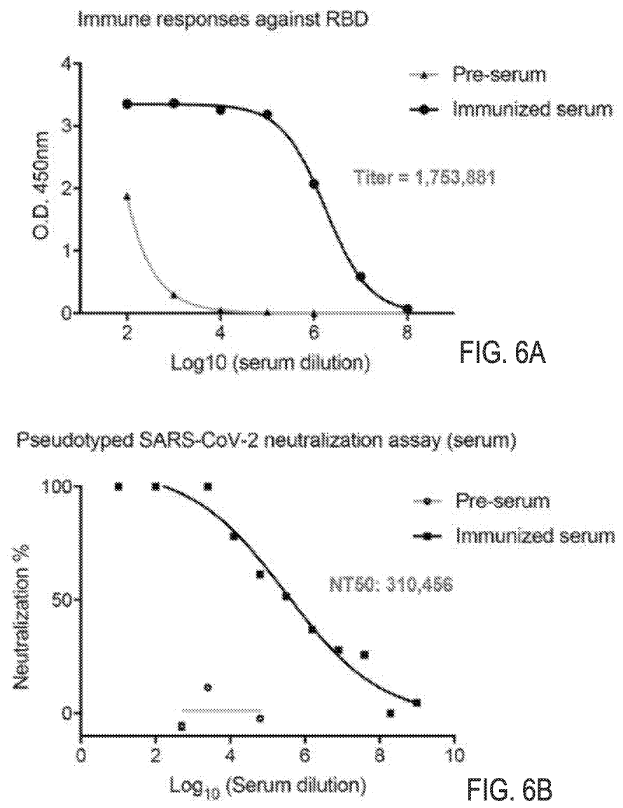

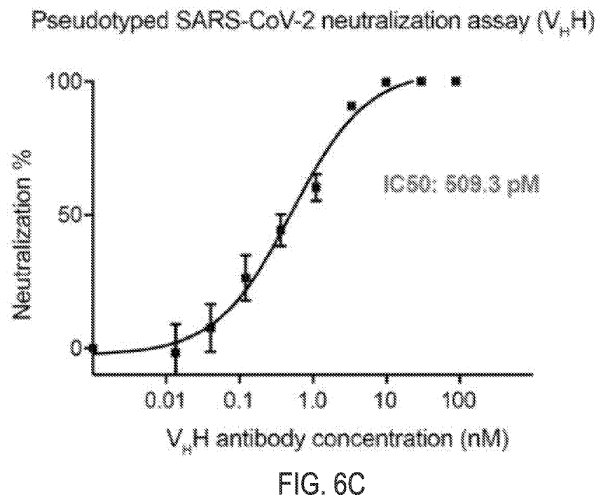

To produce high-quality SARS-CoV-2 neutralizing Nbs, a llama was immunized with the recombinant RBD protein expressed in human 293T cells. Compared to the pre-bleed, after affinity maturation, the post-immunized serum showed potent and specific serologic activities towards RBD binding with a titer of 1.75×10⁶ (FIG. 6A). The serum efficiently neutralized the pseudotyped SARS-CoV-2 at the half-maximal neutralization titer (NT50) of 310,000 (FIG. 6B), orders of magnitude higher than the convalescent sera obtained from recovered COVID-19 patients (Y. Cao et al., 2020; D. F. Robbiani et al., 2020). To further characterize these activities, the single-chain VHH antibodies were separated from the IgG antibodies from the serum. It was confirmed that the single-chain antibodies achieve specific, high-affinity binding to the RBD and possess sub-nM half-maximal inhibitory concentration (IC50=509 μM) against the pseudotyped virus (FIG. 6C).

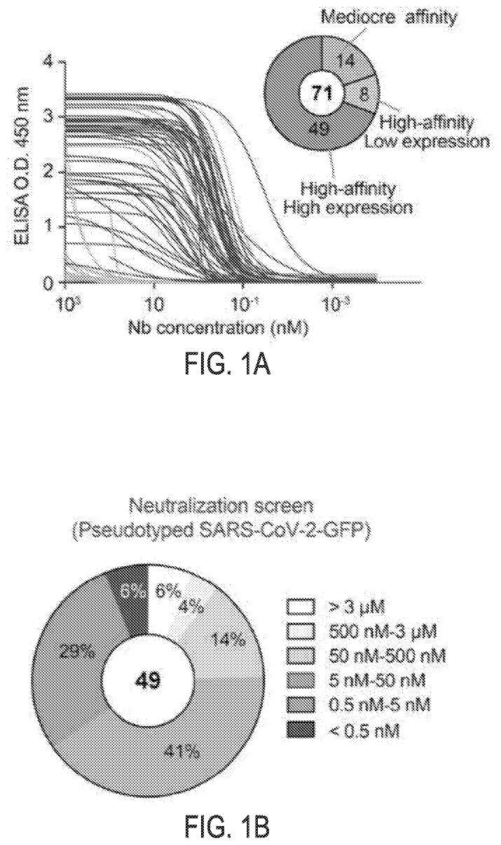

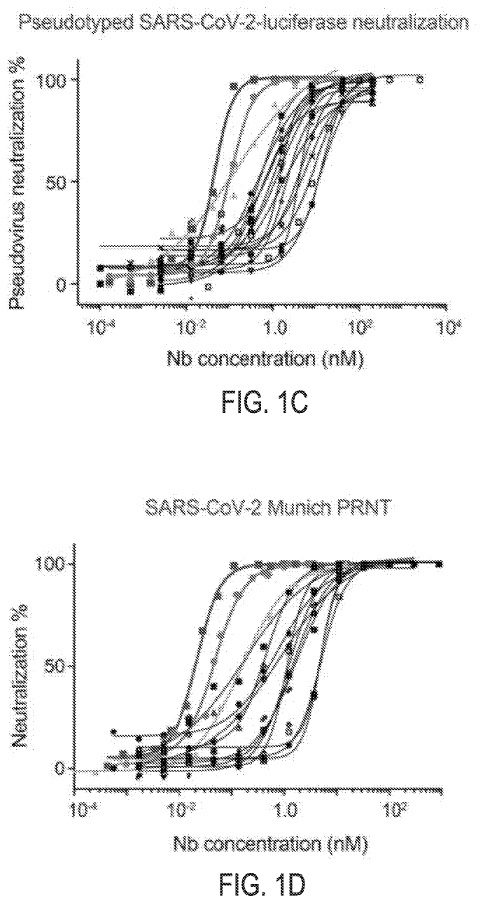

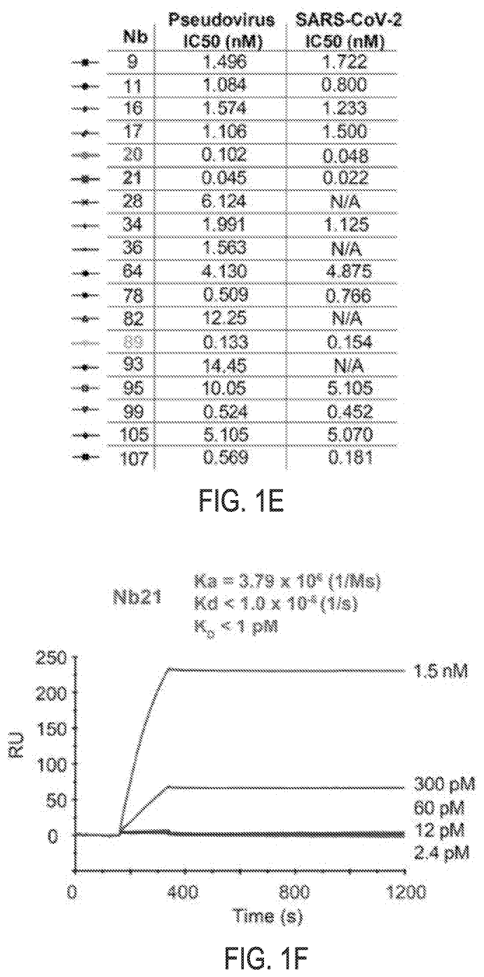

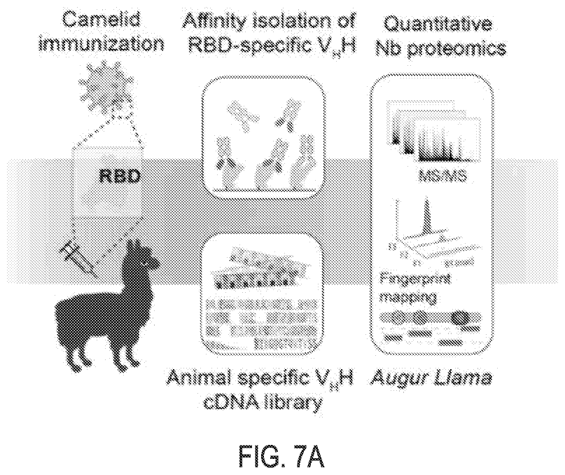

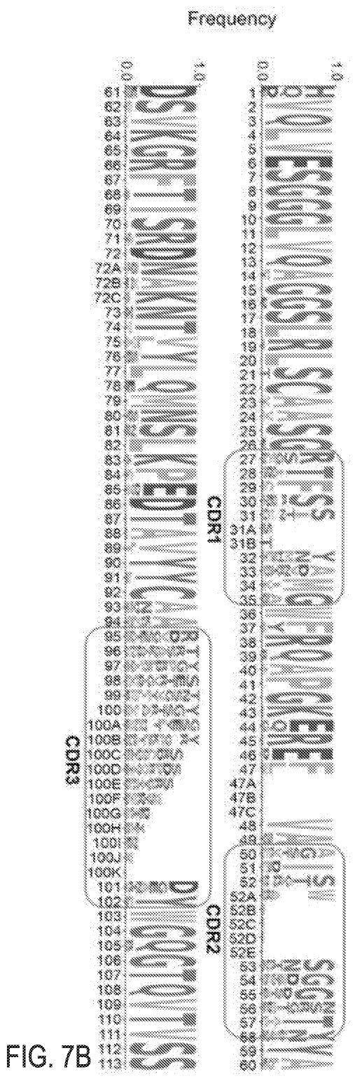

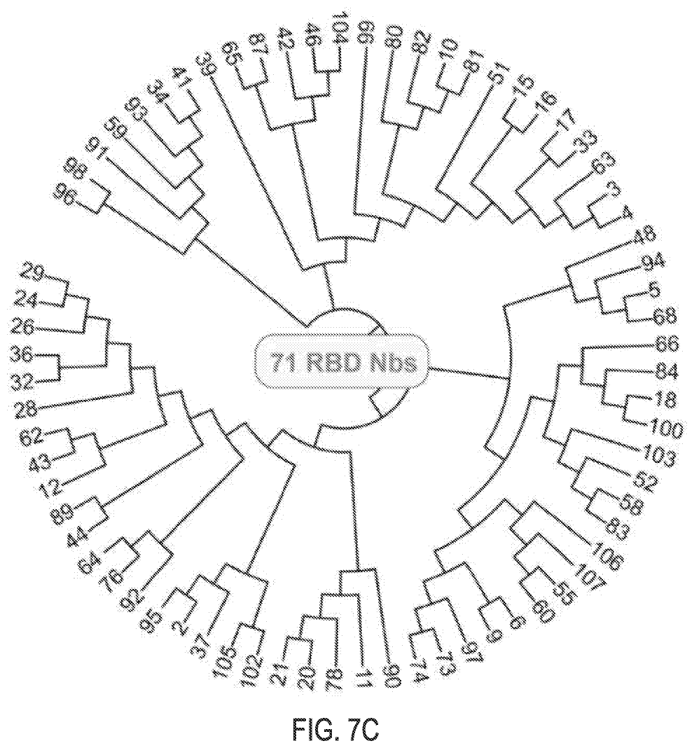

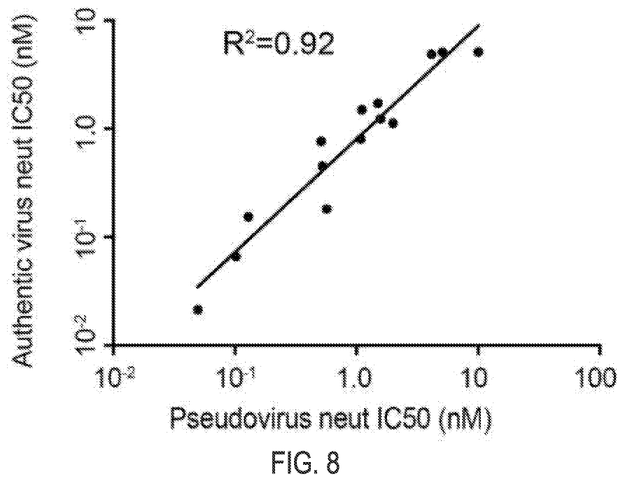

Thousands of high-affinity VHH Nbs from the RBD-immunized llama serum were identified using a robust proteomic strategy (Y. Xiang et al., 2020) (FIG. 7a). This repertoire includes ˜350 unique CDR3s (complementarity-determining regions). For E. coli expression, 109 highly diverse Nb sequences were selected from the repertoire with unique CDR3s to cover various biophysical, structural, and different antiviral properties of the Nb repertoire. Ninety four Nbs were purified and tested for RBD binding by ELISA, from which 71 RBD-specific binders were confirmed (FIG. 7b-7c). Of these RBD-specific binders, 49 Nbs presented high solubility and high-affinity (ELISA IC50 below 30 nM, FIG. 1a), and were candidates for functional characterizations. A SARS-CoV-2-GFP pseudovirus neutralization assay was used to screen and characterize the antiviral activities of these high-affinity Nbs. The vast majority (94%) of the tested Nbs can neutralize the pseudotype virus below 3 μM (FIG. 1b). 90% of them blocked the pseudovirus below 500 nM. Only 20-40% of high-affinity RBD-specific mAbs identified from the patients' sera have been reported to possess comparable potency (Y. Cao et al., 2020; D. F. Robbiani et al., 2020). Over three quarters (76%) of the Nbs efficiently neutralized the pseudovirus below 50 nM, and 6% had neutralization activities below 0.5 nM. Finally, the potential of 14 to neutralize SARS-CoV-2 Munich strain was tested using the PRNT50 assay (W. B. Klimstra et al., 2020). All the Nbs reached 100% neutralization and neutralized the virus in a dose-dependent manner. The IC50s span from single-digit ng/ml to sub- ng/ml, with three unusual neutralizers of Nbs 89, 20, and 21 to be 2.0 ng/ml (0.129 nM), 1.6 ng/ml (0.102 nM), and 0.7 ng/ml (0.045 nM), respectively, based on the pseudovirus assay (FIG. 1c, FIG. 1e). Similar values (0.154 nM, 0.048 nM, and 0.021 nM, for Nbs 89, 20, and 21) were reproducibly obtained using SARS-CoV-2 (FIG. 1d, FIG. 1e). There was an excellent correlation between the two neutralization assays (R²=0.92, FIG. 8).

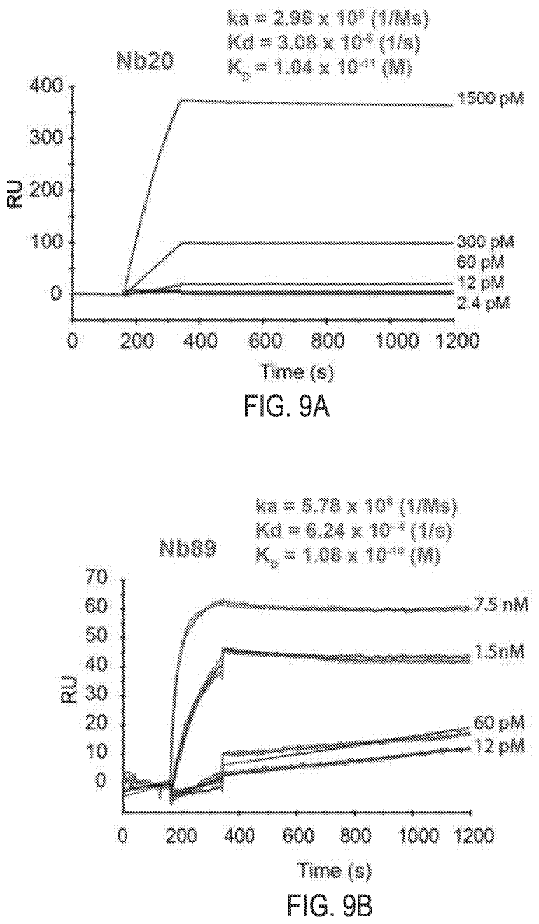

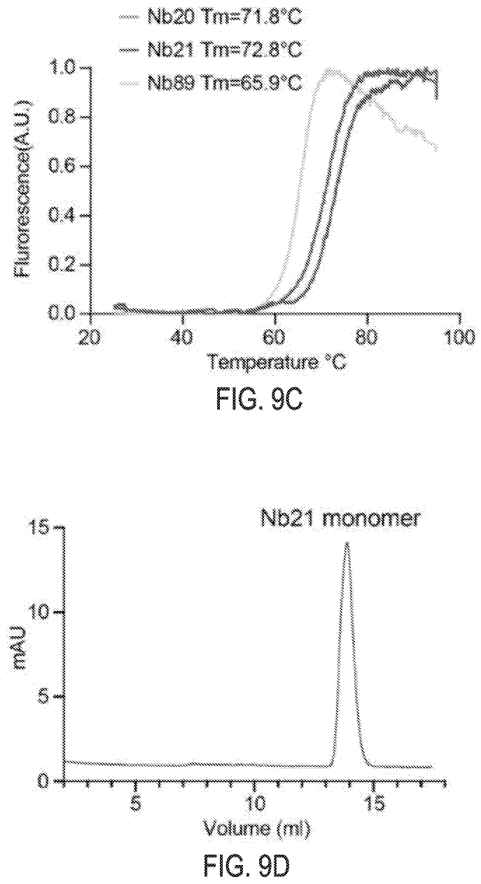

The binding kinetics of Nbs 89, 20, and 21 were measured by surface plasmon resonance (SPR) (FIG. 9a-9b). While Nbs 89 and 20 have an affinity of 108 pM and 10.4 pM, the best-neutralizing Nb21 did not show detectable dissociation from the RBD during 20 min SPR analysis. The femtomolar affinity of Nb21 potentially explains its unusual neutralization potency (FIG. 1f). The experiment determined thermostability of the top three neutralizing Nbs (89, 20, and 21) from the E. coli periplasmic preparations to be 65.9, 71.8, and 72.8° C., respectively (FIG. 9c). Finally, the on-shelf stability of Nb21 was tested, which remained soluble after˜6 weeks of storage at room temperature after purification and can well tolerate lyophilization. No multimeric forms or aggregations were detected by size-exclusion chromatography (SEC) (FIG. 9d). Together these results show that these neutralizing Nbs have the necessary physicochemical properties required for advanced therapeutic applications.

### Integrative Structural Characterization of the Nb Neutralization Epitopes

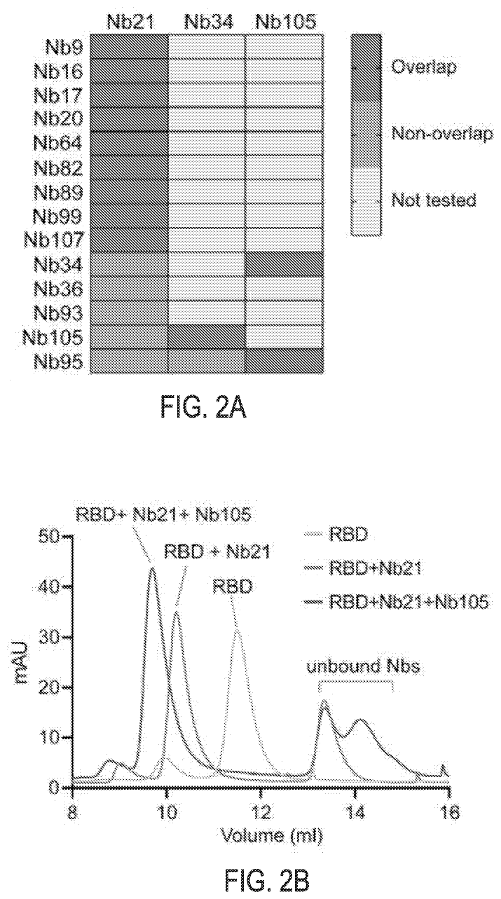

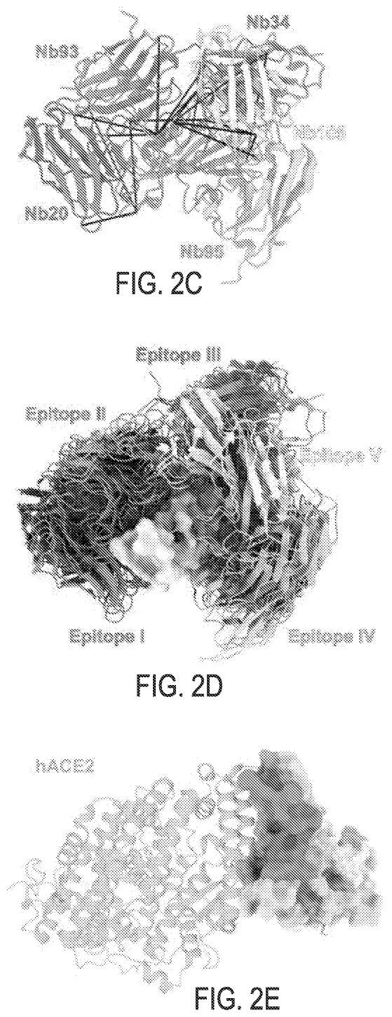

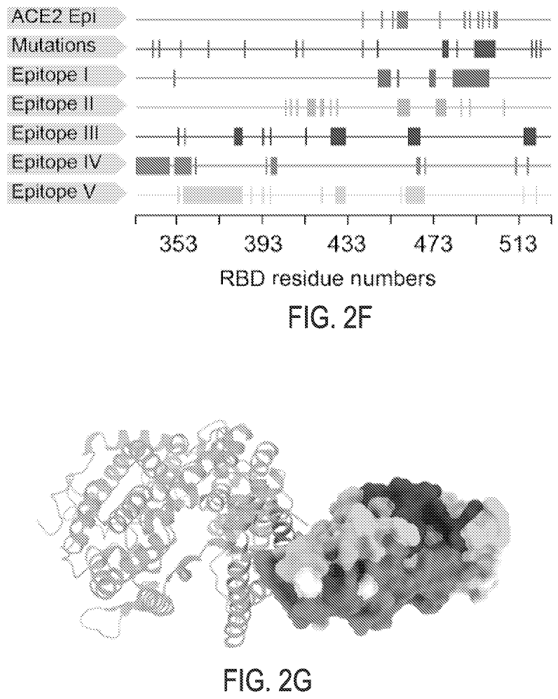

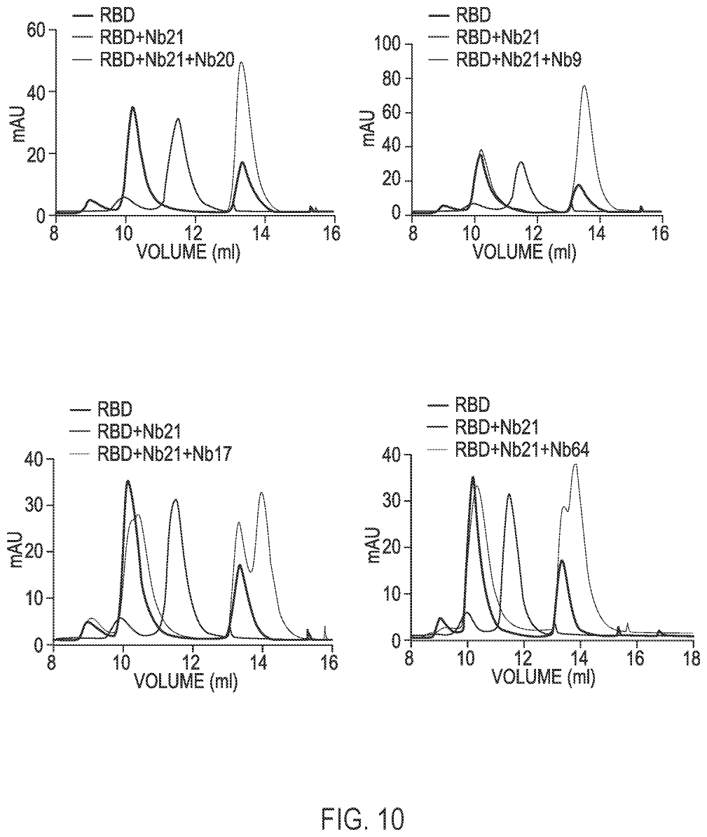

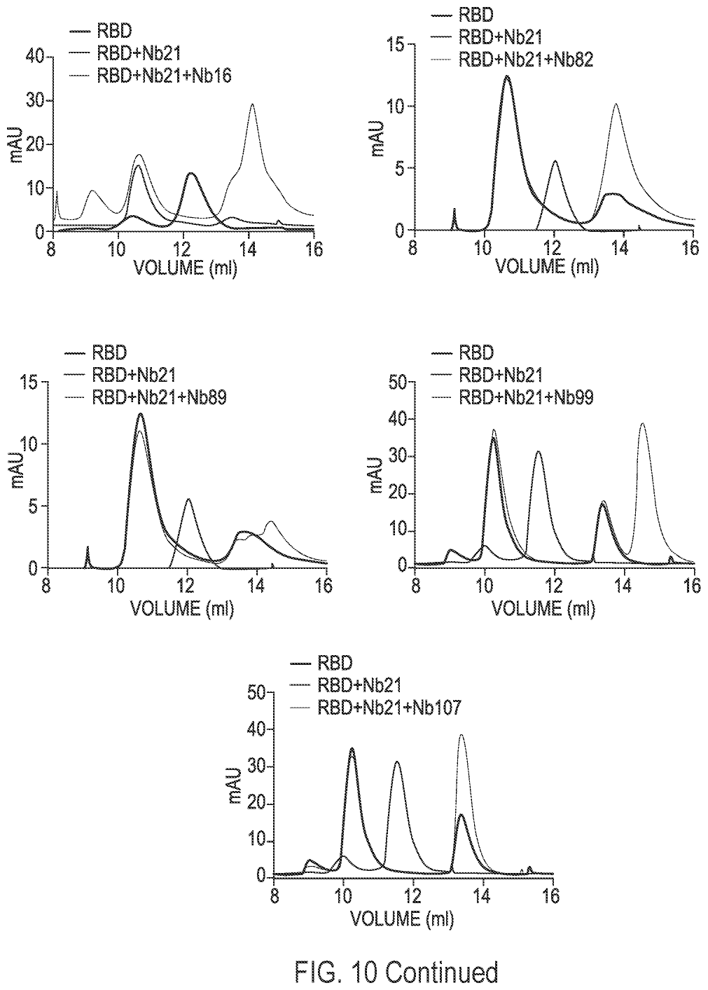

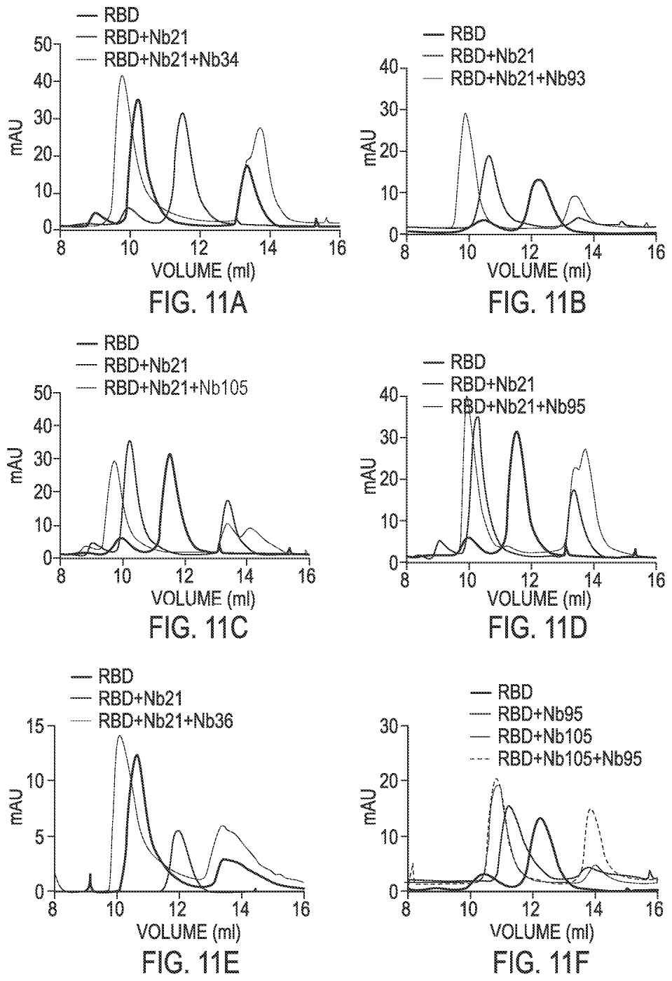

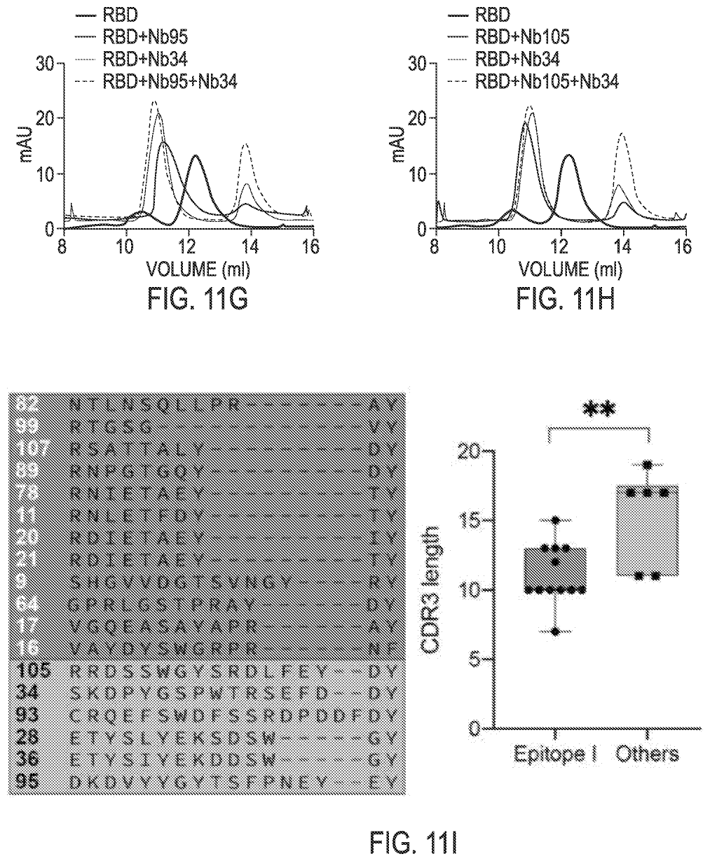

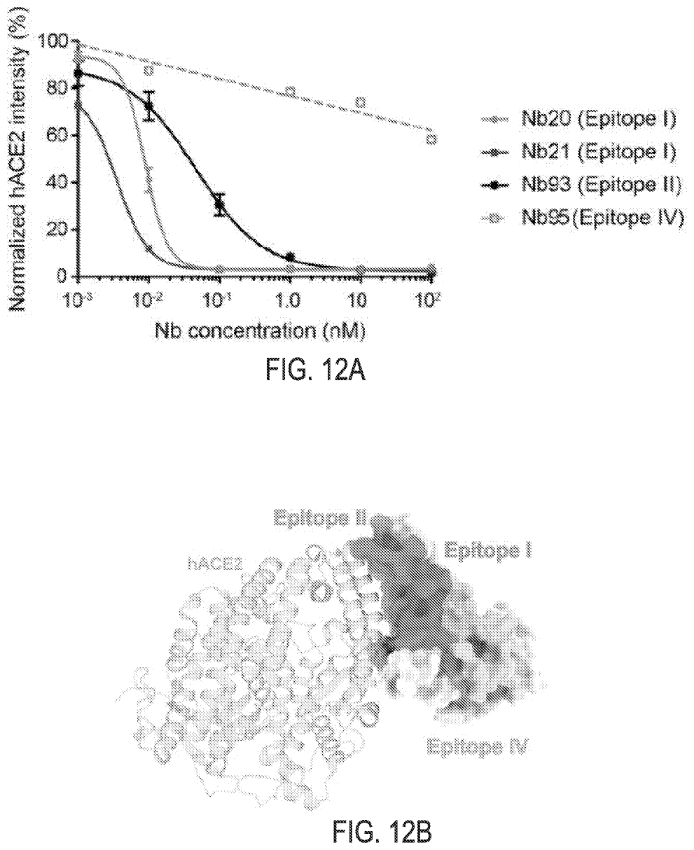

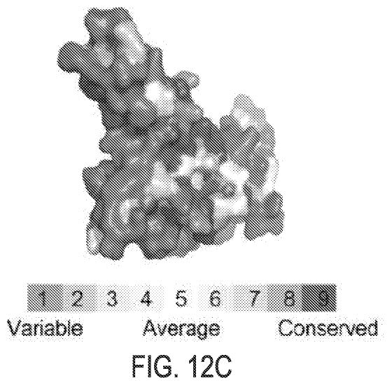

Epitope mapping based on atomic resolution structure determination by X-ray crystallography and Cryo-Electron Microscopy (CryoEM) is highly accurate but low-throughput. Here, information from SEC, cross-linking mass spectrometry (CXMS), shape and physicochemical complementarity, and statistics was integrated to determine structural models of RBD-Nb complexes (M. P. Rout, A. Sali, 2019; C. Yu et al., 2017; A. Leitner, M. Faini, F. 2016; B. T. Chait et. al., 2016). First, SEC experiments were performed to distinguish between Nbs that share the same epitope as Nb21 (thus complete with Nb 21 on an SEC) and those that bind to non-overlapping epitopes. Nbs 9, 16, 17, 20, 64, 82, 89, 99 and 107 competed with Nb21 for RBD binding based on SEC profiles (FIG. 2a, FIG. 10), indicating that their epitopes significantly overlap. In contrast, higher mass species (from early elution volumes) corresponding to the trimeric complexes composed of Nb21, RBD, and one of the Nbs (34, 36, 93, 105, and 95) were evident (FIGS. 2b, 11a-11h). Moreover, Nb105 competed with Nb34 and Nb95, which did not compete for RBD interaction, indicating the presence of two distinct and non-overlapping epitopes. Second, Nb-RBD complexes were cross-linked by DSS (disuccinimidyl suberate) and were identified on average, four intermolecular cross-links by MS for Nbs 20, 93, 34, 95, and 105. The cross-links were used to map the RBD epitopes derived from the SEC data (Methods). The cross-linking models identified five epitopes (I, II, III, IV, and V corresponding to Nbs 20, 93, 34, 95, and 105) (FIG. 2c). The models satisfied 90% of the cross-links with an average precision of 7.8 Å (FIG. 2d). The analysis confirmed the presence of a dominant Epitope I (e.g., epitopes of Nbs 20 and 21) overlapping with the hACE2 binding site. Epitope II also co-localized with the hACE2 binding site, while epitopes III-V did not (FIG. 2e). Epitope I Nbs had significantly shorter CDR3 (four amino acids shorter, p=0.005) than other epitope binders (FIG. 11i). Despite this, the vast majority of the selected Nbs potently inhibited the virus with an IC50 below 30 ng/ml (2 nM).

### Crystal Structure of RBD-Nb20 and MD Analysis of Nbs 20 and 21 for RBD Binding

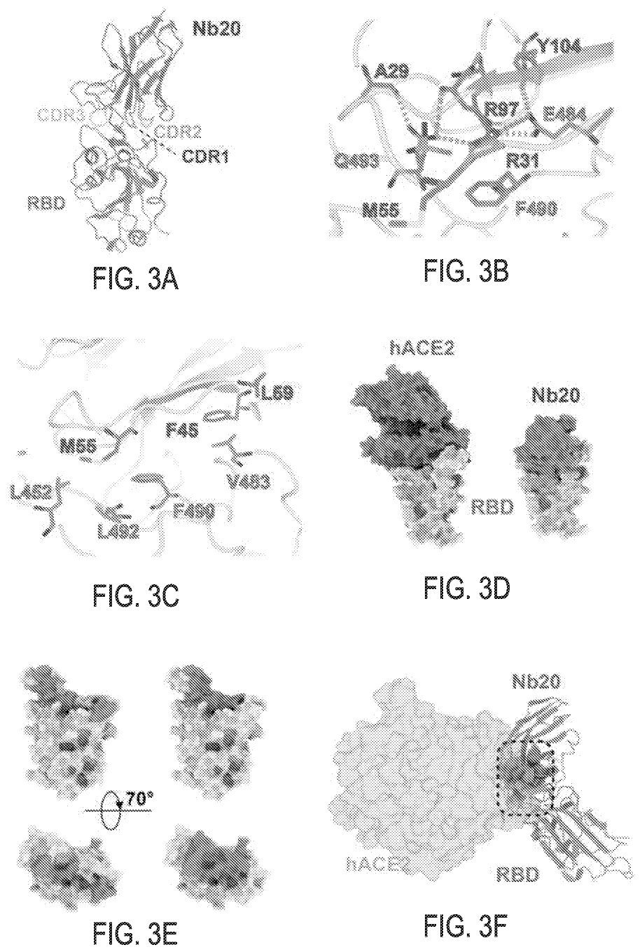

To explore the molecular mechanisms that underlie the unusually potent neutralization activities of Epitope I Nbs, a crystal structure of the RBD-Nb20 complex at a resolution of 3.3 Å was determined by molecular replacement. Most of the residues in RBD (N334-G526) and the entire Nb20, particularly those at the protein interaction interface, are well resolved. There are two copies of RBD-Nb20 complexes in one asymmetric unit, which are almost identical with an RMSD of 0.277 Å over 287 Cα atoms. In the structure, all three CDRs of Nb20 interact with the RBD by binding to its large extended external loop with two short β-strands (FIG. 3a) (Q. H. Wang et al., 2020). E484 of RBD forms hydrogen bonding and ionic interactions with the side chains of R31 (CDR1) and Y104 (CDR3) of Nb20, while Q493 of RBD forms hydrogen bonds with the main chain carbonyl of A29 (CDR1) and the side chain of R97 (CDR3) of Nb20. These interactions constitute a major polar interaction network at the RBD and Nb20 interface. R31 of Nb20 also engages in a cation-π interaction with the side chain of F490 of the RBD (FIG. 3b). In addition, M55 from the CDR2 of Nb20 packs against residues L452, F490, and L492 of RBD to form hydrophobic interactions at the interface). Another small patch of hydrophobic interactions is formed among residues V483 of RBD and F45 and L59 from the framework β-sheet of Nb20 (FIG. 3c).

A small cavity with strong electron density at the RBD and Nb20 interface was observed surrounded by charged residues R31 and R97 of Nb20 and E484 of the RBD. A cacodylate group was modeled a from the protein crystallization conditions to fit the density, forming hydrogen bonds with two main chain amine groups of Nb20 and the RBD. Under physiological conditions, this cavity is occupied by ordered water molecules to mediate extensive hydrogen-bonding interactions with surrounding residues, contributing to the interactions between the RBD and Nb20. Similarly, in two crystal structures of the RBD bound to other Nbs (PDB IDs 6YZ5 and 7C8V0), there are also small molecules such as glycerol modeled at the RBD and Nb interfaces.

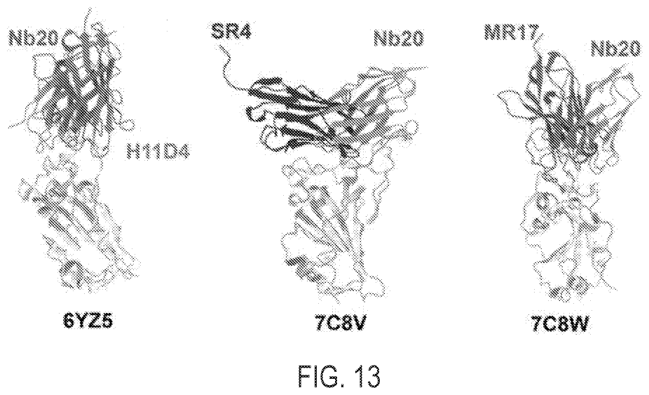

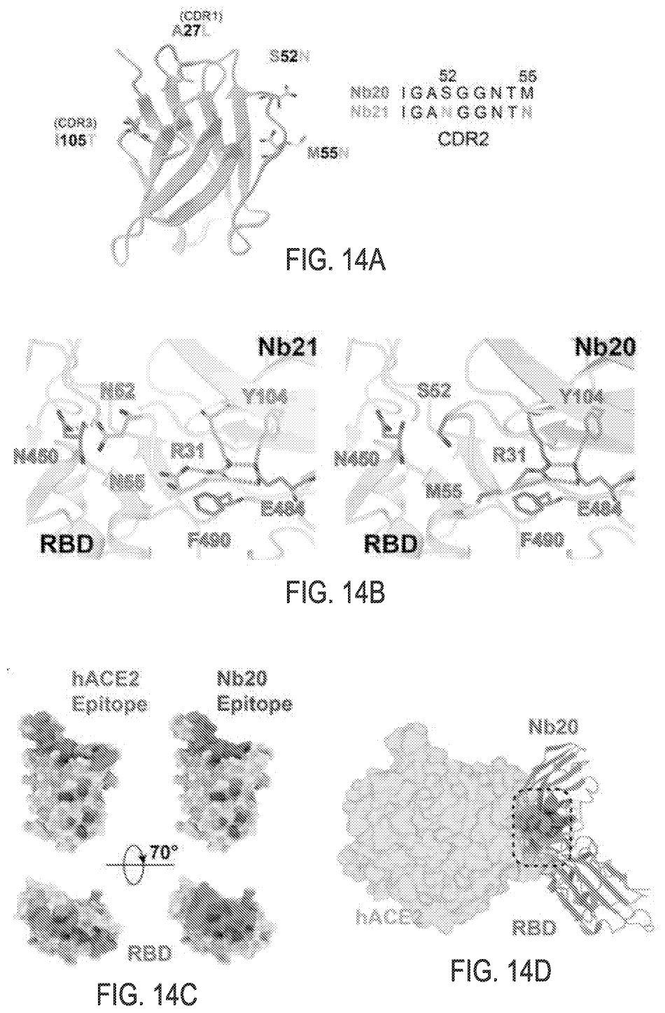

The binding mode of Nb20 to the RBD is distinct from all other reported SARS-CoV-2 neutralizing Nbs, which generally recognize similar epitopes in the RBD external loop region (T. Li et al., 2020; J. D. Walter et al., 2020; J. Huo et al., 2020) (FIG. 13). The extensive hydrophobic and polar interactions (FIGS. 3b-3c) between the RBD and Nb20 stem from the remarkable shape complementarity (FIG. 3d) between all the CDRs and the external RBD loop, leading to ultrahigh-affinity (˜10 pM). The structure of the best neutralizer Nb21 with RBD was further modeled based on the crystal structure (Methods). Only four residues vary between Nb20 and Nb21 (FIG. 14a), all of which are on CDRs. Two substitutions are at the RBD binding interface. S52 and M55 in the CDR2 of Nb20 are replaced by two asparagine residues N52 and N55 in Nb21. In this modeled structure, N52 forms a new H-bond with N450 of RBD (FIG. 14b). While N55 does not engage in additional interactions with RBD, it creates a salt bridge with the side chain of R31, which stabilizes the polar interaction network among R31 and Y104 of Nb21 and Q484 of RBD (FIG. 14b). All of those can contribute to a slower off-rate of Nb21 vs. Nb20 (FIGS. 2f, 9a) and stronger neutralization potency. Structural comparison of RBD-Nb20/21 and RBD-hACE2 (PDB 6LZG) (Q. H. Wang et al., 2020) clearly showed that the interfaces for Nb20/21 and hACE2 partially overlap (FIGS. 3d-3e). Notably, the CDR1 and CDR3 of Nb20/21 can severely clash with the first helix of hACE2, the primary binding site for RBD (FIG. 3f). This high-resolution structural study shows the exceptional binding affinities of epitope I Nbs that contribute to the sub-ng/ml neutralization capability.

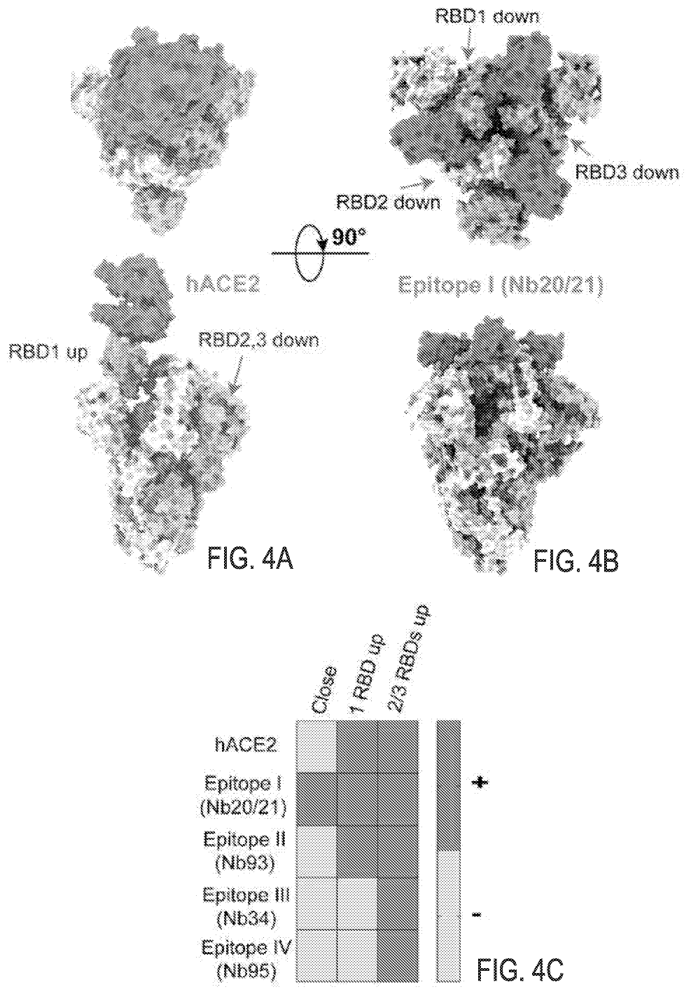

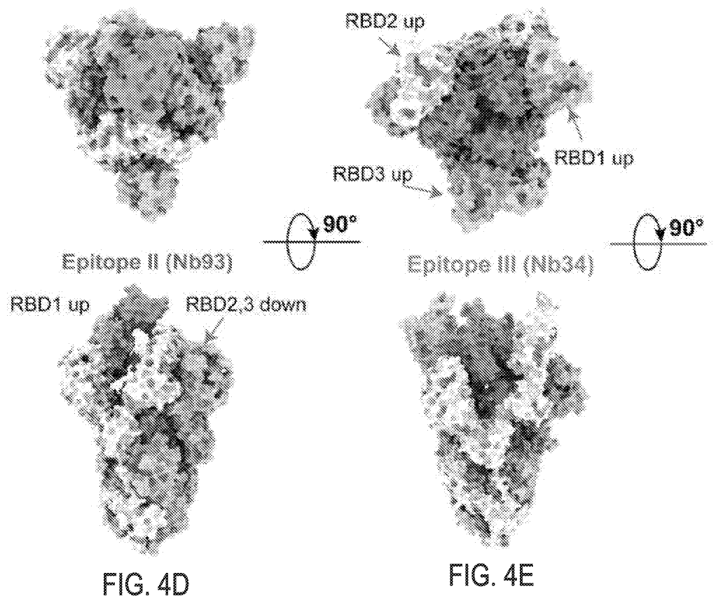

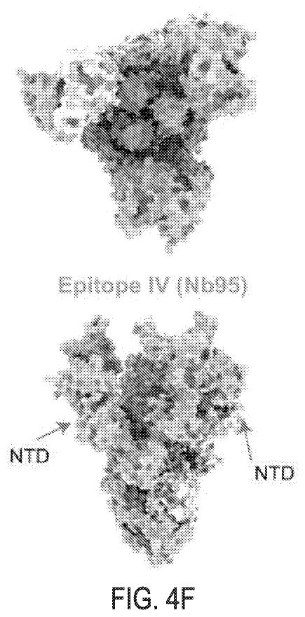

To further explore the molecular mechanisms that underlie the exceptional neutralization activities of our Nbs, an X-ray crystallographic structure of the RBD-Nb20 complex at a resolution of 3.3 Å was determined in which all the residue side chains were resolved. This structure reveals that Nb20 employs an extensive network of hydrophobic and polar interactions to enable sub-nM, high-affinity RBD binding. Consistent to cross-linking, Nb20 interacts with the large cavity on the RBD formed by an extended loop (region 1: residues 432-438 and region 2: residues 464-484, FIG. 4). All three CDR loops are involved in binding. For example, A29 of CDR1 forms hydrophobic interactions with both Y436 and S481, and R31 (the last CDR1 residue) inserts itself into the deep pocket of the RBD cavity, creating a salt bridge with E471 and a cation-π interaction with F477. 5 out of 9 residues on the CDR2 contact the RBD loop by hydrophobic interactions (i.e., pairs of A 48-E 471, A 51-S481, S 52-N436, M55-L479, and N57-F477) facilitating the penetration of its loop into the RBD groove. Despite relatively short CDR3, at least three residues (R94, 196, and Y101) bind directly to the RBD cavity by H-bonds and hydrophobic interactions. Moreover, the interaction between Nb20 and the RBD is further stabilized by a conserved F47 on the FR2 and a residue X on the FR3 that pairs with V483 and XYZ. Together, Nb 20 employs a remarkable array of hydrophobic and polar interactions that enable perfection of shape complementarity between the convex Nb and the RBD groove.

### Potential Mechanisms of SARS-CoV-2 Neutralization by Nbs

To understand the outstanding antiviral efficacy of these Nbs better, RBD-Nb complexes were superimposed to different spike conformations based on cryoEM structures. It was found that three copies of Nb20/21 can simultaneously bind all three RBDs in their “down” conformations (PDB 6VXX) (A. C. Walls et al., 2020) that correspond to the inactive spike (FIG. 4b). This analysis indicates a mechanism by which Nbs 20 and 21 (Epitope I) lock RBDs in their down conformation with ultrahigh affinity. Combined with the steric interference with hACE2 binding in the RBD open conformation (FIG. 4a), these mechanisms can explain the exceptional neutralization potencies of Epitope I Nbs.

Other epitope-binders do not fit into this inactive conformation without steric clashes and appear to utilize different neutralization strategies (FIG. 4c). For example, Epitope II: Nb 93 co-localizes with hACE2 binding site and can bind the spike in the one RBD “up” conformation (FIG. 4d, PDB 6VSB) (D. Wrapp et al., 2020). It can neutralize the virus by blocking the hACE2 binding site. Epitope III and IV Nbs can only bind when two or three RBDs are at their “up” conformations (PDB 6XCN) (C. O. Barnes et al., 2020) where the epitopes are exposed. In the all RBDs “up” conformation, three copies of Nbs can directly interact with the trimeric spike. Through RBD binding, Epitope III: Nb34 can be accommodated on top of the trimer to lock the helices of S2 in the prefusion stage, preventing their large conformational changes for membrane fusion (FIG. 4e). When superimposed onto the all “up” conformation, Epitope IV: Nb95 is proximal to the rigid NTD of the trimer, presumably restricting the flexibility of the spike domains (FIG. 4f).

### Development Of Flexible, Multivalent Nb Formats for Highly Efficient Viral Neutralization

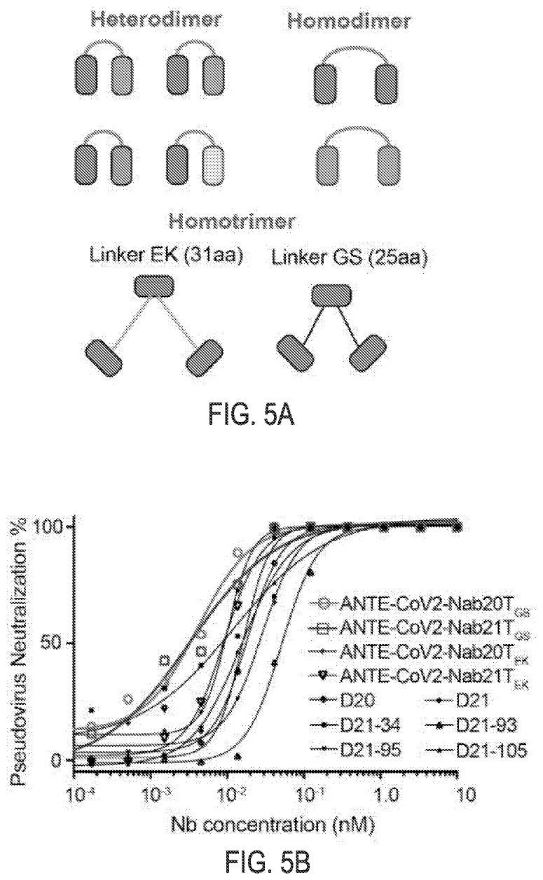

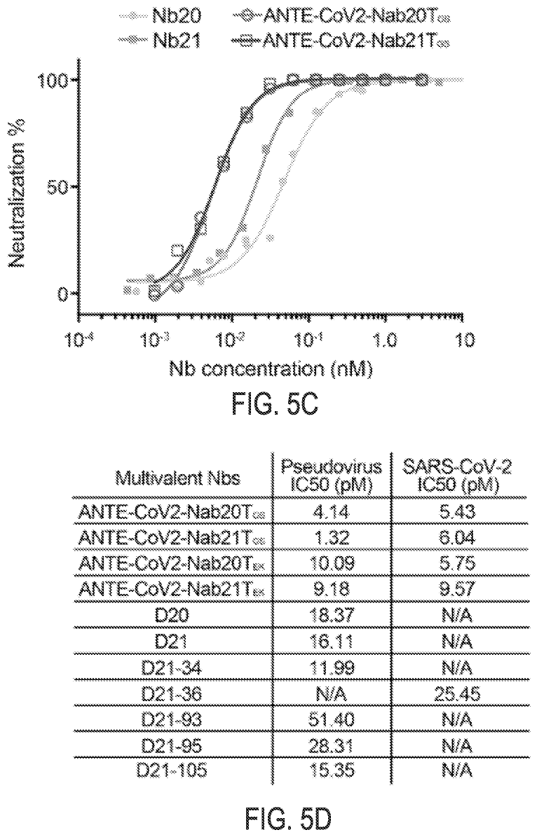

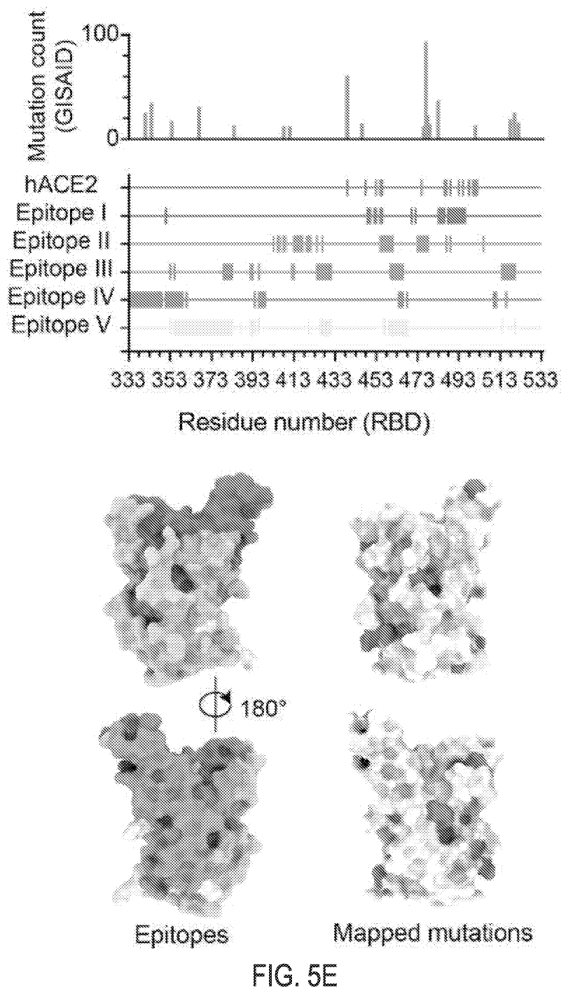

Epitope mapping enabled us to bioengineer a series of multivalent Nbs (FIG. 5a). Specifically, two sets of constructs that build upon the most potent Nbs were designed. The homotrimeric Nbs, in which a flexible linker sequence (either 31 or 25 amino acids, Methods) separates each monomer Nb (such as Nb21 or Nb20), were designed to increase the antiviral activities through avidity binding to the trimeric spike. The heterodimeric forms that conjugate two Nbs of unique, non-overlapping epitopes, through a flexible linker of 12 residues.

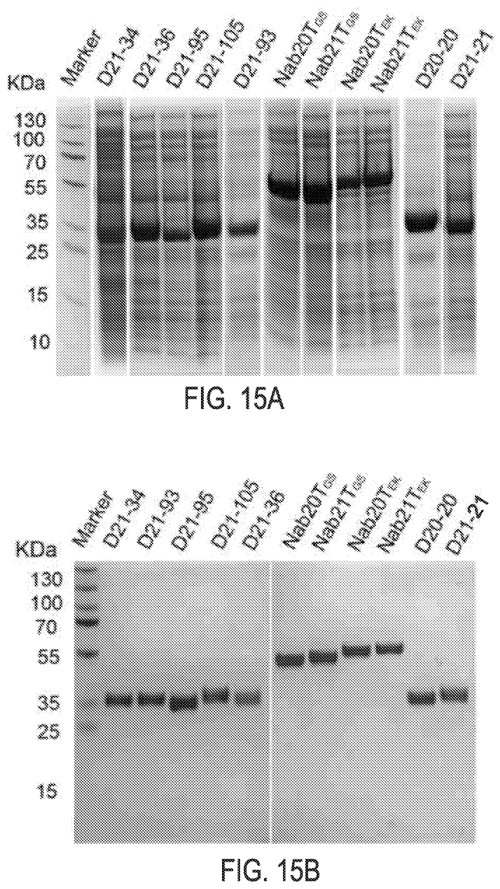

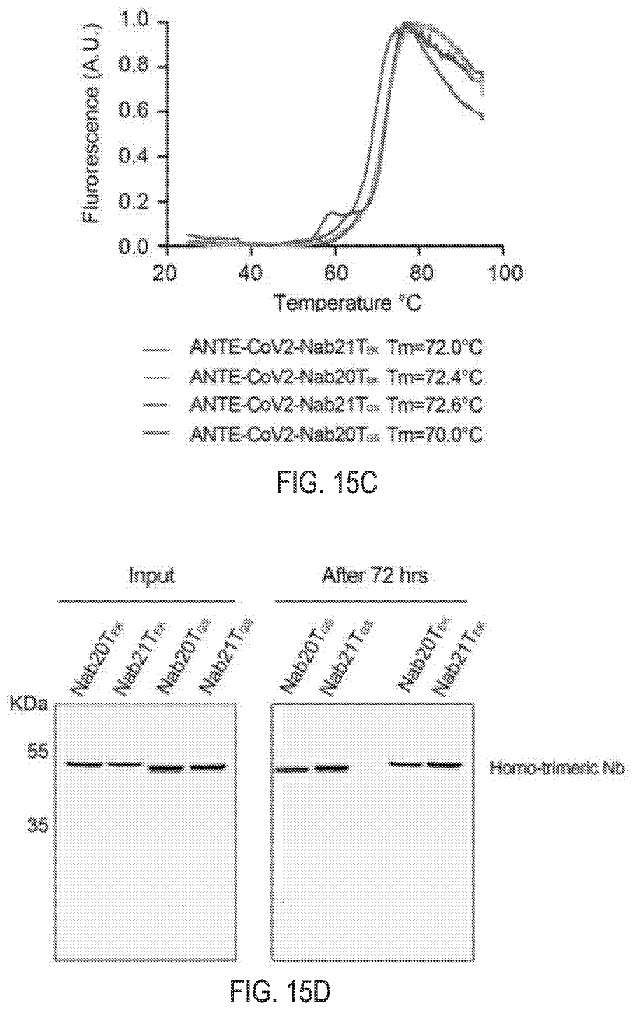

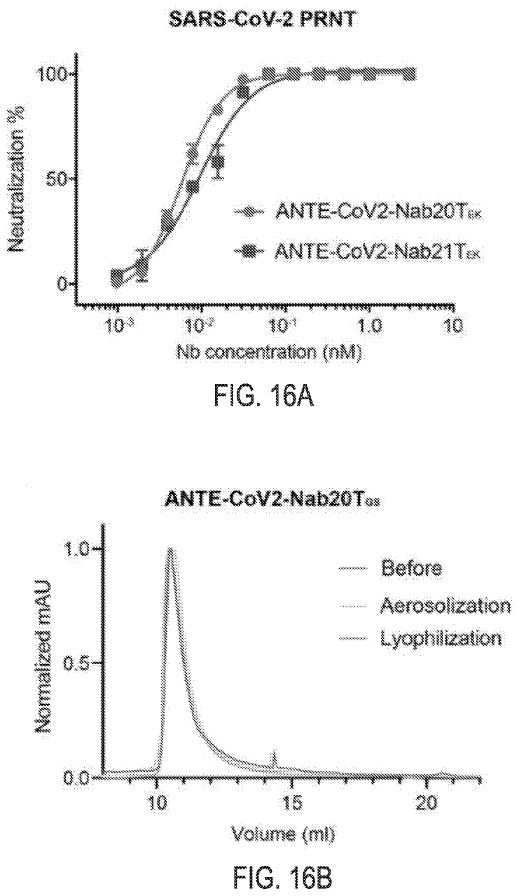

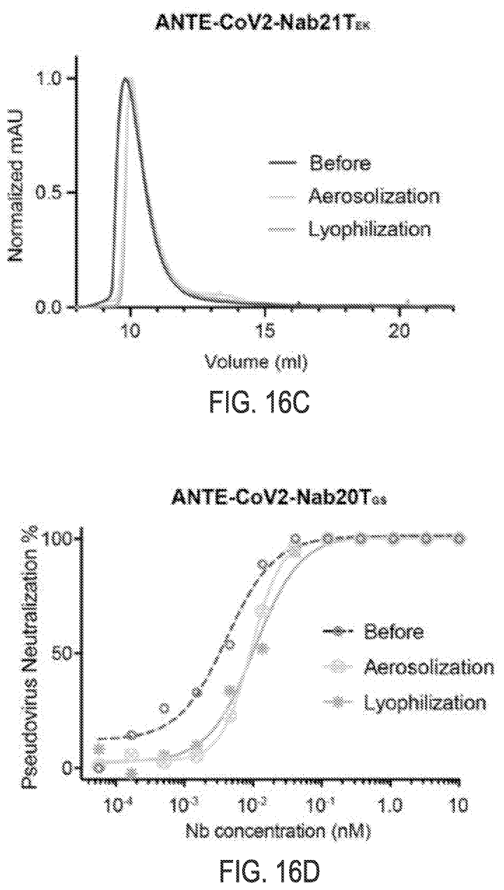

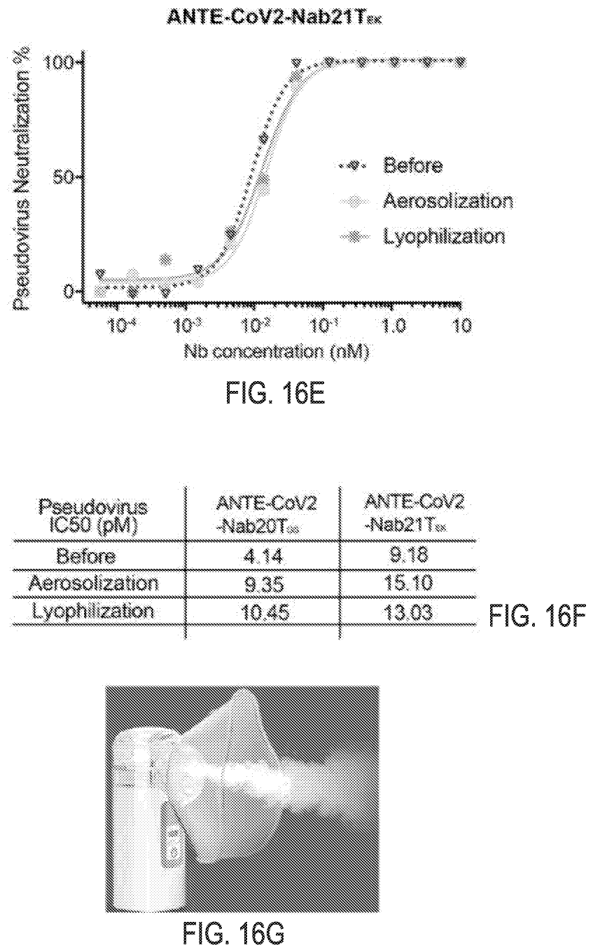

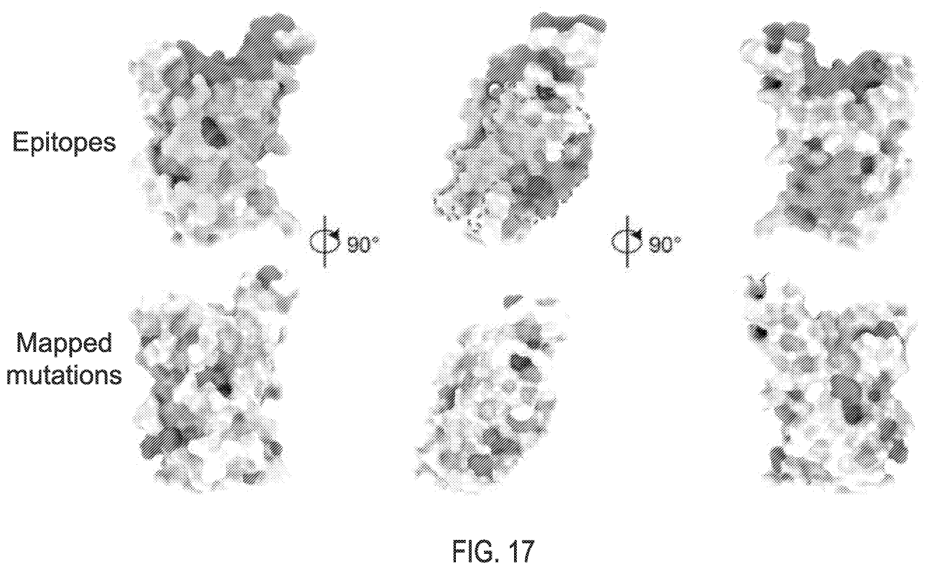

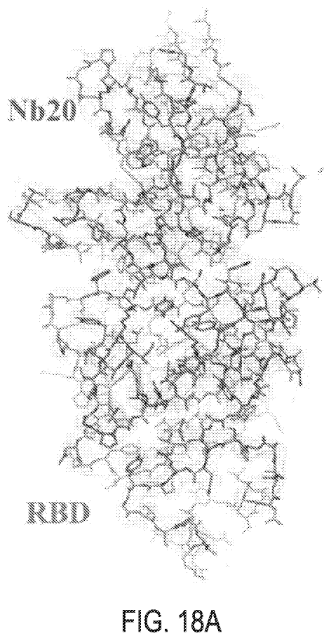

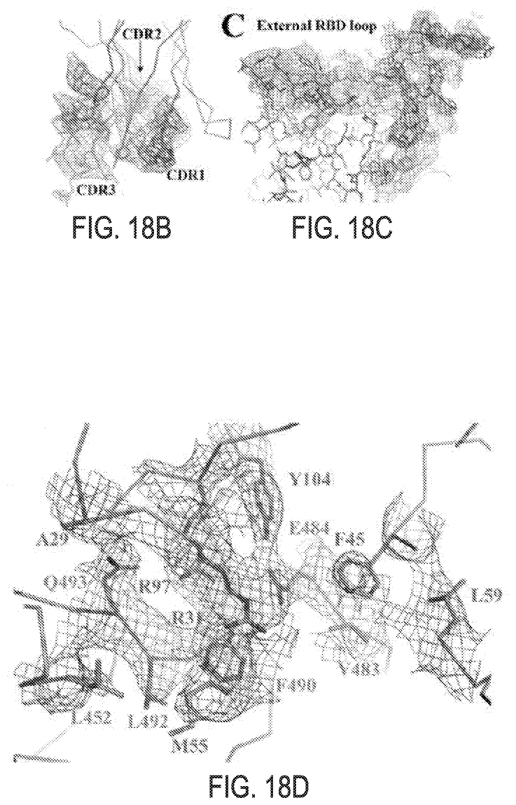

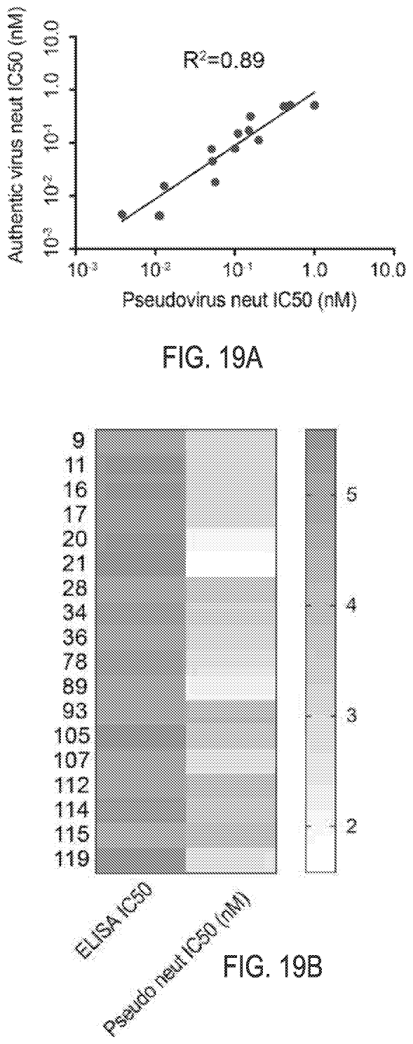

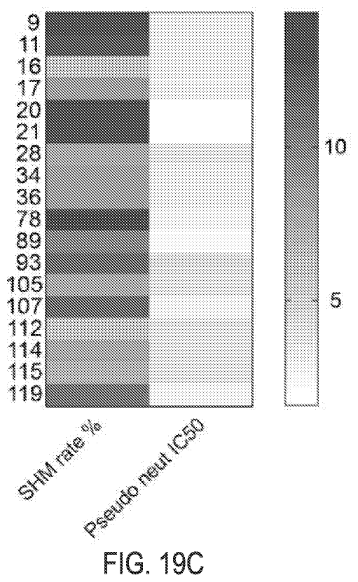

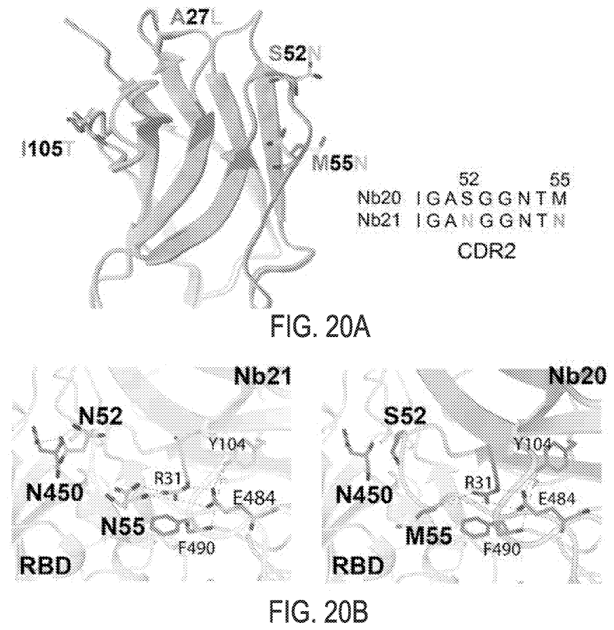

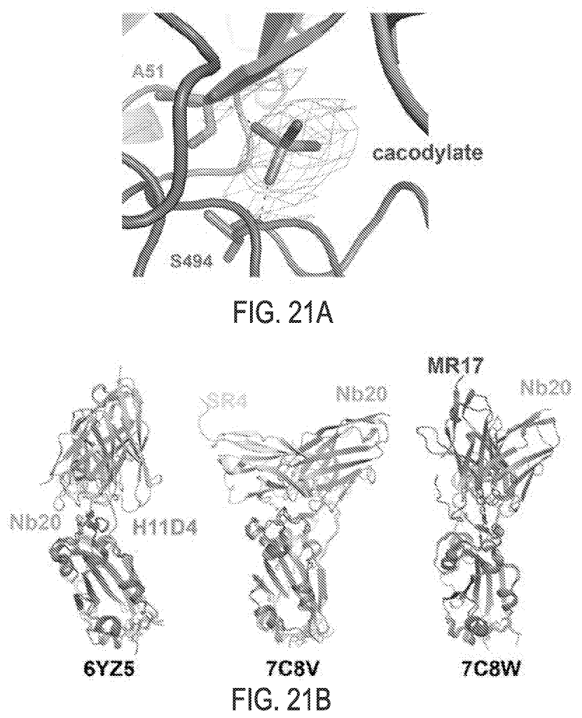

A variety of constructs were synthesized and their neutralization potency was tested. Up to ˜30 fold improvement was found for the homotrimeric constructs of Nb21₃ (IC50=1.3 pM) and Nb20₃ (IC50=3 pM) compared to the respective monomeric form by the pseudovirus luciferase assay (FIG. 5b, FIG. 5d). Similar results were obtained from the SARS-CoV-2 PRNT (FIGS. 5c, 5d, 15a). The improvements are greater than these values indicate, as the measured values can reflect the assay's lower detection limits. For the heterodimeric constructs, up to a 4-fold increase of potency (i.e., Nb21-Nb34) was observed. Importantly, the multivalent constructs retained outstanding physicochemical properties of the monomeric Nbs, including high solubility, yield, and thermostability (FIG. 15). They remained fully active after standard lyophilization and nebulization (Methods, FIGS. 16b-16e), indicating the outstanding stability and flexibility of administration. The majority of the RBD mutations observed in GISAID (Y. L. Shu et al., 2017) are very low in frequency (<0.0025). Therefore, the probability of mutational escape with a cocktail consisting of 2-3 Nbs covering different epitopes is extremely low (FIG. 5e) (J. Hansen et al., 2020).

The development of effective, safe, and inexpensive vaccines and therapeutics are critical to end the COVID-19 pandemic. Here, in vivo (camelid) antibody affinity maturation followed by advanced proteomics (Y. Xiang et al., 2020) enabled rapid identification of a large repertoire of diverse, high-affinity RBD Nbs for the neutralization of SARS-CoV-2. The majority of the high-affinity Nbs efficiently neutralize SARS-CoV-2 and some elite Nbs in their monomeric forms can inhibit the viral infection at single-digit to sub-ng/ml concentrations.

Multiple neutralization epitopes were identified through the integration of biophysics, structural proteomics, modeling, and X-ray crystallography. This study shows the mechanisms by which Nbs target the RBD with femtomolar affinity to achieve remarkable neutralization potency of a low-passage, clinical isolate of SARS-CoV-2. Structural analysis revealed that the hACE2 binding site correlates with immunogenicity and neutralization. While the most potent Nbs inhibit the virus by high-affinity binding to the hACE2 binding site, other neutralization mechanisms through non-hACE2 epitopes were also observed.

A preprint reported an extensively bioengineered, homotrimeric Nb construct reaching antiviral activity comparable to our single monomeric Nb20 (M. Schoof et al., 2020). Here, the present study has developed a collection of novel multivalent Nb constructs with outstanding stability and neutralization potency at double-digit pg/ml. This represents the most potent biotherapeutics for SARS-CoV-2 available to date. The use of multivalent, multi-epitope Nb cocktails can prevent virus escape (A. Baum et al., 2020; Y. Bar-On et al., 2018; M. Marovich et al., 2020). Flexible and efficient administration, such as direct inhalation can be used to improve antiviral drug efficacy and minimize the dose, cost, and potential toxicity for clinical applications. The high sequence similarity between Nbs and IgGs can restrain the immunogenicity (I. Jovcevska et. al., 2020). For intravenous drug delivery, it is possible to fuse our antiviral Nbs with the albumin-Nb constructs (Z. Shen et al., 2020) t already developed to improve the in vivo half-lives. These Nbs can also be applied as rapid point-of-care diagnostics due to the high stability, specificity, and low cost of manufacturing. These high-quality Nb agents contributes to curbing the current pandemic.

### Methods

Camelid immunization and proteomic identification of high-affinity RBD-Nbs. A male Llama “Wally” was immunized with an RBD-Fc fusion protein (Acro Biosystems, Cat #SPD-c5255) at a primary dose of 0.2 mg (with complete Freund's adjuvant), followed by three consecutive boosts of 0.1 mg every 2 weeks. ˜480 ml blood from the animal was collected 10 days after the final boost. All the above procedures were performed by the Capralogics, Inc. following the IACUC protocol. ˜1×10⁹ peripheral mononuclear cells were isolated using Ficoll gradient (Sigma). The mRNA was purified from the mononuclear cells using an RNeasy kit (Qiagen) and was reverse-transcribed into cDNA by the Maxima™ H Minus cDNA Synthesis kit (Thermo). The V_{H}H genes were PCR amplified, and the P5 and P7 adapters were added with the index before sequencing (Y. Xiang et al., 2020). Next-generation sequencing (NGS) of the V_{H}H repertoire was performed by Illumina MiSeq with the 300 bp paired-end model in the UPMC Genome Center. For proteomic analysis of RBD-specific Nbs, plasma was first purified from the immunized blood by the Ficoll gradient (Sigma). V_{H}H antibodies were then isolated from the plasma by a two-step purification protocol using protein G and protein A sepharose beads (Marvelgent) (P. C. Fridy et al., 2014). RBD-specific V_{H}H antibodies were affinity isolated and subsequentially eluted by either increasing stringency of high pH buffer or salt. All the eluted V_{H}Hs were neutralized and dialyzed into 1×DPBS before the quantitative proteomics analysis. RBD-specific V_{H}H antibodies were reduced, alkylated and in-solution digested using either trypsin or chymotrypsin (Y. Xiang et al., 2020). After proteolysis, the peptide mixtures were desalted by self-packed stage-tips or Sep-Pak C18 columns (Waters) and analyzed with a nano-LC 1200 that is coupled online with a Q Exactive™ HF-X Hybrid Quadrupole Orbitrap™ mass spectrometer (Thermo Fisher). Proteomic analysis was performed as previously described and by using the Augur Llama—a dedicated software that we developed to facilitate reliable identification, label-free quantification, and classification of high-affinity Nbs (25). This analysis led to thousands of RBD-specific, high-affinity Nb candidates that belong to ˜350 unique CDR3 families. From these, we selected 109 Nb sequences with unique CDR3s for DNA synthesis and characterizations.

Nb DNA synthesis and cloning. The monomeric Nb genes and the homotrimeric Nbs 20 and 21 with the (GGGGS, (SEQ ID NO: 175))₅ linkers were codon-optimized and synthesized (Synbio). All the Nb DNA sequences were cloned into a pET-21b(+) vector using EcoRI and HindIII restriction sites. The monomeric Nbs 20, 21, and 89, as well as the homotrimeric Nbs 20 and 21, were also cloned into a pET-22b(+) vector at the BamHI and XhoI sites for periplasmic purification.

The Nb genes were codon-optimized for expression in E. coli and in vitro synthesized (Synbio). Different synthesized Nb genes were cloned into a pET-21b (+) vector at BamHI and XhoI restriction sites or pET-22b (+) vector. To produce the heterodimeric Nb formats, DNA fragments of Nbs (such as Nb34), were amplified from the pET21(a+) Nb constructs while new XhoI/HindIII restriction sites plus (GGGGS, (SEQ ID NO: 175))₂ linker sequence were introduced. The fragments were then inserted into pET21(a+)_Nb21 at the XhoI and HindIII restriction sites to produce a heterodimer form [Nb21-(GGGGS, (SEQ ID NO: 175))₂-Nb]. The homotrimeric constructs were either directly synthesized or produced in house by recombinant DNA methods. The DNA fragment of the linker sequence EGKSSGSGSESKSTGGGGSEGKSSGSGSESKST (SEQ ID NO: 2115) was annealed and extended using the following two oligos: CCGCTCGAGTGCTGCGGCCGCGGTGCTTTTGCTTTCGCCGCTACCGCTGCTTTTACCT TCGCTGCCACC (SEQ ID NO: 2122), and CCCAAGCTTGAAGGTAAAAGCAGCGGTAGCGGCGAAAGCAAAAGCACCGGTGGCG GTGGCAGCGAAGGT (SEQ ID NO: 2123) (Integrated DNA Technologies).

The digested XhoI/HindIII linker fragment was then inserted into the corresponding sites on pET21(a+)_Nb21 or pET21(a+)_Nb20. To shuffle the second Nb21 or 20 to this Nb_linker vector, we amplified Nb21 or 20 from pET21(a+) and introduced the XhoI/NotI restriction sites. After digestion, the XhoI/NotI Nb fragment was inserted into the Nb_linker vector to produce a homodimer construct. The new Nb constructs were subsequently sequence verified.

Expression and purification of proteins. Nb DNA constructs were transformed into BL21(DE3) cells and plated on Agar with 50 μg/ml ampicillin at 37° C. overnight. Cells were cultured in an LB broth to reach an O.D. of ˜0.5-0.6 before IPTG (0.5 mM) induction at 16° C. overnight. Cells were then harvested, sonicated, and lysed on ice with a lysis buffer (1×PBS, 150 mM NaCl, 0.2% TX-100 with protease inhibitor). After cell lysis, protein extracts were collected by centrifugation at 15,000×g for 10 mins and the his-tagged Nbs were purified by Cobalt resin and natively eluted by imidazole buffer (Thermo). Eluted Nbs were subsequently dialyzed in a dialysis buffer (e.g., 1×DPBS, pH 7.4). For the periplasmic preparation of Nbs (Nbs 20, 21, and 89 and the homotrimeric constructs), cell pellets were resuspended in the TES buffer (0.1 M Tris-HCl, pH 8.0; 0.25 mM EDTA, pH 8.0; 0.25 M Sucrose) and incubated on ice for 30 min. The supernatants were collected by centrifugation and subsequently dialyzed to DPBS. The resulting Nbs were then purified by Cobalt resin as described above.

The RBD (residues 319-541) of the SARS-CoV-2 S protein was expressed as a secreted protein in Spodoptera frugiperda Sf9 cells (Expression Systems) using the Bac-to-bac baculovirus method (Invitrogen). To facilitate protein purification, a FLAG-tag and an 8×His-tag were fused to its N terminus, and a tobacco etch virus (TEV) protease cleavage site was introduced between the His-tag and RBD. Cells were infected with baculovirus and incubated at 27° C. for 60 h before harvesting. The conditioned media was added with 20 mM Tris pH 7.5 and incubated at RT for 1 h in the presence of 1 mM NiSO4 and 5 mM CaCl₂. The supernatant was collected by centrifugation at 25,000 g for 30 min and then incubated with Nickel-NTA agarose resin (Clontech) overnight at 4° C. After washing with buffer containing 20 mM Hepes pH 7.5, 200 mM NaCl, and 50 mM imidazole, the RBD protein was eluted with the same buffer containing 400 mM Imidazole. Eluted protein was treated by TEV protease overnight to remove extra tags and further purified by size exclusion chromatography using the Superdex 75 column (Fisher) with a buffer containing 20 mM HEPES pH 7.5 and 150 mM NaCl. To obtain RBD and Nb20 complex, purified RBD was mixed with purified Nb20 in a molar ratio of 1:1.5 and then incubated on ice for 2 hours. The complex was further purified using the Superdex 75 column with a buffer containing 20 mM Hepes pH 7.5 and 150 mM NaCl. Purified RBD-Nb20 complex was concentrated to 10-15 mg/ml for crystallization.

ELISA (Enzyme-linked immunosorbent assay). Indirect ELISA was carried out to measure the relative affinities of Nbs. RBD was coated onto a 96-well ELISA plate (R&D system) at two ng/well in coating buffer (15 mM sodium carbonate, 35 mM sodium bicarbonate, pH 9.6) overnight at 4° C. and was blocked with a blocking buffer (DPBS, 0.05% Tween 20, 5% milk) at room temperature for 2 hrs. Nbs were serially 10× diluted in the blocking buffer, starting from 1 μM to 0.1 pM, and 100 μl of each concentration was incubated with RBD-coated plates for 2 hrs. HRP-conjugated secondary antibodies against T7-tag (Thermo) were diluted 1:7500 and incubated with the well for 1 hr at room temperature. After PBST (DPBS, 0.05% Tween 20) washes, the samples were further incubated under dark with freshly prepared w3,3′,5,5′-Tetramethylbenzidine (TMB) substrate for 10 mins to develop the signals. After the STOP solution (R&D system), the plates were read at multiple wavelengths (the optical density at 550 nm wavelength subtracted from the density at 450 nm) on a plate reader (Multiskan GO, Thermo Fisher). A non-binder was defined if any of the following two criteria were met: i) The ELISA signal was under detected at one μM concentration. ii) The ELISA signal could only be detected at a concentration of 1 μM and was under detected at 0.1 μM concentration. The raw data was processed by Prism 7 (GraphPad) to fit into a 4PL curve and to calculate log IC50.

Competitive ELISA with hACE2. The COVID-19 spike-ACE2 binding assay kit was purchased from RayBiotech (Cat #CoV-SACE2-1). A 96-well plate was pre-coated with recombinant RBD. Nbs were 10-fold diluted (from 1 μM to 1 μM) in the assay buffer containing a saturating amount of hACE2 and then incubated with the plate at room temperature for 2.5 hrs. The plate was washed by the washing buffer to remove the unbound hACE2. Goat anti-hACE2 antibodies were incubated with the plate for 1 hr at room temperature. HRP-conjugated anti-goat IgG was added to the plate and incubated for an hour. TMB solution was added to react with the HRP conjugates for 0.5 hr. The reaction was then stopped by the Stop Solution. The signal corresponding to the amount of the bound hACE2 was measured by a plate reader at 450 nm. The resulting data were analyzed by Prism 7 (GraphPad) and plotted.

Pseudotyped SARS-CoV-2 neutralization assay. The 293T-hsACE2 stable cell line (Cat #C-HA101, Lot #TA060720C) and the pseudotyped SARS-CoV-2 (Wuhan-Hu-1 strain) particles with GFP (Cat #RVP-701G, Lot #CG-113A) or luciferase (Cat #RVP-701L, Lot #CL109A, and CL-114A) reporters were purchased from the Integral Molecular. The neutralization assay was carried out according to the manufacturers' protocols. In brief, 10-fold serially diluted Nbs were incubated with the pseudotyped SARS-CoV-2-GFP for 1 hr at 37° C. for screening, while 3- or 5-fold serially diluted Nbs/immunized serum/immunized VHH mixture was incubated with the pseudotyped SARS-CoV-2-luciferase for accurate measurements. At least eight concentrations were tested for each Nb. Pseudovirus in culture media without Nbs was used as a negative control. 100 μl of the mixtures were then incubated with 100 μl 293T-hsACE2 cells at 2.5×10e5 cells/ml in the 96-well plates. The infection took ˜72 hrs at 37° C. with 5% CO2. The GFP signals (ex488/em530) were read using the Tecan Spark 20M with auto-optimal settings, while the luciferase signal was measured using the Renilla-Glo luciferase assay system (Promega, Cat #E2720) with the luminometer at 1 ms integration time. The obtained relative fluorescent/luminescence signals (RFU/RLU) from the negative control wells were normalized and used to calculate the neutralization percentage at each concentration. For SARSCoV-2-GFP screening, the 49 tested Nbs were divided into 6 groups based on their lowest tested concentration of 100% neutralization. For SARS-CoV-2-luciferase, data was processed by Prism7 (GraphPad) to fit into a 4PL curve and to calculate the log IC50 (half-maximal inhibitory concentration).

SARS-CoV-2 Munich plaque reduction neutralization test (PRNT). Nbs were diluted in a 2- or 3-fold series in Opti-MEM (Thermo). Each Nb dilution (110 μl) was mixed with 110 μl of SARS-CoV-2 (Munich strain) containing 100 plaque-forming units (p.f.u.) of the virus in Opti-MEM. The serum-virus mixes (220 μl total) were incubated at 37° C. for 1 h, after which they were added dropwise onto confluent Vero E6 cell (ATCC® CRL-1586™) monolayers in the six-well plates. After incubation at 37° C., 5% (v/v) CO2 for 1 h, 2 ml of 0.1% (w/v) immunodiffusion agarose (MP Biomedicals) in Dulbecco's modified eagle medium (DMEM) (Thermo) with 10% (v/v) FBS and 1× pen-strep was added to each well. The cells were incubated at 37° C., 5% CO2 for 72 hrs. The agarose overlay was removed and the cell monolayer was fixed with 1 ml/well formaldehyde (Fisher) for 20 min at room temperature. Fixative was discarded and 1 ml/well of 1% (w/v) crystal violet in 10% (v/v) methanol was added. Plates were incubated at room temperature for 20 min and rinsed thoroughly with water. Plaques were then enumerated and the 50% plaque reduction neutralization titer (PRNT50) was calculated. A validated SARS-CoV-2 antibody-negative human serum control, a validated NIBSC SARS-CoV-2 plasma control, was obtained from the National Institute for Biological Standards and Control, UK) and an uninfected cells control were also performed to ensure that virus neutralization by antibodies was specific.

Thermostability analysis of Nbs. Nb thermostabilities were measured by differential scanning fluorimetry (DSF). To prepare DSF samples, Nbs were mixed with SYPRO orange dye (Invitrogen) in PBS to reach a final concentration of 2.5-15 μM. The samples were analyzed in triplicate using a 7900HT Fast Real-Time PCR System (Applied Biosystems) as previously described {cite Allen's paper}. The melting point was then calculated by the first derivatives method {Niesen, 2007}.

Surface plasmon resonance (SPR). Surface plasmon resonance (SPR, Biacore 3000 system, GE Healthcare) was used to measure Nb affinities. Briefly, recombinant RBD was immobilized to the flow channels of an activated CM5 sensor-chip. RBD was diluted to 10 μg/ml in 10 mM sodium acetate, pH 4.5, and injected into the SPR system at 5 μl/min for 420 s. The surface was then blocked by 1 M ethanolamine-HCl (pH 8.5). For each Nb analyte, a series of dilution (spanning ˜1,000-fold concentration range) was injected in duplicate, with HBS-EP+ running buffer (GE-Healthcare) at a flow rate of 20-30 μl/min for 120-180 s, followed by a dissociation time of 10-20 mins. Between each injection, the sensor chip surface was regenerated twice with a low pH buffer containing 10 mM glycine-HCl (pH 1.5-2.5) at a flow rate of 40-50 μl/min for 30 s-1 min. Binding sensorgrams for each Nb were processed and analyzed using BIAevaluation by fitting with 1:1 Langmuir model.

Phylogenetic tree analysis and sequence logo. Sequences were first aligned and numbered according to Martin's numbering scheme by ANARCI (J. Dunbar et al., 2016). The phylogenetic tree was constructed from aligned sequences by Molecular Evolutionary Genetics Analysis (MEGA) (S. Kumar et al., 2018) using the Maximum Likelihood method. The sequence logo was plotted from aligned sequences by logomaker (A. Tareen et al., 2020).

Epitope screening by size exclusion chromatography (SEC). Recombinant RBD and Nb proteins were mixed at a ratio of 1:1 (w:w) and incubated at 4° C. for 1 hr. The complexes were analyzed by the SEC (Superdex75, GE Healthcare) at a low rate of 0.4 ml/min for 1 hr using a running buffer of 20 mM HEPES, 150 mM NaCl, pH 7.5. Protein signals were detected by ultraviolet light absorbance at 280 nm.

Clustering and phylogenetic tree analysis. A phylogenetic tree was generated by Clustal Omega {Sievers, 2014} with the input of unique NbHSA CDR3 sequences and the adjacent framework sequences (i.e., YYCAA (SEQ ID NO: 179) to the N-terminus and WGQG (SEQ ID NO: 180) to the C-terminus of CDR3s) to help alignments. The data was plotted by ITol (Interactive Tree of Life) {Letunic, 2007}. Isoelectric points and hydrophobicities of the CDR3s were calculated using the BioPython library. The sequence logo was plotted using WebLogo {Crooks, 2004}.

Chemical cross-linking and mass spectrometry (CXMS). Recombinant Nbs were first pre-incubated with the trypsin resin for approximately 2-5 mins to remove the N terminal T7 tag, which is highly reactive to the crosslinker. Nb was incubated with RBD in PBS at 4° C. for 1 hr to allow the formation of the complex. The reconstituted complexes were then cross-linked with 2 mM disuccinimidyl suberate (DSS, ThermoFisher Scientific) for 25 min at 25° C. with gentle agitation. The reaction was then quenched with 50 mM ammonium bicarbonate (ABC) for 10 min at room temperature. After protein reduction and alkylation, the cross-linked samples were separated by a 4-12% SDS-PAGE gel (NuPAGE, Thermo Fisher). The regions corresponding to the monomeric, cross-linked species (˜45-50 kDa) were sliced and digested in-gel with trypsin and Lys-C, or chymotrypsin {Shi, 2014; Shi, 2015; Xiang, 2020}. After efficient proteolysis, the cross-link peptide mixtures were desalted and analyzed with a nano-LC 1200 (Thermo Fisher) coupled to a Q Exactive™ HF-X Hybrid Quadrupole-Orbitrap™ mass spectrometer (Thermo Fisher). The cross-linked peptides were loaded onto a Picochip column (C18, 3 μm particle size, 300 Å pore size, 50 μm×10.5 cm; New Objective) and eluted using a 60 min LC gradient: 5% B-8% B, 0-5 min; 8% B-32% B, 5-45 min; 32% B-100% B, 45-49 min; 100% B, 49-54 min; 100% B-5% B, 54 min-54 min 10 sec; 5% B, 54 min 10 sec-60 min 10 sec; mobile phase A consisted of 0.1% formic acid (FA), and mobile phase B consisted of 0.1% FA in 80% acetonitrile. The QE HF-X instrument was operated in the data-dependent mode. The top 8 most abundant ions (with the mass range of 380 to 2,000 and the charge state of +3 to +7) were fragmented by high-energy collisional dissociation (normalized HCD energy 27). The target resolution was 120,000 for MS and 15,000 for MS/MS analyses. The quadrupole isolation window was 1.8 Th and the maximum injection time for MS/MS was set at 120 ms. After the MS analysis, the data was searched by pLink for the identification of cross-linked peptides. The mass accuracy was specified as 10 and 20 p.p.m. for MS and MS/MS, respectively. Other search parameters included cysteine carbamidomethylation as a fixed modification and methionine oxidation as a variable modification. A maximum of three trypsin missed-cleavage sites was allowed. Initial search results were obtained using the default 5% false discovery rate, estimated using a target-decoy search strategy. The crosslink spectra were manually checked as previously described {Shi, 2014; Shi, 2015; Xiang, 2020}.

Integrative structural modeling. Structural models for Nbs were obtained using a multi-template comparative modeling protocol of MODELLER {Sali, 1993}. Next, the CDR3 loop {Fiser, 2003} was refined and the top 5 scoring loop conformations were selected for the downstream docking in addition to 5 models from comparative modeling. Each Nb model was then docked to the RBD structure (PDB 61zg) by an antibody-antigen docking protocol of PatchDock software that focuses the search to the CDRs and optimizes CXMS-based distance restraints satisfaction {Schneidman-Duhovny, 2012; Schneidman-Duhovny, 2020}. A restraint was considered satisfied if the Ca-Ca distance between the cross-linked residues was within 28 Å for DSS cross-linkers. The models were then re-scored by a statistical potential SOAP {Dong, 2013}. The antigen interface residues (distance <6 Å from Nb atoms) among the top 10 scoring models, according to the SOAP score, were used to determine the epitopes. The convergence was measured as the average RMSD among the ten top-scoring models.

Crystallization, data collection, and structure determination of RBD-Nb20 complex. Crystallization trials were performed with the Crystal Gryphon robot (Art Robbins). The RBD-Nb20 complex was crystallized using the sitting-drop vapor diffusion method at 17° C. The crystals were obtained in conditions containing 100 mM sodium cacodylate pH 6.5 and 1 M sodium citrate. For data collection, the crystals were transferred to the reservoir solution supplemented with 20% glycerol before freezing in liquid nitrogen. X-ray diffraction data were collected at the Advanced Photon Source (APS) beamline 23IDB of GM/CA with a 10 μm diameter microbeam. The data were processed using HKL2000 (A. J. McCoy et al., 2007). Diffraction data from six crystals were merged to obtain a complete dataset with a resolution of 3.3 Å.

The structure was determined by the molecular replacement method in Phaser (P. D. Adams et al., 2010) using the crystal structures of RBD (PDB 6LZG) and an Nb (VHH-72, PDB 6WAQ) as search models. The initial model was refined in Phenix (P. Emsley et al., 2004) and adjusted in COOT (C. J. Williams et al., 2018). The model quality was checked by MolProbity (T. D. Goddard et al., 2018).

Nb21 comparative modeling was done using the Nb20 structure as a template in MODELLER. All structure visualization figures were prepared using UCSF ChimeraX (F. H. Niesen et al., 2007).

Nb stability test. For the stability test, Nb was eluted and collected in the SEC running buffer (20 mM HEPES, 150 mM NaCl, pH 7.5) and then concentrated to 1 ml (1 mg/ml). 0.5 ml of the concentrated Nb was lyophilized by snap freezing in liquid nitrogen before dried in a speed-vac. ddH2O was then used to reconstitute the Nb. The other 0.5 ml was aerosolized by using a portable mesh atomizer nebulizer (MayLuck). No obvious dead volume was observed. The aerosols were collected in a microcentrifuge tube. SEC analysis and pseudovirus neutralization assays were performed as described above.

Size exclusion chromatography (SEC). The recombinant RBD and selected Nbs were mixed at a molar ratio of 1:2 and incubated at 4° C. for 1 hr in the SEC buffer. The complexes were analyzed by SEC (Superdex75, GE LifeSciences). Protein signals were detected by ultraviolet light absorbance at 280 nm.

Data collection and structure determination. Crystals were collected and were frozen in liquid nitrogen. Data collection was performed at beamline 23-ID of GM/CA@APS at the Advanced Photon Source. Microbeams of 10 or 20 μM diameter were used to acquire all diffraction data.

### Example 2. Methods of Identifying SARS-CoV-2 Neutralizing Nanobodies and Computer Implemented Methods

The SARS-CoV-2 neutralizing nanobodies disclosed herein were developed using our integrative proteomic platform for in-depth discovery, classification, and high-throughput structural characterization of antigen-engaged Nb repertoires. This platform comprises a method of identifying a group of complementarity determining region (CDR)3 region SARS-CoV-2 nanobody amino acid sequences (CDR3 sequences) wherein a reduced number of the CDR3 sequences are false positives as compared to a control, the method comprising:
a. obtaining a blood sample from a camelid immunized with a SARS-CoV-2 antigen;
b. using the blood sample to obtain a nanobody cDNA library;
c. identifying the sequence of each cDNA in the library;
d. isolating nanobodies from the same or a second blood sample from the camelid immunized with the antigen;
e. digesting the nanobodies with trypsin or chymotrypsin to create a group of digestion products;
f. performing a mass spectrometry analysis of the digestion products to obtain mass spectrometry data;
g. selecting sequences identified in step c. that correlate with the mass spectrometry data;
h. identifying sequences of CDR3 regions in the sequences from step g.; and
i. selecting from the CDR3 region sequences of step h. those sequences having equal to or more than a required fragmentation coverage percentage; wherein the selected sequences comprise a group having the reduced number of false positive CDR3 sequences.

In some embodiments, the method further comprises creating a CDR3 peptide having a sequence identified in step i. The CDR3 peptide can comprises a sequence selected from the group consisting of SEQ ID NO:82 through SEQ ID NO:152. In some embodiments, the method further comprises creating a SARS-CoV-2 neutralizing nanobody comprising a CDR3 region having a sequence identified in step i. The SARS-CoV-2 neutralizing nanobody can comprise a sequence selected from the group consisting of SEQ ID NO:1 through SEQ ID NO:81.

Also included herein are computer-implemented methods, comprising:
a. receiving a SARS-CoV-2 nanobody peptide sequence;
b. identifying a plurality of complementarity-determining region (CDR) regions of the nanobody peptide sequence, the CDR regions including CDR3 regions;
c. applying a fragmentation filter to discard one or more false positive CDR3 regions of the nanobody peptide sequence;
d. quantifying an abundance of one or more non-discarded CDR3 regions of the nanobody peptide sequence; and
e. inferring an antigen affinity based on the quantified abundance of the one or more non-discarded CDR3 regions of the nanobody peptide sequence.

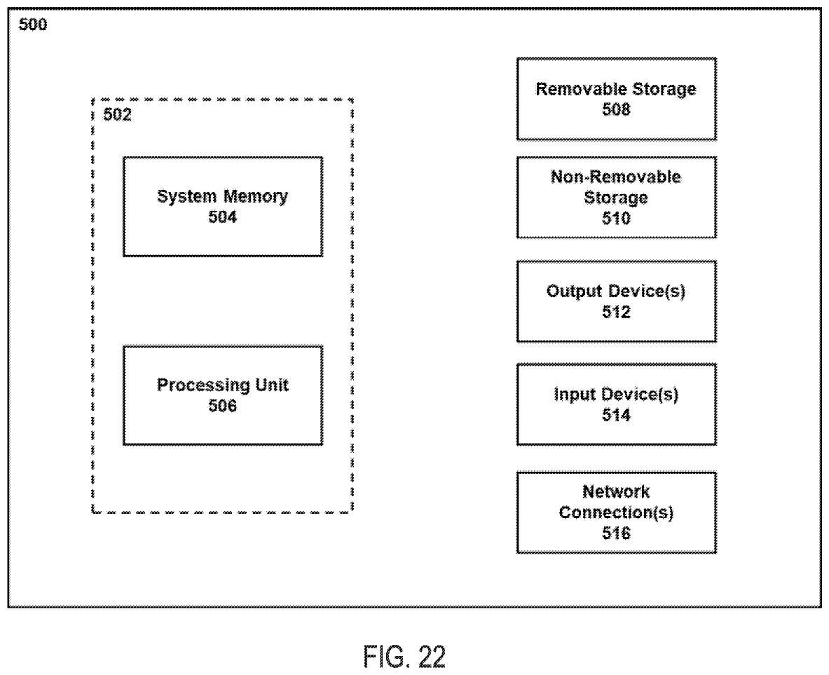

It should be appreciated that the logical operations described herein with respect to the various figures may be implemented (1) as a sequence of computer implemented acts or program modules (i.e., software) running on a computing device (e.g., the computing device described in FIG. 22), (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special purpose digital logic, and any combination thereof. It should also be appreciated that more or fewer operations may be performed than shown in the figures and described herein. These operations may also be performed in a different order than those described herein.

Referring to FIG. 22, an example computing device 500 upon which the methods described herein may be implemented is illustrated. It should be understood that the example computing device 500 is only one example of a suitable computing environment upon which the methods described herein may be implemented. Optionally, the computing device 500 can be a well-known computing system including, but not limited to, personal computers, servers, handheld or laptop devices, multiprocessor systems, microprocessor-based systems, network personal computers (PCs), minicomputers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media.

In its most basic configuration, computing device 500 typically includes at least one processing unit 506 and system memory 504. Depending on the exact configuration and type of computing device, system memory 504 may be volatile (such as random access memory (RAM)), non-volatile (such as read-only memory (ROM), flash memory, etc.), or some combination of the two. This most basic configuration is illustrated in FIG. 22 by dashed line 502. The processing unit 506 may be a standard programmable processor that performs arithmetic and logic operations necessary for operation of the computing device 500. The computing device 500 may also include a bus or other communication mechanism for communicating information among various components of the computing device 500.

Computing device 500 may have additional features/functionality. For example, computing device 500 may include additional storage such as removable storage 508 and non-removable storage 510 including, but not limited to, magnetic or optical disks or tapes. Computing device 500 may also contain network connection(s) 516 that allow the device to communicate with other devices. Computing device 500 may also have input device(s) 514 such as a keyboard, mouse, touch screen, etc. Output device(s) 512 such as a display, speakers, printer, etc. may also be included. The additional devices may be connected to the bus in order to facilitate communication of data among the components of the computing device 500. All these devices are well known in the art and need not be discussed at length here.

The processing unit 506 may be configured to execute program code encoded in tangible, computer-readable media. Tangible, computer-readable media refers to any media that is capable of providing data that causes the computing device 500 (i.e., a machine) to operate in a particular fashion. Various computer-readable media may be utilized to provide instructions to the processing unit 506 for execution. Example tangible, computer-readable media may include, but is not limited to, volatile media, non-volatile media, removable media and non-removable media implemented in any method or technology for storage of information such as computer readable instructions, data structures, program modules or other data. System memory 504, removable storage 508, and non-removable storage 510 are all examples of tangible, computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 506 may execute program code stored in the system memory 504. For example, the bus may carry data to the system memory 504, from which the processing unit 506 receives and executes instructions. The data received by the system memory 504 may optionally be stored on the removable storage 508 or the non-removable storage 510 before or after execution by the processing unit 506.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high level procedural or object-oriented programming language to communicate with a computer system. However, the program(s) can be implemented in assembly or machine language, if desired. In any case, the language may be a compiled or interpreted language and it may be combined with hardware implementations.

### Example 3. Super-Immunity by Broadly Protective Sarbecovirus Nanobodies

### Methods and Materials

### Purification of Recombinant Sarbecovirus RBDs and SARS-CoV-2 Spike

The mammalian expression vectors encoding the RBDs of RaTG13-CoV (GenBank QHR63300; S protein residues 319-541), SHCO14-CoV (GenBank KC881005; residues 307-524), Rs4081-CoV (GenBank KY417143; residues 310-515), pangolin17-CoV (GenBank QIA48632; residues 317-539), RmYNO2-CoV (GSAID EPI_ISL_412977; residues 298-503), Rf1-CoV (GenBank DQ412042; residues 310-515), W1V1-CoV (GenBank KF367457; residues 307-528), Yun11-CoV (GenBank JX993988; residues 310-515), BM-4831-CoV (GenBank NC014470; residues 310-530), BtkY72-CoV (GenBank KY352407; residues 309-530) with an N-terminal Mu phosphatase signal peptide and C-terminal His-tag were a kind gift from Pamela J. Bjorkman's lab, Caltech. Plasmids of the RBDs for the following sarbecovirus strains were synthesized from Synbio Technologies in a similar way: SARS-CoV-2 (GenBank MN985325.1; S protein residues 319-539), SARS-CoV (GenBank AAP13441.1; residues 318-510), Rs7327-CoV (GenBank KY417151.1; residues 319-518), Rs4092-CoV (GenBank KY417145.1; residues 314-496), YN2013-CoV (GenBank KJ473816.1; residues 314-496), ZC45-CoV (GenBank MG772933.1; residues 327-509), HKU3-1-CoV (GenBank DQ022305.2; residues 322-505), Shaanxi2011-CoV (GenBank JX993987.1; residues 321-502) and Rp3-CoV (GenBank DQ071615.1; residues 322-504). The cDNA encoding SARS-CoV-2 spike HexaPro (S) was obtained from Addgene (Cat #154754). To express the proteins, Expi293F cells were transiently transfected with the plasmid using the ExpiFectamine 293 kit (Thermo, Cat #A14635). After 24 hrs of transfection, enhancers were added to further boost protein expression. Cell culture was harvested 5-6 days after transfection and the supernatant was collected by high-speed centrifugation at 21,000×g for 30 min. The secreted proteins in the supernatant were purified using His-Cobalt resin (Thermo). Eluted proteins were then concentrated and further purified by size-exclusion chromatography using a Superose 6 10/300 (for S) or Superdex 75 column (for RBDs, Cytiva) in a buffer composed of 20 mM Hepes pH 7.5 and 150 mM NaCl. SARS-CoV-2 RBD variants are obtained from the Acro Biosystems.

Camelid Immunization and Proteomic Identification of psNbs

A Llama “Wally” was previously immunized as described {reference} and the collection bleed is referred to as the first bleed. Three weeks after the blood collection, the same llama was immunized again by four consecutive boosts every 2 weeks. Blood was collected 10 days after the final boost and is referred to as 2^{nd} bleed or booster. All the above procedures were performed by Capralogics, Inc. following the IACUC protocol. Next-generation sequencing (NGS) of the V_{H}H repertoire of the 2^{nd} bleed was performed by Illumina MiSeq in the UPMC Genome Center as described.

Plasma was first purified from the two immunized bleeds by the Ficoll gradient (Sigma). Polyclonal V_{H}Hs were then isolated from the plasma by a two-step purification using protein G and protein A sepharose beads (Marvelgent). RBD_{SARS-CoV-2}, RBD_{SARS-CoV}, RBD_{RmYNO2} and RBD_{BM-4831} are coupled to the resin as affinity handles to isolate each RBD-specific V_{H}Hs. After proteolysis by trypsin and chymotrypsin, peptide mixtures were desalted and analyzed with a nano-LC 1200 that is coupled online with a Q Exactive™ HF-X Hybrid Quadrupole Orbitrap™ mass spectrometer. Proteomic analysis was performed as previously described and AugurLlama was used to facilitate reliable identification, label-free quantification, and classification of high-affinity pan-sarbecovirus Nbs.

### Nb DNA Synthesis and Cloning

The monomeric Nb genes were codon-optimized and synthesized (Synbio). All the Nb DNA sequences were cloned into a pET-21b(+) vector using EcoRI and HindIII restriction sites. The monomeric Nbs 5P-118 and 5P-132 were also cloned into a pET-22b(+) vector at the BamHI and XhoI sites for periplasmic expression. To produce a heterodimeric Nb 132-118, the DNA fragment of the monomeric Nb 5P-118 was first PCR amplified from the pET-21b(+) vector to introduce a linker sequence and two restriction sites of XhoI and HindIII that facilitate cloning (Primer1: cccAAGCTTggtggtggtggtagtggtggtggtggtagtggtggtggtggtagtCAaGTTCAACTGGTTGAATCT G (SEQ ID NO: 2128);

Primer2: ccgCTCGAGTGCGGCCGCcagtttGCTACTAACGGTAACTT) (SEQ ID NO: 2129). The PCR fragment was then inserted into the 5P-132 pET-21b(+) vector at the same restriction sites to produce the heterodimer 5P-132-(GGGGS)₃-5P-118.

### Purification of Nbs

Nb DNA constructs were transformed into BL21(DE3) cells and plated on Agar with 50 μg/ml ampicillin at 37° C. overnight. Cells were cultured in an LB broth to reach an O.D. of ˜0.5-0.6 before IPTG (0.5-1 mM) induction at 16/20° C. overnight. Cells were then harvested, sonicated, and lysed on ice with a lysis buffer (1×PBS, 150 mM NaCl, 0.2% TX-100 with protease inhibitor). After cell lysis, protein extracts were collected by centrifugation at 21,000×g for 10 mins and the his-tagged Nbs were purified by the Cobalt resin (Thermo) and natively eluted with a buffer containing 150 mM imidazole buffer. Eluted Nbs were subsequently dialyzed in a dialysis buffer (e.g., 1×DPBS, pH 7.4 or SEC buffer).

For the periplasmic preparation of Nbs (5P-118 and 5P-132), cell pellets were resuspended in the TES buffer (0.1 M Tris-HCl, pH 8.0; 0.25 mM EDTA, pH 8.0; 0.25 M Sucrose) and incubated on ice for 30 min. The supernatants were collected by centrifugation and subsequently dialyzed to DPBS. The resulting Nbs were then purified by Cobalt resin as described above.

### ELISA (Enzyme-Linked Immunosorbent Assay)

Indirect ELISA was carried out to evaluate the camelid immune responses of the total single-chain only antibody (VHH) to an RBD and to quantify the relative affinities of psNbs. The 96-well ELISA plate (R&D system) was coated with the RBD protein or the HEK-293T cell lysate at an amount of approximately 3-5 ng per well in a coating buffer (15 mM sodium carbonate, 35 mM sodium bicarbonate, pH 9.6) overnight at 4° C., with subsequent blockage with a blocking buffer (DPBS, 0.05% Tween 20, 5% milk) at room temperature for 2 hours. To test the immune response, the total VHH was serially 5-fold diluted in the blocking buffer and then incubated with the RBD-coated wells at room temperature for 2 hours. HRP-conjugated secondary antibodies against llama Fc were diluted 1:75,00 in the blocking buffer and incubated with each well for an additional 1 hour at room temperature. For the initial screening of Nb binding against 4 RBDs, scramble Nbs that do not bind the RBDs were used for negative controls. Nbs were serially 10-fold diluted from 1 μM to 1 nM in the blocking buffer. For the Nb affinity measurements against 24 RBDs, Nbs were serially 4-fold diluted. Nb dilutions were incubated for 2 hours at room temperature. HRP-conjugated secondary antibodies against the T7-tag were diluted at 1:5,000 or 1:75,00 in the blocking buffer and incubated for 1 hour at room temperature. Three washes with 1×PBST (DPBS v/v, 0.05% Tween 20) were carried out to remove nonspecific absorbances between each incubation. After the final wash, the samples were further incubated under dark with freshly prepared w3,3′,5,5′-Tetramethylbenzidine (TMB) substrate for 10 mins at room temperature to develop the signals. After the STOP solution (R&D system), the plates were read at multiple wavelengths (450 nm and 550 nm) on a plate reader (Multiskan GO, Thermo Fisher). The raw data was processed by Prism 9 (GraphPad) to fit into a 4PL curve and to calculate log IC50.

Competitive ELISA with Recombinant hACE2

A 96-well plate was pre-coated with recombinant spike-6P at 2 μg/ml at 4° C. overnight. Nbs were 5-fold diluted (from 0.2/1/5 μM to 12.8/64/320 pM) in the assay buffer with a final amount of 50 ng biotinylated hACE2 at each concentration and then incubated with the plate at room temperature for 2 hrs. The plate was washed by the washing buffer to remove the unbound hACE2. 1:6000 diluted Pierce™ High Sensitivity NeutrAvidin™-HRP antibodies (Thermo Fisher cat #31030) were incubated with the plate for 1 hour at room temperature. TMB solution was added to react with the HRP conjugates for 10 minutes. The reaction was then stopped by the Stop Solution. The signal corresponding to the amount of the bound hACE2 was measured by a plate reader at 450 nm and 550 nm. The wells without Nbs were used as control to calculate the percentage of hACE2 signal. The resulting data were analyzed by Prism 9 (GraphPad) and plotted.

psNb Epitope Analysis by Competitive Size Exclusion Chromatography (SEC)

Analytical size exclusion chromatography was performed with a superdex 75 increase GL column (column volume: 24 mL, Cytiva) on a Shimadzu HPLC system equipped with a multi-wavelength UV detector at a flow rate of 0.3 mL/min. The column was connected and placed in a column oven set to 15° C., and the SEC running buffer was 10 mM HEPES pH 7.1, 150 mM NaCl. A reference SEC profile for RBD with class I (Nb21), class II (Nb105 or 5p34) and class III (Nb36) was performed after column equilibration. Subsequently, three separate runs with RBD and specified psNb were performed after mixing with (i) class I and class II, (ii) class II and class III and (iii) class I and class III nanobodies. A supershift of the peak, at the same or to the left of the RBD and three Nbs peak in the reference profile, in run (i) but not run (ii) and (iii) sorts a psNb into class III. Similarly, psNb with a supershift in run (ii) but not run (i) and (iii) belongs to class I, and a supershift in run (iii) but not run (i) and (ii) indicates a class II epitope binder. When supershifts were observed in all three runs, the psNb was sorted into a new class.

### Nb Affinity Measurement by SPR

Surface plasmon resonance (SPR, Biacore 3000 system, GE Healthcare) was used to measure Nb affinities. RBD proteins were_immobilized on the activated CM5 sensor-chip in pH 4.0 10 mM sodium acetate buffer. The surface of the sensor chip was blocked by 1 M Tris-HCl (pH 8.5). For each Nb analyte, a series of concentration dilutions was injected in HBS-EP running buffer (GE-Healthcare), at a flow rate of 20 μl/min for 180s, followed by a dissociation time of 15 mins. Between each injection, the sensor chip surface was regenerated with the low pH buffer containing 10 mM glycine-HCl (pH 1.5-2.0). The regeneration was performed with a flow rate of 30-40 μl/min for 30-45 s. The measurements were duplicated, and only highly reproducible data was used for analysis. Binding sensorgrams for each Nb were processed and analyzed using BIAevaluation by fitting with the 1:1 Langmuir model or the 1:1 Langmuir model with the drifting baseline.

### Pseudotyped SARS-CoV-2 Neutralization Assay

The 293T-hsACE2 stable cell line (Cat #C-HA101, Lot #TA060720C) and the pseudotyped SARS-CoV-2 (Wuhan-Hu-1 strain, D614G, Alpha, Beta, Lambda, Delta and Omicron) particles with luciferase reporters were purchased from the Integral Molecular. The neutralization assay was carried out according to the manufacturers' protocols. In brief, 3- or 5-fold serially diluted Nbs/immunized V_{H}H mixture was incubated with the pseudotyped SARS-CoV-2-luciferase for accurate measurements. At least seven concentrations were tested for each Nb and at least two repeats of each Nb were done. Pseudovirus in culture media without Nbs was used as a negative control. 100 μl of the mixtures were then incubated with 100 μl 293T-hsACE2 cells at 2.5×10e⁵ cells/ml in the 96-well plates. The infection took ˜72 hrs at 37° C. with 5% CO₂. The luciferase signal was measured using the Renilla-Glo luciferase assay system (Promega, Cat #E2720) with the luminometer at 1 ms integration time. The obtained relative luminescence signals (RLU) from the negative control wells were normalized and used to calculate the neutralization percentage at each concentration. Data was processed by Prism 9 (GraphPad) to fit into a 4PL curve and to calculate the log IC₅₀ (half-maximal inhibitory concentration).

### SARS-CoV-2 Munich and Delta Plaque Reduction Neutralization Test (PRNT)

Nbs were diluted in a 3- or 5-fold series in Opti-MEM (Thermo). Each Nb dilution (110 l) was mixed with 110 μl of SARS-CoV-2 (Munich strain) containing 100 plaque-forming units (p.f.u.) or 110 μl of SARS-CoV-2 (Delta strain, BEI Cat #NR55611) containing 50 p.f.u. of the virus in Opti-MEM. The Nb-virus mixes (220W total) were incubated at 37° C. for 1 h, after which they were added dropwise onto confluent Vero E6 cell (ATCC® CRL-1586™, for Munich) or Vero E6-TMPRSS2-T2A-ACE2 cells (BEI cat #NR-54970, for Delta) monolayers in the six-well plates. After incubation at 37° C., 5% (v/v) CO₂ for 1 h, 2 ml of 0.1% (w/v) immunodiffusion agarose (MP Biomedicals) for Munich strain and 0.25% (w/v) immunodiffusion agarose for Delta strain in Dulbecco's modified eagle medium (DMEM) (Thermo) with 10% (v/v) FBS and 1× pen-strep was added to each well. The cells were incubated at 37° C., 5% CO₂ for 72 hrs. The agarose overlay was removed and the cell monolayer was fixed with 1 ml/well formaldehyde (Fisher) for 20 min at room temperature. Fixative was discarded and 1 ml/well of 1% (w/v) crystal violet in 10% (v/v) methanol was added. Plates were incubated at room temperature for 20 min and rinsed thoroughly with water. Plaques were then enumerated and the 50% plaque reduction neutralization titer (PRNT₅₀) was calculated. A validated SARS-CoV-2 antibody-negative human serum control, a validated NIBSC SARS-CoV-2 plasma control, was obtained from the National Institute for Biological Standards and Control, UK) and an uninfected cells control were also performed to ensure that virus neutralization by antibodies was specific.

### Aerosolization of PiN Using a Soft-Mist Inhaler

Nb was eluted and collected in the SEC running buffer (20 mM HEPES, 150 mM NaCl, pH 7.5) and then concentrated to 0.5 ml (1 mg/ml). Nb was aerosolized by using a portable soft mist inhaler Pulmospray@(Resyca). Around 0.1-0.15 ml dead volume was observed in the syringe and connector. The aerosols were collected in a 50 ml falcon tube and SEC analysis was performed as described above.

### Cryo-Electron Microscopy Data Collection and Image Processing

SARS-CoV-2 HexaPro spike at 1.1 mg/mL was incubated with 1.5-fold molar excess of specified Nbs at room temperature for two hours. 3.5 μL of 1 to 3 dilution sample using HBS buffer with 1% glycerol was applied onto a freshly glow discharged UltraAuFoil R1.2/1.3 grid (300 mesh) and plunge frozen using a vitrobot Mark IV (Thermo Fisher Scientific) with a blot force of 0 and 2.5 s blot time at 100% humidity at 4° C. The cryoEM datasets were collected at either CWRU or PNCC.

For CWRU datasets, movie stacks were recorded using an FEI Titan Krios transmission electron microscope G3i operated at 300 keV and equipped with a Gatan K3 direct electron detector and Gatan BioQuantum image filter operated in zero-loss mode with a slit width of 20 eV. Automated data collection was carried out using serialEM at a nominal magnification of 81,000× with a physical pixel size of 1.07 Å/pixel (0.535 Å/pixel at super-resolution) for spike:nanobody complexes and 165,000× with a physical pixel size of 0.52 Å/pixel (0.26 Å/pixel at super-resolution) for RBD:nanobodies complexes. Each movie stack was collected with a dose rate of 18 electron/pixel/s in super-resolution mode and fractionated in 40 frames with two-second exposure, resulting in a total dose of ˜31.4 e/Å2 for 81,000× and in 32 frames with 1.5-second exposure, resulting in a total dose of ˜103.4 e/Å2 for 81,000×. The number of movies for a specified dataset was listed in Supplementary Table X. The defocus range was set to between −0.5 and −2 μm.

For PNCC datasets, movie stacks were recorded using an FEI Titan Krios transmission electron microscope G3i operated at 300 keV and equipped with a Gatan K3 direct electron detector and Gatan BioContinuum image filter operated in zero-loss mode with a slit width of 20 eV. Automated data collection was carried out using serialEM at a nominal magnification of 64,000× with a physical pixel size of 1.329 Å/pixel (0.6645 Å/pixel at super-resolution). The dose rate was determined over a sample hole to calculate the exposure time resulting in a total dose of 40 e/Å2 and the exposures were fractionated into a total of 40 frames. The number of movies for a specified dataset was listed in Supplementary Table X. The defocus range was set to between −0.5 and −2 μm.

Image processing was performed on-the-fly using CryoSPARC Live version 3.2. The particles were automatically picked using the blob picker with 240 Å or 100 Å diameter for spike:nanobody or RBD:nanobodies, respectively. Reference-free 2D classification was performed in streaming with 200 classes and limited maximum resolution to 18 Å. Upon the completion of data collection, the selected particles from the good 2D class averages were subjected for another round of 2D classification with 200 classes, and particles from classes with resolution better than 10 Å and ECA less than 2 were selected for subsequent analysis. These 2D classes were submitted for a “rebalance 2D” job type to trim particles from dominant views. The rebalanced particle set was then used for ab-initio reconstruction to generate the initial volume. 3D refinement was first carried out using non-uniform refinement using ab-initio volume as the reference without mask. To resolve the density for nanobodies, an RBD structure (PDB 6MOJ) was docked into the cryoEM density and the structural model for nanobody was manually placed into the additional density accounted for nanobodies to ensure the correct orientation. The resulting RBD:nanobody model was used to generate the mask for focused 3D classification in CryoSPARC version 3.3.1 with six classes and target resolution of 6 Å, and PCA was chosen as the initialization mode with the parameter “number of components” set for 2. 3D classes with both RBD and nanobody densities well resolved were selected for sequent new local refinement with the mask around RBD and nanobody. The gold-standard Fourier shell correlation (FSC) of 0.143 criterion was used to report the resolution and the FSC curves were corrected for the effects of a soft mask using high-resolution noise substitution.

### Model Building and Refinement

PDB entry, 7CAK, was used as the initial model for spike excluding RBD and the missing fragment for residue 621 to 640 was modeled de novo in Coot. RBD from PDB entry, 6MOJ, was used as the initial model and nanobodies structures were generated using ColabFold. After assembling individual components into a single PDB file, the models were refined into the composite map using phenix.real_space_refine. The glycans were built using the carbohydrate module in Coot. Models were manually corrected in Coot (version 9.6.0) between rounds of read-space refinement in Phenix. Figure panels depicting cryo-EM maps or atomic models generated using ChimeraX (Pettersen et al., 2021). Maps colored by local resolution were generated using RELION 3.1 (Zivanov et al., 2018).

Crystallographic Analysis of psNbs with RBD

The receptor-binding domain (RBD) of the SARS-CoV-2 spike (S) protein (GenBank: QHD43416.1) used in crystallographic study, was cloned into a customized pFastBac vector, and fused with an N-terminal gp67 signal peptide and C-terminal His6 tag. Recombinant bacmids encoding each RBDs were generated using the Bac-to-Bac system (Thermo Fisher Scientific) followed by transfection into Sf9 cells using FuGENE HD (Promega) to produce baculoviruses for RBD expression. RBD protein was expressed in High Five cells (Thermo Fisher Scientific) with suspension culture shaking at 110 r.p.m. at 28° C. for 72 hours after the baculovirus transduction at an MOI of 5 to 10. Supernatant containing RBD protein was then concentrated using a 10 kDa MW cutoff Centramate cassette (Pall Corporation) followed by affinity chromatography using Ni-NTA resin (QIAGEN) and size exclusion chromatography using a HiLoad Superdex 200 pg column (Cytiva). The purified protein sample was buffer exchanged into 20 mM Tris-HCl pH 7.4 and 150 mM NaCl and concentrated for binding analysis and crystallographic studies.

Nb117+SARS-CoV-2 RBD and 5p118+SARS-CoV-2 RBD+CC12.1 complex were formed by mixing each of the protein components in an equimolar ratio and incubating overnight at 4° C. 384 conditions of the JCSG Core Suite (Qiagen) were used for setting-up trays for screening the Nb117 complex (12 mg/ml) and 5p-118 complex (14.3 mg/ml) on our robotic CrystalMation system (Rigaku) at Scripps Research. Crystallization trials were set-up by the vapor diffusion method in sitting drops containing 0.1 μl of protein complex and 0.1 μl of reservoir solution. Crystals appeared on day 3, were harvested on day 12, pre-equilibrated in cryoprotectant containing 0-10% ethylene glycol, and then flash cooled and stored in liquid nitrogen until data collection. X-ray diffraction data were collected at cryogenic temperature (100 K) at beamlines 23-ID-D of the Advanced Photon Source (APS) at Argonne National Laboratory and were collected from crystals grown in drops containing 20% polyethylene glycol 8000, 0.1 M NaCl, 0.1M CAPS pH 10.5 for the Nb117 complex and drops containing 40% MPD, 0.1M cacodylate pH 6.5, 5% (w/v) polyethylene glycol 8000 for the 5p118 complex. Collected data were processed with HKL2000. X-ray structures were solved by molecular replacement (MR) using PHASER with original MR models for the RBD and Nanobody from PDB 7JMW and PDB 7KN5. Iterative model building and refinement were carried out in COOT and PHENIX, respectively.

### RBD Epitope Analysis by ScanNet

The epitope propensity profile of SARS-CoV-2 RBD (PDB 7jvb) was computed by ScanNet, a state-of-the-art geometric deep learning model for structure-based protein binding site prediction. We used the B-cell epitope network (ScanNet-BCE) without evolutionary information, that was not trained on any SARS-CoV-1/2 antibody/antigen complex.

### Conservation and ScanNet Analysis of Viral Antigens

Dataset preparation. We collected all viral antibody—antigen complexes in PDB. Antigens were clustered at 70% sequence identity with a minimum length coverage of 15% using CD-HIT, the Bio.align pairwise sequence alignment module and CATH domain identifiers. Briefly, we found that (i) ngram-based clustering using CD-HIT with default parameters overestimated the number of clusters, while (ii) computing all the entries of the pairwise sequence similarity matrix was intractable for our set of several thousands structures. We instead proceeded as follows: for a given sequence identity cut-off T (e.g. 100), sequences were clustered with CD-HIT, and the resulting representatives were further clustered at T-20. For each pair of T-representatives with identical CATH identifier or same T-20 cluster, the sequence identity and coverage were evaluated by pairwise alignment and the corresponding T-clusters were merged if necessary. The process was iterated at T=100%, 95%, 90%, 70% sequence identity cut-offs and yielded satisfactory clusters. We further grouped together the following antigen clusters that had lower sequence identity but high structure similarity: (i) Influenza Hemagglutinin from strands H1N1, H3N2, H5N1, H7N9, H2N2 and (ii) Envelope protein of Dengue 1, Dengue 2, Dengue 4 and Zika. Only clusters with at least 7 unique antibodies were retained for further analysis, yielding 11 viral antigens.

Antibody hit rate calculation. We constructed a multiple sequence alignment for each antigen cluster using MAFFT and selected a representative structure with highest structure coverage and resolution. For each column of the alignment, the antibody hit rate was calculated as the fraction of unique antibodies binding it. For each complex, we identified the epitope residues as the antigen residues having at least one heavy atom within 4 Å of at least one antibody heavy atom. Our analysis was performed on the antigen surface residues (relative accessible surface area >=0.25, computed within the biological assembly for multimeric antigens using Bio.DSSP). Finally, the column-wise antibody hit rate was projected back onto the surface residues representative structure.

Calculation of antigen conservation and epitope propensity. For each representative antigen chain, a multiple sequence alignment was constructed by homology search on the UniRef30_2020_06 sequence database using HHBlits (4 iterations, default parameters). The alignment was deduplicated, hits with high gap content (>=25% of the alignment) were discarded and the 10K best hits were retained based on sequence identity. Each sequence was assigned a weight inversely proportional to the number of similar sequences found in the alignment (90% sequence identity cut-off). The amino acid frequency at each site fᵢ(a) was subsequently calculated and the residue-wise conservation score was defined as Cᵢ=Ln(20)−S(fᵢ) where S(fᵢ)=. Conservation scores range from 0 to ln(20)=2.99, higher is more conserved. We checked that this protocol correlated well with the ConSurf method based on phylogenetic trees {reference}. Epitope propensity scores were calculated with ScanNet-BCE.

RMSF calculation. Root mean square fluctuation of each RBD residue was calculated based on 100 ns Molecular Dynamics simulation trajectory. The simulation was run starting from the RBD structure (PDB 61zg) using Gromacs 2020 version with the CHARMM36m force field {Huang, 2017 #148}. The RBD structure was solvated in transferable intermolecular potential with 3 points (TIP3P) water molecules and ions were added to equalize the total system charge.

The steepest descent algorithm was used for initial energy minimization until the system converged at Fmax<1,000 kJ/(mol·nm). Then water and ions were allowed to equilibrate around the protein in a two-step equilibration process. The first part of equilibration was at a constant number of particles, volume, and temperature (NVT). The second part of equilibration was at a constant number of particles, pressure, and temperature (NPT). For both MD equilibration parts, positional restraints of k=1,000 kJ/(mol·nm2) were applied to heavy atoms of the protein, and the system was allowed to equilibrate at a reference temperature of 300 K, or reference pressure of 1 bar for 100 ps at a time step of 2 fs. Altogether 10,000 frames were saved for the RMSF analysis at intervals of 10 ps. To estimate average epitope RMSF, we defined epitope residues as residues with at least one atom within 4 Å from the Nb atom and averaged RMSF over epitope residues.

### Identification and Characterization of a Large Repertoire of Potent Pan-Sarbecovirus Nbs.

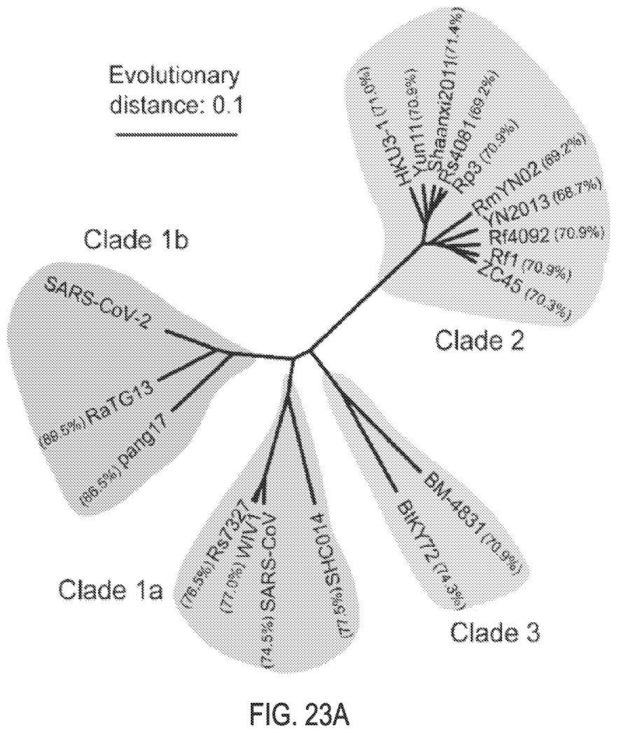

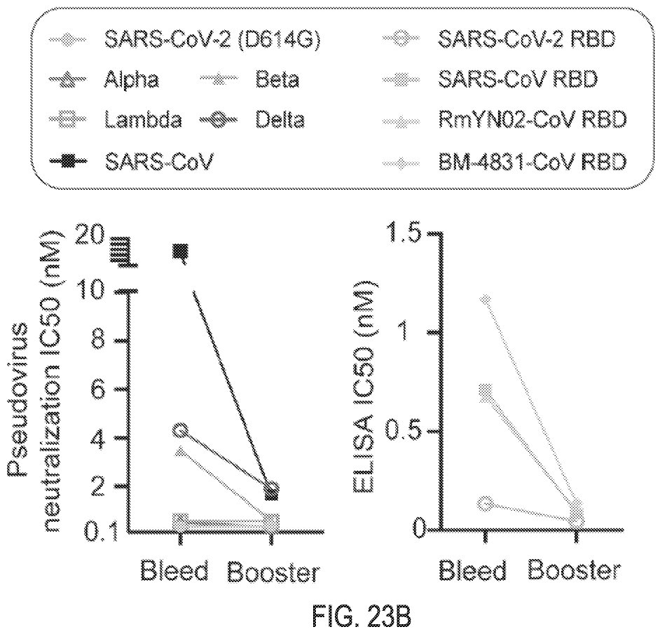

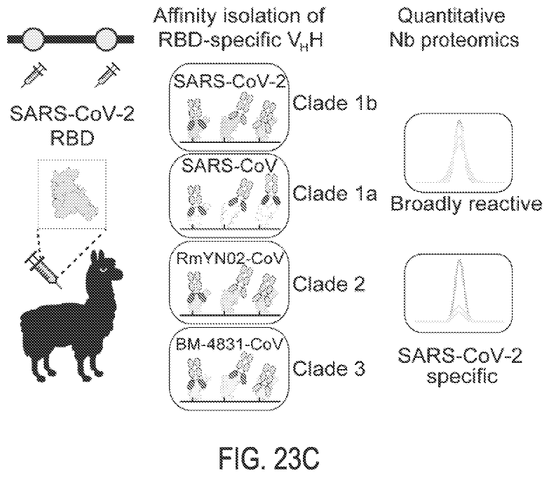

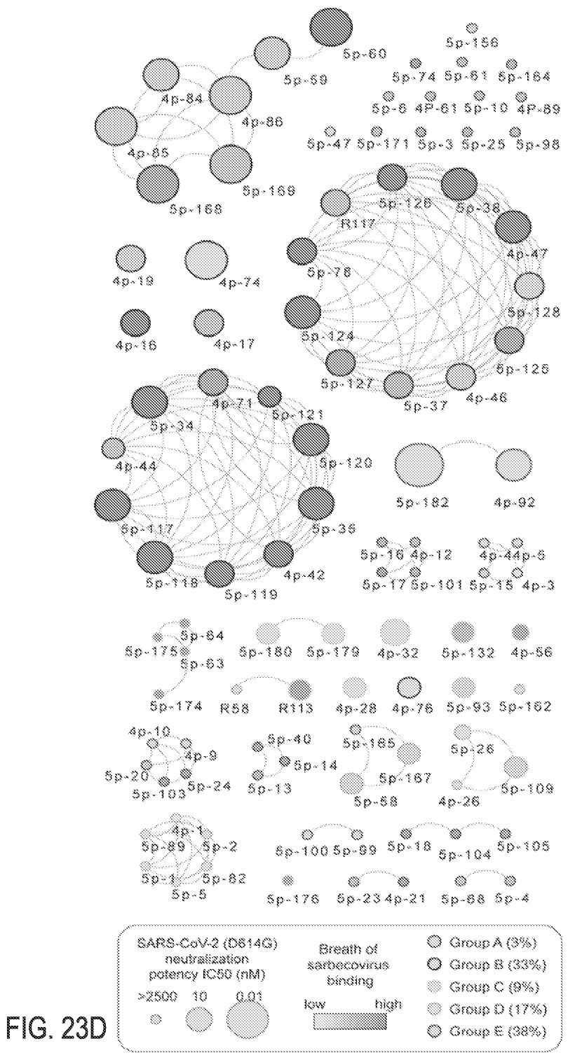

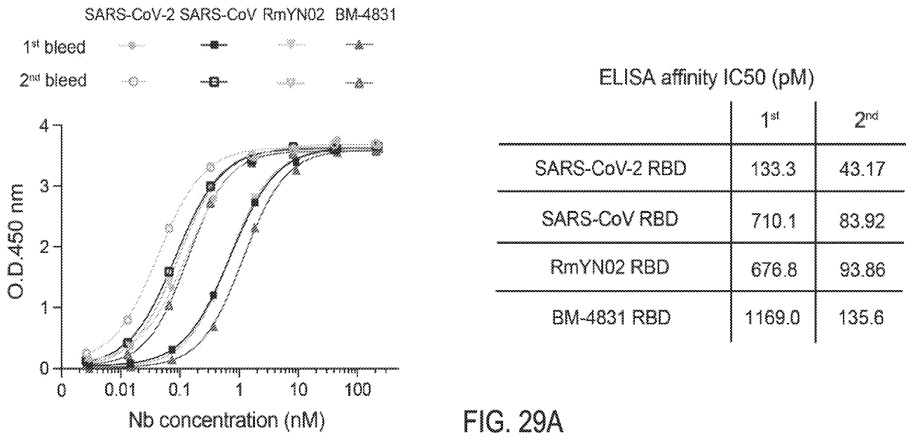

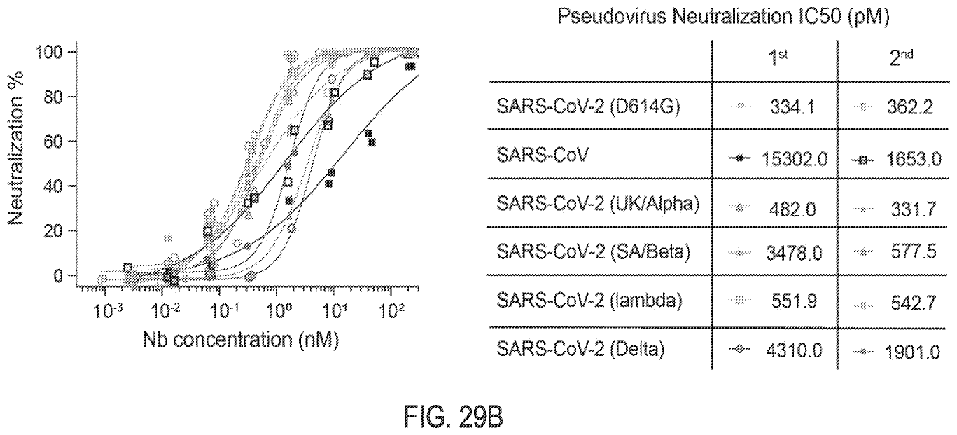

Single-chain VHH antibodies were isolated from two consecutive immunization bleeds. Immunizations span 6 months in which the first bleed was collected approximately 2 months after priming, followed by additional boosts for 3 months before the second bleed was collected (Methods). Polyclonal VHH mixtures show comparably high affinities to RBD_{SARS-CoV-2} between the first and second bleeds with overall ELISA IC50s of 0.13 nM to 0.04 nM, respectively (FIG. 23b, FIG. 29a). They also possess excellent neutralization potencies (IC50s between 0.3-0.6 nM) against the Wuhan-Hu-1 strain as well as Alpha and Lambda VOCs (FIG. 29b). Notably, compared to the initial VHHs which confer limited potencies against Beta, Delta, and SARS-COV, the potencies were substantially improved after the booster by 6.0, 2.3, and 9.3 folds, respectively, to 0.58, 1.90 and 1.65 nM (FIG. 29b). Surprisingly, the booster was also associated with strong and broad activities against the full spectrum of sarbeoviruses (FIG. 23a-23b). The relative binding affinities (ELISA IC50s) for RBDs from clade 1a (RBD_{SARS-CoV}), 2 (RBD_{RmYNO2-CoV}), and 3 (RBD_{BM-4831-CoV}) are 0.08, 0.09, and 0.14 nM, respectively, which correspond to the improvements of 8.5, 7.2, and 8.6 folds compared to the initial bleed (FIG. 29a).

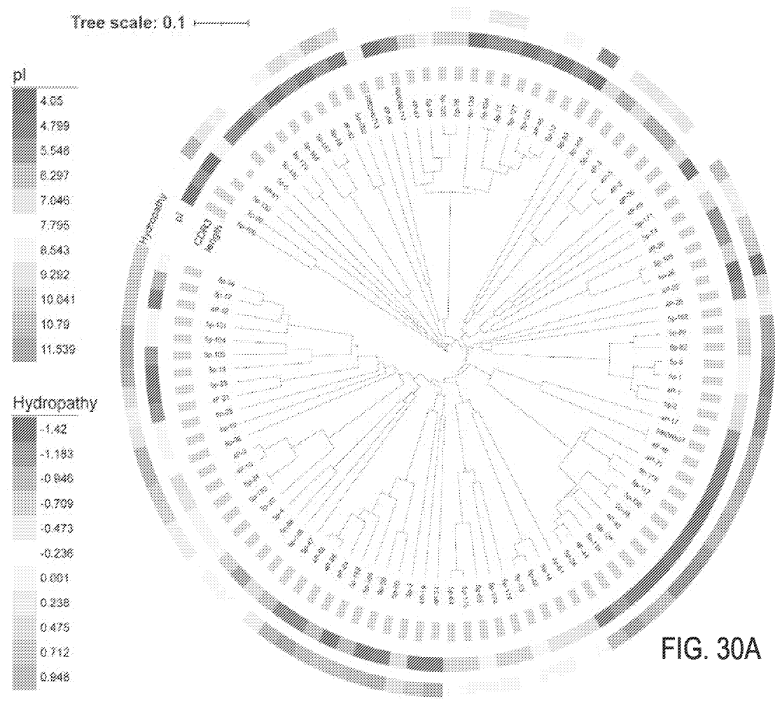

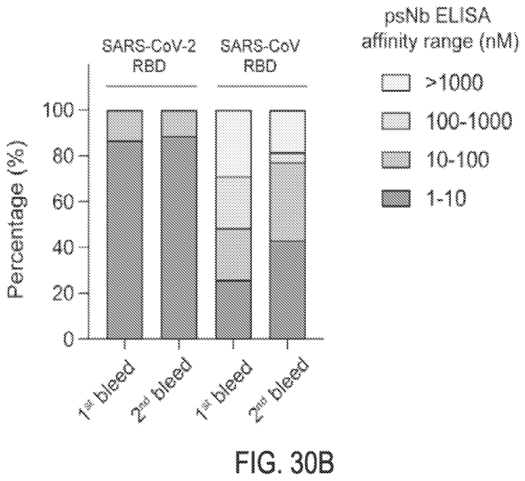

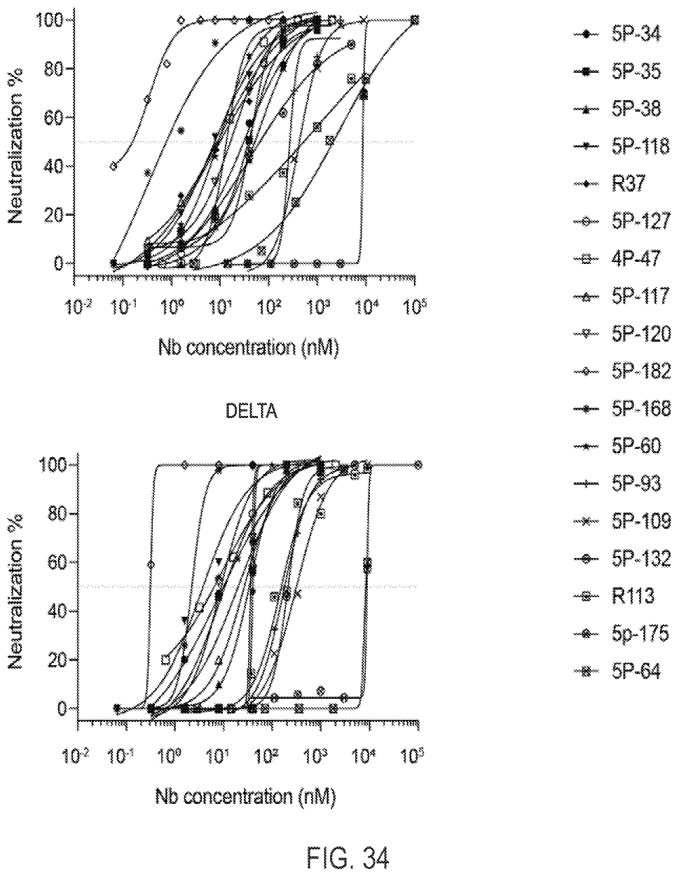

Next, quantitative Nb proteomics were employed to identify high-affinity psNbs that confer broad-spectrum activities in immunized sera (Methods). This technology rapidly identified hundreds of distinct CDR3 families, from which a fraction of highly divergent Nbs were expressed and characterized. A total of 100 Nbs that interact strongly with RBD_{SARS-CoV-2} (Clade 1b) were confirmed to cross-react with other sarbecovirus clades (FIG. 23c, Table 2). Of these, 23%, 35%, and 42% were found to bind two, three, and all four sarbecovirus clades, respectively. Consistent with total polyclonal VHH activities, psNbs isolated from the booster show broader pan-sarbecovirus activities than the initial bleed (FIG. 30b). A substantial fraction (42%) can potently neutralize SARS-CoV-2 below 500 nM, with the best IC50 of 1 ng/ml (77 pM, 5p-182) and a median of 0.25 μg/ml (17 nM) (Table 2). Network analysis reveals that psNbs are dominated by at least seven clusters that span a large spectrum of physicochemical properties including isoelectric point (pI) and hydropathy (FIG. 23d, FIG. 30a). The three largest clusters are each composed of potent neutralizers that bind strongly to at least three sarbecovirus clades (Table 2). The other two clusters (represented by 5p-17, and 5p-175) show broad activities, yet only weakly neutralize SARS-CoV-2 in vitro (IC50>30 μg/ml, FIG. 34).

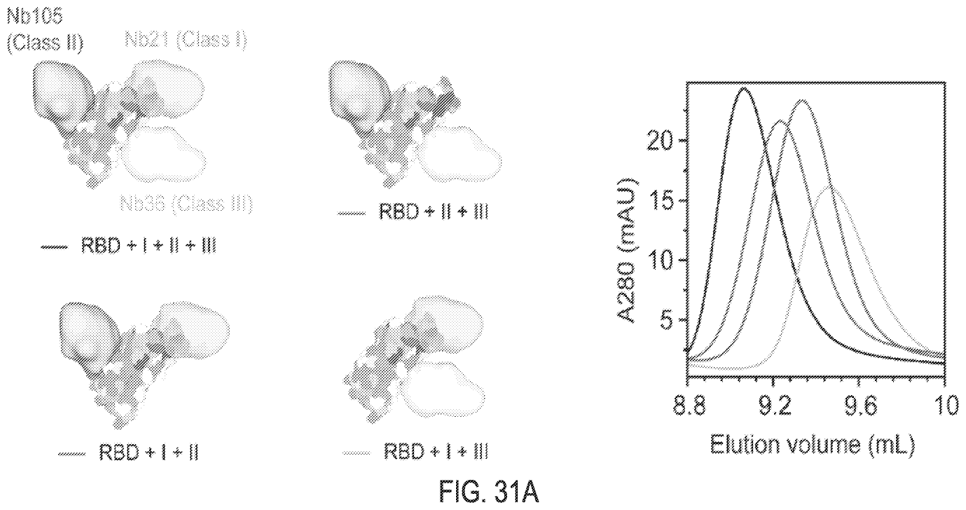

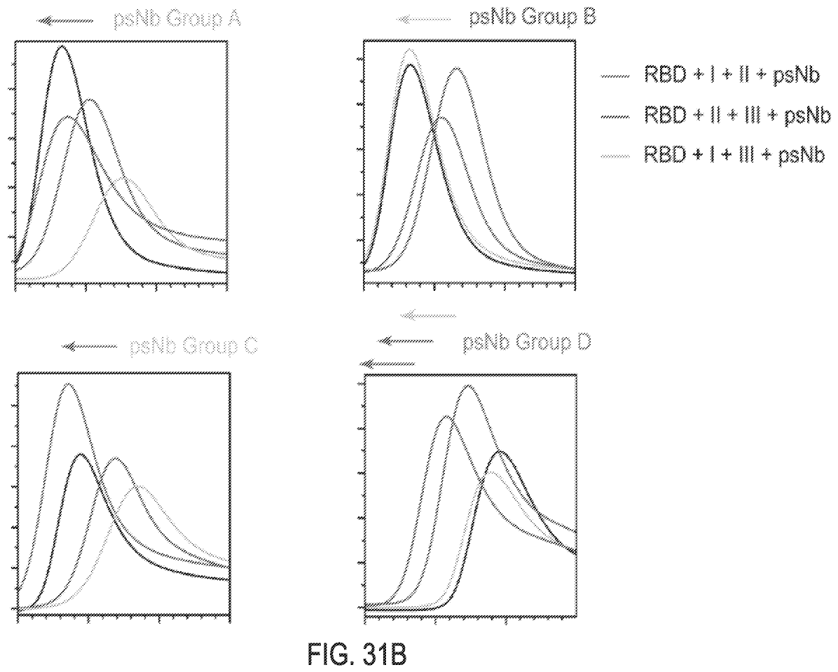

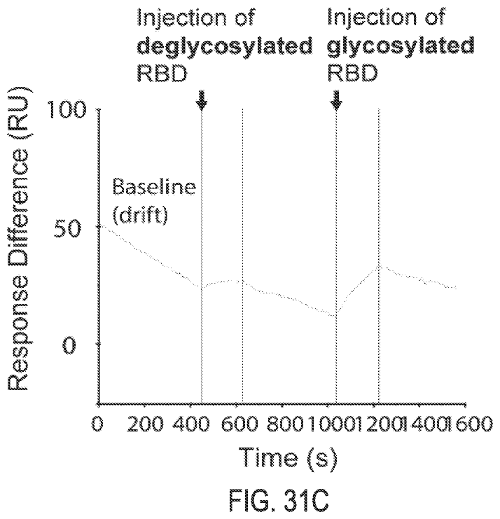

psNbs were further classified by epitope binning using size exclusion chromatography (SEC). psNb-RBD_{SARS-CoV-2} Complexes were competed with high-affinity benchmark Nbs (Nb21, Nb105, and Nb36) that bind distinct and well-characterized epitopes (FIG. 31a-31b, Methods). psNbs fall into five groups: groups B (33%) competes with Nb105, group C (9%) competes with Nb36, group D (17%) does not compete with any benchmark Nbs, and group A (3%) competes with Nb21 that targets RBS. Approximately one third of psNbs (Group E, 38%) bind strongly to RBDs based on ELISA, but do not neutralize pseudotyped SARS-CoV-2 efficiently and can dissociate from RBD on SEC. To investigate this discrepancy, a psNb (5p-156) was randomly selected and evaluated its binding in the presence and absence of RBD glycosylation by surface plasmon resonance (SPR). Analysis confirms that 5p-156 binds strongly to the fully glycosylated but not the de-glycosylated form (FIG. 31c, Methods). This data indicates that some group E psNbs targets a novel epitope encompassing the conserved, glycosylated RBD residue(s) N331 and/or N343. Together, these experiments demonstrate that a large cohort of high-affinity and divergent psNbs targeting multiple dominant RBD epitopes contribute to the broadly neutralizing activities in the immunized serum.

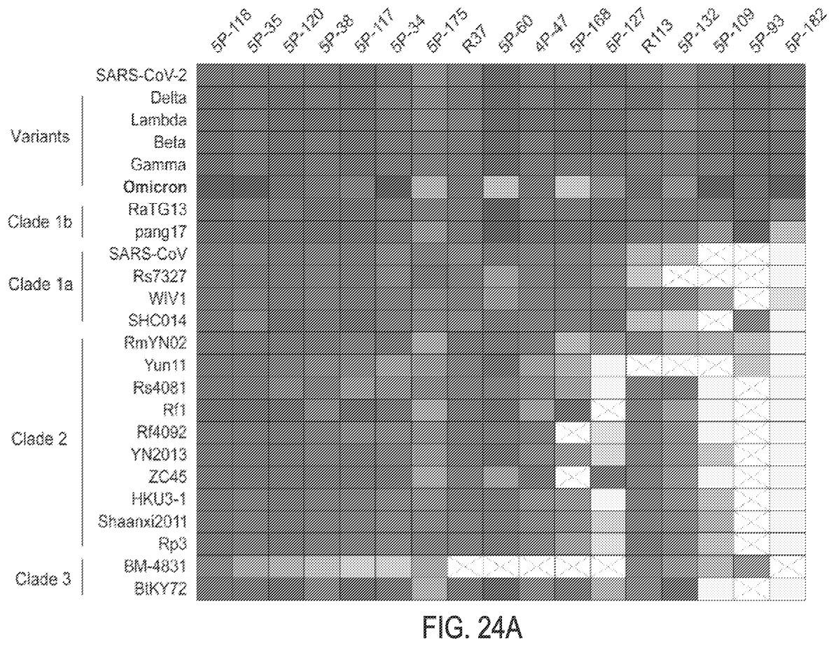

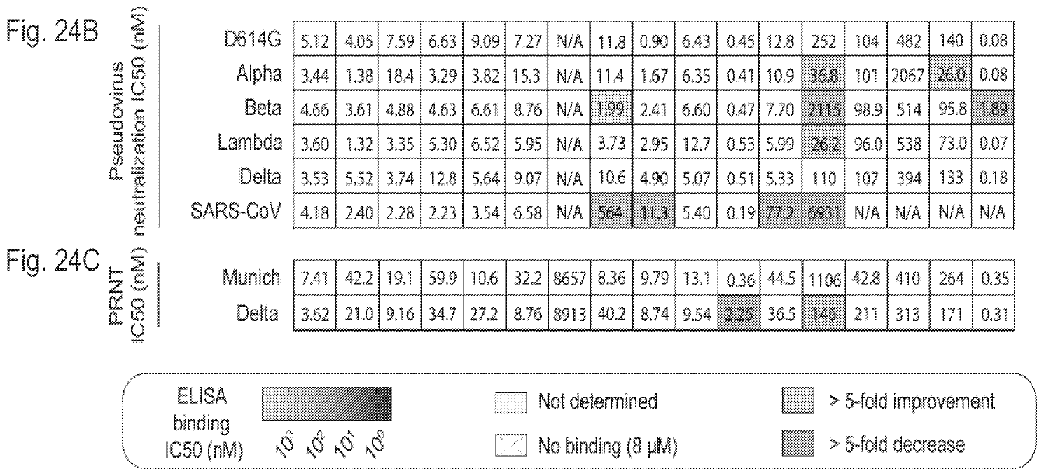

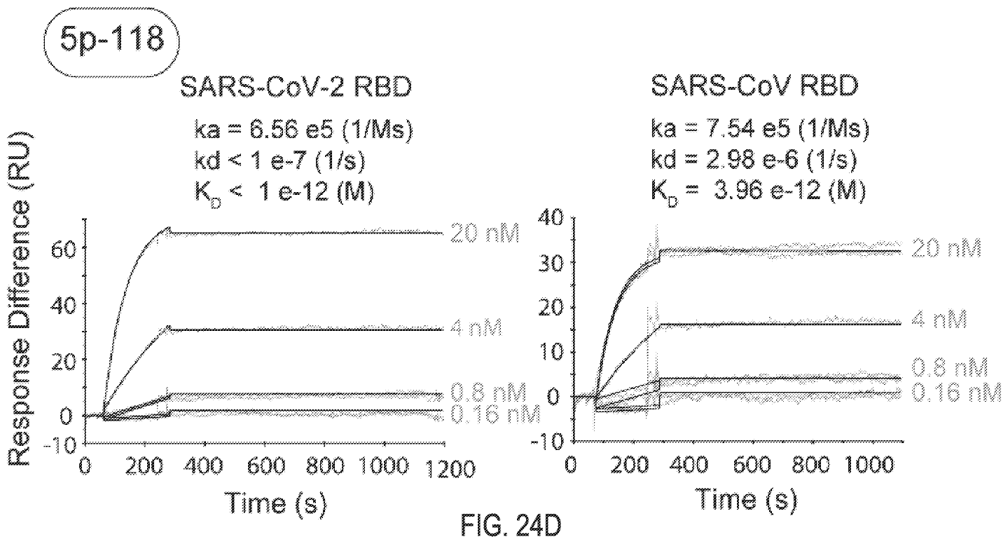

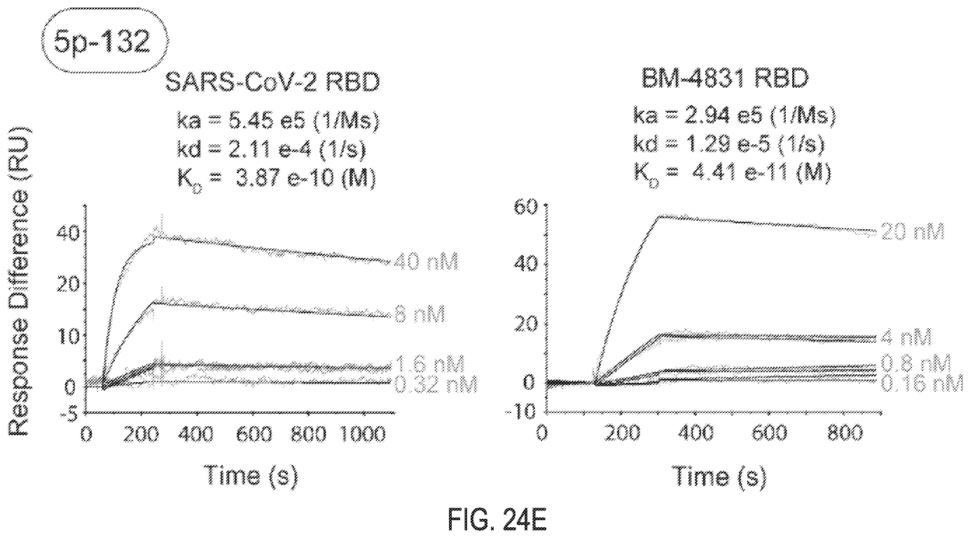

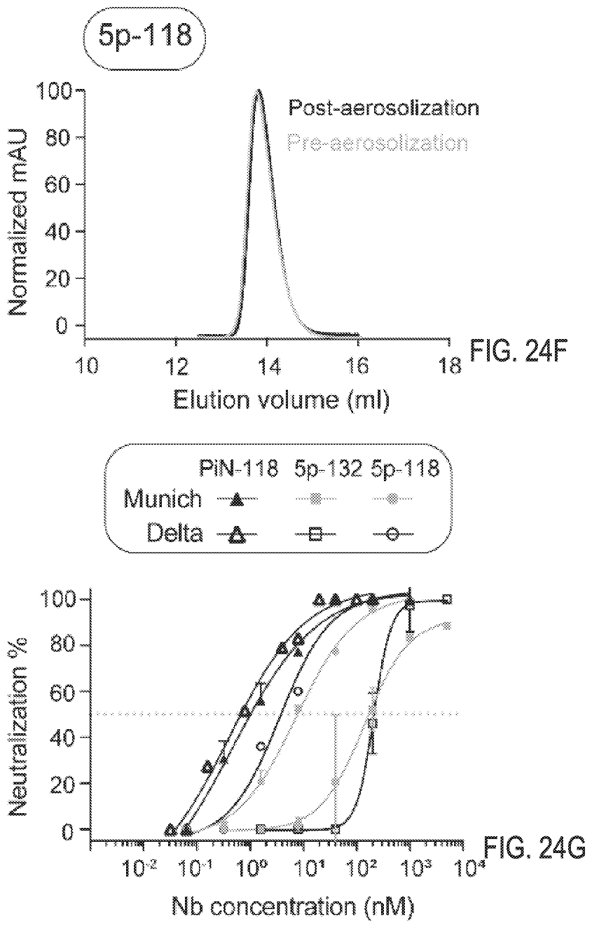

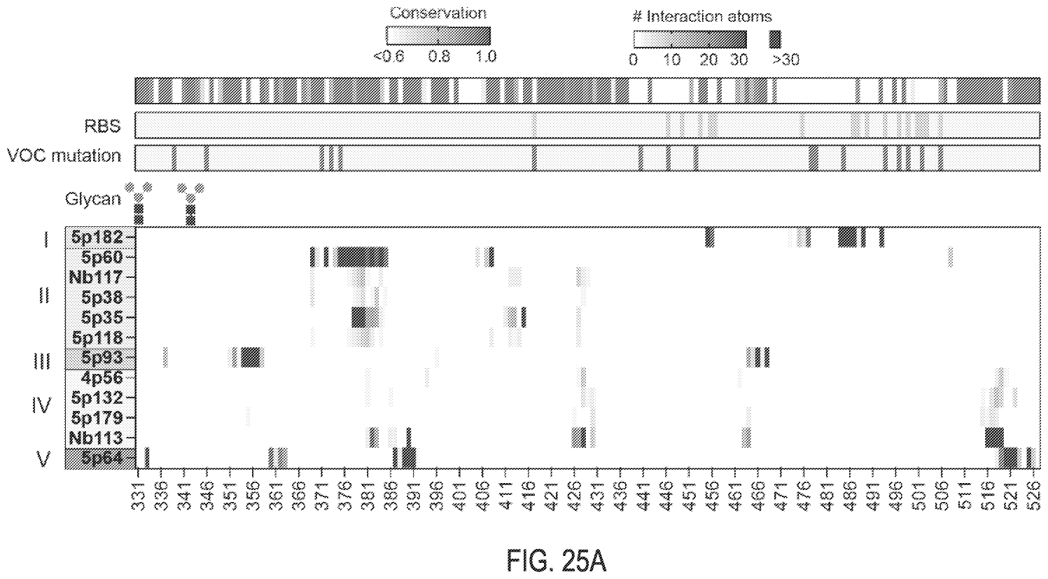

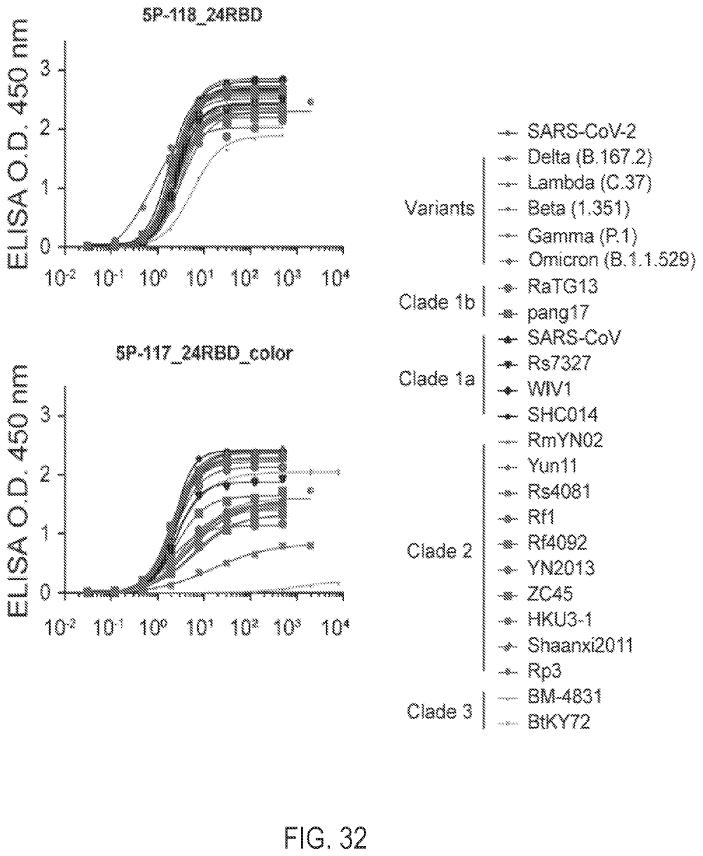

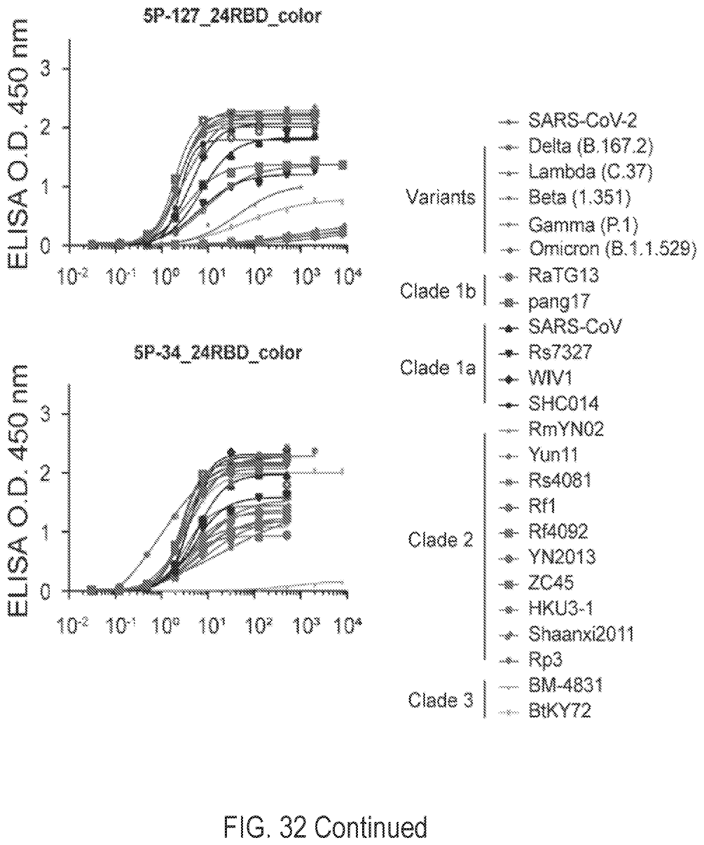

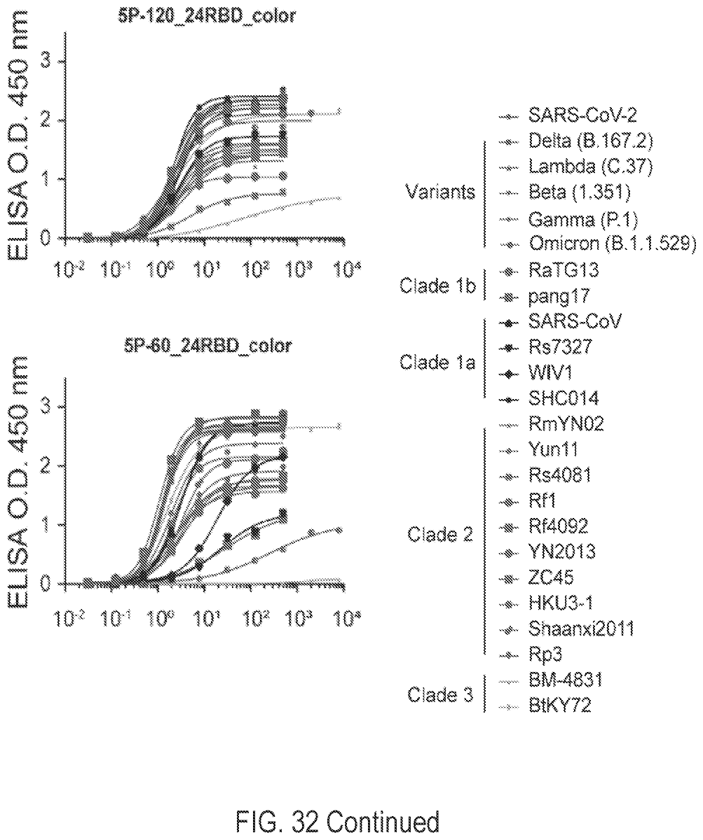

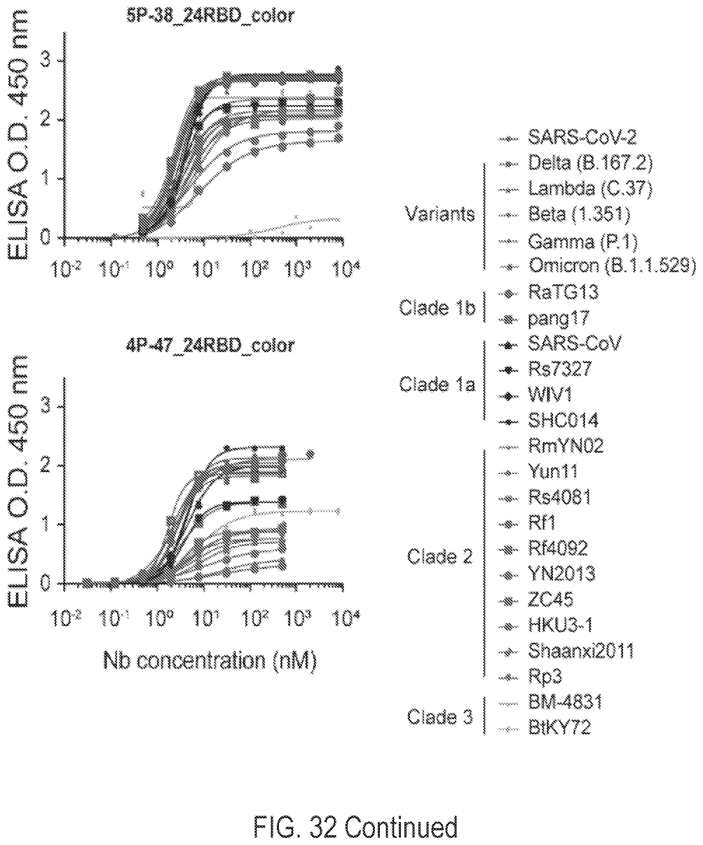

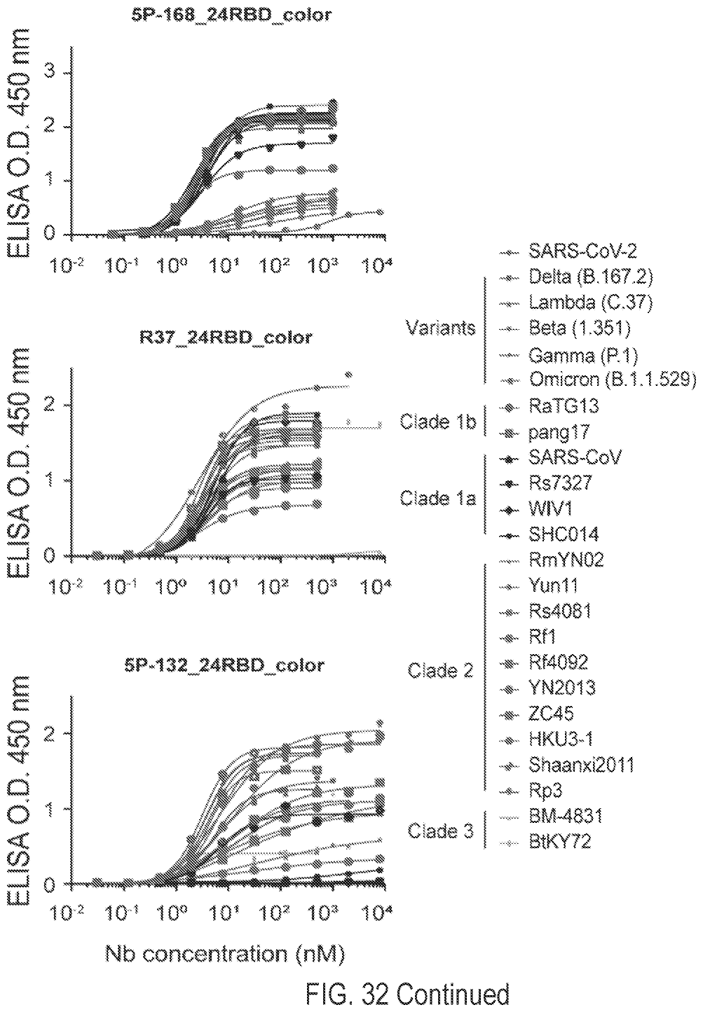

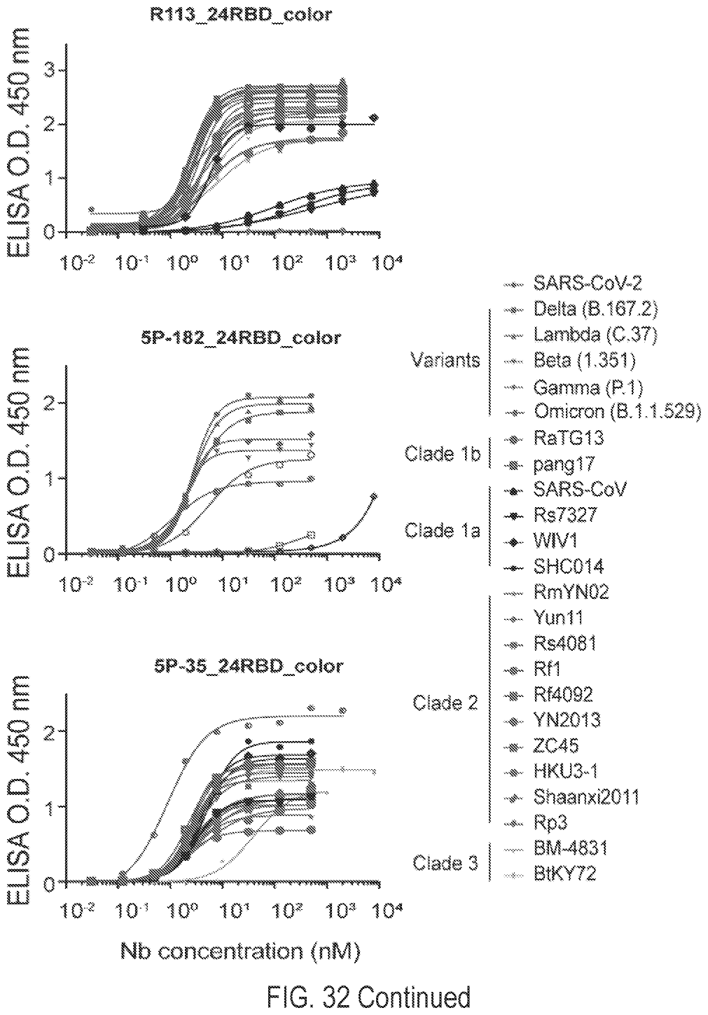

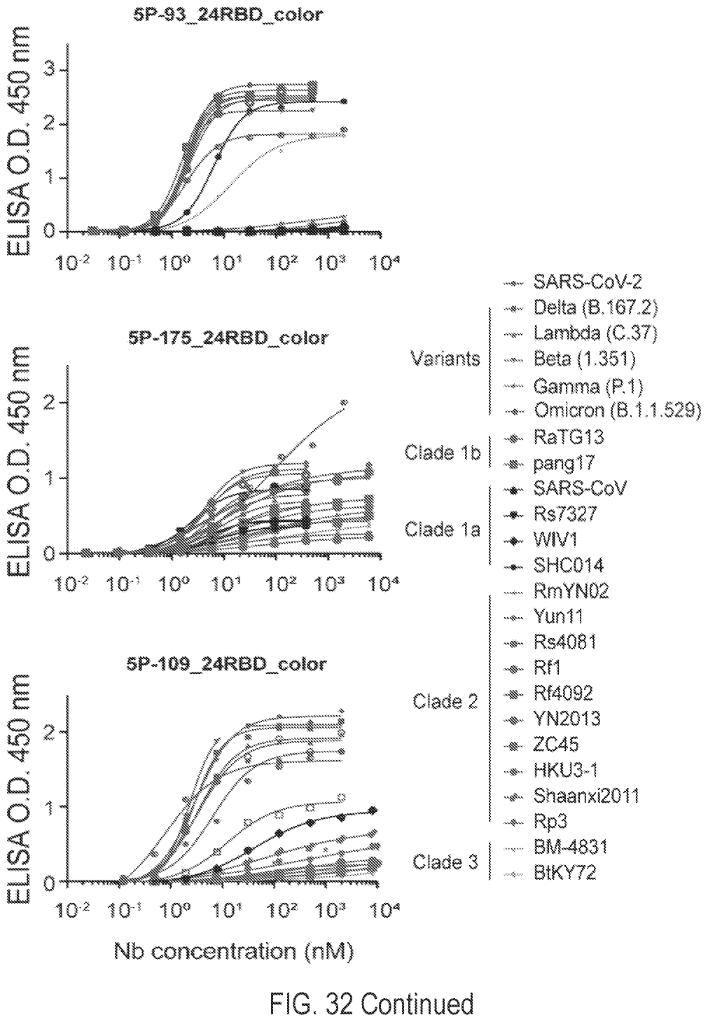

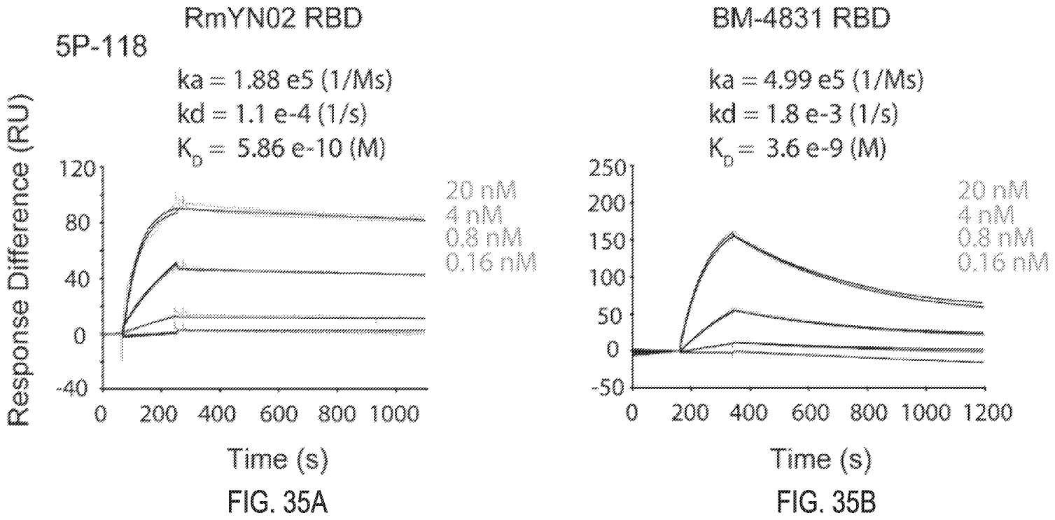

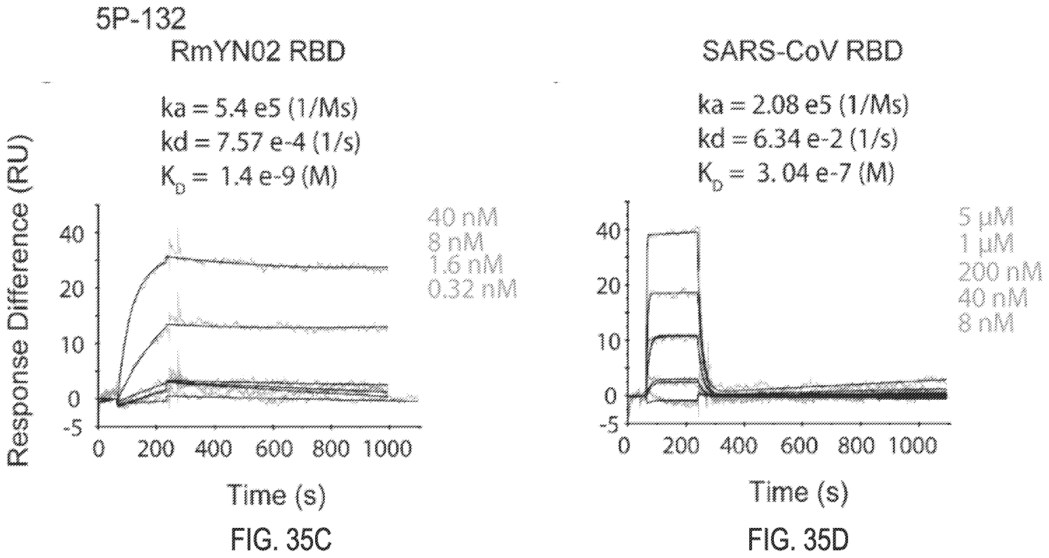

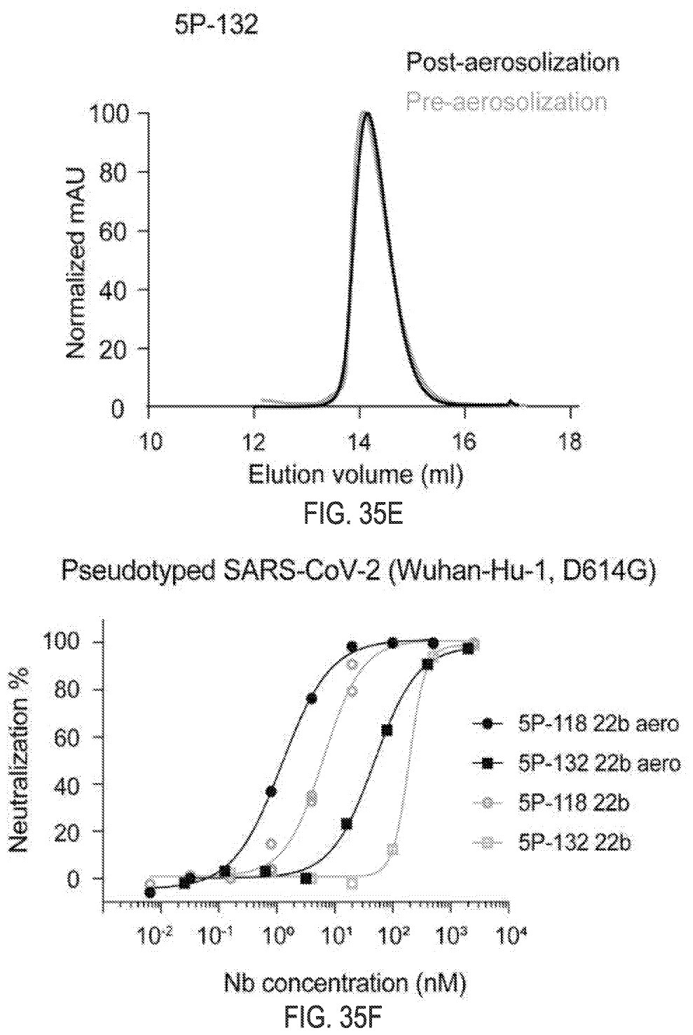

17 psNbs spanning four SEC groups (A-D) were selected for characterization against five critical RBD mutants derived from SARS-CoV-2 VOCs including the Omicron that is spreading quickly around the world, and 18 different sarbecovirus RBDs. These psNbs bind strongly to all the VOCs that were evaluated. 16 of the 17 psNbs bind to all four clades (FIG. 24a). Seven psNbs (such as 5p-118 and 5p-132) have exceptionally broad activities that bind strongly to all 24 RBDs with the median ELISA IC50 of 3 nM (FIG. 24a, FIG. 32, Table 2), which usually corresponds to sub-nM dissociation constant (K_{D}) of Nb binding. Two representative psNbs were selected for binding kinetic measurements against all four clades by SPR. The K_{D}S of 5p-118 for RBD_{SARS-CoV-2} (Clade 1b), RBD_{SARS-CoV}(clade 1a), RBD_{RmYNO2-CoV} (clade 2), RBD_{BM-4831-CoV} (clade 3) are sub-pM, 3.96 pM, 0.59 nM and 3.60 nM (FIG. 24d, FIGS. 35a-35b), respectively. 5p-132 strongly binds clades 1b, 2 and 3 (respectively 0.39 nM, 1.4 nM and 0.044 nM) while moderately binding clade 1a (304 nM) (FIG. 24e, FIG. 35c-35f). Of note, these psNbs are highly specific and do not cross-react with the human whole protein extract at high concentrations (up to 8 μM).

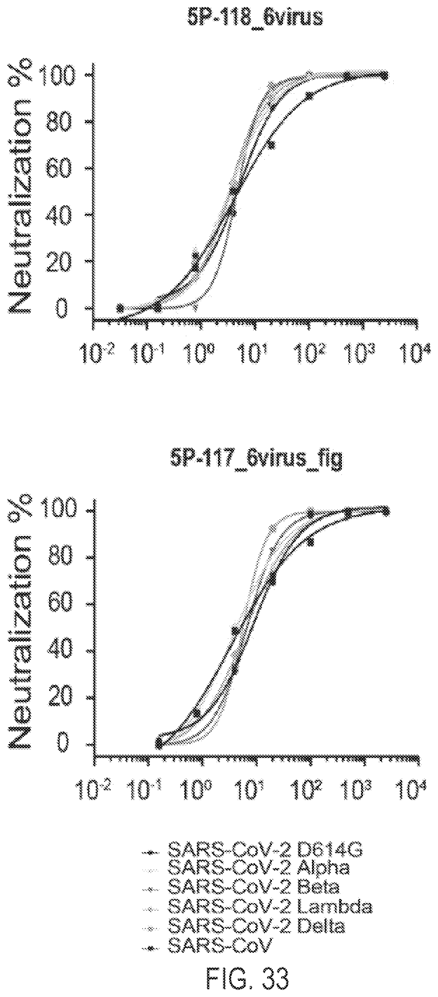

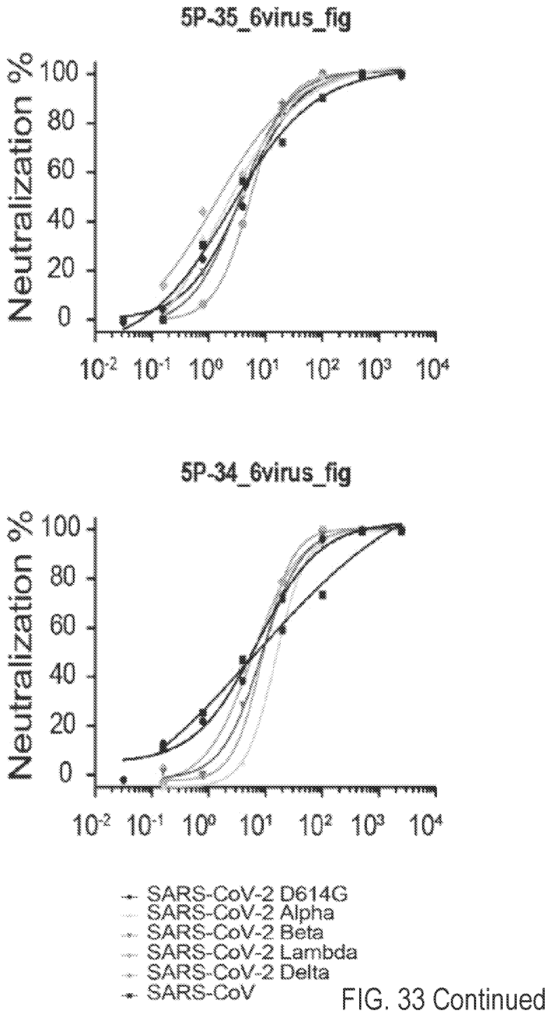

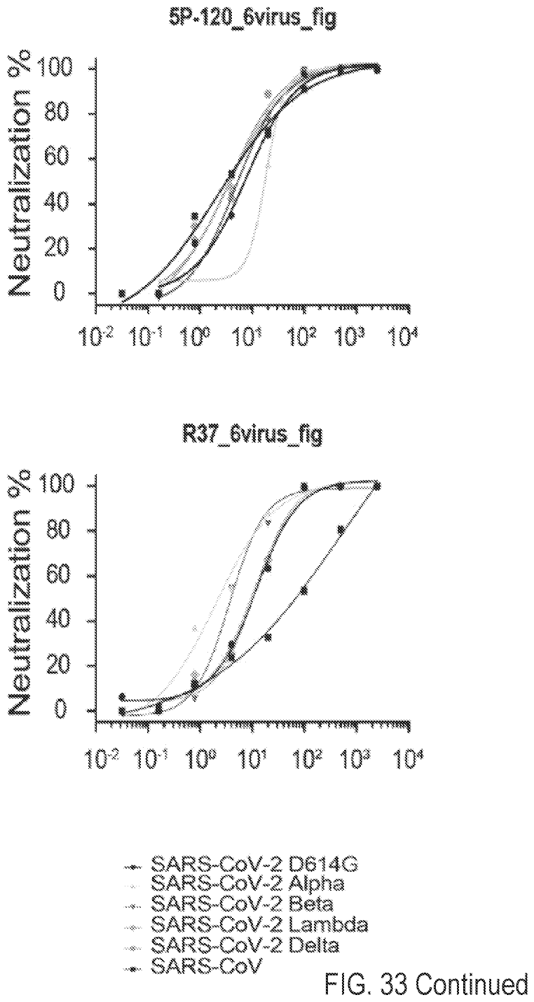

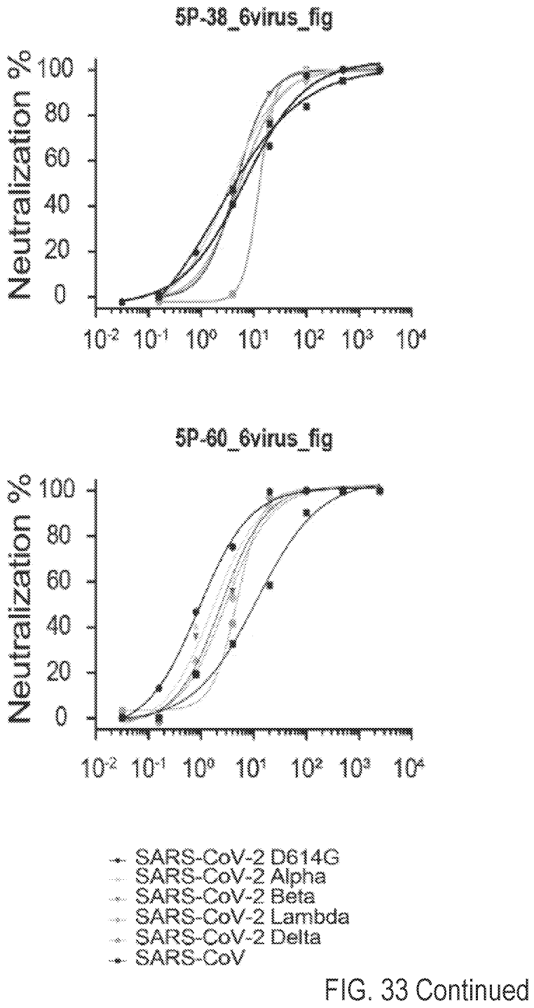

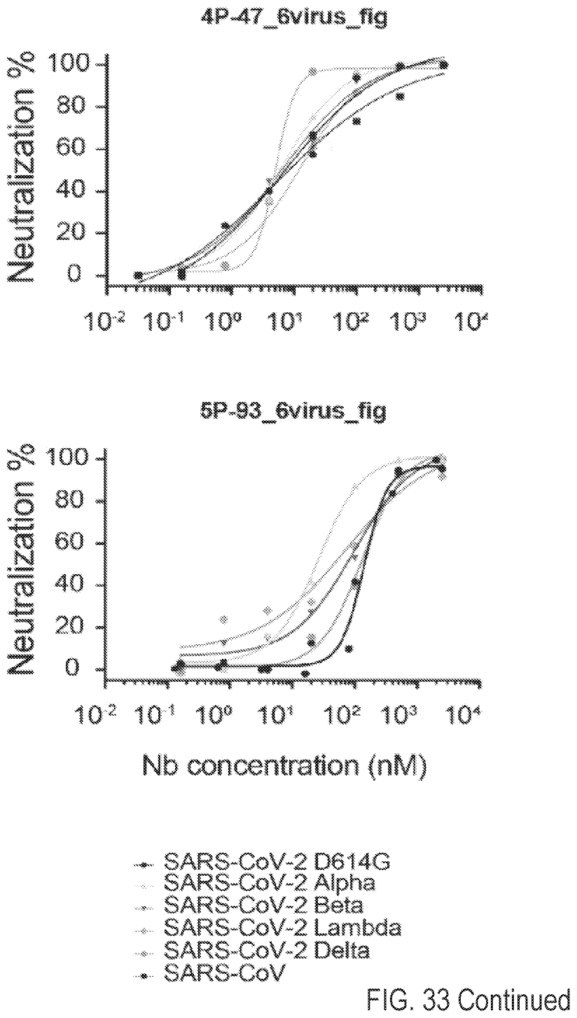

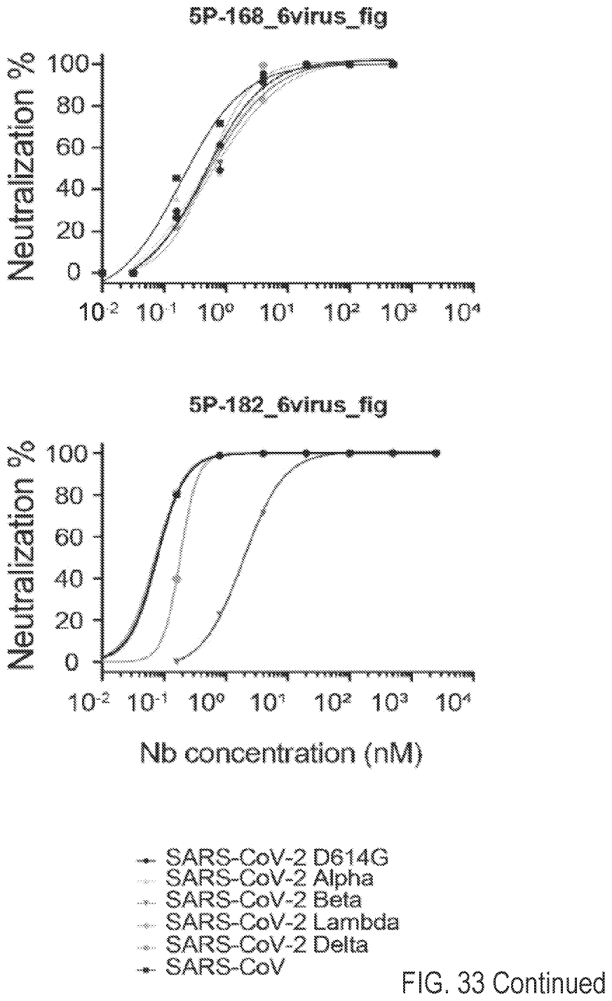

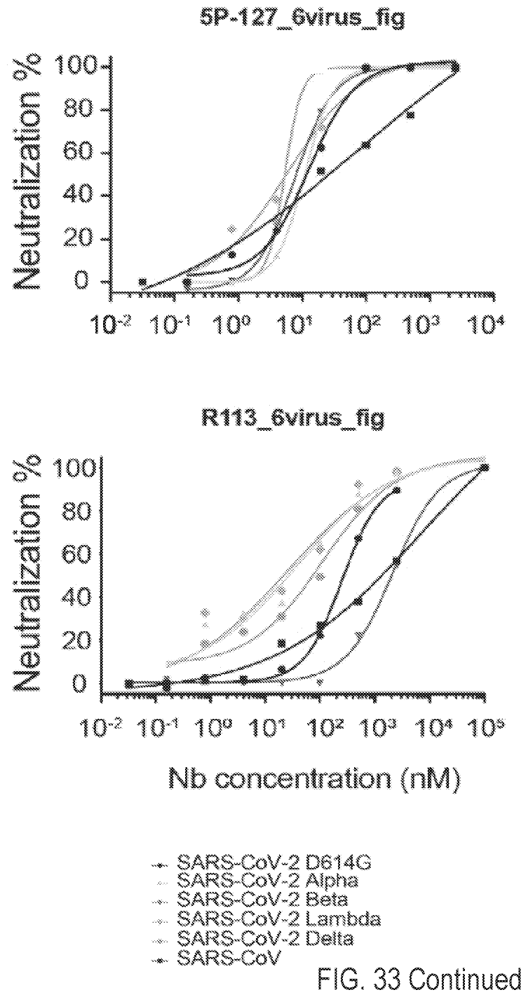

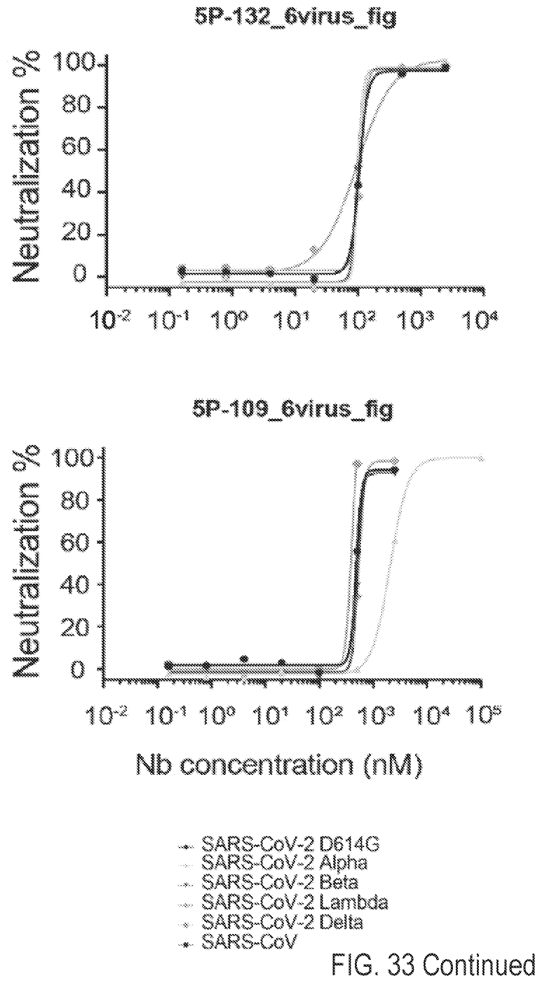

All but one psNbs can potently inhibit SARS-CoV-2 and the VOCs in vitro, based on both a pseudovirus assay and a plaque reduction neutralization test (PRNT) against clinical isolates of the Munich strain and Delta VOC (FIG. 24b-24c, FIGS. 33-34, Methods). Group A Nb 5p-182, which preferentially binds to clade 1b, is extremely potent for SARS-CoV-2 and VOCs with a median neutralization potency of 1.2 ng/ml. Group C (R113, 5p-93 and 5p-132) neutralizes VOCs at single-digit μg/ml and is less effective against SARS-CoV. Group D (5p-109 and 5p-175) has an exceptional breadth yet only weakly neutralizes SARS-CoV-2 (IC50 7-132 μg/ml). The remaining Nbs belong to group B and show comparably high potency against SARS-CoV. The median potency for SARS-CoV-2 and VOCs is 86 ng/ml with the most potent one (5p-168) at 6 ng/ml. Notably, psNbs (such as 5p-118 and 5p-132) are usually highly stable and can withstand aerosolization without compromising activities (FIG. 24f, FIGS. 35e-35f).

This study developed a bispecific construct (PiN-118) by fusing two potent and stable psNbs (5p-118 and 5p-132), which cover two distinct and highly conserved epitopes (II and IV). Compared to the monomeric Nbs, the potency of PiN-118 improves by an order of magnitude to 12.8 ng/ml (0.4 nM) based on the PRNT assay using the Delta VOC (FIG. 24g). PiN-118 remains fully active after aerosolization using a portable and inexpensive nebulizer for clinical development (Methods).

Diversity, Convergence and Evolution of Broadly Neutralizing psNbs

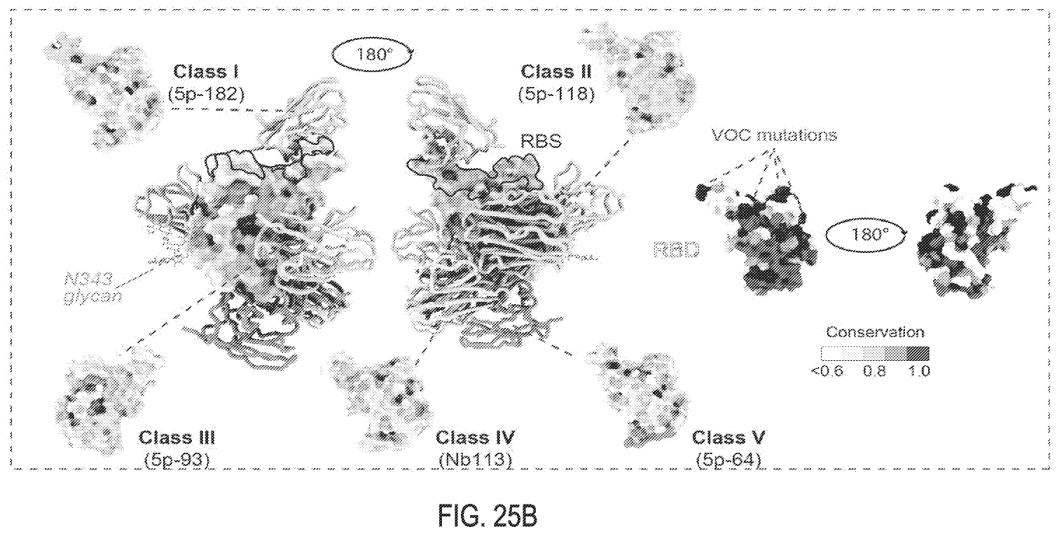

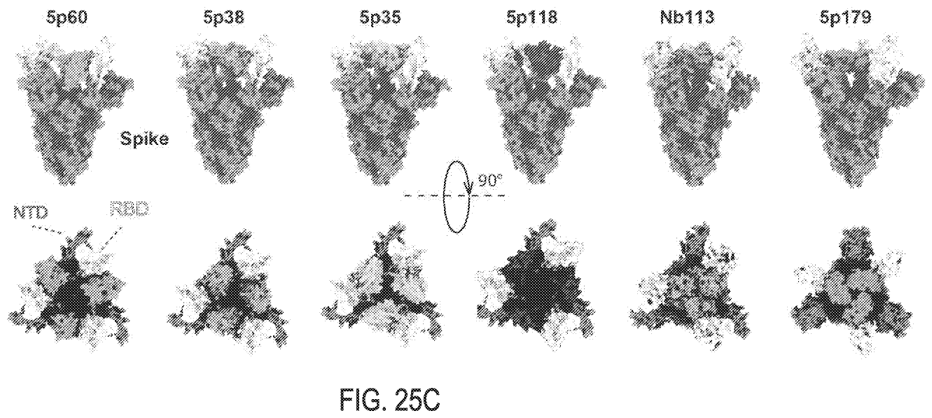

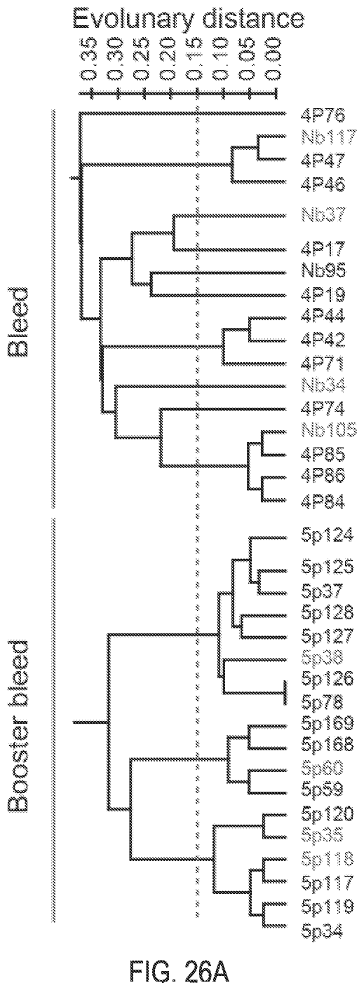

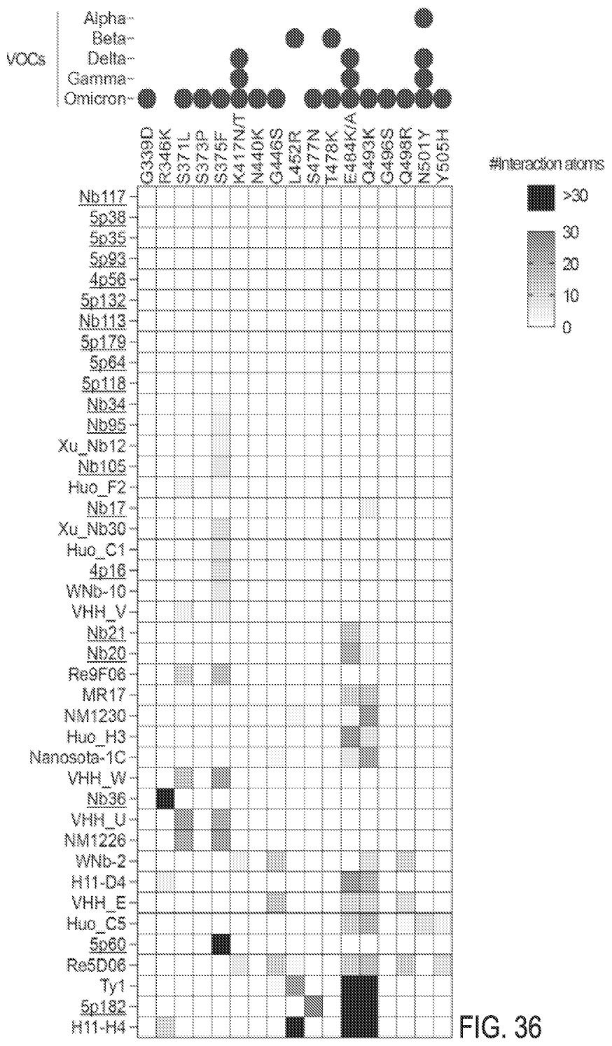

To understand the mechanisms of broad neutralization, this study used cryoEM to determine the structures of 11 psNbs in complex with the SARS-CoV-2 spike or RBD. Additionally, 2 atomic psNb: RBD structures were determined by X-ray crystallography. Epitope clustering based on high-resolution structures reveals 5 main epitopes (FIG. 25a), which do not overlap with critical RBD mutations from the VOCs including Alpha, Beta, Delta, and the emerging Omicron (FIG. 25b, FIG. 36). Most psNbs, except for 5p-182, do not bind to RBS. Three quarters of the solvent-exposed and highly conserved RBD residues (sequence identity 85%) are covered by psNbs (Methods). Superposition to spike structure reveals that psNbs can lock RBDs preferentially in the 3-up conformation (FIG. 25c). Despite having different epitopes and orientations, however, the small size facilitates simultaneous binding of three copies to the spike trimer in a distinct and highly symmetric configuration.

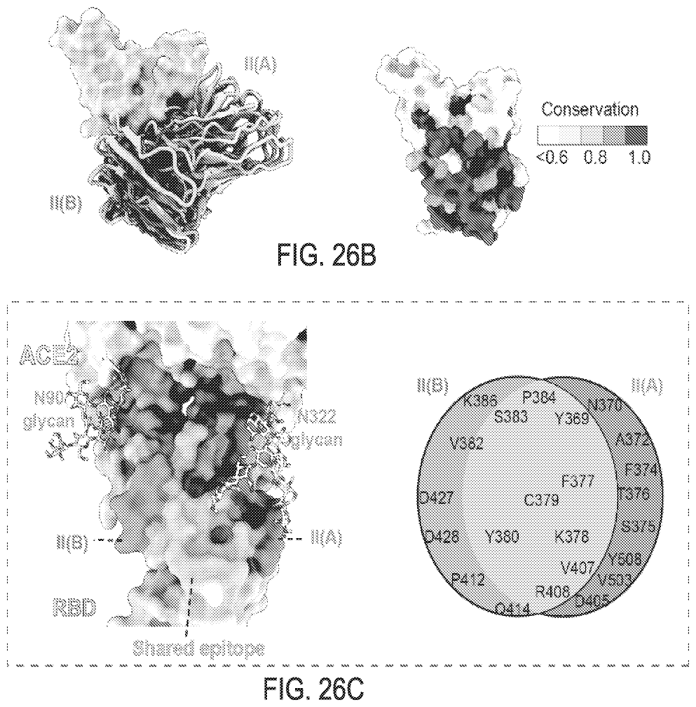

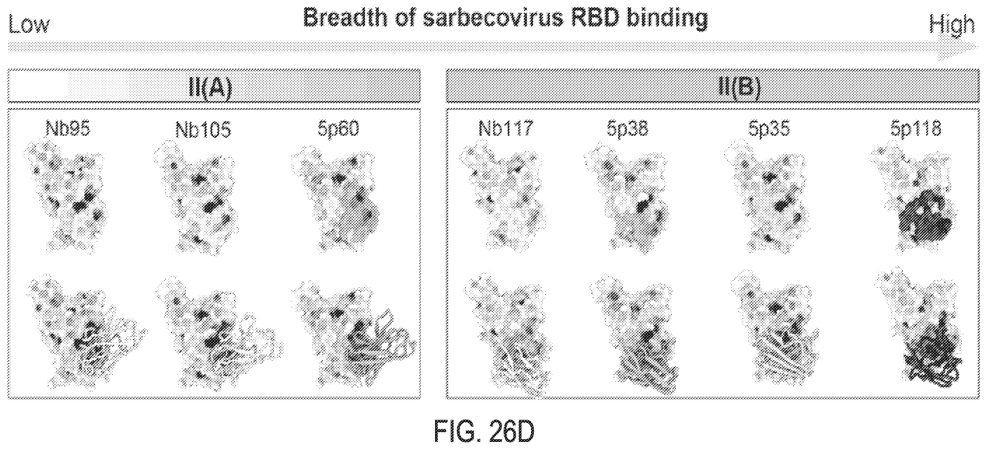

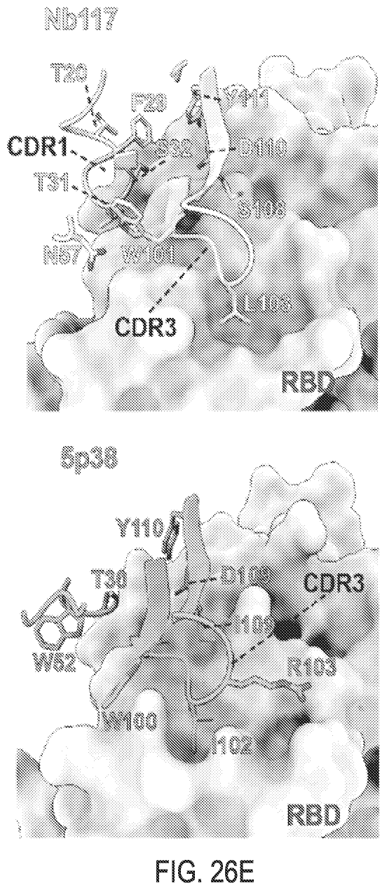

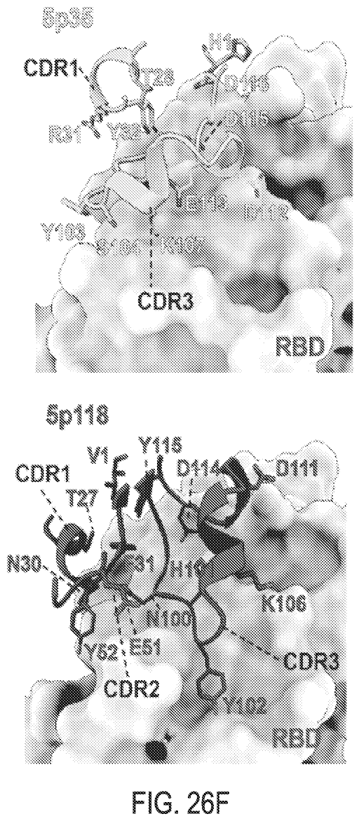

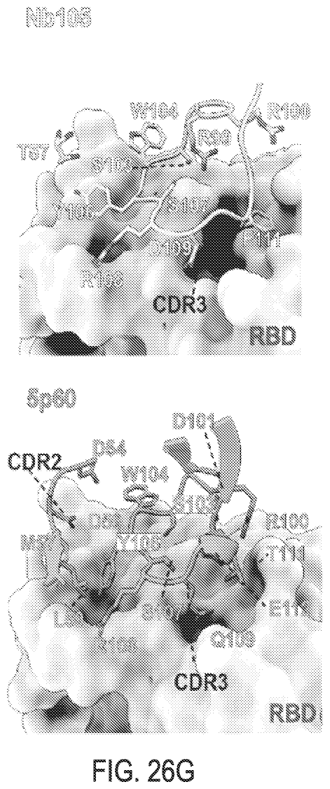

Class II psNbs are overrepresented in our collection. Phylogenetic analysis reveals that while psNbs are diverse, those isolated from the booster are more converged than the initial bleed (FIG. 26a). 5 psNbs were structurally determined with the spike or RBD at high resolutions by X-ray crystallography and cryoEM (Methods). Analysis unveils two subclasses (A and B) based on epitopes and Nb scaffold orientations (FIG. 26b, 26d). II(A) and II(B) psNbs share a conserved hydrophobic core epitope (aa 377-386) (FIG. 26c) containing two bulk hydrophobic residues (F377 and Y380), and are stabilized by a disulfide bond (C379-C432). The improved breadth in class II(B) psNbs, especially for the clade 3, RBD_{BM-4831-CoV} lies in its ability to target an additional conserved region with peripheral charge residues (aa 412-415, 427-429), wherever class II(A) surveys a less conserved epitope of primarily hydrophobic residues (e.g., L368, A372, and Y508) (FIG. 26c). Interestingly, A372 residue on SARS-CoV-2 (clade 1a) is substituted to S/T in other clades, forming a consensus glycosylation motif (³⁷⁰NST/S³⁷²), which may explain the reduced neutralization potency of class II (A) psNbs but not class (B) against SARS-CoV (FIG. 24b, 26b).

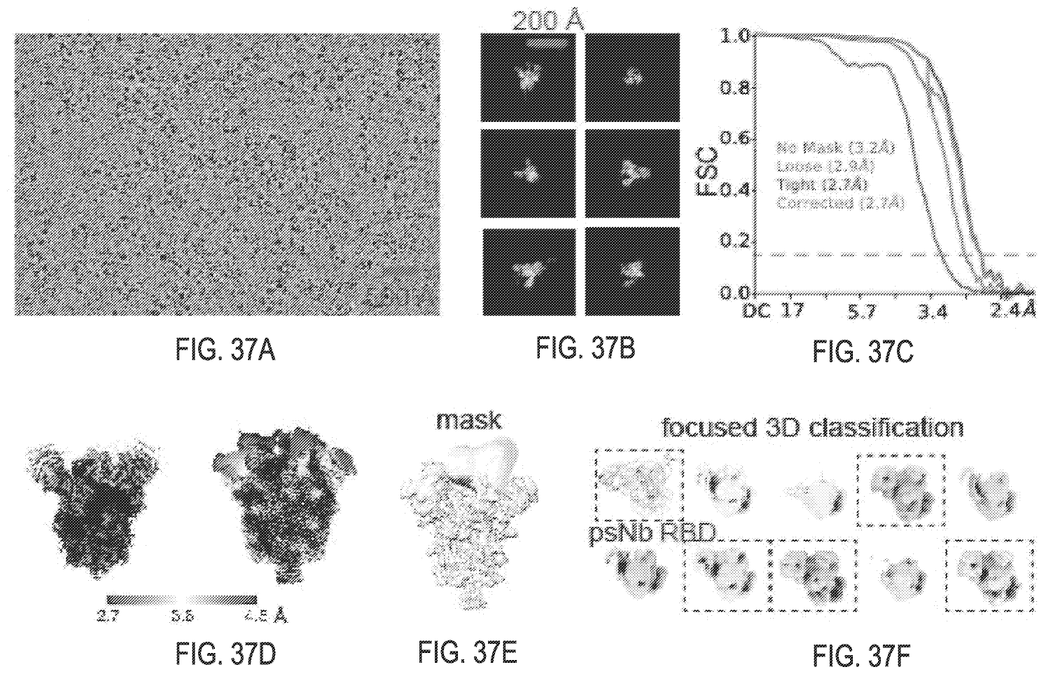

Class II(B) psNbs comprise two large clusters possessing the best breadth and potency (FIG. 23d). Their epitopes largely overlap but interactions vary substantially. Two related psNbs (5p-38 and Nb117) are characterized by a short CDR3 (13 aa) forming a β finger conformation. psNb 5p38 is an all-clade binder while Nb117 binds strongly to all but RBD_{BM-4831-CoV} (clade 3) due to a substitution K378Q, which disrupts a critical salt bridge between D110 (Nb117) and K378 (RBD_{SARS-CoV-2}) (FIG. 26e, FIG. 37, 38a-38b, and 44). 5p-35 and 5p-118 (from the other dominant cluster) bind all 24 RBDs strongly with potent antiviral activities. The interactions are predominantly mediated by a long CDR3 loop (18 aa) with a small helix. These Nbs form hydrophobic interactions and also bind strongly to conserved charged residues (such as D427 and R408 on the rims of epitopes), forming electrostatic interactions (FIG. 26f, FIG. 37, 38c-38d, and 44).

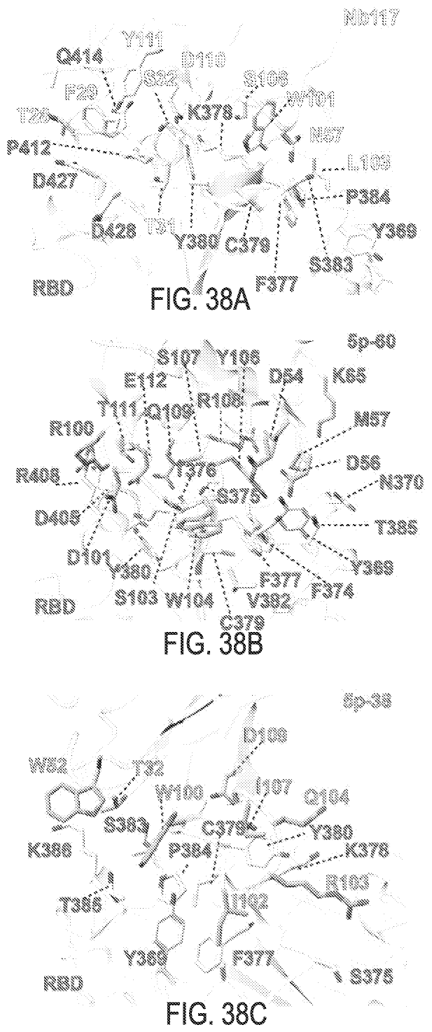

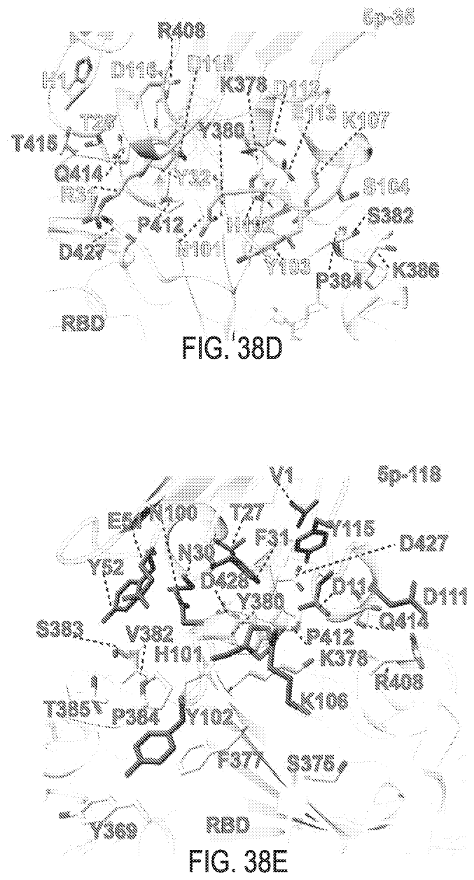

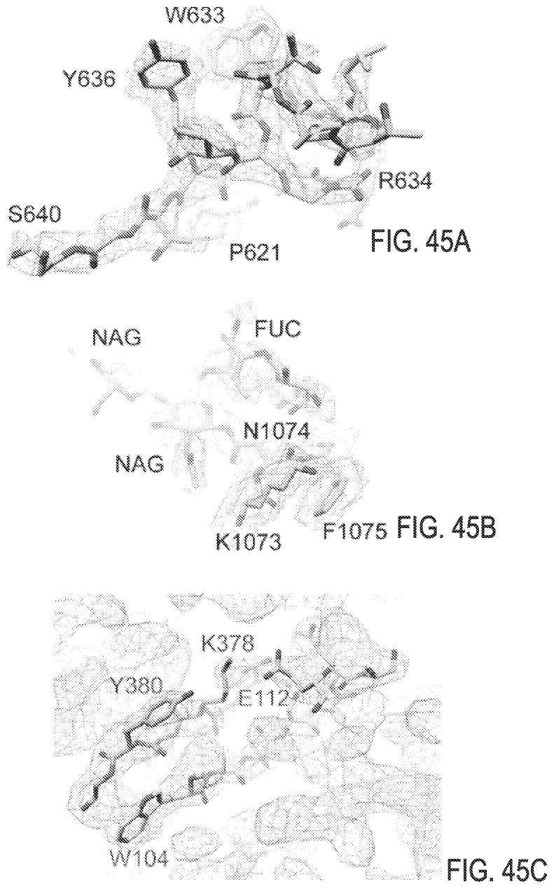

Class II(A) psNb (5p-60) shares high sequence similarity with Nb105 (isolated from the initial bleed). The major difference lies in their CDR3 heads. Here, a small CDR3 loop of ¹⁰⁹DLF¹¹¹ in Nb105 is substituted by ¹⁰⁹QST¹¹¹ in 5p60, which inserts into two conserved pockets (RBD residues F377, Y369, F374 and residues S375, T376, Y508, V407) forming extensive hydrophobic interactions with the RBD (FIGS. 26g, 38E, and 45). This substitution allows psNb 5p-60 to tolerate sequence variation better, exhibiting over 100-fold higher affinities for clade 2 RBDs (Table 2).

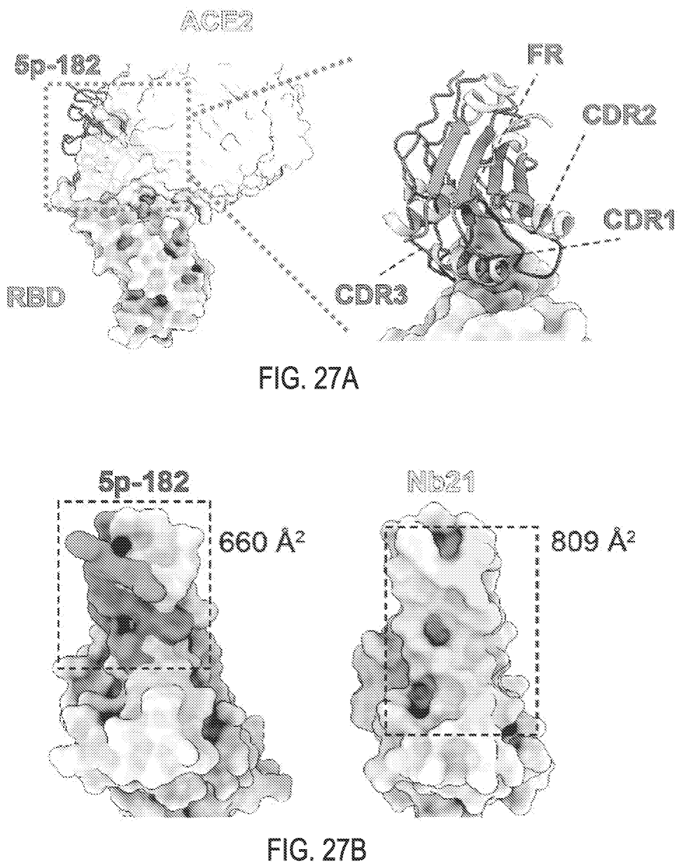

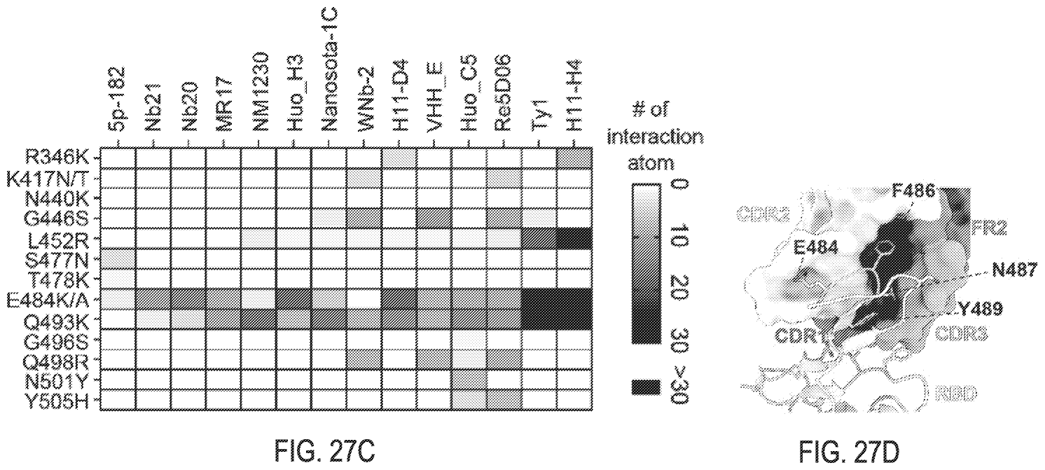

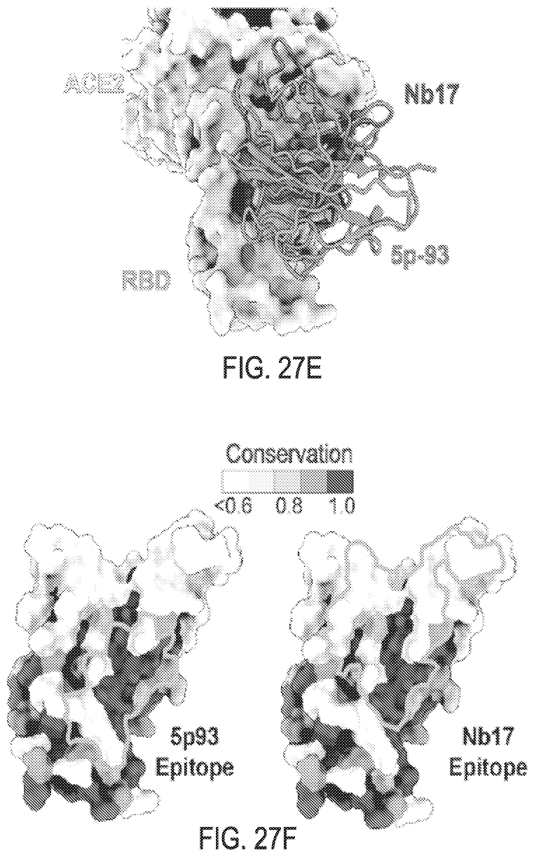

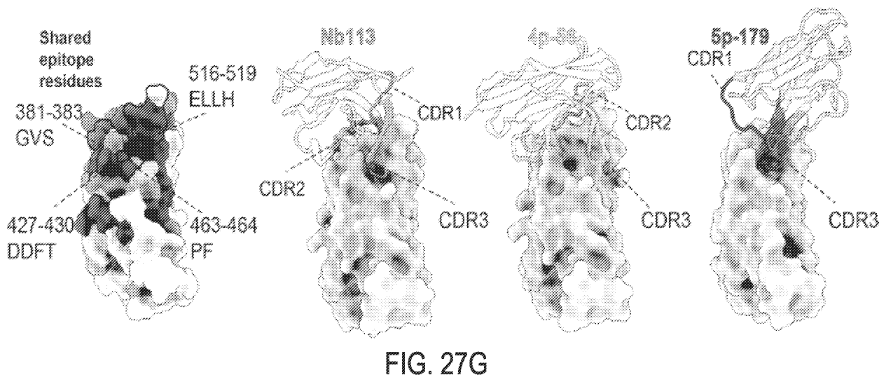

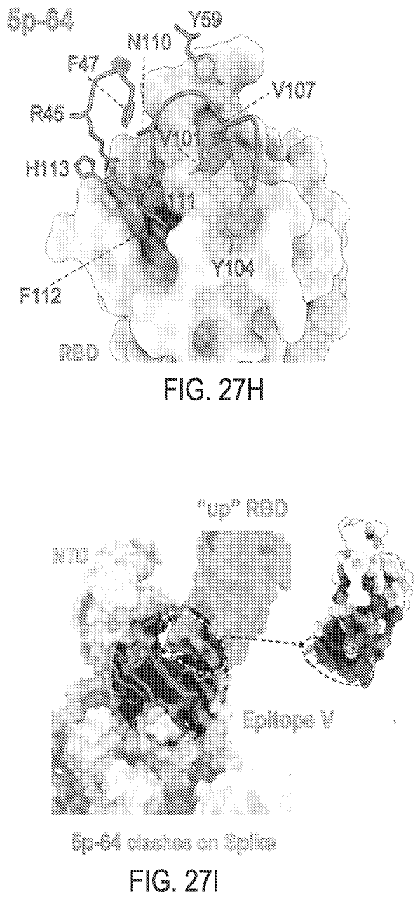

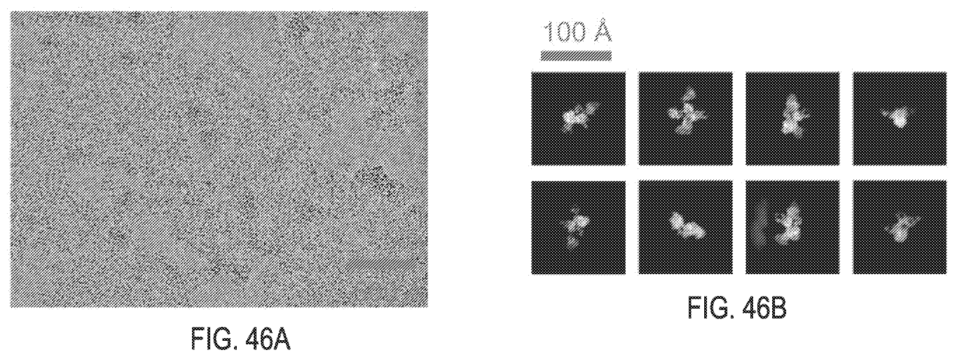

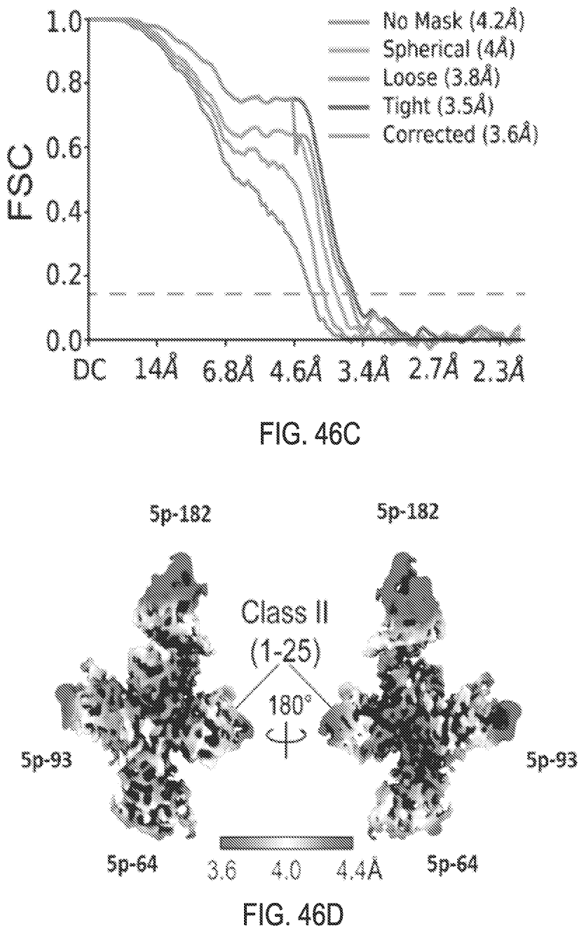

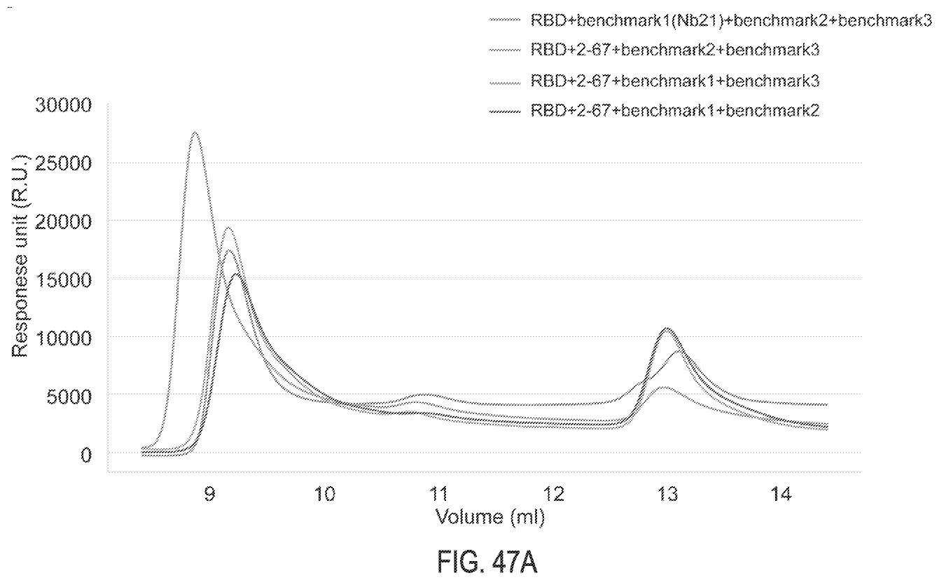

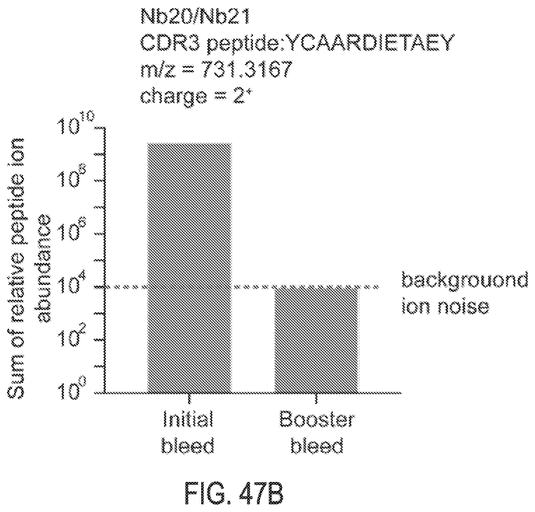

Class I psNbs that bind RBS are extremely potent yet rare (˜3%). The structure of the ultrapotent 5p182 was resolved by cryoEM with the spike (FIG. 46). Superposition of 5p-182:RBD and ACE2:RBD (PDB 6MOJ) structures revealed major clashes between all three Nb framework regions and two CDRs (2 and 3) with the minor helix of hACE2 (aa 26-37, 66-87, 91-109, 187-194) (FIG. 27a). 5p-182 targets a small epitope (BSA 546 Å²) on the protruded RBS loop (aa 472-490) (FIG. 27c). Compared to the epitopes of other RBS Nbs that can be easily evaded by the circulating variants, most RBD mutations do not localize on the 5p-182 epitope (FIG. 27d). Here, 5p-182 utilizes all CDRs that form extensive networks of hydrophobic and polar interactions with two critical RBD residues that are absent in VOC mutations (F486 and Y489), and therefore, can achieve ultrahigh affinity for SARS-CoV-2 and retain strong binding and neutralization against the variants (FIG. 27c). 5p-182 can outcompete an ultrahigh-affinity Nb21 for binding to RBS (FIG. 47a). Interestingly, quantitative MS analysis reveals that multiple ultrapotent RBS binders (Nbs 20, 21 and 89) identified from the initial bleed were absent in the booster serum, indicating they are sourced from short-lived plasma cells (FIG. 47b).

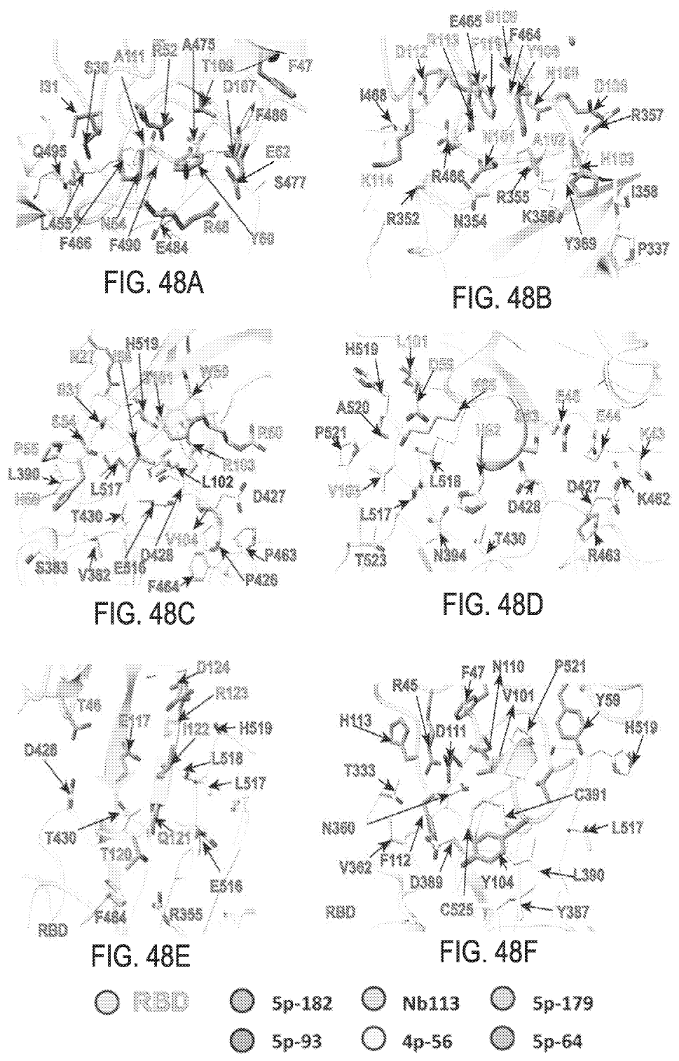

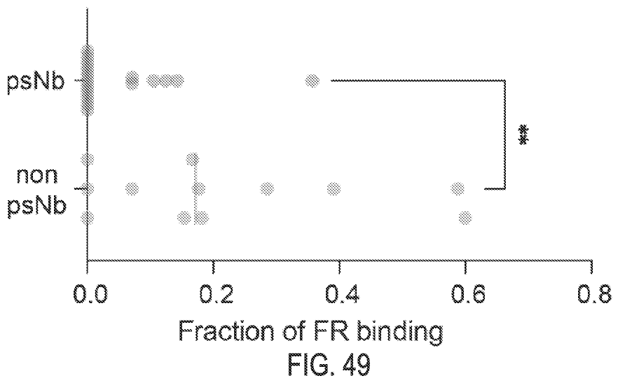

Class III psNb (5p-93) can destabilize the spike in vitro. The structure was resolved by reconstituting an RBD: 4 Nbs complex that helps epitope determination by cryoEM (Methods, FIG. 46). 5p-93 targets a rare non-RBS epitope and overlaps with Nb17 (FIG. 27e) isolated from the initial bleed that is partially effective against variants. Class III psNb binds strongly to RBD variants and clade 1b and shows selectivity within other clades. Compared to Nb17 with limited breadth, the improved breadth of class III psNb is contributed by shifting (from residues 356-357 to residues 449-450, 481-484 and 490-494) towards a smaller yet more conserved epitope (FIG. 27f, FIG. 48).

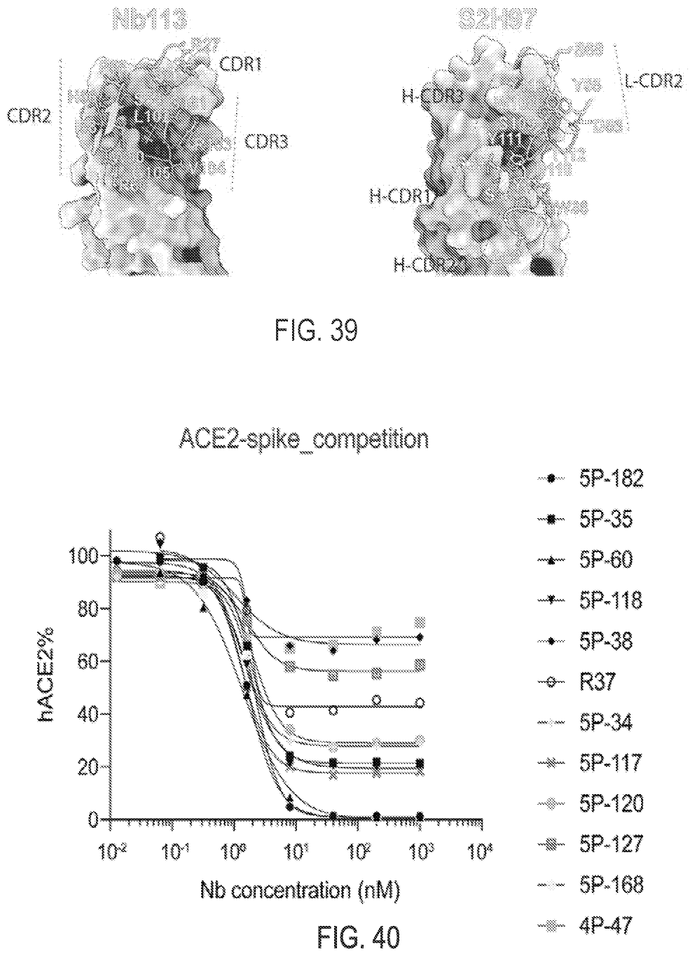

Four Class IV psNbs (Nb113, 4p-56, 5p-179 and 5p-132) were resolved with the spike by cryoEM (Methods). These psNbs employ a plethora of mechanisms including distinct scaffold orientations, CDR combinations and molecular interactions. Class IV shares a highly conserved and cryptic epitope only accessible in the RBD-up conformation (FIG. 25a, 25c, 25g, FIG. 48). The epitope is characterized by charged and hydrophobic residues (D427/D428, T430, F464, ₅₁₆ELLH₅₁₉) forming a cavity (FIG. 25g). CDR3s of class IV adopt a “hairpin” conformation that inserts into the RBD cavity forming extensive hydrophobic and polar interactions. Nb113 and 4p56 are all-clade binders and can also recognize multiple other conserved RBD residues (L390, P426 and P463 for Nb113 and Y369 for 4p56) through hydrophobic interactions (FIG. 48C-48D). 5p-179 shows selectivity towards clade 1b and clade 3 likely due to a unique salt bridge between D124 (CDR3) and the semi-conserved H519 on RBDs (FIG. 48D). Notably, class IV epitope partially overlaps with a newly discovered broadly neutralizing antibody S2H97 and targets RBD from a distinct angle (FIG. 39). Compared to S2H97 which uses extensively bulky aromatic side chains (W, F. and Y) for interactions, the binding of Nb113 is primarily mediated by hydrophobic, non-aromatic residues (L, I, and V) and basic residues (R and H). Facilitated by a unique orientation, Class V psNbs (5p-64, which shares high CDR similarity with 5p-175) targets RBD through a conserved epitope (T333, L387,₃₈₉DLC₃₉₁, C525, L517, and P521) completely buried in the spike and a region marginally overlapping with class IV psNbs (i.e. L517 and H519) (FIG. 27h-27i). While 5p-64 can bind strongly to the RBD it does not bind the prefusion spike and therefore only neutralizes the virus poorly in vitro (FIG. 34).

Mechanisms of Broad Neutralization by psNbs

These comprehensive structural results provide direct evidence that with successive immunization, antibodies have emerged in circulation that concurrently and almost exhaustively target diverse and conserved physicochemical and geometric properties on RBD. For ultrahigh-affinity Nbs (i.e., sub-nM), their neutralization potencies are inversely correlated, almost perfectly with the distance of epitopes to the RBS (Methods). Ultrapotent class I psNbs neutralize the virus at single-digit ng/ml by binding to RBS to block hACE2 binding (FIG. 40). Class II psNbs still efficiently neutralize the virus (dozens ng/ml) primarily by sterically interfering with the receptor binding. In particular, their RBD binding can clash with the glycan moiety at N322 of ACE2, especially bulky complex-types (FIG. 40). Class III, IV and V possess substantially weaker potencies. Their epitopes are distant from RBS and do not compete with ACE2 in vitro.

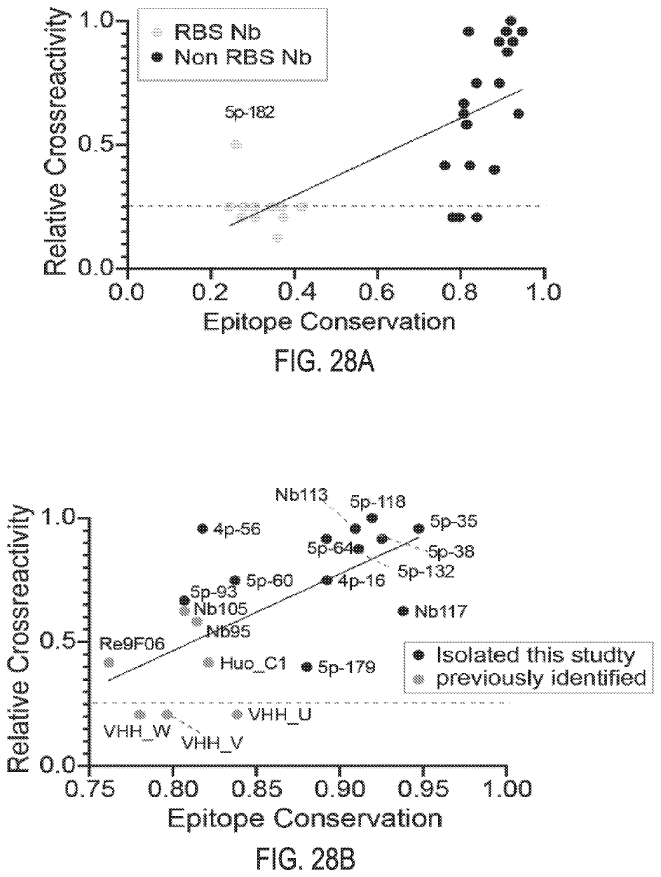

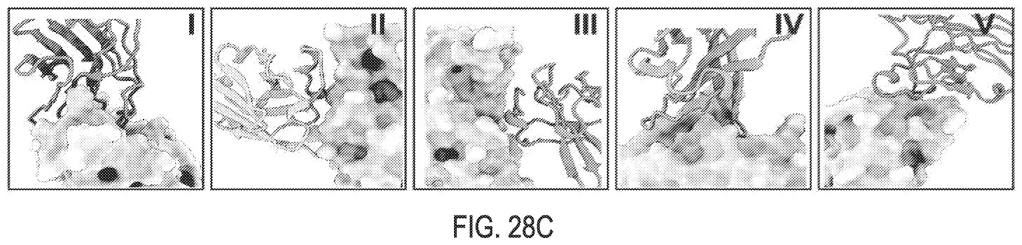

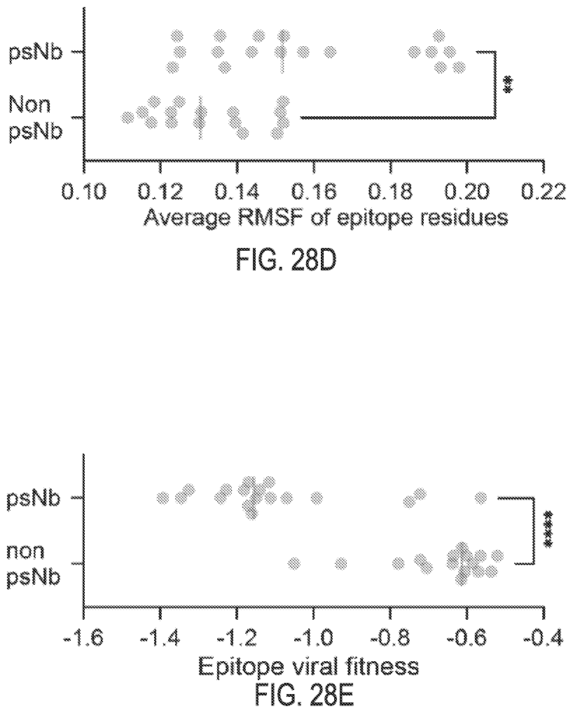

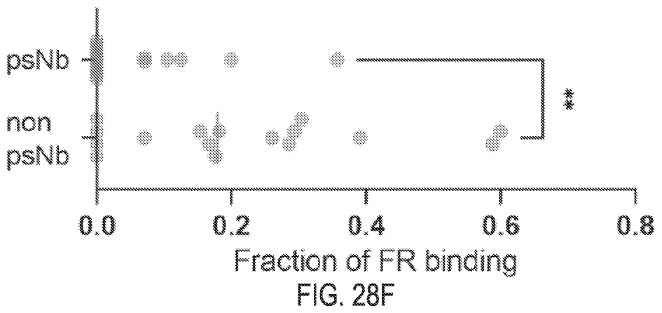

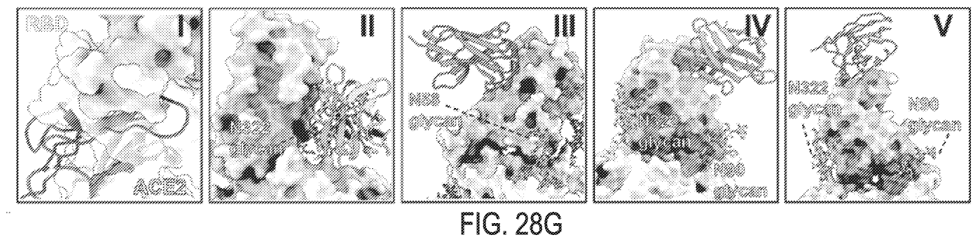

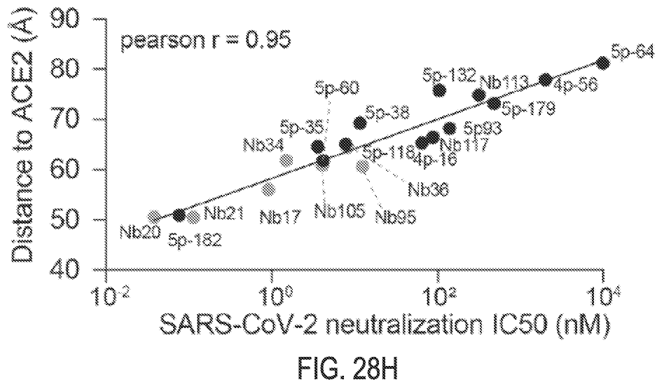

To better understand the broad activities of psNbs, a set of high-affinity RBD Nbs available were also expressed from the PDB and their cross-reactivity was evaluated by ELISA (Methods). Nbs fall into two groups based on RBS occupancy (FIG. 28a). Overall, their cross-reactivities positively correlate with the epitope sequence conservation (FIG. 28a). psNbs that do not bind RBS can be clearly separated based on high epitope conservation (>0.85) with an exception of class III psNb (5p-93) (FIG. 28b). Critical mutations from the concerning variants are predominantly identified on RBS but also recently on a small patch of relatively conserved residues (e.g., residues 371, 373 and 375) from the emerging VOC Omicron (B.1.1.529). Additionally, the virus can mutate on A372S/T to acquire a N-linked glycan (N370) similar to other sarbeoviruses to blunt host antibody response. While most Nbs and human antibodies directly interact with these residues, psNbs are barely affected (FIG. 24a, FIG. 33). Mutations on these highly conserved epitopes can cause RBD instability reducing viral fitness (FIG. 28e).

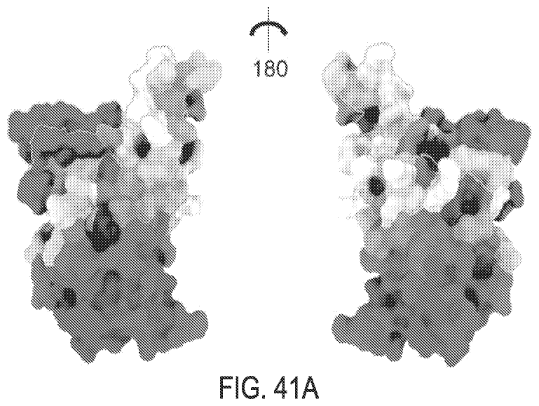

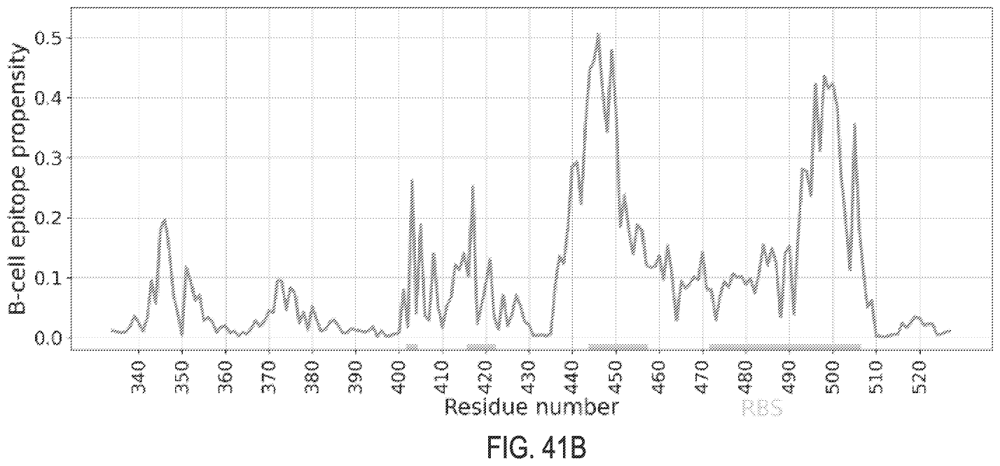

psNbs target significantly smaller epitopes that are characterized by flat (class II and III) or convex (class I and IV) structures (FIG. 28c-28d). Compared to non-broadly neutralizing RBS Nbs, PsNb epitopes are significantly more flexible (FIG. 28d). Together, these geometric and physicochemical properties may render high-affinity binding particularly challenging. Consistently, ScanNet, a geometric deep-learning model reveals that RBS epitopes are predominantly targeted by Nbs (and IgG antibodies) while the psNb epitopes are hardly recognized (FIG. 41). Moreover, compared to non-psNbs, psNbs utilize almost exclusively hypervariable CDR loops. Conceivably, extensive affinity maturation is required to bind these conserved yet difficult-to-bind epitopes (FIG. 28f).

SARS-CoV-2 continues to evolve, producing a string of variants with high transmissibility and potential to evade host immunity. New evidence indicates that natural infection or vaccination by booster can improve the host antibody response against SARS-CoV-2 variants. This study shows that successive immunization of a camelid by recombinant RBD can enhance the development of super-immunity. Integrative proteomics facilitated the rapid identification of a large repertoire of high-affinity VHH antibodies (Nbs) from the immunized sera against SARS-CoV-2 VOCs and the full spectrum of divergent sarbecovirus family. CryoEM and X-ray crystallography were used to systematically map broadly neutralizing epitopes and interactions, providing insights into the structural basis and evolution of serologic activities. These data support that RBD structure alone can drive this remarkable evolution reshaping the immunogenic landscape towards conserved epitopes. The initial immune response predominantly targets RBS due to its favorable properties for protein-protein interactions (both host receptor and antibody binding). Here, high-affinity antibodies can evolve to saturate this critical region with the highest neutralization potency. However, antibodies that target conserved and difficult to bind epitopes emerge with unprecedented diversity and can continuously evolve with improved affinities and breadth. Their neutralization potencies vary substantially yet are strongly and negatively correlated with the distance of epitopes to the RBS— central for viral entry and effective host immunity. Together, the findings herein inform the development of safe and broadly protecting interventions including vaccines and therapeutics.

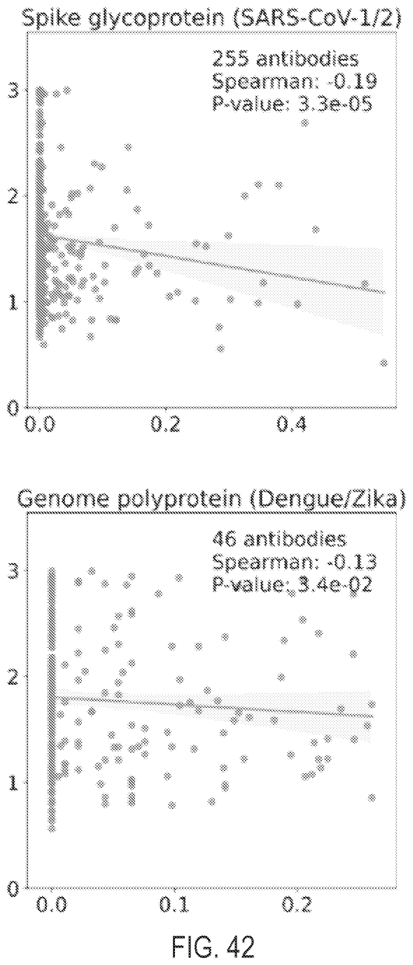

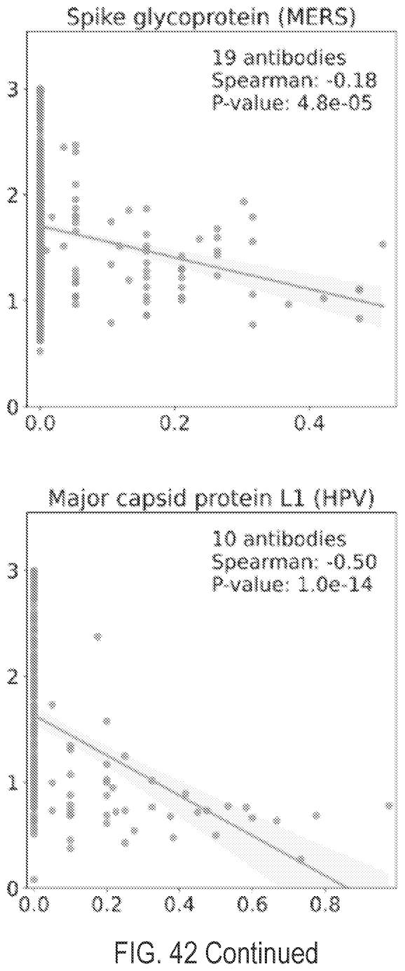

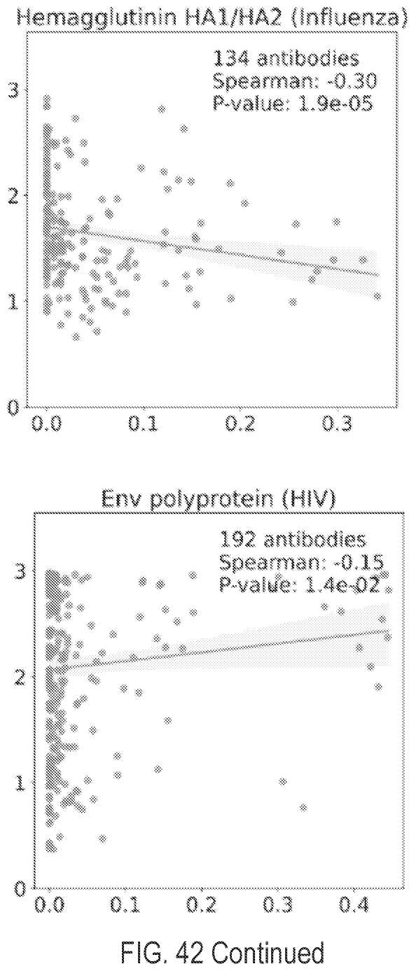

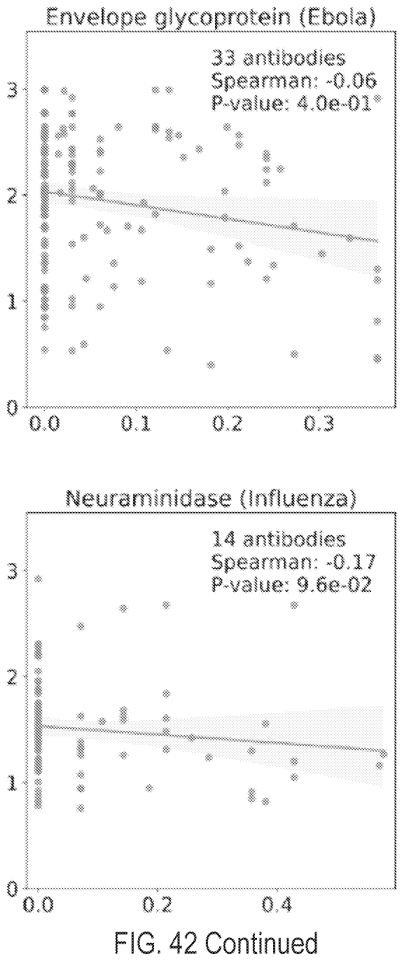

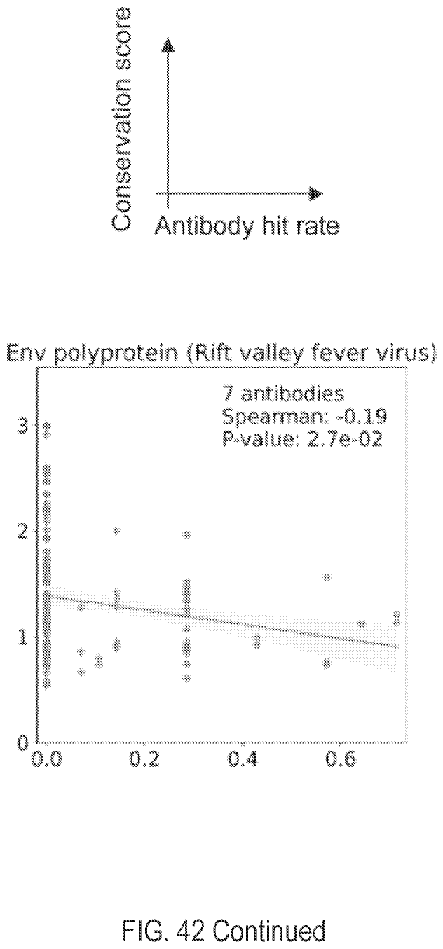

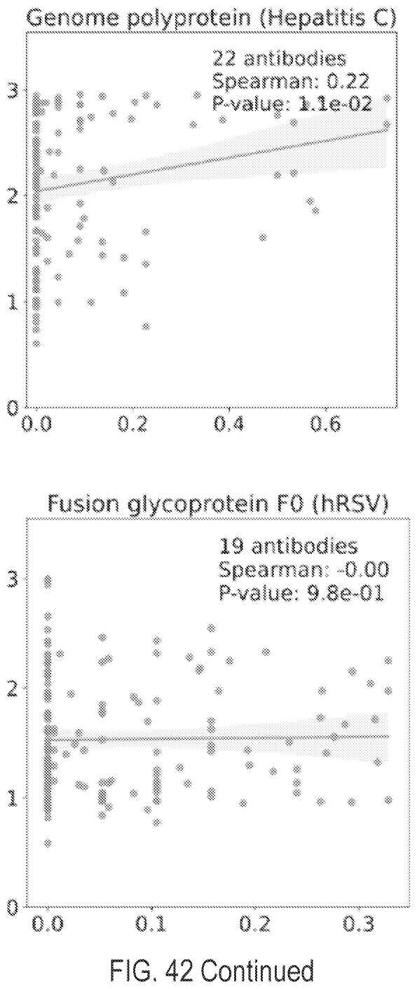

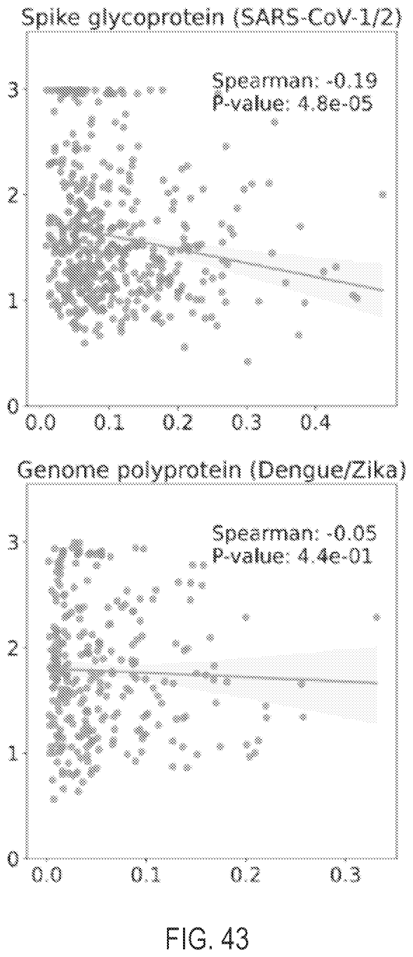

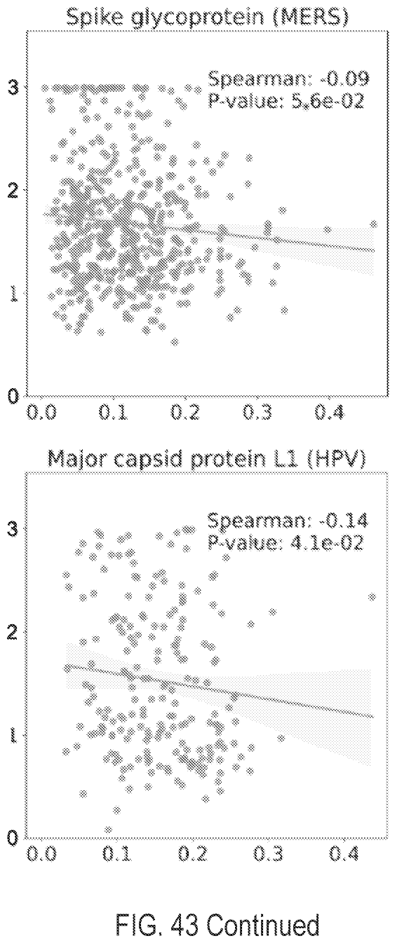

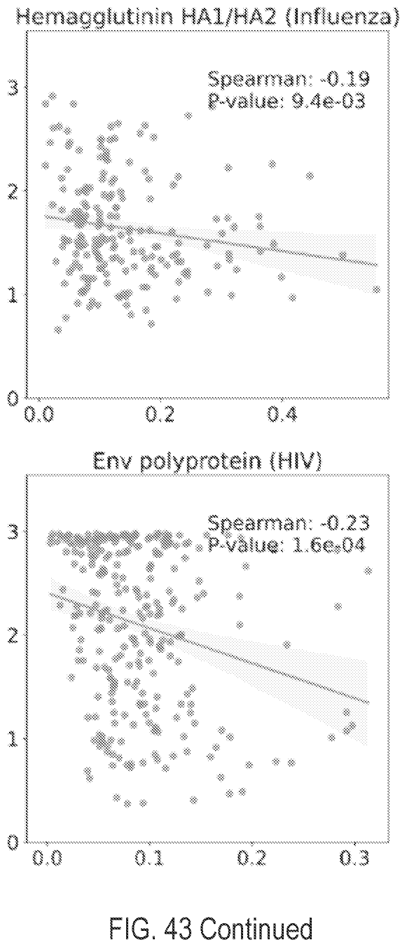

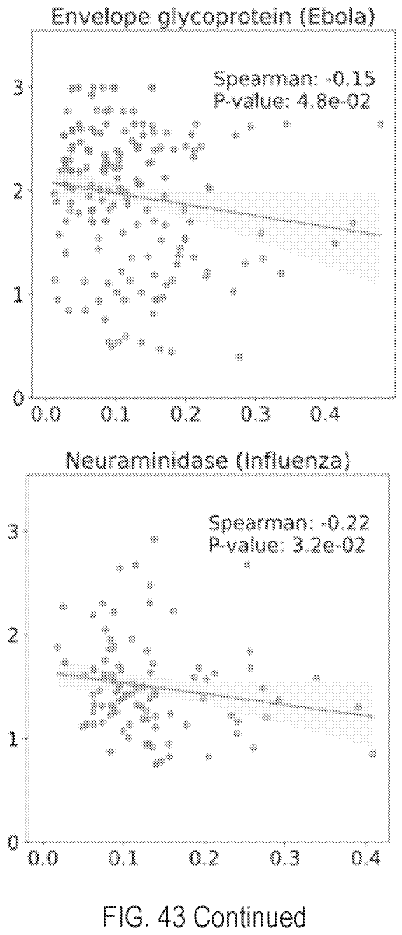

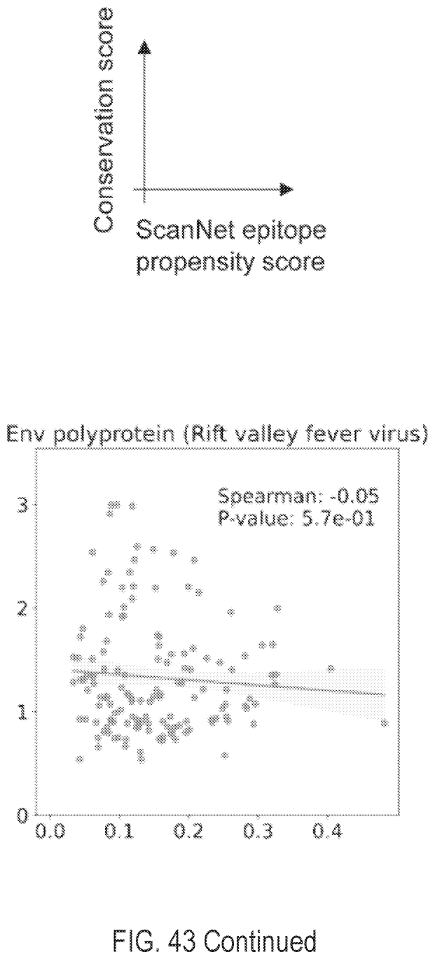

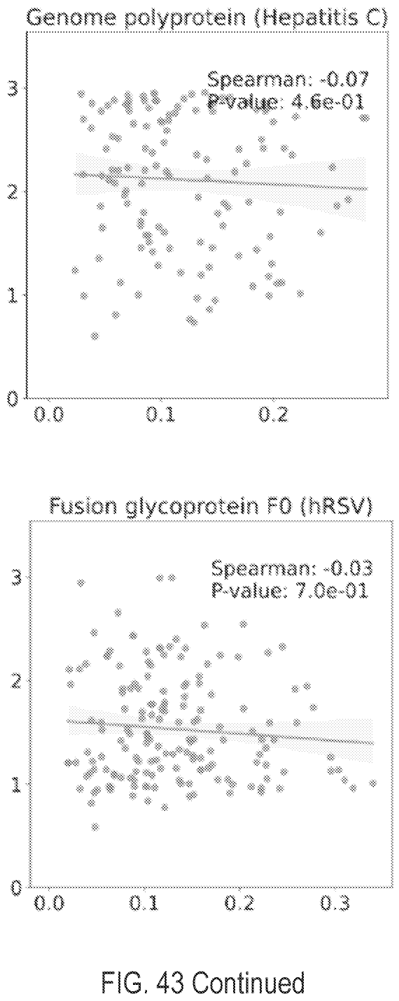

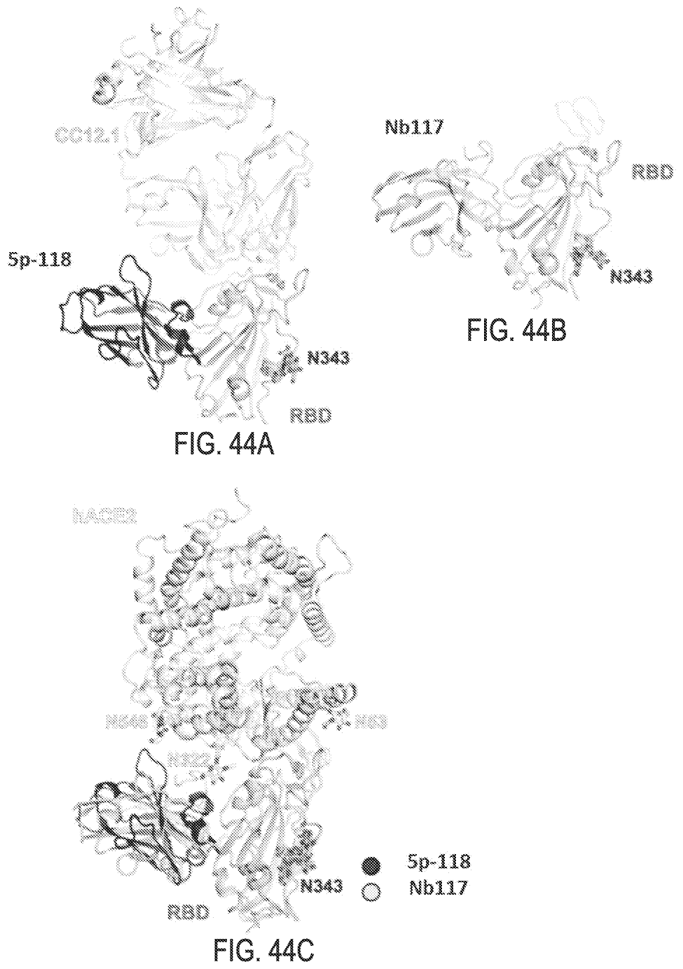

Preference for non-conserved epitopes is also observed in other viral antigen structures (FIG. 42). Evolutionary conservation imposes constraints on spatio-chemical surface properties, which in turn constraints the immunogenicity of epitopes, as supported by a negative correlation between conservation and predicted epitope propensity (FIG. 43). However, antibody repertoires are dynamic and can evolve towards conserved epitopes. In addition to COVID-19 super-immunity, broadly neutralizing antibodies have also been observed in HIV elite controllers, although they usually take years to evolve.

Highly selected psNbs can bind strongly and specifically to all sarbeoviruses for potent neutralization with the best median antiviral potencies at single-digit ng/ml, which is extremely rare for cross-reactive antibodies. Multivalent constructs (such as PiN-118) that target distinct and conserved epitopes and their cocktails may provide comprehensive coverage against future SARS-CoV-2 antigenic drift and new sarbecovirus challenges. The low production costs and marked stability of Nbs (and other miniproteins) allow for more equitable and efficient distributions globally, particularly for developing countries and regions that are vulnerable to spillovers. Combined with small size (high effective dose and bioavailability) and flexible administration routes that protect both upper and lower respiratory tracts to limit airborne transmissions, ultrapotent and inhalable psNbs are therefore highly complementary to vaccines, small molecule drugs, and monoclonal antibody therapeutics. Prospects of racing against future outbreaks will rely on the fast development and equitable distribution of an arsenal of broadly protecting, cost-effective and convenient technologies.

TABLE 1
Nanobodies and the corresponding sequences
of CDR3s and amino acid sequences.

| | | Representative amino |
|---|---|---|
| Nb name | CDR3 | acid Sequence |
| 5-39794-30 | SEQ ID NO: 1 | SEQ ID NO: 1058 |
| 5-14179-7 | SEQ ID NO: 2 | SEQ ID NO: 1059 |
| 5-78318-21 | SEQ ID NO: 3 | SEQ ID NO: 1060 |
| 5-102900-4 | SEQ ID NO: 4 | SEQ ID NO: 1061 |
| 5-23383-106 | SEQ ID NO: 5 | SEQ ID NO: 1062 |
| 5-730537-2 | SEQ ID NO: 6 | SEQ ID NO: 1063 |
| 5-9009-164 | SEQ ID NO: 7 | SEQ ID NO: 1064 |
| 5-10975-157 | SEQ ID NO: 8 | SEQ ID NO: 1065 |
| 5-30903-18 | SEQ ID NO: 9 | SEQ ID NO: 1066 |
| 5-1900414-232 | SEQ ID NO: 10 | SEQ ID NO: 1067 |
| 5-1109652-2 | SEQ ID NO: 11 | SEQ ID NO: 1068 |
| 5-1108791-32 | SEQ ID NO: 12 | SEQ ID NO: 1069 |
| 5-2526959-389 | SEQ ID NO: 13 | SEQ ID NO: 1070 |
| 5-1133032-5 | SEQ ID NO: 14 | SEQ ID NO: 1071 |
| 5-3107926-2 | SEQ ID NO: 15 | SEQ ID NO: 1072 |
| 5-2847738-2 | SEQ ID NO: 16 | SEQ ID NO: 1073 |
| 5-8769-74 | SEQ ID NO: 17 | SEQ ID NO: 1074 |
| 5-1413846-2 | SEQ ID NO: 18 | SEQ ID NO: 1075 |
| 5-1838762-2 | SEQ ID NO: 19 | SEQ ID NO: 1076 |
| 5-394886-2 | SEQ ID NO: 20 | SEQ ID NO: 1077 |
| 5-102503-3 | SEQ ID NO: 21 | SEQ ID NO: 1078 |
| 5-2551664-138 | SEQ ID NO: 22 | SEQ ID NO: 1079 |
| 5-2838419-3 | SEQ ID NO: 23 | SEQ ID NO: 1080 |
| 5-16102-4 | SEQ ID NO: 24 | SEQ ID NO: 1081 |
| 5-1013371-278 | SEQ ID NO: 25 | SEQ ID NO: 1082 |
| 5-14972-84 | SEQ ID NO: 26 | SEQ ID NO: 1083 |
| 5-36836-83 | SEQ ID NO: 27 | SEQ ID NO: 1084 |
| 5-571698-2 | SEQ ID NO: 28 | SEQ ID NO: 1085 |
| 5-329785-4 | SEQ ID NO: 29 | SEQ ID NO: 1086 |
| 5-14492-49 | SEQ ID NO: 30 | SEQ ID NO: 1087 |
| 5-1141754-6 | SEQ ID NO: 31 | SEQ ID NO: 1088 |
| 5-1044153-14 | SEQ ID NO: 32 | SEQ ID NO: 1089 |
| 4-4886354-26 | SEQ ID NO: 33 | SEQ ID NO: 1090 |
| 4-4813103-18 | SEQ ID NO: 34 | SEQ ID NO: 1091 |
| 4-4652444-35 | SEQ ID NO: 35 | SEQ ID NO: 1092 |
| 4-5024890-28 | SEQ ID NO: 36 | SEQ ID NO: 1093 |
| 4-6738235-2 | SEQ ID NO: 37 | SEQ ID NO: 1094 |
| 4-2399101-2 | SEQ ID NO: 38 | SEQ ID NO: 1095 |
| 4-6250396-10 | SEQ ID NO: 39 | SEQ ID NO: 1096 |
| 4-1859660-41 | SEQ ID NO: 40 | SEQ ID NO: 1097 |
| 4-8547239-20 | SEQ ID NO: 41 | SEQ ID NO: 1098 |
| 4-1967764-2 | SEQ ID NO: 42 | SEQ ID NO: 1099 |
| 4-765578-3 | SEQ ID NO: 43 | SEQ ID NO: 1100 |
| 4-101491-2 | SEQ ID NO: 44 | SEQ ID NO: 1101 |
| 4-8258415-2 | SEQ ID NO: 45 | SEQ ID NO: 1102 |
| 4-4695694-139 | SEQ ID NO: 46 | SEQ ID NO: 1103 |
| 4-2080196-11 | SEQ ID NO: 47 | SEQ ID NO: 1104 |
| 4-8542-164 | SEQ ID NO: 48 | SEQ ID NO: 1105 |
| 4-1779285-28 | SEQ ID NO: 49 | SEQ ID NO: 1106 |
| 4-2442780-60 | SEQ ID NO: 50 | SEQ ID NO: 1107 |
| 5-1164169-19 | SEQ ID NO: 51 | SEQ ID NO: 1108 |
| 5-2886632-2 | SEQ ID NO: 52 | SEQ ID NO: 1109 |
| 5-2559667-11 | SEQ ID NO: 53 | SEQ ID NO: 1110 |
| 5-1008455-104 | SEQ ID NO: 54 | SEQ ID NO: 1111 |
| 5-1086510-9 | SEQ ID NO: 55 | SEQ ID NO: 1112 |
| 5-162797-4 | SEQ ID NO: 56 | SEQ ID NO: 1113 |
| 5-2003166-4 | SEQ ID NO: 57 | SEQ ID NO: 1114 |
| 5-1032995-47 | SEQ ID NO: 58 | SEQ ID NO: 1115 |
| 5-1091941-13 | SEQ ID NO: 59 | SEQ ID NO: 1116 |
| 5-2201307-2 | SEQ ID NO: 60 | SEQ ID NO: 1117 |
| 5-2004369-2 | SEQ ID NO: 61 | SEQ ID NO: 1118 |
| 5-2545555-11 | SEQ ID NO: 62 | SEQ ID NO: 1119 |
| 5-71439-2 | SEQ ID NO: 63 | SEQ ID NO: 1120 |
| 5-184834-4 | SEQ ID NO: 64 | SEQ ID NO: 1121 |
| 5-664328-2 | SEQ ID NO: 65 | SEQ ID NO: 1122 |
| 5-36941-14 | SEQ ID NO: 66 | SEQ ID NO: 1123 |
| 5-203629-16 | SEQ ID NO: 67 | SEQ ID NO: 1124 |
| 5-748013-2 | SEQ ID NO: 68 | SEQ ID NO: 1125 |
| 5-1488982-2 | SEQ ID NO: 69 | SEQ ID NO: 1126 |
| 5-7538-125 | SEQ ID NO: 70 | SEQ ID NO: 1127 |
| 5-1021404-105 | SEQ ID NO: 71 | SEQ ID NO: 1128 |
| 5-1183153-5 | SEQ ID NO: 72 | SEQ ID NO: 1129 |
| 5-16666-24 | SEQ ID NO: 73 | SEQ ID NO: 1130 |
| 5-1923420-2 | SEQ ID NO: 74 | SEQ ID NO: 1131 |
| 5-91479-3 | SEQ ID NO: 75 | SEQ ID NO: 1132 |
| 5-401288-3 | SEQ ID NO: 76 | SEQ ID NO: 1133 |
| 5-6679-554 | SEQ ID NO: 77 | SEQ ID NO: 1134 |
| 5-2178053-2 | SEQ ID NO: 78 | SEQ ID NO: 1135 |
| 5-1989624-5 | SEQ ID NO: 79 | SEQ ID NO: 1136 |
| 5-1285961-2 | SEQ ID NO: 80 | SEQ ID NO: 1137 |
| 5-707174-3 | SEQ ID NO: 81 | SEQ ID NO: 1138 |
| 5-1136487-2 | SEQ ID NO: 82 | SEQ ID NO: 1139 |
| 5-2747983-3 | SEQ ID NO: 83 | SEQ ID NO: 1140 |
| 5-222289-8 | SEQ ID NO: 84 | SEQ ID NO: 1141 |
| 5-10519-382 | SEQ ID NO: 85 | SEQ ID NO: 1142 |
| 5-2610818-2 | SEQ ID NO: 86 | SEQ ID NO: 1143 |
| 5-1157289-2 | SEQ ID NO: 87 | SEQ ID NO: 1144 |
| 5-2625886-7 | SEQ ID NO: 88 | SEQ ID NO: 1145 |
| 5-2537388-104 | SEQ ID NO: 89 | SEQ ID NO: 1146 |
| 5-26593-46 | SEQ ID NO: 90 | SEQ ID NO: 1147 |
| 5-59087-28 | SEQ ID NO: 91 | SEQ ID NO: 1148 |
| 5-1131442-8 | SEQ ID NO: 92 | SEQ ID NO: 1149 |
| 5-2823876-4 | SEQ ID NO: 93 | SEQ ID NO: 1150 |
| 5-38836-59 | SEQ ID NO: 94 | SEQ ID NO: 1151 |
| 5-1906337-430 | SEQ ID NO: 95 | SEQ ID NO: 1152 |
| 5-1141754-6 | SEQ ID NO: 96 | SEQ ID NO: 1153 |
| 5-1143805-6 | SEQ ID NO: 97 | SEQ ID NO: 1154 |
| 5-82516-7 | SEQ ID NO: 98 | SEQ ID NO: 1155 |
| 5-1931110-4 | SEQ ID NO: 99 | SEQ ID NO: 1156 |
| 5-1065288-6 | SEQ ID NO: 100 | SEQ ID NO: 1157 |
| 5-2525750-813 | SEQ ID NO: 101 | SEQ ID NO: 1158 |
| 5-2547451-2 | SEQ ID NO: 102 | SEQ ID NO: 1159 |
| 5-2581500-3 | SEQ ID NO: 103 | SEQ ID NO: 1160 |
| 5-199676-4 | SEQ ID NO: 104 | SEQ ID NO: 1161 |
| 5-423822-2 | SEQ ID NO: 105 | SEQ ID NO: 1162 |
| 5-7632-31 | SEQ ID NO: 106 | SEQ ID NO: 1163 |
| 5-21526-67 | SEQ ID NO: 107 | SEQ ID NO: 1164 |
| 5-1928020-43 | SEQ ID NO: 108 | SEQ ID NO: 1165 |
| 5-1011284-458 | SEQ ID NO: 109 | SEQ ID NO: 1166 |
| 5-1550639-2 | SEQ ID NO: 110 | SEQ ID NO: 1167 |
| 5-2591036-2 | SEQ ID NO: 111 | SEQ ID NO: 1168 |
| 5-30916-15 | SEQ ID NO: 112 | SEQ ID NO: 1169 |
| 5-1382556-3 | SEQ ID NO: 113 | SEQ ID NO: 1170 |
| 5-2804995-2 | SEQ ID NO: 114 | SEQ ID NO: 1171 |
| 5-2539334-42 | SEQ ID NO: 115 | SEQ ID NO: 1172 |
| 5-316812-2 | SEQ ID NO: 116 | SEQ ID NO: 1173 |
| 5-48940-15 | SEQ ID NO: 117 | SEQ ID NO: 1174 |
| 5-1020089-113 | SEQ ID NO: 118 | SEQ ID NO: 1175 |
| 5-1080805-3 | SEQ ID NO: 119 | SEQ ID NO: 1176 |
| 5-1025130-333 | SEQ ID NO: 120 | SEQ ID NO: 1177 |
| 5-136494-2 | SEQ ID NO: 121 | SEQ ID NO: 1178 |
| 5-1011192-81 | SEQ ID NO: 122 | SEQ ID NO: 1179 |
| 5-2920582-2 | SEQ ID NO: 123 | SEQ ID NO: 1180 |
| 5-2523238-1186 | SEQ ID NO: 124 | SEQ ID NO: 1181 |
| 5-1022580-11 | SEQ ID NO: 125 | SEQ ID NO: 1182 |
| 5-3096069-3 | SEQ ID NO: 126 | SEQ ID NO: 1183 |
| 5-2534790-54 | SEQ ID NO: 127 | SEQ ID NO: 1184 |
| 5-1912390-63 | SEQ ID NO: 128 | SEQ ID NO: 1185 |
| 5-2534689-20 | SEQ ID NO: 129 | SEQ ID NO: 1186 |
| 5-1930836-13 | SEQ ID NO: 130 | SEQ ID NO: 1187 |
| 5-260759-11 | SEQ ID NO: 131 | SEQ ID NO: 1188 |
| 5-66535-4 | SEQ ID NO: 132 | SEQ ID NO: 1189 |
| 5-888206-2 | SEQ ID NO: 133 | SEQ ID NO: 1190 |
| 5-3009418-2 | SEQ ID NO: 134 | SEQ ID NO: 1191 |
| 5-290490-2 | SEQ ID NO: 135 | SEQ ID NO: 1192 |
| 5-2525729-31 | SEQ ID NO: 136 | SEQ ID NO: 1193 |
| 5-1008440-227 | SEQ ID NO: 137 | SEQ ID NO: 1194 |
| 5-285392-2 | SEQ ID NO: 138 | SEQ ID NO: 1195 |
| 5-313839-4 | SEQ ID NO: 139 | SEQ ID NO: 1196 |
| 5-2569271-9 | SEQ ID NO: 140 | SEQ ID NO: 1197 |
| 5-2548136-20 | SEQ ID NO: 141 | SEQ ID NO: 1198 |
| 5-159124-6 | SEQ ID NO: 142 | SEQ ID NO: 1199 |
| 5-7606-45 | SEQ ID NO: 143 | SEQ ID NO: 1200 |
| 5-4197-8 | SEQ ID NO: 144 | SEQ ID NO: 1201 |
| 5-87906-2 | SEQ ID NO: 145 | SEQ ID NO: 1202 |
| 5-1381257-2 | SEQ ID NO: 146 | SEQ ID NO: 1203 |
| 5-119896-2 | SEQ ID NO: 147 | SEQ ID NO: 1204 |
| 5-2045969-5 | SEQ ID NO: 148 | SEQ ID NO: 1205 |
| 5-1456715-2 | SEQ ID NO: 149 | SEQ ID NO: 1206 |
| 5-7049-253 | SEQ ID NO: 150 | SEQ ID NO: 1207 |
| 5-1401597-4 | SEQ ID NO: 151 | SEQ ID NO: 1208 |
| 5-1391-91 | SEQ ID NO: 152 | SEQ ID NO: 1209 |
| 5-1045786-32 | SEQ ID NO: 153 | SEQ ID NO: 1210 |
| 5-1898733-64 | SEQ ID NO: 154 | SEQ ID NO: 1211 |
| 5-1899-188 | SEQ ID NO: 155 | SEQ ID NO: 1212 |
| 5-1904653-45 | SEQ ID NO: 156 | SEQ ID NO: 1213 |
| 5-501185-2 | SEQ ID NO: 157 | SEQ ID NO: 1214 |
| 5-1050935-8 | SEQ ID NO: 158 | SEQ ID NO: 1215 |
| 5-391946-4 | SEQ ID NO: 159 | SEQ ID NO: 1216 |
| 5-2529215-7 | SEQ ID NO: 160 | SEQ ID NO: 1217 |
| 5-54711-21 | SEQ ID NO: 161 | SEQ ID NO: 1218 |
| 5-1315866-2 | SEQ ID NO: 162 | SEQ ID NO: 1219 |
| 4-459573-5 | SEQ ID NO: 163 | SEQ ID NO: 1220 |
| 4-1768361-154 | SEQ ID NO: 164 | SEQ ID NO: 1221 |
| 4-13096425-2 | SEQ ID NO: 165 | SEQ ID NO: 1222 |
| 4-7244663-2 | SEQ ID NO: 166 | SEQ ID NO: 1223 |
| 4-5553420-2 | SEQ ID NO: 167 | SEQ ID NO: 1224 |
| 4-13845232-2 | SEQ ID NO: 168 | SEQ ID NO: 1225 |
| 4-13339139-5 | SEQ ID NO: 169 | SEQ ID NO: 1226 |
| 4-8550695-4 | SEQ ID NO: 170 | SEQ ID NO: 1227 |
| 4-2097055-32 | SEQ ID NO: 171 | SEQ ID NO: 1228 |
| 4-7261410-2 | SEQ ID NO: 172 | SEQ ID NO: 1229 |
| 4-1815677-2 | SEQ ID NO: 173 | SEQ ID NO: 1230 |
| 4-3434681-3 | SEQ ID NO: 174 | SEQ ID NO: 1231 |
| 4-10700225-2 | SEQ ID NO: 175 | SEQ ID NO: 1232 |
| 4-2563169-12 | SEQ ID NO: 176 | SEQ ID NO: 1233 |
| 4-4633968-222 | SEQ ID NO: 177 | SEQ ID NO: 1234 |
| 4-1783819-2 | SEQ ID NO: 178 | SEQ ID NO: 1235 |
| 4-1277549-5 | SEQ ID NO: 179 | SEQ ID NO: 1236 |
| 4-9000521-4 | SEQ ID NO: 180 | SEQ ID NO: 1237 |
| 4-5704963-3 | SEQ ID NO: 181 | SEQ ID NO: 1238 |
| 4-3290480-2 | SEQ ID NO: 182 | SEQ ID NO: 1239 |
| 4-2343719-73 | SEQ ID NO: 183 | SEQ ID NO: 1240 |
| 4-2904900-18 | SEQ ID NO: 184 | SEQ ID NO: 1241 |
| 4-54624-57 | SEQ ID NO: 185 | SEQ ID NO: 1242 |
| 4-1380946-8 | SEQ ID NO: 186 | SEQ ID NO: 1243 |
| 4-1863754-148 | SEQ ID NO: 187 | SEQ ID NO: 1244 |
| 4-2397452-2 | SEQ ID NO: 188 | SEQ ID NO: 1245 |
| 4-437807-22 | SEQ ID NO: 189 | SEQ ID NO: 1246 |
| 4-2576489-13 | SEQ ID NO: 190 | SEQ ID NO: 1247 |
| 4-2391037-10 | SEQ ID NO: 191 | SEQ ID NO: 1248 |
| 4-6778424-2 | SEQ ID NO: 192 | SEQ ID NO: 1249 |
| 4-9871969-2 | SEQ ID NO: 193 | SEQ ID NO: 1250 |
| 4-12222-45 | SEQ ID NO: 194 | SEQ ID NO: 1251 |
| 4-2095401-49 | SEQ ID NO: 195 | SEQ ID NO: 1252 |
| 4-4779103-56 | SEQ ID NO: 196 | SEQ ID NO: 1253 |
| 4-2719802-4 | SEQ ID NO: 197 | SEQ ID NO: 1254 |
| 4-844927-2 | SEQ ID NO: 198 | SEQ ID NO: 1255 |
| 4-5099507-6 | SEQ ID NO: 199 | SEQ ID NO: 1256 |
| 4-4669689-415 | SEQ ID NO: 200 | SEQ ID NO: 1257 |
| 4-2805103-2 | SEQ ID NO: 201 | SEQ ID NO: 1258 |
| 4-8938700-2 | SEQ ID NO: 202 | SEQ ID NO: 1259 |
| 5-2538360-85 | SEQ ID NO: 203 | SEQ ID NO: 1260 |
| 5-1009969-217 | SEQ ID NO: 204 | SEQ ID NO: 1261 |
| 5-2813766-5 | SEQ ID NO: 205 | SEQ ID NO: 1262 |
| 5-549715-2 | SEQ ID NO: 206 | SEQ ID NO: 1263 |
| 5-110110-15 | SEQ ID NO: 207 | SEQ ID NO: 1264 |
| 5-1993009-8 | SEQ ID NO: 208 | SEQ ID NO: 1265 |
| 5-1735016-2 | SEQ ID NO: 209 | SEQ ID NO: 1266 |
| 5-1010853-156 | SEQ ID NO: 210 | SEQ ID NO: 1267 |
| 5-341922-2 | SEQ ID NO: 211 | SEQ ID NO: 1268 |
| 5-1900948-64 | SEQ ID NO: 212 | SEQ ID NO: 1269 |
| 5-81672-47 | SEQ ID NO: 213 | SEQ ID NO: 1270 |
| 5-2896693-3 | SEQ ID NO: 214 | SEQ ID NO: 1271 |
| 5-1033653-3 | SEQ ID NO: 215 | SEQ ID NO: 1272 |
| 5-205785-2 | SEQ ID NO: 216 | SEQ ID NO: 1273 |
| 5-2528191-66 | SEQ ID NO: 217 | SEQ ID NO: 1274 |
| 4-13181314-2 | SEQ ID NO: 218 | SEQ ID NO: 1275 |
| 4-8643638-15 | SEQ ID NO: 219 | SEQ ID NO: 1276 |
| 4-3351570-3 | SEQ ID NO: 220 | SEQ ID NO: 1277 |
| 4-3529447-4 | SEQ ID NO: 221 | SEQ ID NO: 1278 |
| 4-5882402-2 | SEQ ID NO: 222 | SEQ ID NO: 1279 |
| 4-9634958-3 | SEQ ID NO: 223 | SEQ ID NO: 1280 |
| 4-7004900-2 | SEQ ID NO: 224 | SEQ ID NO: 1281 |
| 4-9855676-3 | SEQ ID NO: 225 | SEQ ID NO: 1282 |
| 4-4612853-137 | SEQ ID NO: 226 | SEQ ID NO: 1283 |
| 4-4218110-19 | SEQ ID NO: 227 | SEQ ID NO: 1284 |
| 4-5081457-2 | SEQ ID NO: 228 | SEQ ID NO: 1285 |
| 4-8551389-14 | SEQ ID NO: 229 | SEQ ID NO: 1286 |
| 4-7288090-3 | SEQ ID NO: 230 | SEQ ID NO: 1287 |
| 4-5376326-10 | SEQ ID NO: 231 | SEQ ID NO: 1288 |
| 4-3394499-7 | SEQ ID NO: 232 | SEQ ID NO: 1289 |
| 4-5833422-2 | SEQ ID NO: 233 | SEQ ID NO: 1290 |
| 4-9291002-4 | SEQ ID NO: 234 | SEQ ID NO: 1291 |
| 4-5986046-8 | SEQ ID NO: 235 | SEQ ID NO: 1292 |
| 4-13991477-4 | SEQ ID NO: 236 | SEQ ID NO: 1293 |
| 4-6074765-7 | SEQ ID NO: 237 | SEQ ID NO: 1294 |
| 4-3593440-23 | SEQ ID NO: 238 | SEQ ID NO: 1295 |
| 4-2473437-11 | SEQ ID NO: 239 | SEQ ID NO: 1296 |
| 4-9700858-3 | SEQ ID NO: 240 | SEQ ID NO: 1297 |
| 4-1750311-445 | SEQ ID NO: 241 | SEQ ID NO: 1298 |
| 4-2094988-13 | SEQ ID NO: 242 | SEQ ID NO: 1299 |
| 4-4676422-12 | SEQ ID NO: 243 | SEQ ID NO: 1300 |
| 4-143351-3 | SEQ ID NO: 244 | SEQ ID NO: 1301 |
| 4-2083246-3 | SEQ ID NO: 245 | SEQ ID NO: 1302 |
| 4-1715713-2 | SEQ ID NO: 246 | SEQ ID NO: 1303 |
| 4-7051442-2 | SEQ ID NO: 247 | SEQ ID NO: 1304 |
| 4-5961115-4 | SEQ ID NO: 248 | SEQ ID NO: 1305 |
| 4-139696-5 | SEQ ID NO: 249 | SEQ ID NO: 1306 |
| 4-9295701-2 | SEQ ID NO: 250 | SEQ ID NO: 1307 |
| 4-3233807-2 | SEQ ID NO: 251 | SEQ ID NO: 1308 |
| 4-710183-4 | SEQ ID NO: 252 | SEQ ID NO: 1309 |
| 4-12800385-2 | SEQ ID NO: 253 | SEQ ID NO: 1310 |
| 4-2420002-3 | SEQ ID NO: 254 | SEQ ID NO: 1311 |
| 4-824260-2 | SEQ ID NO: 255 | SEQ ID NO: 1312 |
| 4-857253-4 | SEQ ID NO: 256 | SEQ ID NO: 1313 |
| 4-4084959-2 | SEQ ID NO: 257 | SEQ ID NO: 1314 |
| 4-1267404-2 | SEQ ID NO: 258 | SEQ ID NO: 1315 |
| 4-2105342-2 | SEQ ID NO: 259 | SEQ ID NO: 1316 |
| 4-9761654-2 | SEQ ID NO: 260 | SEQ ID NO: 1317 |
| 4-4335814-12 | SEQ ID NO: 261 | SEQ ID NO: 1318 |
| 4-12814759-2 | SEQ ID NO: 262 | SEQ ID NO: 1319 |
| 4-4609075-3 | SEQ ID NO: 263 | SEQ ID NO: 1320 |
| 4-3012497-5 | SEQ ID NO: 264 | SEQ ID NO: 1321 |
| 4-5261934-28 | SEQ ID NO: 265 | SEQ ID NO: 1322 |
| 4-13767854-2 | SEQ ID NO: 266 | SEQ ID NO: 1323 |
| 4-814525-2 | SEQ ID NO: 267 | SEQ ID NO: 1324 |
| 4-2344236-3 | SEQ ID NO: 268 | SEQ ID NO: 1325 |
| 4-3649887-34 | SEQ ID NO: 269 | SEQ ID NO: 1326 |
| 4-3818143-3 | SEQ ID NO: 270 | SEQ ID NO: 1327 |
| 4-3737337-6 | SEQ ID NO: 271 | SEQ ID NO: 1328 |
| 4-3925360-5 | SEQ ID NO: 272 | SEQ ID NO: 1329 |
| 4-2941965-2 | SEQ ID NO: 273 | SEQ ID NO: 1330 |
| 4-10032717-2 | SEQ ID NO: 274 | SEQ ID NO: 1331 |
| 4-6016609-2 | SEQ ID NO: 275 | SEQ ID NO: 1332 |
| 4-12750542-4 | SEQ ID NO: 276 | SEQ ID NO: 1333 |
| 4-864572-15 | SEQ ID NO: 277 | SEQ ID NO: 1334 |
| 4-384864-2 | SEQ ID NO: 278 | SEQ ID NO: 1335 |
| 4-604064-2 | SEQ ID NO: 279 | SEQ ID NO: 1336 |
| 4-1957109-20 | SEQ ID NO: 280 | SEQ ID NO: 1337 |
| 4-8510753-2 | SEQ ID NO: 281 | SEQ ID NO: 1338 |
| 4-5242183-16 | SEQ ID NO: 282 | SEQ ID NO: 1339 |
| 4-6064593-4 | SEQ ID NO: 283 | SEQ ID NO: 1340 |
| 4-2372995-4 | SEQ ID NO: 284 | SEQ ID NO: 1341 |
| 4-1001836-3 | SEQ ID NO: 285 | SEQ ID NO: 1342 |
| 4-91907-6 | SEQ ID NO: 286 | SEQ ID NO: 1343 |
| 4-6480286-9 | SEQ ID NO: 287 | SEQ ID NO: 1344 |
| 4-4754376-4 | SEQ ID NO: 288 | SEQ ID NO: 1345 |
| 4-11888335-6 | SEQ ID NO: 289 | SEQ ID NO: 1346 |
| 4-6789020-4 | SEQ ID NO: 290 | SEQ ID NO: 1347 |
| 4-302306-2 | SEQ ID NO: 291 | SEQ ID NO: 1348 |
| 4-5325863-2 | SEQ ID NO: 292 | SEQ ID NO: 1349 |
| 4-4642331-98 | SEQ ID NO: 293 | SEQ ID NO: 1350 |
| 4-3583449-13 | SEQ ID NO: 294 | SEQ ID NO: 1351 |
| 4-3180289-2 | SEQ ID NO: 295 | SEQ ID NO: 1352 |
| 4-13570405-2 | SEQ ID NO: 296 | SEQ ID NO: 1353 |
| 4-6186665-2 | SEQ ID NO: 297 | SEQ ID NO: 1354 |
| 4-12819666-2 | SEQ ID NO: 298 | SEQ ID NO: 1355 |
| 4-1234606-2 | SEQ ID NO: 299 | SEQ ID NO: 1356 |
| 4-6227837-13 | SEQ ID NO: 300 | SEQ ID NO: 1357 |
| 4-12773807-5 | SEQ ID NO: 301 | SEQ ID NO: 1358 |
| 4-13040705-2 | SEQ ID NO: 302 | SEQ ID NO: 1359 |
| 4-10083057-2 | SEQ ID NO: 303 | SEQ ID NO: 1360 |
| 4-1923132-28 | SEQ ID NO: 304 | SEQ ID NO: 1361 |
| 4-11148717-5 | SEQ ID NO: 305 | SEQ ID NO: 1362 |
| 4-6192964-2 | SEQ ID NO: 306 | SEQ ID NO: 1363 |
| 4-4991813-2 | SEQ ID NO: 307 | SEQ ID NO: 1364 |
| 4-5275824-2 | SEQ ID NO: 308 | SEQ ID NO: 1365 |
| 4-11545721-3 | SEQ ID NO: 309 | SEQ ID NO: 1366 |
| 4-409235-20 | SEQ ID NO: 310 | SEQ ID NO: 1367 |
| 4-1755477-2 | SEQ ID NO: 311 | SEQ ID NO: 1368 |
| 4-2955780-7 | SEQ ID NO: 312 | SEQ ID NO: 1369 |
| 4-1444120-2 | SEQ ID NO: 313 | SEQ ID NO: 1370 |
| 4-4650213-58 | SEQ ID NO: 314 | SEQ ID NO: 1371 |
| 4-2039149-2 | SEQ ID NO: 315 | SEQ ID NO: 1372 |
| 4-13019912-2 | SEQ ID NO: 316 | SEQ ID NO: 1373 |
| 4-3768935-16 | SEQ ID NO: 317 | SEQ ID NO: 1374 |
| 4-1219519-9 | SEQ ID NO: 318 | SEQ ID NO: 1375 |
| 4-11748771-5 | SEQ ID NO: 319 | SEQ ID NO: 1376 |
| 4-3727520-5 | SEQ ID NO: 320 | SEQ ID NO: 1377 |
| 4-3067663-5 | SEQ ID NO: 321 | SEQ ID NO: 1378 |
| 4-10110380-3 | SEQ ID NO: 322 | SEQ ID NO: 1379 |
| 4-9722169-2 | SEQ ID NO: 323 | SEQ ID NO: 1380 |
| 4-11642188-2 | SEQ ID NO: 324 | SEQ ID NO: 1381 |
| 4-6077766-2 | SEQ ID NO: 325 | SEQ ID NO: 1382 |
| 4-13997327-2 | SEQ ID NO: 326 | SEQ ID NO: 1383 |
| 4-246277-8 | SEQ ID NO: 327 | SEQ ID NO: 1384 |
| 4-8680685-2 | SEQ ID NO: 328 | SEQ ID NO: 1385 |
| 4-463361-4 | SEQ ID NO: 329 | SEQ ID NO: 1386 |
| 4-8604351-7 | SEQ ID NO: 330 | SEQ ID NO: 1387 |
| 4-4742374-7 | SEQ ID NO: 331 | SEQ ID NO: 1388 |
| 4-8704424-4 | SEQ ID NO: 332 | SEQ ID NO: 1389 |
| 4-1037296-2 | SEQ ID NO: 333 | SEQ ID NO: 1390 |
| 4-3505355-20 | SEQ ID NO: 334 | SEQ ID NO: 1391 |
| 4-887759-33 | SEQ ID NO: 335 | SEQ ID NO: 1392 |
| 4-5581221-2 | SEQ ID NO: 336 | SEQ ID NO: 1393 |
| 4-2235693-16 | SEQ ID NO: 337 | SEQ ID NO: 1394 |
| 4-1592082-2 | SEQ ID NO: 338 | SEQ ID NO: 1395 |
| 4-9325676-3 | SEQ ID NO: 339 | SEQ ID NO: 1396 |
| 4-2059129-3 | SEQ ID NO: 340 | SEQ ID NO: 1397 |
| 4-4027223-4 | SEQ ID NO: 341 | SEQ ID NO: 1398 |
| 4-9094581-9 | SEQ ID NO: 342 | SEQ ID NO: 1399 |
| 4-3291922-9 | SEQ ID NO: 343 | SEQ ID NO: 1400 |
| 4-1921786-5 | SEQ ID NO: 344 | SEQ ID NO: 1401 |
| 4-8496356-2 | SEQ ID NO: 345 | SEQ ID NO: 1402 |
| 4-11254889-8 | SEQ ID NO: 346 | SEQ ID NO: 1403 |
| 4-11203284-2 | SEQ ID NO: 347 | SEQ ID NO: 1404 |
| 4-6006398-2 | SEQ ID NO: 348 | SEQ ID NO: 1405 |
| 4-8540975-8 | SEQ ID NO: 349 | SEQ ID NO: 1406 |
| 4-13832531-2 | SEQ ID NO: 350 | SEQ ID NO: 1407 |
| 4-5521511-2 | SEQ ID NO: 351 | SEQ ID NO: 1408 |
| 4-11469871-2 | SEQ ID NO: 352 | SEQ ID NO: 1409 |
| 4-4104768-2 | SEQ ID NO: 353 | SEQ ID NO: 1410 |
| 4-2899623-15 | SEQ ID NO: 354 | SEQ ID NO: 1411 |
| 4-4608783-2 | SEQ ID NO: 355 | SEQ ID NO: 1412 |
| 4-8554098-2 | SEQ ID NO: 356 | SEQ ID NO: 1413 |
| 4-5828627-2 | SEQ ID NO: 357 | SEQ ID NO: 1414 |
| 4-12069155-2 | SEQ ID NO: 358 | SEQ ID NO: 1415 |
| 4-3530394-14 | SEQ ID NO: 359 | SEQ ID NO: 1416 |
| 4-4958693-2 | SEQ ID NO: 360 | SEQ ID NO: 1417 |
| 4-4413884-3 | SEQ ID NO: 361 | SEQ ID NO: 1418 |
| 4-3691216-5 | SEQ ID NO: 362 | SEQ ID NO: 1419 |
| 4-13355128-2 | SEQ ID NO: 363 | SEQ ID NO: 1420 |
| 4-4975142-8 | SEQ ID NO: 364 | SEQ ID NO: 1421 |
| 4-4827207-2 | SEQ ID NO: 365 | SEQ ID NO: 1422 |
| 4-3592965-2 | SEQ ID NO: 366 | SEQ ID NO: 1423 |
| 4-4469229-2 | SEQ ID NO: 367 | SEQ ID NO: 1424 |
| 4-7656645-2 | SEQ ID NO: 368 | SEQ ID NO: 1425 |
| 4-8930464-3 | SEQ ID NO: 369 | SEQ ID NO: 1426 |
| 4-6114869-2 | SEQ ID NO: 370 | SEQ ID NO: 1427 |
| 4-12357012-2 | SEQ ID NO: 371 | SEQ ID NO: 1428 |
| 4-4956154-3 | SEQ ID NO: 372 | SEQ ID NO: 1429 |
| 4-309472-5 | SEQ ID NO: 373 | SEQ ID NO: 1430 |
| 4-5566103-2 | SEQ ID NO: 374 | SEQ ID NO: 1431 |
| 4-6966458-2 | SEQ ID NO: 375 | SEQ ID NO: 1432 |
| 4-4762875-2 | SEQ ID NO: 376 | SEQ ID NO: 1433 |
| 4-5280394-23 | SEQ ID NO: 377 | SEQ ID NO: 1434 |
| 4-12280870-2 | SEQ ID NO: 378 | SEQ ID NO: 1435 |
| 4-6387478-2 | SEQ ID NO: 379 | SEQ ID NO: 1436 |
| 4-4140467-3 | SEQ ID NO: 380 | SEQ ID NO: 1437 |
| 4-8491270-3 | SEQ ID NO: 381 | SEQ ID NO: 1438 |
| 4-857327-9 | SEQ ID NO: 382 | SEQ ID NO: 1439 |
| 4-1177872-2 | SEQ ID NO: 383 | SEQ ID NO: 1440 |
| 4-2051185-5 | SEQ ID NO: 384 | SEQ ID NO: 1441 |
| 4-12892690-2 | SEQ ID NO: 385 | SEQ ID NO: 1442 |
| 4-3633881-17 | SEQ ID NO: 386 | SEQ ID NO: 1443 |
| 4-12820097-3 | SEQ ID NO: 387 | SEQ ID NO: 1444 |
| 4-12985944-3 | SEQ ID NO: 388 | SEQ ID NO: 1445 |
| 4-11458388-2 | SEQ ID NO: 389 | SEQ ID NO: 1446 |
| 4-11201525-2 | SEQ ID NO: 390 | SEQ ID NO: 1447 |
| 4-803066-2 | SEQ ID NO: 391 | SEQ ID NO: 1448 |
| 4-2026448-3 | SEQ ID NO: 392 | SEQ ID NO: 1449 |
| 4-8643669-4 | SEQ ID NO: 393 | SEQ ID NO: 1450 |
| 4-1841440-2 | SEQ ID NO: 394 | SEQ ID NO: 1451 |
| 4-14202295-2 | SEQ ID NO: 395 | SEQ ID NO: 1452 |
| 4-5274756-2 | SEQ ID NO: 396 | SEQ ID NO: 1453 |
| 4-1813655-18 | SEQ ID NO: 397 | SEQ ID NO: 1454 |
| 4-3910110-2 | SEQ ID NO: 398 | SEQ ID NO: 1455 |
| 4-13064342-3 | SEQ ID NO: 399 | SEQ ID NO: 1456 |
| 4-12755556-3 | SEQ ID NO: 400 | SEQ ID NO: 1457 |
| 4-458984-4 | SEQ ID NO: 401 | SEQ ID NO: 1458 |
| 4-9065335-3 | SEQ ID NO: 402 | SEQ ID NO: 1459 |
| 4-2055720-4 | SEQ ID NO: 403 | SEQ ID NO: 1460 |
| 4-12806037-2 | SEQ ID NO: 404 | SEQ ID NO: 1461 |
| 4-3542860-8 | SEQ ID NO: 405 | SEQ ID NO: 1462 |
| 4-3549132-36 | SEQ ID NO: 406 | SEQ ID NO: 1463 |
| 4-5785557-5 | SEQ ID NO: 407 | SEQ ID NO: 1464 |
| 4-2619438-3 | SEQ ID NO: 408 | SEQ ID NO: 1465 |
| 4-55403-24 | SEQ ID NO: 409 | SEQ ID NO: 1466 |
| 4-8592714-2 | SEQ ID NO: 410 | SEQ ID NO: 1467 |
| 4-1822841-8 | SEQ ID NO: 411 | SEQ ID NO: 1468 |
| 4-4705102-3 | SEQ ID NO: 412 | SEQ ID NO: 1469 |
| 4-3571266-27 | SEQ ID NO: 413 | SEQ ID NO: 1470 |
| 4-2356644-50 | SEQ ID NO: 414 | SEQ ID NO: 1471 |
| 4-3623627-83 | SEQ ID NO: 415 | SEQ ID NO: 1472 |
| 4-104794-22 | SEQ ID NO: 416 | SEQ ID NO: 1473 |
| 4-3849839-2 | SEQ ID NO: 417 | SEQ ID NO: 1474 |
| 4-4955760-5 | SEQ ID NO: 418 | SEQ ID NO: 1475 |
| 4-9684484-2 | SEQ ID NO: 419 | SEQ ID NO: 1476 |
| 4-2229631-38 | SEQ ID NO: 420 | SEQ ID NO: 1477 |
| 4-12800770-2 | SEQ ID NO: 421 | SEQ ID NO: 1478 |
| 4-379824-29 | SEQ ID NO: 422 | SEQ ID NO: 1479 |
| 4-1814560-26 | SEQ ID NO: 423 | SEQ ID NO: 1480 |
| 4-11137042-2 | SEQ ID NO: 424 | SEQ ID NO: 1481 |
| 4-2837615-2 | SEQ ID NO: 425 | SEQ ID NO: 1482 |
| 4-5301672-16 | SEQ ID NO: 426 | SEQ ID NO: 1483 |
| 4-4662850-41 | SEQ ID NO: 427 | SEQ ID NO: 1484 |
| 4-4367876-2 | SEQ ID NO: 428 | SEQ ID NO: 1485 |
| 4-3655837-28 | SEQ ID NO: 429 | SEQ ID NO: 1486 |
| 4-4554413-2 | SEQ ID NO: 430 | SEQ ID NO: 1487 |
| 4-6928846-2 | SEQ ID NO: 431 | SEQ ID NO: 1488 |
| 4-4850546-4 | SEQ ID NO: 432 | SEQ ID NO: 1489 |
| 4-9271814-2 | SEQ ID NO: 433 | SEQ ID NO: 1490 |
| 4-930187-3 | SEQ ID NO: 434 | SEQ ID NO: 1491 |
| 4-1891607-18 | SEQ ID NO: 435 | SEQ ID NO: 1492 |
| 4-8759327-2 | SEQ ID NO: 436 | SEQ ID NO: 1493 |
| 4-11671362-3 | SEQ ID NO: 437 | SEQ ID NO: 1494 |
| 4-9453382-4 | SEQ ID NO: 438 | SEQ ID NO: 1495 |
| 4-12010665-2 | SEQ ID NO: 439 | SEQ ID NO: 1496 |
| 4-8541709-2 | SEQ ID NO: 440 | SEQ ID NO: 1497 |
| 4-13208089-2 | SEQ ID NO: 441 | SEQ ID NO: 1498 |
| 4-3691991-2 | SEQ ID NO: 442 | SEQ ID NO: 1499 |
| 4-14340382-2 | SEQ ID NO: 443 | SEQ ID NO: 1500 |
| 4-9575068-2 | SEQ ID NO: 444 | SEQ ID NO: 1501 |
| 4-6345686-2 | SEQ ID NO: 445 | SEQ ID NO: 1502 |
| 4-4767859-4 | SEQ ID NO: 446 | SEQ ID NO: 1503 |
| 4-6083486-4 | SEQ ID NO: 447 | SEQ ID NO: 1504 |
| 4-6915682-2 | SEQ ID NO: 448 | SEQ ID NO: 1505 |
| 4-12763665-5 | SEQ ID NO: 449 | SEQ ID NO: 1506 |
| 4-5316151-3 | SEQ ID NO: 450 | SEQ ID NO: 1507 |
| 4-1457754-2 | SEQ ID NO: 451 | SEQ ID NO: 1508 |
| 4-13080345-3 | SEQ ID NO: 452 | SEQ ID NO: 1509 |
| 4-5562115-2 | SEQ ID NO: 453 | SEQ ID NO: 1510 |
| 4-8491269-2 | SEQ ID NO: 454 | SEQ ID NO: 1511 |
| 4-306028-2 | SEQ ID NO: 455 | SEQ ID NO: 1512 |
| 4-8928663-4 | SEQ ID NO: 456 | SEQ ID NO: 1513 |
| 4-8887332-2 | SEQ ID NO: 457 | SEQ ID NO: 1514 |
| 4-5358098-2 | SEQ ID NO: 458 | SEQ ID NO: 1515 |
| 4-8778218-2 | SEQ ID NO: 459 | SEQ ID NO: 1516 |
| 4-6074750-2 | SEQ ID NO: 460 | SEQ ID NO: 1517 |
| 4-4858767-9 | SEQ ID NO: 461 | SEQ ID NO: 1518 |
| 4-2057069-3 | SEQ ID NO: 462 | SEQ ID NO: 1519 |
| 4-7559770-4 | SEQ ID NO: 463 | SEQ ID NO: 1520 |
| 4-5256250-2 | SEQ ID NO: 464 | SEQ ID NO: 1521 |
| 4-3342406-2 | SEQ ID NO: 465 | SEQ ID NO: 1522 |
| 4-1434290-5 | SEQ ID NO: 466 | SEQ ID NO: 1523 |
| 4-13228696-2 | SEQ ID NO: 467 | SEQ ID NO: 1524 |
| 4-4233749-2 | SEQ ID NO: 468 | SEQ ID NO: 1525 |
| 4-5434139-5 | SEQ ID NO: 469 | SEQ ID NO: 1526 |
| 4-11515477-5 | SEQ ID NO: 470 | SEQ ID NO: 1527 |
| 4-6198566-2 | SEQ ID NO: 471 | SEQ ID NO: 1528 |
| 4-41791-3 | SEQ ID NO: 472 | SEQ ID NO: 1529 |
| 4-4828189-2 | SEQ ID NO: 473 | SEQ ID NO: 1530 |
| 4-5732731-2 | SEQ ID NO: 474 | SEQ ID NO: 1531 |
| 4-2871272-5 | SEQ ID NO: 475 | SEQ ID NO: 1532 |
| 4-5187317-2 | SEQ ID NO: 476 | SEQ ID NO: 1533 |
| 4-3174909-2 | SEQ ID NO: 477 | SEQ ID NO: 1534 |
| 4-3655165-4 | SEQ ID NO: 478 | SEQ ID NO: 1535 |
| 4-317502-2 | SEQ ID NO: 479 | SEQ ID NO: 1536 |
| 4-11435519-2 | SEQ ID NO: 480 | SEQ ID NO: 1537 |
| 4-3075969-12 | SEQ ID NO: 481 | SEQ ID NO: 1538 |
| 4-12753692-2 | SEQ ID NO: 482 | SEQ ID NO: 1539 |
| 4-3639656-12 | SEQ ID NO: 483 | SEQ ID NO: 1540 |
| 4-5390580-23 | SEQ ID NO: 484 | SEQ ID NO: 1541 |
| 4-11456736-4 | SEQ ID NO: 485 | SEQ ID NO: 1542 |
| 4-6888851-2 | SEQ ID NO: 486 | SEQ ID NO: 1543 |
| 4-9503046-2 | SEQ ID NO: 487 | SEQ ID NO: 1544 |
| 4-9425129-2 | SEQ ID NO: 488 | SEQ ID NO: 1545 |
| 4-2654006-2 | SEQ ID NO: 489 | SEQ ID NO: 1546 |
| 4-4818200-13 | SEQ ID NO: 490 | SEQ ID NO: 1547 |
| 4-4332759-2 | SEQ ID NO: 491 | SEQ ID NO: 1548 |
| 4-2002208-28 | SEQ ID NO: 492 | SEQ ID NO: 1549 |
| 4-1861724-2 | SEQ ID NO: 493 | SEQ ID NO: 1550 |
| 4-6151797-2 | SEQ ID NO: 494 | SEQ ID NO: 1551 |
| 4-12889516-9 | SEQ ID NO: 495 | SEQ ID NO: 1552 |
| 4-37618-4 | SEQ ID NO: 496 | SEQ ID NO: 1553 |
| 4-4138911-26 | SEQ ID NO: 497 | SEQ ID NO: 1554 |
| 4-5172072-2 | SEQ ID NO: 498 | SEQ ID NO: 1555 |
| 4-4474995-7 | SEQ ID NO: 499 | SEQ ID NO: 1556 |
| 4-2910890-20 | SEQ ID NO: 500 | SEQ ID NO: 1557 |
| 4-8553413-4 | SEQ ID NO: 501 | SEQ ID NO: 1558 |
| 4-6202770-2 | SEQ ID NO: 502 | SEQ ID NO: 1559 |
| 4-1204174-2 | SEQ ID NO: 503 | SEQ ID NO: 1560 |
| 4-6189913-2 | SEQ ID NO: 504 | SEQ ID NO: 1561 |
| 4-3820419-14 | SEQ ID NO: 505 | SEQ ID NO: 1562 |
| 4-3030848-2 | SEQ ID NO: 506 | SEQ ID NO: 1563 |
| 4-2652596-3 | SEQ ID NO: 507 | SEQ ID NO: 1564 |
| 4-7652765-2 | SEQ ID NO: 508 | SEQ ID NO: 1565 |
| 4-1511825-2 | SEQ ID NO: 509 | SEQ ID NO: 1566 |
| 4-495325-2 | SEQ ID NO: 510 | SEQ ID NO: 1567 |
| 4-4790144-2 | SEQ ID NO: 511 | SEQ ID NO: 1568 |
| 4-9531730-2 | SEQ ID NO: 512 | SEQ ID NO: 1569 |
| 4-10604235-2 | SEQ ID NO: 513 | SEQ ID NO: 1570 |
| 4-457480-5 | SEQ ID NO: 514 | SEQ ID NO: 1571 |
| 4-2084932-13 | SEQ ID NO: 515 | SEQ ID NO: 1572 |
| 4-6256390-2 | SEQ ID NO: 516 | SEQ ID NO: 1573 |
| 4-3362597-2 | SEQ ID NO: 517 | SEQ ID NO: 1574 |
| 4-4231535-3 | SEQ ID NO: 518 | SEQ ID NO: 1575 |
| 4-4764333-4 | SEQ ID NO: 519 | SEQ ID NO: 1576 |
| 4-14846-3 | SEQ ID NO: 520 | SEQ ID NO: 1577 |
| 4-656941-3 | SEQ ID NO: 521 | SEQ ID NO: 1578 |
| 4-13200123-2 | SEQ ID NO: 522 | SEQ ID NO: 1579 |
| 4-5342212-5 | SEQ ID NO: 523 | SEQ ID NO: 1580 |
| 4-9238016-3 | SEQ ID NO: 524 | SEQ ID NO: 1581 |
| 4-813594-27 | SEQ ID NO: 525 | SEQ ID NO: 1582 |
| 4-5402398-2 | SEQ ID NO: 526 | SEQ ID NO: 1583 |
| 4-4648528-7 | SEQ ID NO: 527 | SEQ ID NO: 1584 |
| 4-5706894-2 | SEQ ID NO: 528 | SEQ ID NO: 1585 |
| 4-2017044-25 | SEQ ID NO: 529 | SEQ ID NO: 1586 |
| 4-3610202-9 | SEQ ID NO: 530 | SEQ ID NO: 1587 |
| 4-1409229-2 | SEQ ID NO: 531 | SEQ ID NO: 1588 |
| 4-523698-5 | SEQ ID NO: 532 | SEQ ID NO: 1589 |
| 4-9160654-3 | SEQ ID NO: 533 | SEQ ID NO: 1590 |
| 4-7013429-3 | SEQ ID NO: 534 | SEQ ID NO: 1591 |
| 4-4922921-8 | SEQ ID NO: 535 | SEQ ID NO: 1592 |
| 4-589059-2 | SEQ ID NO: 536 | SEQ ID NO: 1593 |
| 4-1012998-3 | SEQ ID NO: 537 | SEQ ID NO: 1594 |
| 4-6191771-7 | SEQ ID NO: 538 | SEQ ID NO: 1595 |
| 4-947738-9 | SEQ ID NO: 539 | SEQ ID NO: 1596 |
| 4-10347787-2 | SEQ ID NO: 540 | SEQ ID NO: 1597 |
| 4-5487145-2 | SEQ ID NO: 541 | SEQ ID NO: 1598 |
| 4-1436238-5 | SEQ ID NO: 542 | SEQ ID NO: 1599 |
| 4-6061062-2 | SEQ ID NO: 543 | SEQ ID NO: 1600 |
| 4-3715847-8 | SEQ ID NO: 544 | SEQ ID NO: 1601 |
| 4-4855542-6 | SEQ ID NO: 545 | SEQ ID NO: 1602 |
| 4-10601470-2 | SEQ ID NO: 546 | SEQ ID NO: 1603 |
| 4-648180-2 | SEQ ID NO: 547 | SEQ ID NO: 1604 |
| 4-1924630-2 | SEQ ID NO: 548 | SEQ ID NO: 1605 |
| 4-11226934-4 | SEQ ID NO: 549 | SEQ ID NO: 1606 |
| 4-6085115-2 | SEQ ID NO: 550 | SEQ ID NO: 1607 |
| 4-4413708-8 | SEQ ID NO: 551 | SEQ ID NO: 1608 |
| 4-2164399-17 | SEQ ID NO: 552 | SEQ ID NO: 1609 |
| 4-2840415-12 | SEQ ID NO: 553 | SEQ ID NO: 1610 |
| 4-10011522-2 | SEQ ID NO: 554 | SEQ ID NO: 1611 |
| 4-2727434-28 | SEQ ID NO: 555 | SEQ ID NO: 1612 |
| 4-11302862-2 | SEQ ID NO: 556 | SEQ ID NO: 1613 |
| 4-11848302-3 | SEQ ID NO: 557 | SEQ ID NO: 1614 |
| 4-1915025-53 | SEQ ID NO: 558 | SEQ ID NO: 1615 |
| 4-1624090-2 | SEQ ID NO: 559 | SEQ ID NO: 1616 |
| 4-7676197-2 | SEQ ID NO: 560 | SEQ ID NO: 1617 |
| 4-2687435-2 | SEQ ID NO: 561 | SEQ ID NO: 1618 |
| 4-3095685-2 | SEQ ID NO: 562 | SEQ ID NO: 1619 |
| 4-8069674-2 | SEQ ID NO: 563 | SEQ ID NO: 1620 |
| 4-5376323-2 | SEQ ID NO: 564 | SEQ ID NO: 1621 |
| 4-9558086-2 | SEQ ID NO: 565 | SEQ ID NO: 1622 |
| 4-14175261-2 | SEQ ID NO: 566 | SEQ ID NO: 1623 |
| 4-7338078-2 | SEQ ID NO: 567 | SEQ ID NO: 1624 |
| 4-8527490-11 | SEQ ID NO: 568 | SEQ ID NO: 1625 |
| 4-161300-3 | SEQ ID NO: 569 | SEQ ID NO: 1626 |
| 4-8993715-2 | SEQ ID NO: 570 | SEQ ID NO: 1627 |
| 4-1616718-6 | SEQ ID NO: 571 | SEQ ID NO: 1628 |
| 4-11522705-3 | SEQ ID NO: 572 | SEQ ID NO: 1629 |
| 4-3242990-2 | SEQ ID NO: 573 | SEQ ID NO: 1630 |
| 4-8666515-2 | SEQ ID NO: 574 | SEQ ID NO: 1631 |
| 4-12796552-7 | SEQ ID NO: 575 | SEQ ID NO: 1632 |
| 4-2625622-5 | SEQ ID NO: 576 | SEQ ID NO: 1633 |
| 4-4155587-2 | SEQ ID NO: 577 | SEQ ID NO: 1634 |
| 4-13156013-2 | SEQ ID NO: 578 | SEQ ID NO: 1635 |
| 4-13580555-3 | SEQ ID NO: 579 | SEQ ID NO: 1636 |
| 4-96616-15 | SEQ ID NO: 580 | SEQ ID NO: 1637 |
| 4-5041579-11 | SEQ ID NO: 581 | SEQ ID NO: 1638 |
| 4-6517845-3 | SEQ ID NO: 582 | SEQ ID NO: 1639 |
| 4-6774106-2 | SEQ ID NO: 583 | SEQ ID NO: 1640 |
| 4-6450274-5 | SEQ ID NO: 584 | SEQ ID NO: 1641 |
| 4-8512678-2 | SEQ ID NO: 585 | SEQ ID NO: 1642 |
| 4-8597863-2 | SEQ ID NO: 586 | SEQ ID NO: 1643 |
| 4-4637162-2 | SEQ ID NO: 587 | SEQ ID NO: 1644 |
| 4-10253885-2 | SEQ ID NO: 588 | SEQ ID NO: 1645 |
| 4-243065-14 | SEQ ID NO: 589 | SEQ ID NO: 1646 |
| 4-11380738-2 | SEQ ID NO: 590 | SEQ ID NO: 1647 |
| 4-14075705-2 | SEQ ID NO: 591 | SEQ ID NO: 1648 |
| 4-5967379-2 | SEQ ID NO: 592 | SEQ ID NO: 1649 |
| 4-12419326-3 | SEQ ID NO: 593 | SEQ ID NO: 1650 |
| 4-13091749-2 | SEQ ID NO: 594 | SEQ ID NO: 1651 |
| 4-195248-11 | SEQ ID NO: 595 | SEQ ID NO: 1652 |
| 4-4786709-2 | SEQ ID NO: 596 | SEQ ID NO: 1653 |
| 4-5303454-3 | SEQ ID NO: 597 | SEQ ID NO: 1654 |
| 4-2230237-17 | SEQ ID NO: 598 | SEQ ID NO: 1655 |
| 4-11964353-3 | SEQ ID NO: 599 | SEQ ID NO: 1656 |
| 4-4788889-3 | SEQ ID NO: 600 | SEQ ID NO: 1657 |
| 4-6964778-2 | SEQ ID NO: 601 | SEQ ID NO: 1658 |
| 4-6896134-2 | SEQ ID NO: 602 | SEQ ID NO: 1659 |
| 4-6087863-14 | SEQ ID NO: 603 | SEQ ID NO: 1660 |
| 4-10026563-2 | SEQ ID NO: 604 | SEQ ID NO: 1661 |
| 4-5539032-9 | SEQ ID NO: 605 | SEQ ID NO: 1662 |
| 4-9690250-4 | SEQ ID NO: 606 | SEQ ID NO: 1663 |
| 4-8210248-2 | SEQ ID NO: 607 | SEQ ID NO: 1664 |
| 4-3732589-19 | SEQ ID NO: 608 | SEQ ID NO: 1665 |
| 4-2964718-6 | SEQ ID NO: 609 | SEQ ID NO: 1666 |
| 4-3813395-8 | SEQ ID NO: 610 | SEQ ID NO: 1667 |
| 4-247424-2 | SEQ ID NO: 611 | SEQ ID NO: 1668 |
| 4-9014657-2 | SEQ ID NO: 612 | SEQ ID NO: 1669 |
| 4-2775294-5 | SEQ ID NO: 613 | SEQ ID NO: 1670 |
| 4-10464607-2 | SEQ ID NO: 614 | SEQ ID NO: 1671 |
| 4-13175347-2 | SEQ ID NO: 615 | SEQ ID NO: 1672 |
| 4-11511701-2 | SEQ ID NO: 616 | SEQ ID NO: 1673 |
| 4-11616893-2 | SEQ ID NO: 617 | SEQ ID NO: 1674 |
| 4-12948939-3 | SEQ ID NO: 618 | SEQ ID NO: 1675 |
| 4-13483989-2 | SEQ ID NO: 619 | SEQ ID NO: 1676 |
| 4-8506183-2 | SEQ ID NO: 620 | SEQ ID NO: 1677 |
| 4-6483216-2 | SEQ ID NO: 621 | SEQ ID NO: 1678 |
| 4-6131305-7 | SEQ ID NO: 622 | SEQ ID NO: 1679 |
| 4-9581201-2 | SEQ ID NO: 623 | SEQ ID NO: 1680 |
| 4-3091012-2 | SEQ ID NO: 624 | SEQ ID NO: 1681 |
| 4-6809018-2 | SEQ ID NO: 625 | SEQ ID NO: 1682 |
| 4-1143729-2 | SEQ ID NO: 626 | SEQ ID NO: 1683 |
| 4-5018770-2 | SEQ ID NO: 627 | SEQ ID NO: 1684 |
| 4-2082086-2 | SEQ ID NO: 628 | SEQ ID NO: 1685 |
| 4-558751-2 | SEQ ID NO: 629 | SEQ ID NO: 1686 |
| 4-6665135-2 | SEQ ID NO: 630 | SEQ ID NO: 1687 |
| 4-3651780-5 | SEQ ID NO: 631 | SEQ ID NO: 1688 |
| 4-11347961-2 | SEQ ID NO: 632 | SEQ ID NO: 1689 |
| 4-2010097-3 | SEQ ID NO: 633 | SEQ ID NO: 1690 |
| 4-9356905-3 | SEQ ID NO: 634 | SEQ ID NO: 1691 |
| 4-161517-6 | SEQ ID NO: 635 | SEQ ID NO: 1692 |
| 4-9005681-3 | SEQ ID NO: 636 | SEQ ID NO: 1693 |
| 4-349495-2 | SEQ ID NO: 637 | SEQ ID NO: 1694 |
| 4-2279246-4 | SEQ ID NO: 638 | SEQ ID NO: 1695 |
| 4-5718875-2 | SEQ ID NO: 639 | SEQ ID NO: 1696 |
| 4-9202788-3 | SEQ ID NO: 640 | SEQ ID NO: 1697 |
| 4-11932452-3 | SEQ ID NO: 641 | SEQ ID NO: 1698 |
| 4-8719052-6 | SEQ ID NO: 642 | SEQ ID NO: 1699 |
| 4-13589827-2 | SEQ ID NO: 643 | SEQ ID NO: 1700 |
| 4-1583398-6 | SEQ ID NO: 644 | SEQ ID NO: 1701 |
| 4-5611265-2 | SEQ ID NO: 645 | SEQ ID NO: 1702 |
| 4-11418307-2 | SEQ ID NO: 646 | SEQ ID NO: 1703 |
| 4-4482198-2 | SEQ ID NO: 647 | SEQ ID NO: 1704 |
| 4-3789491-2 | SEQ ID NO: 648 | SEQ ID NO: 1705 |
| 4-5795560-2 | SEQ ID NO: 649 | SEQ ID NO: 1706 |
| 4-712744-2 | SEQ ID NO: 650 | SEQ ID NO: 1707 |
| 4-2623643-2 | SEQ ID NO: 651 | SEQ ID NO: 1708 |
| 4-2032915-2 | SEQ ID NO: 652 | SEQ ID NO: 1709 |
| 4-10098193-2 | SEQ ID NO: 653 | SEQ ID NO: 1710 |
| 4-7383866-2 | SEQ ID NO: 654 | SEQ ID NO: 1711 |
| 4-5013350-2 | SEQ ID NO: 655 | SEQ ID NO: 1712 |
| 4-4720581-3 | SEQ ID NO: 656 | SEQ ID NO: 1713 |
| 4-9991639-2 | SEQ ID NO: 657 | SEQ ID NO: 1714 |
| 4-106394-2 | SEQ ID NO: 658 | SEQ ID NO: 1715 |
| 4-3157200-4 | SEQ ID NO: 659 | SEQ ID NO: 1716 |
| 4-3576581-2 | SEQ ID NO: 660 | SEQ ID NO: 1717 |
| 4-6583593-2 | SEQ ID NO: 661 | SEQ ID NO: 1718 |
| 4-9667098-4 | SEQ ID NO: 662 | SEQ ID NO: 1719 |
| 4-4626312-4 | SEQ ID NO: 663 | SEQ ID NO: 1720 |
| 4-8712204-2 | SEQ ID NO: 664 | SEQ ID NO: 1721 |
| 4-4414504-3 | SEQ ID NO: 665 | SEQ ID NO: 1722 |
| 4-7023003-3 | SEQ ID NO: 666 | SEQ ID NO: 1723 |
| 4-10147520-2 | SEQ ID NO: 667 | SEQ ID NO: 1724 |
| 4-147641-2 | SEQ ID NO: 668 | SEQ ID NO: 1725 |
| 4-2618723-2 | SEQ ID NO: 669 | SEQ ID NO: 1726 |
| 4-4653676-2 | SEQ ID NO: 670 | SEQ ID NO: 1727 |
| 4-13100536-2 | SEQ ID NO: 671 | SEQ ID NO: 1728 |
| 4-570180-3 | SEQ ID NO: 672 | SEQ ID NO: 1729 |
| 4-6116434-2 | SEQ ID NO: 673 | SEQ ID NO: 1730 |
| 4-3371101-3 | SEQ ID NO: 674 | SEQ ID NO: 1731 |
| 4-6521725-7 | SEQ ID NO: 675 | SEQ ID NO: 1732 |
| 4-11718170-2 | SEQ ID NO: 676 | SEQ ID NO: 1733 |
| 4-11159205-2 | SEQ ID NO: 677 | SEQ ID NO: 1734 |
| 4-13407986-2 | SEQ ID NO: 678 | SEQ ID NO: 1735 |
| 4-4292125-2 | SEQ ID NO: 679 | SEQ ID NO: 1736 |
| 4-6268471-3 | SEQ ID NO: 680 | SEQ ID NO: 1737 |
| 4-6367327-3 | SEQ ID NO: 681 | SEQ ID NO: 1738 |
| 4-5278922-5 | SEQ ID NO: 682 | SEQ ID NO: 1739 |
| 4-1833015-3 | SEQ ID NO: 683 | SEQ ID NO: 1740 |
| 4-1432020-2 | SEQ ID NO: 684 | SEQ ID NO: 1741 |
| 4-6203153-3 | SEQ ID NO: 685 | SEQ ID NO: 1742 |
| 4-6488890-2 | SEQ ID NO: 686 | SEQ ID NO: 1743 |
| 4-2298899-2 | SEQ ID NO: 687 | SEQ ID NO: 1744 |
| 4-4695406-2 | SEQ ID NO: 688 | SEQ ID NO: 1745 |
| 4-2628870-2 | SEQ ID NO: 689 | SEQ ID NO: 1746 |
| 4-11708708-2 | SEQ ID NO: 690 | SEQ ID NO: 1747 |
| 4-1860788-3 | SEQ ID NO: 691 | SEQ ID NO: 1748 |
| 4-3229401-2 | SEQ ID NO: 692 | SEQ ID NO: 1749 |
| 4-4603394-3 | SEQ ID NO: 693 | SEQ ID NO: 1750 |
| 4-13507081-2 | SEQ ID NO: 694 | SEQ ID NO: 1751 |
| 4-1007788-2 | SEQ ID NO: 695 | SEQ ID NO: 1752 |
| 4-2730370-2 | SEQ ID NO: 696 | SEQ ID NO: 1753 |
| 4-2654962-3 | SEQ ID NO: 697 | SEQ ID NO: 1754 |
| 4-9042945-2 | SEQ ID NO: 698 | SEQ ID NO: 1755 |
| 4-3215161-2 | SEQ ID NO: 699 | SEQ ID NO: 1756 |
| 4-5104773-2 | SEQ ID NO: 700 | SEQ ID NO: 1757 |
| 4-11599478-2 | SEQ ID NO: 701 | SEQ ID NO: 1758 |
| 4-6539104-2 | SEQ ID NO: 702 | SEQ ID NO: 1759 |
| 4-6527299-4 | SEQ ID NO: 703 | SEQ ID NO: 1760 |
| 4-2136022-12 | SEQ ID NO: 704 | SEQ ID NO: 1761 |
| 4-5346916-2 | SEQ ID NO: 705 | SEQ ID NO: 1762 |
| 4-5503066-2 | SEQ ID NO: 706 | SEQ ID NO: 1763 |
| 4-2969169-2 | SEQ ID NO: 707 | SEQ ID NO: 1764 |
| 4-5422432-2 | SEQ ID NO: 708 | SEQ ID NO: 1765 |
| 4-7527833-2 | SEQ ID NO: 709 | SEQ ID NO: 1766 |
| 4-12911216-3 | SEQ ID NO: 710 | SEQ ID NO: 1767 |
| 4-8531908-2 | SEQ ID NO: 711 | SEQ ID NO: 1768 |
| 4-5390013-17 | SEQ ID NO: 712 | SEQ ID NO: 1769 |
| 4-2455525-2 | SEQ ID NO: 713 | SEQ ID NO: 1770 |
| 4-12923413-2 | SEQ ID NO: 714 | SEQ ID NO: 1771 |
| 4-7027184-2 | SEQ ID NO: 715 | SEQ ID NO: 1772 |
| 4-2997533-2 | SEQ ID NO: 716 | SEQ ID NO: 1773 |
| 4-14244201-2 | SEQ ID NO: 717 | SEQ ID NO: 1774 |
| 4-1363486-2 | SEQ ID NO: 718 | SEQ ID NO: 1775 |
| 4-11297702-2 | SEQ ID NO: 719 | SEQ ID NO: 1776 |
| 4-13318642-3 | SEQ ID NO: 720 | SEQ ID NO: 1777 |
| 4-13387560-2 | SEQ ID NO: 721 | SEQ ID NO: 1778 |
| 4-6984109-2 | SEQ ID NO: 722 | SEQ ID NO: 1779 |
| 4-12906253-4 | SEQ ID NO: 723 | SEQ ID NO: 1780 |
| 4-6875993-2 | SEQ ID NO: 724 | SEQ ID NO: 1781 |
| 4-2480865-2 | SEQ ID NO: 725 | SEQ ID NO: 1782 |
| 4-9107845-2 | SEQ ID NO: 726 | SEQ ID NO: 1783 |
| 4-12000519-2 | SEQ ID NO: 727 | SEQ ID NO: 1784 |
| 4-13983010-2 | SEQ ID NO: 728 | SEQ ID NO: 1785 |
| 4-2422760-2 | SEQ ID NO: 729 | SEQ ID NO: 1786 |
| 4-4158211-2 | SEQ ID NO: 730 | SEQ ID NO: 1787 |
| 4-2806535-5 | SEQ ID NO: 731 | SEQ ID NO: 1788 |
| 4-4828379-3 | SEQ ID NO: 732 | SEQ ID NO: 1789 |
| 4-11411601-3 | SEQ ID NO: 733 | SEQ ID NO: 1790 |
| 4-3635766-2 | SEQ ID NO: 734 | SEQ ID NO: 1791 |
| 4-8587973-2 | SEQ ID NO: 735 | SEQ ID NO: 1792 |
| 4-4865191-2 | SEQ ID NO: 736 | SEQ ID NO: 1793 |
| 4-6775722-2 | SEQ ID NO: 737 | SEQ ID NO: 1794 |
| 4-4855645-2 | SEQ ID NO: 738 | SEQ ID NO: 1795 |
| 4-6838997-2 | SEQ ID NO: 739 | SEQ ID NO: 1796 |
| 4-5331867-2 | SEQ ID NO: 740 | SEQ ID NO: 1797 |
| 4-6889938-2 | SEQ ID NO: 741 | SEQ ID NO: 1798 |
| 4-6769533-2 | SEQ ID NO: 742 | SEQ ID NO: 1799 |
| 4-3099528-2 | SEQ ID NO: 743 | SEQ ID NO: 1800 |
| 4-13291027-2 | SEQ ID NO: 744 | SEQ ID NO: 1801 |
| 4-11293195-2 | SEQ ID NO: 745 | SEQ ID NO: 1802 |
| 4-2618135-2 | SEQ ID NO: 746 | SEQ ID NO: 1803 |
| 4-12900160-2 | SEQ ID NO: 747 | SEQ ID NO: 1804 |
| 4-6486522-2 | SEQ ID NO: 748 | SEQ ID NO: 1805 |
| 4-3381696-2 | SEQ ID NO: 749 | SEQ ID NO: 1806 |
| 4-6102410-3 | SEQ ID NO: 750 | SEQ ID NO: 1807 |
| 4-10485598-2 | SEQ ID NO: 751 | SEQ ID NO: 1808 |
| 4-14528374-2 | SEQ ID NO: 752 | SEQ ID NO: 1809 |
| 4-6910586-2 | SEQ ID NO: 753 | SEQ ID NO: 1810 |
| 4-1175262-3 | SEQ ID NO: 754 | SEQ ID NO: 1811 |
| 4-12795613-3 | SEQ ID NO: 755 | SEQ ID NO: 1812 |
| 4-9244418-2 | SEQ ID NO: 756 | SEQ ID NO: 1813 |
| 4-6482247-3 | SEQ ID NO: 757 | SEQ ID NO: 1814 |
| 4-5219438-4 | SEQ ID NO: 758 | SEQ ID NO: 1815 |
| 4-11922365-2 | SEQ ID NO: 759 | SEQ ID NO: 1816 |
| 4-609357-2 | SEQ ID NO: 760 | SEQ ID NO: 1817 |
| 4-2398287-2 | SEQ ID NO: 761 | SEQ ID NO: 1818 |
| 4-4545492-2 | SEQ ID NO: 762 | SEQ ID NO: 1819 |
| 4-13238753-3 | SEQ ID NO: 763 | SEQ ID NO: 1820 |
| 4-11112882-3 | SEQ ID NO: 764 | SEQ ID NO: 1821 |
| 4-8588049-2 | SEQ ID NO: 765 | SEQ ID NO: 1822 |
| 4-12753525-2 | SEQ ID NO: 766 | SEQ ID NO: 1823 |
| 4-48251-2 | SEQ ID NO: 767 | SEQ ID NO: 1824 |
| 4-7181238-2 | SEQ ID NO: 768 | SEQ ID NO: 1825 |
| 4-7595088-2 | SEQ ID NO: 769 | SEQ ID NO: 1826 |
| 4-10466330-2 | SEQ ID NO: 770 | SEQ ID NO: 1827 |
| 4-8792031-2 | SEQ ID NO: 771 | SEQ ID NO: 1828 |
| 4-6373021-2 | SEQ ID NO: 772 | SEQ ID NO: 1829 |
| 4-8648860-2 | SEQ ID NO: 773 | SEQ ID NO: 1830 |
| 4-13568679-2 | SEQ ID NO: 774 | SEQ ID NO: 1831 |
| 4-8004216-2 | SEQ ID NO: 775 | SEQ ID NO: 1832 |
| 4-6794114-2 | SEQ ID NO: 776 | SEQ ID NO: 1833 |
| 4-6962898-3 | SEQ ID NO: 777 | SEQ ID NO: 1834 |
| 4-5481102-3 | SEQ ID NO: 778 | SEQ ID NO: 1835 |
| 4-2717399-2 | SEQ ID NO: 779 | SEQ ID NO: 1836 |
| 4-4159662-3 | SEQ ID NO: 780 | SEQ ID NO: 1837 |
| 5-2829056-3 | SEQ ID NO: 781 | SEQ ID NO: 1838 |
| 5-565972-2 | SEQ ID NO: 782 | SEQ ID NO: 1839 |
| 5-2120962-3 | SEQ ID NO: 783 | SEQ ID NO: 1840 |
| 5-1020879-11 | SEQ ID NO: 784 | SEQ ID NO: 1841 |
| 5-749568-2 | SEQ ID NO: 785 | SEQ ID NO: 1842 |
| 5-1054065-21 | SEQ ID NO: 786 | SEQ ID NO: 1843 |
| 5-854-158 | SEQ ID NO: 787 | SEQ ID NO: 1844 |
| 5-1170708-3 | SEQ ID NO: 788 | SEQ ID NO: 1845 |
| 5-50360-15 | SEQ ID NO: 789 | SEQ ID NO: 1846 |
| 5-1383597-2 | SEQ ID NO: 790 | SEQ ID NO: 1847 |
| 5-2705599-2 | SEQ ID NO: 791 | SEQ ID NO: 1848 |
| 5-1130896-2 | SEQ ID NO: 792 | SEQ ID NO: 1849 |
| 5-1038048-2 | SEQ ID NO: 793 | SEQ ID NO: 1850 |
| 5-1135283-2 | SEQ ID NO: 794 | SEQ ID NO: 1851 |
| 5-2893875-2 | SEQ ID NO: 795 | SEQ ID NO: 1852 |
| 5-3186217-2 | SEQ ID NO: 796 | SEQ ID NO: 1853 |
| 5-1012006-2 | SEQ ID NO: 797 | SEQ ID NO: 1854 |
| 5-1037600-4 | SEQ ID NO: 798 | SEQ ID NO: 1855 |
| 5-1402975-3 | SEQ ID NO: 799 | SEQ ID NO: 1856 |
| 5-2631492-5 | SEQ ID NO: 800 | SEQ ID NO: 1857 |
| 5-1637513-2 | SEQ ID NO: 801 | SEQ ID NO: 1858 |
| 5-1376127-2 | SEQ ID NO: 802 | SEQ ID NO: 1859 |
| 5-2007790-4 | SEQ ID NO: 803 | SEQ ID NO: 1860 |
| 5-13204-283 | SEQ ID NO: 804 | SEQ ID NO: 1861 |
| 5-2587052-13 | SEQ ID NO: 805 | SEQ ID NO: 1862 |
| 5-1195003-2 | SEQ ID NO: 806 | SEQ ID NO: 1863 |
| 5-2670630-2 | SEQ ID NO: 807 | SEQ ID NO: 1864 |
| 5-1901924-10 | SEQ ID NO: 808 | SEQ ID NO: 1865 |
| 5-23953-5 | SEQ ID NO: 809 | SEQ ID NO: 1866 |
| 5-217863-2 | SEQ ID NO: 810 | SEQ ID NO: 1867 |
| 5-1571958-2 | SEQ ID NO: 811 | SEQ ID NO: 1868 |
| 5-1959833-18 | SEQ ID NO: 812 | SEQ ID NO: 1869 |
| 5-1031826-27 | SEQ ID NO: 813 | SEQ ID NO: 1870 |
| 5-41960-97 | SEQ ID NO: 814 | SEQ ID NO: 1871 |
| 5-1923420-2 | SEQ ID NO: 815 | SEQ ID NO: 1872 |
| 5-1092647-6 | SEQ ID NO: 816 | SEQ ID NO: 1873 |
| 5-2552779-4 | SEQ ID NO: 817 | SEQ ID NO: 1874 |
| 5-686-192 | SEQ ID NO: 818 | SEQ ID NO: 1875 |
| 5-2944422-2 | SEQ ID NO: 819 | SEQ ID NO: 1876 |
| 5-2529089-143 | SEQ ID NO: 820 | SEQ ID NO: 1877 |
| 5-38164-2 | SEQ ID NO: 821 | SEQ ID NO: 1878 |
| 5-1502750-3 | SEQ ID NO: 822 | SEQ ID NO: 1879 |
| 5-2857200-2 | SEQ ID NO: 823 | SEQ ID NO: 1880 |
| 5-32716-2 | SEQ ID NO: 824 | SEQ ID NO: 1881 |
| 5-1928401-49 | SEQ ID NO: 825 | SEQ ID NO: 1882 |
| 5-1212566-4 | SEQ ID NO: 826 | SEQ ID NO: 1883 |
| 5-1122882-9 | SEQ ID NO: 827 | SEQ ID NO: 1884 |
| 5-51576-5 | SEQ ID NO: 828 | SEQ ID NO: 1885 |
| 5-118624-5 | SEQ ID NO: 829 | SEQ ID NO: 1886 |
| 5-2048039-2 | SEQ ID NO: 830 | SEQ ID NO: 1887 |
| 5-1013605-11 | SEQ ID NO: 831 | SEQ ID NO: 1888 |
| 5-394268-2 | SEQ ID NO: 832 | SEQ ID NO: 1889 |
| 5-1924606-22 | SEQ ID NO: 833 | SEQ ID NO: 1890 |
| 5-2842754-2 | SEQ ID NO: 834 | SEQ ID NO: 1891 |
| 5-1061053-2 | SEQ ID NO: 835 | SEQ ID NO: 1892 |
| 5-2532260-2 | SEQ ID NO: 836 | SEQ ID NO: 1893 |
| 5-2593-2 | SEQ ID NO: 837 | SEQ ID NO: 1894 |
| 5-2592305-11 | SEQ ID NO: 838 | SEQ ID NO: 1895 |
| 5-661674-2 | SEQ ID NO: 839 | SEQ ID NO: 1896 |
| 5-3070158-2 | SEQ ID NO: 840 | SEQ ID NO: 1897 |
| 5-306996-2 | SEQ ID NO: 841 | SEQ ID NO: 1898 |
| 5-5105-177 | SEQ ID NO: 842 | SEQ ID NO: 1899 |
| 5-2523605-47 | SEQ ID NO: 843 | SEQ ID NO: 1900 |
| 5-1901007-7 | SEQ ID NO: 844 | SEQ ID NO: 1901 |
| 5-3146146-2 | SEQ ID NO: 845 | SEQ ID NO: 1902 |
| 5-1519916-3 | SEQ ID NO: 846 | SEQ ID NO: 1903 |
| 5-1931939-4 | SEQ ID NO: 847 | SEQ ID NO: 1904 |
| 5-1329045-2 | SEQ ID NO: 848 | SEQ ID NO: 1905 |
| 5-2636281-6 | SEQ ID NO: 849 | SEQ ID NO: 1906 |
| 5-2677818-2 | SEQ ID NO: 850 | SEQ ID NO: 1907 |
| 5-2006146-2 | SEQ ID NO: 851 | SEQ ID NO: 1908 |
| 5-104887-42 | SEQ ID NO: 852 | SEQ ID NO: 1909 |
| 5-1917751-2 | SEQ ID NO: 853 | SEQ ID NO: 1910 |
| 5-117495-3 | SEQ ID NO: 854 | SEQ ID NO: 1911 |
| 5-2526096-56 | SEQ ID NO: 855 | SEQ ID NO: 1912 |
| 5-523992-3 | SEQ ID NO: 856 | SEQ ID NO: 1913 |
| 5-2915546-3 | SEQ ID NO: 857 | SEQ ID NO: 1914 |
| 5-2528072-143 | SEQ ID NO: 858 | SEQ ID NO: 1915 |
| 5-1032651-190 | SEQ ID NO: 859 | SEQ ID NO: 1916 |
| 5-1198817-2 | SEQ ID NO: 860 | SEQ ID NO: 1917 |
| 5-12970-57 | SEQ ID NO: 861 | SEQ ID NO: 1918 |
| 5-1987237-2 | SEQ ID NO: 862 | SEQ ID NO: 1919 |
| 5-2216357-2 | SEQ ID NO: 863 | SEQ ID NO: 1920 |
| 5-2355075-2 | SEQ ID NO: 864 | SEQ ID NO: 1921 |
| 5-1079490-17 | SEQ ID NO: 865 | SEQ ID NO: 1922 |
| 5-3009197-2 | SEQ ID NO: 866 | SEQ ID NO: 1923 |
| 5-1921849-10 | SEQ ID NO: 867 | SEQ ID NO: 1924 |
| 5-1913908-4 | SEQ ID NO: 868 | SEQ ID NO: 1925 |
| 5-2676962-7 | SEQ ID NO: 869 | SEQ ID NO: 1926 |
| 5-46711-25 | SEQ ID NO: 870 | SEQ ID NO: 1927 |
| 5-1918507-18 | SEQ ID NO: 871 | SEQ ID NO: 1928 |
| 5-3238118-2 | SEQ ID NO: 872 | SEQ ID NO: 1929 |
| 5-2629877-4 | SEQ ID NO: 873 | SEQ ID NO: 1930 |
| 5-134597-7 | SEQ ID NO: 874 | SEQ ID NO: 1931 |
| 5-230952-2 | SEQ ID NO: 875 | SEQ ID NO: 1932 |
| 5-2672385-2 | SEQ ID NO: 876 | SEQ ID NO: 1933 |
| 5-1486985-2 | SEQ ID NO: 877 | SEQ ID NO: 1934 |
| 5-460730-2 | SEQ ID NO: 878 | SEQ ID NO: 1935 |
| 5-411642-3 | SEQ ID NO: 879 | SEQ ID NO: 1936 |
| 5-2528289-2 | SEQ ID NO: 880 | SEQ ID NO: 1937 |
| 5-111537-4 | SEQ ID NO: 881 | SEQ ID NO: 1938 |
| 5-17288-89 | SEQ ID NO: 882 | SEQ ID NO: 1939 |
| 5-151431-5 | SEQ ID NO: 883 | SEQ ID NO: 1940 |
| 5-2051967-2 | SEQ ID NO: 884 | SEQ ID NO: 1941 |
| 5-596955-2 | SEQ ID NO: 885 | SEQ ID NO: 1942 |
| 5-16888-28 | SEQ ID NO: 886 | SEQ ID NO: 1943 |
| 5-1145273-4 | SEQ ID NO: 887 | SEQ ID NO: 1944 |
| 5-1905626-5 | SEQ ID NO: 888 | SEQ ID NO: 1945 |
| 5-2074121-7 | SEQ ID NO: 889 | SEQ ID NO: 1946 |
| 5-2063525-7 | SEQ ID NO: 890 | SEQ ID NO: 1947 |
| 5-1967544-2 | SEQ ID NO: 891 | SEQ ID NO: 1948 |
| 5-44097-3 | SEQ ID NO: 892 | SEQ ID NO: 1949 |
| 5-2621779-2 | SEQ ID NO: 893 | SEQ ID NO: 1950 |
| 5-611475-2 | SEQ ID NO: 894 | SEQ ID NO: 1951 |
| 5-1229307-3 | SEQ ID NO: 895 | SEQ ID NO: 1952 |
| 5-206606-4 | SEQ ID NO: 896 | SEQ ID NO: 1953 |
| 5-889050-2 | SEQ ID NO: 897 | SEQ ID NO: 1954 |
| 5-3581-203 | SEQ ID NO: 898 | SEQ ID NO: 1955 |
| 5-1905882-9 | SEQ ID NO: 899 | SEQ ID NO: 1956 |
| 5-1902084-35 | SEQ ID NO: 900 | SEQ ID NO: 1957 |
| 5-1104533-2 | SEQ ID NO: 901 | SEQ ID NO: 1958 |
| 5-1430263-2 | SEQ ID NO: 902 | SEQ ID NO: 1959 |
| 5-1266707-2 | SEQ ID NO: 903 | SEQ ID NO: 1960 |
| 5-1943151-5 | SEQ ID NO: 904 | SEQ ID NO: 1961 |
| 5-2705097-2 | SEQ ID NO: 905 | SEQ ID NO: 1962 |
| 5-2543477-18 | SEQ ID NO: 906 | SEQ ID NO: 1963 |
| 5-432252-3 | SEQ ID NO: 907 | SEQ ID NO: 1964 |
| 5-1259595-10 | SEQ ID NO: 908 | SEQ ID NO: 1965 |
| 5-2567948-19 | SEQ ID NO: 909 | SEQ ID NO: 1966 |
| 5-299244-3 | SEQ ID NO: 910 | SEQ ID NO: 1967 |
| 5-1099746-5 | SEQ ID NO: 911 | SEQ ID NO: 1968 |
| 5-1052667-6 | SEQ ID NO: 912 | SEQ ID NO: 1969 |
| 5-21817-5 | SEQ ID NO: 913 | SEQ ID NO: 1970 |
| 5-2676010-9 | SEQ ID NO: 914 | SEQ ID NO: 1971 |
| 5-22014-3 | SEQ ID NO: 915 | SEQ ID NO: 1972 |
| 5-85116-2 | SEQ ID NO: 916 | SEQ ID NO: 1973 |
| 5-1404040-3 | SEQ ID NO: 917 | SEQ ID NO: 1974 |
| 5-2596440-4 | SEQ ID NO: 918 | SEQ ID NO: 1975 |
| 5-276868-4 | SEQ ID NO: 919 | SEQ ID NO: 1976 |
| 5-26437-2 | SEQ ID NO: 920 | SEQ ID NO: 1977 |
| 5-1931690-44 | SEQ ID NO: 921 | SEQ ID NO: 1978 |
| 5-1017692-94 | SEQ ID NO: 922 | SEQ ID NO: 1979 |
| 5-2540817-50 | SEQ ID NO: 923 | SEQ ID NO: 1980 |
| 5-2742127-2 | SEQ ID NO: 924 | SEQ ID NO: 1981 |
| 5-630548-4 | SEQ ID NO: 925 | SEQ ID NO: 1982 |
| 5-14201-89 | SEQ ID NO: 926 | SEQ ID NO: 1983 |
| 5-1026744-24 | SEQ ID NO: 927 | SEQ ID NO: 1984 |
| 5-1340661-2 | SEQ ID NO: 928 | SEQ ID NO: 1985 |
| 5-2537215-71 | SEQ ID NO: 929 | SEQ ID NO: 1986 |
| 5-77902-2 | SEQ ID NO: 930 | SEQ ID NO: 1987 |
| 5-2464979-2 | SEQ ID NO: 931 | SEQ ID NO: 1988 |
| 5-2079914-3 | SEQ ID NO: 932 | SEQ ID NO: 1989 |
| 5-261958-5 | SEQ ID NO: 933 | SEQ ID NO: 1990 |
| 5-78727-3 | SEQ ID NO: 934 | SEQ ID NO: 1991 |
| 5-2909528-5 | SEQ ID NO: 935 | SEQ ID NO: 1992 |
| 5-1083544-6 | SEQ ID NO: 936 | SEQ ID NO: 1993 |
| 5-2535307-11 | SEQ ID NO: 937 | SEQ ID NO: 1994 |
| 5-1072522-20 | SEQ ID NO: 938 | SEQ ID NO: 1995 |
| 5-2959014-2 | SEQ ID NO: 939 | SEQ ID NO: 1996 |
| 5-1471554-2 | SEQ ID NO: 940 | SEQ ID NO: 1997 |
| 5-1072458-2 | SEQ ID NO: 941 | SEQ ID NO: 1998 |
| 5-1310308-2 | SEQ ID NO: 942 | SEQ ID NO: 1999 |
| 5-1168709-3 | SEQ ID NO: 943 | SEQ ID NO: 2000 |
| 5-1040915-6 | SEQ ID NO: 944 | SEQ ID NO: 2001 |
| 5-1109831-8 | SEQ ID NO: 945 | SEQ ID NO: 2002 |
| 5-1066115-14 | SEQ ID NO: 946 | SEQ ID NO: 2003 |
| 5-2529013-51 | SEQ ID NO: 947 | SEQ ID NO: 2004 |
| 5-2366950-2 | SEQ ID NO: 948 | SEQ ID NO: 2005 |
| 5-2699971-6 | SEQ ID NO: 949 | SEQ ID NO: 2006 |
| 5-553990-2 | SEQ ID NO: 950 | SEQ ID NO: 2007 |
| 5-1382556-3 | SEQ ID NO: 951 | SEQ ID NO: 2008 |
| 5-1950979-26 | SEQ ID NO: 952 | SEQ ID NO: 2009 |
| 5-467037-3 | SEQ ID NO: 953 | SEQ ID NO: 2010 |
| 5-386603-2 | SEQ ID NO: 954 | SEQ ID NO: 2011 |
| 5-1483917-2 | SEQ ID NO: 955 | SEQ ID NO: 2012 |
| 5-2728374-2 | SEQ ID NO: 956 | SEQ ID NO: 2013 |
| 5-1044027-43 | SEQ ID NO: 957 | SEQ ID NO: 2014 |
| 5-165461-22 | SEQ ID NO: 958 | SEQ ID NO: 2015 |
| 5-1183372-7 | SEQ ID NO: 959 | SEQ ID NO: 2016 |
| 5-2030894-2 | SEQ ID NO: 960 | SEQ ID NO: 2017 |
| 5-2528718-86 | SEQ ID NO: 961 | SEQ ID NO: 2018 |
| 5-249597-3 | SEQ ID NO: 962 | SEQ ID NO: 2019 |
| 5-2234117-2 | SEQ ID NO: 963 | SEQ ID NO: 2020 |
| 5-676040-4 | SEQ ID NO: 964 | SEQ ID NO: 2021 |
| 5-2633578-5 | SEQ ID NO: 965 | SEQ ID NO: 2022 |
| 5-3075427-2 | SEQ ID NO: 966 | SEQ ID NO: 2023 |
| 5-2475184-2 | SEQ ID NO: 967 | SEQ ID NO: 2024 |
| 5-2663669-2 | SEQ ID NO: 968 | SEQ ID NO: 2025 |
| 5-1327278-2 | SEQ ID NO: 969 | SEQ ID NO: 2026 |
| 5-1905648-43 | SEQ ID NO: 970 | SEQ ID NO: 2027 |
| 5-1981142-2 | SEQ ID NO: 971 | SEQ ID NO: 2028 |
| 5-670280-2 | SEQ ID NO: 972 | SEQ ID NO: 2029 |
| 5-126801-3 | SEQ ID NO: 973 | SEQ ID NO: 2030 |
| 5-2027639-2 | SEQ ID NO: 974 | SEQ ID NO: 2031 |
| 5-3067631-2 | SEQ ID NO: 975 | SEQ ID NO: 2032 |
| 5-113579-2 | SEQ ID NO: 976 | SEQ ID NO: 2033 |
| 5-2569664-2 | SEQ ID NO: 977 | SEQ ID NO: 2034 |
| 5-36294-3 | SEQ ID NO: 978 | SEQ ID NO: 2035 |
| 5-545155-2 | SEQ ID NO: 979 | SEQ ID NO: 2036 |
| 5-1232366-2 | SEQ ID NO: 980 | SEQ ID NO: 2037 |
| 5-306980-2 | SEQ ID NO: 981 | SEQ ID NO: 2038 |
| 5-118199-3 | SEQ ID NO: 982 | SEQ ID NO: 2039 |
| 5-1678433-2 | SEQ ID NO: 983 | SEQ ID NO: 2040 |
| 5-42118-2 | SEQ ID NO: 984 | SEQ ID NO: 2041 |
| 5-420970-3 | SEQ ID NO: 985 | SEQ ID NO: 2042 |
| 5-2716127-2 | SEQ ID NO: 986 | SEQ ID NO: 2043 |
| 5-1555113-2 | SEQ ID NO: 987 | SEQ ID NO: 2044 |
| 5-1270243-2 | SEQ ID NO: 988 | SEQ ID NO: 2045 |
| 5-1450438-3 | SEQ ID NO: 989 | SEQ ID NO: 2046 |
| 5-2939255-2 | SEQ ID NO: 990 | SEQ ID NO: 2047 |
| 5-2742093-2 | SEQ ID NO: 991 | SEQ ID NO: 2048 |
| 5-517535-3 | SEQ ID NO: 992 | SEQ ID NO: 2049 |
| 5-1023472-25 | SEQ ID NO: 993 | SEQ ID NO: 2050 |
| 5-360426-2 | SEQ ID NO: 994 | SEQ ID NO: 2051 |
| 5-1962210-2 | SEQ ID NO: 995 | SEQ ID NO: 2052 |
| 5-2109609-6 | SEQ ID NO: 996 | SEQ ID NO: 2053 |
| 5-390720-2 | SEQ ID NO: 997 | SEQ ID NO: 2054 |
| 5-1265740-2 | SEQ ID NO: 998 | SEQ ID NO: 2055 |
| 5-1201165-2 | SEQ ID NO: 999 | SEQ ID NO: 2056 |
| 5-2691520-2 | SEQ ID NO: 1000 | SEQ ID NO: 2057 |
| 5-42433-18 | SEQ ID NO: 1001 | SEQ ID NO: 2058 |
| 5-77301-2 | SEQ ID NO: 1002 | SEQ ID NO: 2059 |
| 5-1271723-3 | SEQ ID NO: 1003 | SEQ ID NO: 2060 |
| 5-2558584-2 | SEQ ID NO: 1004 | SEQ ID NO: 2061 |
| 5-1979919-2 | SEQ ID NO: 1005 | SEQ ID NO: 2062 |
| 5-1291905-2 | SEQ ID NO: 1006 | SEQ ID NO: 2063 |
| 5-1215029-2 | SEQ ID NO: 1007 | SEQ ID NO: 2064 |
| 5-510644-4 | SEQ ID NO: 1008 | SEQ ID NO: 2065 |
| 5-2088084-2 | SEQ ID NO: 1009 | SEQ ID NO: 2066 |
| 5-886686-2 | SEQ ID NO: 1010 | SEQ ID NO: 2067 |
| 5-490241-2 | SEQ ID NO: 1011 | SEQ ID NO: 2068 |
| 5-889172-2 | SEQ ID NO: 1012 | SEQ ID NO: 2069 |
| 5-1204407-2 | SEQ ID NO: 1013 | SEQ ID NO: 2070 |
| 5-1048785-17 | SEQ ID NO: 1014 | SEQ ID NO: 2071 |
| 5-2638944-2 | SEQ ID NO: 1015 | SEQ ID NO: 2072 |
| 5-1341138-2 | SEQ ID NO: 1016 | SEQ ID NO: 2073 |
| 5-585203-2 | SEQ ID NO: 1017 | SEQ ID NO: 2074 |
| 5-98324-3 | SEQ ID NO: 1018 | SEQ ID NO: 2075 |
| 5-2974173-2 | SEQ ID NO: 1019 | SEQ ID NO: 2076 |
| 5-1262349-2 | SEQ ID NO: 1020 | SEQ ID NO: 2077 |
| 5-1527609-2 | SEQ ID NO: 1021 | SEQ ID NO: 2078 |
| 5-1402594-2 | SEQ ID NO: 1022 | SEQ ID NO: 2079 |
| 5-1585933-2 | SEQ ID NO: 1023 | SEQ ID NO: 2080 |
| 5-2536647-3 | SEQ ID NO: 1024 | SEQ ID NO: 2081 |
| 5-1047394-9 | SEQ ID NO: 1025 | SEQ ID NO: 2082 |
| 5-3166391-2 | SEQ ID NO: 1026 | SEQ ID NO: 2083 |
| 5-1104151-2 | SEQ ID NO: 1027 | SEQ ID NO: 2084 |
| 5-1166523-6 | SEQ ID NO: 1028 | SEQ ID NO: 2085 |
| 5-1240577-4 | SEQ ID NO: 1029 | SEQ ID NO: 2086 |
| 5-1930341-3 | SEQ ID NO: 1030 | SEQ ID NO: 2087 |
| 5-361729-2 | SEQ ID NO: 1031 | SEQ ID NO: 2088 |
| 5-1431176-2 | SEQ ID NO: 1032 | SEQ ID NO: 2089 |
| 5-1282370-2 | SEQ ID NO: 1033 | SEQ ID NO: 2090 |
| 5-1959556-2 | SEQ ID NO: 1034 | SEQ ID NO: 2091 |
| 5-1144154-2 | SEQ ID NO: 1035 | SEQ ID NO: 2092 |
| 5-752607-2 | SEQ ID NO: 1036 | SEQ ID NO: 2093 |
| 5-3140019-2 | SEQ ID NO: 1037 | SEQ ID NO: 2094 |
| 5-2625202-2 | SEQ ID NO: 1038 | SEQ ID NO: 2095 |
| 5-1961020-2 | SEQ ID NO: 1039 | SEQ ID NO: 2096 |
| 5-1944760-2 | SEQ ID NO: 1040 | SEQ ID NO: 2097 |
| 5-477551-4 | SEQ ID NO: 1041 | SEQ ID NO: 2098 |
| 5-661227-2 | SEQ ID NO: 1042 | SEQ ID NO: 2099 |
| 5-1402163-2 | SEQ ID NO: 1043 | SEQ ID NO: 2100 |
| 5-2578715-2 | SEQ ID NO: 1044 | SEQ ID NO: 2101 |
| 5-2556404-2 | SEQ ID NO: 1045 | SEQ ID NO: 2102 |
| 5-63662-3 | SEQ ID NO: 1046 | SEQ ID NO: 2103 |
| 5-576971-2 | SEQ ID NO: 1047 | SEQ ID NO: 2104 |
| 5-1943740-2 | SEQ ID NO: 1048 | SEQ ID NO: 2105 |
| 5-175719-2 | SEQ ID NO: 1049 | SEQ ID NO: 2106 |
| 5-1059042-2 | SEQ ID NO: 1050 | SEQ ID NO: 2107 |
| 5-1122382-3 | SEQ ID NO: 1051 | SEQ ID NO: 2108 |
| 5-2703852-2 | SEQ ID NO: 1052 | SEQ ID NO: 2109 |
| 5-2542749-2 | SEQ ID NO: 1053 | SEQ ID NO: 2110 |
| 5-189359-4 | SEQ ID NO: 1054 | SEQ ID NO: 2111 |
| 5-2961307-2 | SEQ ID NO: 1055 | SEQ ID NO: 2112 |
| 5-649316-3 | SEQ ID NO: 1056 | SEQ ID NO: 2113 |
| 5-2158968-2 | SEQ ID NO: 1057 | SEQ ID NO: 2114 |

TABLE 2

| | | | | | | | | | | Pseudo typed | PRNT | PRNT | Amino |
|---|---|---|---|---|---|---|---|---|---|---|---|---|---|
| | | | | | | | ELISA | | | SARS-CoV-2 | SARS- | SARS- | Acid |
| | ELISA of | | ELISA of | | ELISA of | | of BM- | | | (D614G, | CoV-2 | CoV-2 | (Protein) |
| | SARS- | | SARS- | | RmYN02- | | 4831- | | | Wuhan-Hu-1) | Munich | Delta | Sequence |
| | CoV-2 | | CoV-1 | | CoV | | CoV | | | neutralization | IC50 | IC50 | (SEQ ID |
| Nanobody | RBD | | RBD | | RBD | | RBD | | SEC | IC50 (nM) | (nM) | (nM) | NO) |
| 5p-1 | 10-100 | nM | 0.1-1 | μM | 1-8 | μM | 1-8 | μM | Group D | >2500 | | | SEQ ID NO: |
| | | | | | | | | | | | | | 1058 |
| 5p-2 | 10-100 | nM | 10-100 | nM | 1-8 | μM | 1-8 | μM | Group D | >2500 | | | SEQ ID NO: |
| | | | | | | | | | | | | | 1059 |
| 5p-3 | <10 | nM | 10-100 | nM | 10-100 | nM | 10-100 | nM | Group E | >2500 | | | SEQ ID NO: |
| | | | | | | | | | | | | | 1060 |
| 5p-4 | <10 | nM | 10-100 | nM | 0.1-1 | μM | >8 | μM | Group E | >2500 | | | SEQ ID NO: |
| | | | | | | | | | | | | | 1061 |
| 5p-5 | 10-100 | nM | 10-100 | nM | >8 | μM | >8 | μM | Group D | >2500 | | | SEQ ID NO: |
| | | | | | | | | | | | | | 1062 |
| 5p-6 | <10 | nM | 10-100 | nM | 0.1-1 | μM | 1-8 | μM | Group E | >2500 | | | SEQ ID NO: |
| | | | | | | | | | | | | | 1260 |
| 5p-10 | <10 | nM | 10-100 | nM | 0.1-1 | μM | >8 | μM | Group E | >2500 | | | SEQ ID NO: |
| | | | | | | | | | | | | | 1063 |
| 5p-13 | <10 | nM | <10 | nM | 0.1-1 | μM | >8 | μM | Group E | >2500 | | | SEQ ID NO: |
| | | | | | | | | | | | | | 1261 |
| 5p-14 | <10 | nM | <10 | nM | 10-100 | nM | >8 | μM | Group E | >2500 | | | SEQ ID NO: |
| | | | | | | | | | | | | | 1064 |
| 5p-15 | 10-100 | nM | 10-100 | nM | 0.1-1 | μM | 1-8 | μM | Group E | >2500 | | | SEQ ID NO: |
| | | | | | | | | | | | | | 1065 |
| 5p-16 | <10 | nM | 10-100 | nM | 10-100 | nM | 10-100 | nM | Group E | >2500 | | | SEQ ID NO: |
| | | | | | | | | | | | | | 1262 |
| 5p-17 | <10 | nM | <10 | nM | 10-100 | nM | 10-100 | nM | Group E | >2500 | | | SEQ ID NO: |
| | | | | | | | | | | | | | 1066 |
| 5p-18 | <10 | nM | <10 | nM | 10-100 | nM | 0.1-1 | μM | Group E | >2500 | | | SEQ ID NO: |
| | | | | | | | | | | | | | 1067 |
| 5p-20 | <10 | nM | 0.1-1 | μM | 0.1-1 | μM | >8 | μM | Group E | >2500 | | | SEQ ID NO: |
| | | | | | | | | | | | | | 1068 |
| 5p-23 | <10 | nM | 10-100 | nM | 0.1-1 | μM | >8 | μM | Group E | >2500 | | | SEQ ID NO: |
| | | | | | | | | | | | | | 1069 |
| 5p-24 | <10 | nM | <10 | nM | 10-100 | nM | 1-8 | μM | Group E | >2500 | | | SEQ ID NO: |
| | | | | | | | | | | | | | 1070 |
| 5p-25 | <10 | nM | <10 | nM | 0.1-1 | μM | >8 | μM | Group E | >2500 | | | SEQ ID NO: |
| | | | | | | | | | | | | | 1071 |
| 5p-26 | <10 | nM | >8 | μM | 1-8 | μM | 1-8 | μM | Group D | 2000 | | | SEQ ID NO: |
| | | | | | | | | | | | | | 1072 |
| 5p-34 | <10 | nM | <10 | nM | <10 | nM | 1-8 | μM | Group B | 7.27 | 32.2 | 8.76 | SEQ ID NO: |
| | | | | | | | | | | | | | 1074 |
| 5p-35 | <10 | nM | <10 | nM | <10 | nM | 10-100 | nM | Group B | 4.05 | 42.2 | 21 | SEQ ID NO: |
| | | | | | | | | | | | | | 1075 |
| 5p-37 | <10 | nM | 10-100 | nM | 10-100 | nM | >8 | μM | Group B | 14.4 | | | SEQ ID NO: |
| | | | | | | | | | | | | | 1077 |
| 5p-38 | <10 | nM | <10 | nM | <10 | nM | 0.1-1 | μM | Group B | 6.63 | 59.9 | 34.7 | SEQ ID NO: |
| | | | | | | | | | | | | | 1078 |
| 5p-40 | <10 | nM | <10 | nM | 10-100 | nM | 1-8 | μM | Group E | >2500 | | | SEQ ID NO: |
| | | | | | | | | | | | | | 1079 |
| 5p-47 | <10 | nM | 1-8 | μM | >8 | μM | >8 | μM | Group E | >2500 | | | SEQ ID NO: |
| | | | | | | | | | | | | | 1080 |
| 5p-58 | <10 | nM | 1-8 | μM | 0.1-1 | μM | >8 | μM | Group D | 493 | | | SEQ ID NO: |
| | | | | | | | | | | | | | 1082 |
| 5p-59 | <10 | nM | >8 | μM | <10 | nM | >8 | μM | Group B | 6.2 | | | SEQ ID NO: |
| | | | | | | | | | | | | | 1083 |
| 5p-60 | <10 | nM | <10 | nM | 10 | nM | >8 | μM | Group B | 0.9 | 9.79 | 8.74 | SEQ ID NO: |
| | | | | | | | | | | | | | 1084 |
| 5p-61 | <10 | nM | 10-100 | nM | 0.1-1 | μM | >8 | μM | Group E | >2500 | | | SEQ ID NO: |
| | | | | | | | | | | | | | 1085 |
| 5p-63 | <10 | nM | <10 | nM | 10-100 | nM | 0.1-1 | μM | Group C | >2500 | | | SEQ ID NO: |
| | | | | | | | | | | | | | 1271 |
| 5p-64 | <10 | nM | <10 | nM | 10-100 | nM | 10-100 | nM | Group D | >2500 | to be | to be | SEQ ID NO: |
| | | | | | | | | | | | done | done | 1086 |
| 5p-68 | <10 | nM | 10-100 | nM | 0.1-1 | μM | 1-8 | μM | Group E | >2500 | | | SEQ ID NO: |
| | | | | | | | | | | | | | 1087 |
| 5p-74 | <10 | nM | <10 | nM | 10-100 | nM | 0.1-1 | μM | Group E | >2500 | | | SEQ ID NO: |
| | | | | | | | | | | | | | 1274 |
| 5p-78 | <10 | nM | <10 | nM | <10 | nM | 1-8 | μM | Group B | 11.4 | | | SEQ ID NO: |
| | | | | | | | | | | | | | 1089 |
| 5p-82 | 10-100 | nM | 10-100 | nM | >8 | μM | 1-8 | μM | Group D | >2500 | | | SEQ ID NO: |
| | | | | | | | | | | | | | 1111 |
| 5p-89 | 10-100 | nM | 10-100 | nM | >8 | μM | 0.1-1 | μM | Group D | >2500 | | | SEQ ID NO: |
| | | | | | | | | | | | | | 1118 |
| 5p-93 | <10 | nM | >8 | μM | 10-100 | nM | 10-100 | nM | Group C | 140 | 264 | 171 | SEQ ID NO: |
| | | | | | | | | | | | | | 1122 |
| 5p-98 | <10 | nM | 10-100 | nM | 0.1-1 | μM | 1-8 | μM | Group E | >2500 | | | SEQ ID NO: |
| | | | | | | | | | | | | | 1127 |
| 5p-99 | <10 | nM | 1-8 | μM | >8 | μM | >8 | μM | Group E | >2500 | | | SEQ ID NO: |
| | | | | | | | | | | | | | 1128 |
| 5p-109 | <10 | nM | >8 | μM | 10-100 | nM | 0.1-1 | μM | Group D | 482 | 410 | 313 | SEQ ID NO: |
| | | | | | | | | | | | | | 1129 |
| 5p-100 | <10 | nM | 1-8 | μM | >8 | μM | >8 | μM | Group A | >2500 | | | SEQ ID NO: |
| | | | | | | | | | | | | | 1130 |
| 5p-101 | <10 | nM | 10-100 | nM | 10-100 | nM | 10-100 | nM | Group E | >2500 | | | SEQ ID NO: |
| | | | | | | | | | | | | | 1132 |
| 5p-103 | 10-100 | nM | 10-100 | nM | 0.1-1 | μM | 1-8 | μM | Group E | >2500 | | | SEQ ID NO: |
| | | | | | | | | | | | | | 1133 |
| 5p-104 | <10 | nM | <10 | nM | 10-100 | nM | 0.1-1 | μM | Group E | >2500 | | | SEQ ID NO: |
| | | | | | | | | | | | | | 1134 |
| 5p-105 | <10 | nM | <10 | nM | 10-100 | nM | 1-8 | μM | Group E | >2500 | | | SEQ ID NO: |
| | | | | | | | | | | | | | 1138 |
| 5p-117 | <10 | nM | <10 | nM | <10 | nM | 1-8 | μM | Group B | 9.09 | 10.6 | 27.2 | SEQ ID NO: |
| | | | | | | | | | | | | | 1146 |
| 5p-118 | <10 | nM | <10 | nM | <10 | nM | <10 | nM | Group B | 5.12 | 7.41 | 3.62 | SEQ ID NO: |
| | | | | | | | | | | | | | 1147 |
| 5p-119 | <10 | nM | <10 | nM | <10 | nM | 0.1-1 | μM | Group B | 19 | | | SEQ ID NO: |
| | | | | | | | | | | | | | 1148 |
| 5p-120 | <10 | nM | <10 | nM | <10 | nM | 10-100 | nM | Group B | 7.59 | 19.1 | 9.16 | SEQ ID NO: |
| | | | | | | | | | | | | | 1149 |
| 5p-121 | <10 | nM | <10 | nM | <10 | nM | >8 | μM | Group B | 166 | | | SEQ ID NO: |
| | | | | | | | | | | | | | 1150 |
| 5p-124 | <10 | nM | <10 | nM | 10 | nM | >8 | μM | Group B | 4.6 | | | SEQ ID NO: |
| | | | | | | | | | | | | | 1153 |
| 5p-125 | <10 | nM | 10-100 | nM | <10 | nM | >8 | μM | Group B | 22 | | | SEQ ID NO: |
| | | | | | | | | | | | | | 1154 |
| 5p-126 | <10 | nM | <10 | nM | <10 | nM | 1-8 | μM | Group B | 21 | | | SEQ ID NO: |
| | | | | | | | | | | | | | 1155 |
| 5p-127 | <10 | nM | <10 | nM | 10-100 | nM | >8 | μM | Group B | 12.8 | 44.5 | 36.5 | SEQ ID NO: |
| | | | | | | | | | | | | | 1156 |
| 5p-128 | <10 | nM | 10-100 | nM | >8 | μM | >8 | μM | Group B | 43.6 | | | SEQ ID NO: |
| | | | | | | | | | | | | | 1157 |
| 5p-132 | <10 | nM | 0.1-1 | μM | <10 | nM | <10 | nM | Group C | 104 | 42.8 | 211 | SEQ ID NO: |
| | | | | | | | | | | | | | 1161 |
| 5p-156 | <10 | nM | 10-100 | nM | >8 | μM | >8 | μM | Group E | >2500 | | | SEQ ID NO: |
| | | | | | | | | | | | | | 1185 |
| 5p-162 | 10-100 | nM | 10-100 | nM | 1-8 | μM | 0.1-1 | μM | Group C | >2500 | | | SEQ ID NO: |
| | | | | | | | | | | | | | 1191 |
| 5p-164 | <10 | nM | 10-100 | nM | 10-100 | nM | >8 | μM | Group E | >2500 | | | SEQ ID NO: |
| | | | | | | | | | | | | | 1193 |
| 5p-165 | <10 | nM | 10-100 | nM | >8 | μM | >8 | μM | Group E | >2500 | | | SEQ ID NO: |
| | | | | | | | | | | | | | 1194 |
| 5p-167 | <10 | nM | >8 | μM | 10-100 | nM | >8 | μM | Group D | 185 | 263 | 74.5 | SEQ ID NO: |
| | | | | | | | | | | | | | 1196 |
| 5p-168 | <10 | nM | <10 | nM | 10-100 | nM | >8 | μM | Group B | 0.45 | 0.36 | 2.25 | SEQ ID NO: |
| | | | | | | | | | | | | | 1197 |
| 5p-169 | <10 | nM | <10 | nM | 1-8 | μM | >8 | μM | Group B | 0.8 | | | SEQ ID NO: |
| | | | | | | | | | | | | | 1198 |
| 5p-171 | <10 | nM | 10-100 | nM | 0.1-1 | μM | 10-100 | nM | Group E | >2500 | | | SEQ ID NO: |
| | | | | | | | | | | | | | 1200 |
| 5p-174 | <10 | nM | <10 | nM | 10-100 | nM | 10-100 | nM | Group D | >2500 | | | SEQ ID NO: |
| | | | | | | | | | | | | | 1203 |
| 5p-175 | <10 | nM | <10 | nM | 10-100 | nM | 10-100 | nM | Group C | >2500 | 8657 | 8913 | SEQ ID NO: |
| | | | | | | | | | | | | | 1204 |
| 5p-176 | <10 | nM | <10 | nM | 10-100 | nM | 10-100 | nM | Group D | >2500 | | | SEQ ID NO: |
| | | | | | | | | | | | | | 1205 |
| 5p-179 | <10 | nM | >8 | μM | >8 | μM | 0.1-1 | μM | Group C | 480 | | | SEQ ID NO: |
| | | | | | | | | | | | | | 1208 |
| 5p-180 | <10 | nM | >8 | μM | >8 | μM | 1-8 | μM | Group C | 512 | | | SEQ ID NO: |
| | | | | | | | | | | | | | 1209 |
| 5p-182 | <10 | nM | 1-8 | μM | 1-8 | μM | >8 | μM | Group A | 0.077 | 0.35 | 0.31 | SEQ ID NO: |
| | | | | | | | | | | | | | 1211 |
| 4P-1 | 10-100 | nM | 1-8 | μM | >8 | μM | >8 | μM | Group D | >2500 | | | SEQ ID NO: |
| | | | | | | | | | | | | | 1090 |
| 4P-3 | 10-100 | nM | 1-8 | μM | >8 | μM | >8 | μM | Group E | >2500 | | | SEQ ID NO: |
| | | | | | | | | | | | | | 1091 |
| 4P-4 | 10-100 | nM | 0.1-1 | μM | >8 | μM | >8 | μM | Group E | >2500 | | | SEQ ID NO: |
| | | | | | | | | | | | | | 1092 |
| 4P-5 | 10-100 | nM | 0.1-1 | μM | >8 | μM | >8 | μM | Group E | >2500 | | | SEQ ID NO: |
| | | | | | | | | | | | | | 1093 |
| 4P-9 | <10 | nM | 0.1-1 | μM | >8 | μM | >8 | μM | Group E | >2500 | | | SEQ ID NO: |
| | | | | | | | | | | | | | 1220 |
| 4P-10 | <10 | nM | 0.1-1 | μM | >8 | μM | >8 | μM | Group E | >2500 | | | SEQ ID NO: |
| | | | | | | | | | | | | | 1221 |
| 4P-12 | <10 | nM | 10-100 | nM | 0.1-1 | μM | 0.1-1 | μM | Group E | >2500 | | | SEQ ID NO: |
| | | | | | | | | | | | | | 1222 |
| 4P-17 | <10 | nM | 1-8 | μM | <10 | nM | >8 | μM | Group B | 20 | | | SEQ ID NO: |
| | | | | | | | | | | | | | 1097 |
| 4P-19 | <10 | nM | 0.1-1 | μM | 0.1-1 | μM | >8 | μM | Group B | 58 | | | SEQ ID NO: |
| | | | | | | | | | | | | | 1098 |
| 4P-21 | <10 | nM | <10 | nM | 10-100 | nM | 1-8 | μM | Group E | >2500 | | | SEQ ID NO: |
| | | | | | | | | | | | | | 1099 |
| 4P-26 | <10 | nM | >8 | μM | 1-8 | μM | >8 | μM | Group D | >2500 | | | SEQ ID NO: |
| | | | | | | | | | | | | | 1229 |
| 4P-28 | 10-100 | nM | 1-8 | μM | 0.1-1 | μM | 1-8 | μM | Group C | 236 | | | SEQ ID NO: |
| | | | | | | | | | | | | | 1100 |
| 4P-32 | <10 | nM | 1-8 | μM | >8 | μM | >8 | μM | Group C | 95 | | | SEQ ID NO: |
| | | | | | | | | | | | | | 1234 |
| 4P-42 | <10 | nM | <10 | nM | <10 | nM | 1-8 | μM | Group B | 18.6 | | | SEQ ID NO: |
| | | | | | | | | | | | | | 1237 |
| 4P-44 | <10 | nM | 0.1-1 | μM | 10-100 | nM | >8 | μM | Group B | 128 | | | SEQ ID NO: |
| | | | | | | | | | | | | | 1103 |
| 4P-46 | <10 | nM | 10-100 | nM | 1-8 | μM | >8 | μM | Group B | 86.4 | | | SEQ ID NO: |
| | | | | | | | | | | | | | 1240 |
| 4P-47 | <10 | nM | <10 | nM | <10 | nM | >8 | μM | Group B | 6.43 | 13.1 | 9.54 | SEQ ID NO: |
| | | | | | | | | | | | | | 1241 |
| 4P-56 | <10 | nM | 10-100 | nM | <10 | nM | <10 | nM | Group D | 2000 | | | SEQ ID NO: |
| | | | | | | | | | | | | | 1249 |
| 4P-61 | <10 | nM | 10-100 | nM | 0.1-1 | μM | >8 | μM | Group E | >2500 | | | SEQ ID NO: |
| | | | | | | | | | | | | | 1105 |
| 4P-71 | <10 | nM | 10-100 | nM | <10 | nM | >8 | μM | Group B | 33 | | | SEQ ID NO: |
| | | | | | | | | | | | | | 1106 |
| 4P-74 | <10 | nM | >8 | μM | >8 | μM | 10-100 | nM | Group B | 0.478 | | | SEQ ID NO: |
| | | | | | | | | | | | | | 1276 |
| 4P-76 | 10-100 | nM | 1-8 | μM | 1-8 | μM | 10-100 | nM | Group B | 900 | | | SEQ ID NO: |
| | | | | | | | | | | | | | 1278 |
| 4P-84 | <10 | nM | <10 | nM | >8 | μM | >8 | μM | Group B | 4 | | | SEQ ID NO: |
| | | | | | | | | | | | | | 1286 |
| 4P-85 | <10 | nM | <10 | nM | >8 | μM | >8 | μM | Group B | 0.67 | | | SEQ ID NO: |
| | | | | | | | | | | | | | 1287 |
| 4P-86 | <10 | nM | <10 | nM | >8 | μM | >8 | μM | Group B | 0.72 | | | SEQ ID NO: |
| | | | | | | | | | | | | | 1288 |
| 4P-89 | <10 | nM | 10-100 | nM | 0.1-1 | μM | 0.1-1 | μM | Group E | >2500 | | | SEQ ID NO: |
| | | | | | | | | | | | | | 1291 |
| 4P-92 | <10 | nM | 1-8 | μM | 1-8 | μM | >8 | μM | Group A | 4.6 | | | SEQ ID NO: |
| | | | | | | | | | | | | | 1294 |
| R113 | <10 | nM | 10-100 | nM | <10 | nM | <10 | nM | Group D | 252 | 1106 | 146 | SEQ ID NO: |
| | | | | | | | | | | | | | 2117 |
| R37 | <10 | nM | <10 | nM | <10 | nM | >8 | μM | Group B | 11.8 | 8.36 | 40.2 | SEQ ID NO: |
| | | | | | | | | | | | | | 1096 |
| R117 | <10 | nM | 0.1-1 | μM | 10-100 | nM | >8 | μM | Group B | 88 | | | SEQ ID NO: |
| | | | | | | | | | | | | | 2118 |
| R58 | 10-100 | nM | 1-8 | μM | 0.1-1 | μM | 1-8 | μM | Group D | >2500 | | | SEQ ID NO: |
| | | | | | | | | | | | | | 2119 |

1. A nanobody comprising one or more complementarity determining regions 3 (CDR3), wherein the CDR3 comprises an amino acid sequence selected from the group consisting of SEQ ID NO: 1 through SEQ ID NO: 1057.

2. The nanobody of claim 1, wherein the nanobody comprises a multimer of one or more CDR3s, wherein the CDR3 comprises an amino acid sequence selected from the group consisting of SEQ ID NO:1 through SEQ ID NO: 1057.

3. The nanobody of claim 1, wherein the nanobody comprises a multimer of one or more amino acid sequences comprising a sequence selected from the group consisting of SEQ ID NO: 1058 through SEQ ID NO: 2114 and SEQ ID NO: 2117 through SEQ ID NO: 2119.

4. The nanobody of claim 1, wherein the nanobody comprises a multimer of one or more amino acid sequences comprising a sequence selected from the group consisting of SEQ ID NO: 1058-1072, 1074, 1075, 1077, 1078, 1079, 1080, 1082, 1083, 1084, 1085, 1086, 1087, 1089, 1090, 1091, 1092, 1093, 1096, 1097, 1098, 1099, 1100, 1103, 1105, 1106, 1111, 1118, 1122, 1127, 1128, 1129, 1130, 1132, 1133, 1134, 1138, 1146, 1147, 1148, 1149, 1150, 1153, 1154, 1155, 1156, 1157, 1161, 1185, 1191, 1193, 1194, 1196, 1197, 1198, 1200, 1203, 1204, 1205, 1208, 1209, 1211, 1220, 1221, 1222, 1229, 1234, 1237, 1240, 1241, 1249, 1260, 1261, 1262, 1271, 1274, 1276, 1278, 1286, 1287, 1288, 1291, 1294, 2117, 2118, and 2119.

5. The nanobody of claim 1, wherein the nanobody is a homotrimer comprising three copies of one amino acid sequence, wherein the one amino acid sequence comprises a sequence selected from the group consisting of SEQ ID NO: 1058 through SEQ ID NO: 2114 and SEQ ID NO: 2117 through SEQ ID NO: 2119.

6. The nanobody of claim 5, wherein the nanobody is a homotrimer comprising three copies of one amino acid sequence, wherein the one amino acid sequence comprises a sequence selected from the group consisting of SEQ ID NO: 1058-1072, 1074, 1075, 1077, 1078, 1079, 1080, 1082, 1083, 1084, 1085, 1086, 1087, 1089, 1090, 1091, 1092, 1093, 1096, 1097, 1098, 1099, 1100, 1103, 1105, 1106, 1111, 1118, 1122, 1127, 1128, 1129, 1130, 1132, 1133, 1134, 1138, 1146, 1147, 1148, 1149, 1150, 1153, 1154, 1155, 1156, 1157, 1161, 1185, 1191, 1193, 1194, 1196, 1197, 1198, 1200, 1203, 1204, 1205, 1208, 1209, 1211, 1220, 1221, 1222, 1229, 1234, 1237, 1240, 1241, 1249, 1260, 1261, 1262, 1271, 1274, 1276, 1278, 1286, 1287, 1288, 1291, 1294, 2117, 2118, and 2119.

7. The nanobody of claim 5, wherein the nanobody comprises a sequence of SEQ ID NO: 1147.

8. The nanobody of claim 5, wherein the nanobody comprises a sequence of SEQ ID NO: 1161.

9. The nanobody of claim 1, wherein the nanobody is a heterotrimer comprising three different amino acid sequences, wherein each different amino acid sequence comprises a sequence selected from the group consisting of SEQ ID NO: 1058 through SEQ ID NO: 2114 and SEQ ID NO: 2117 through SEQ ID NO: 2119.

10. The nanobody of claim 9, wherein the nanobody is a heterotrimer comprising three different amino acid sequences, wherein each different amino acid sequence comprises a sequence selected from the group consisting of SEQ ID NO: 1058-1072, 1074, 1075, 1077, 1078, 1079, 1080, 1082, 1083, 1084, 1085, 1086, 1087, 1089, 1090, 1091, 1092, 1093, 1096, 1097, 1098, 1099, 1100, 1103, 1105, 1106, 1111, 1118, 1122, 1127, 1128, 1129, 1130, 1132, 1133, 1134, 1138, 1146, 1147, 1148, 1149, 1150, 1153, 1154, 1155, 1156, 1157, 1161, 1185, 1191, 1193, 1194, 1196, 1197, 1198, 1200, 1203, 1204, 1205, 1208, 1209, 1211, 1220, 1221, 1222, 1229, 1234, 1237, 1240, 1241, 1249, 1260, 1261, 1262, 1271, 1274, 1276, 1278, 1286, 1287, 1288, 1291, 1294, 2117, 2118, and 2119.

11. The nanobody of claim 1, wherein the nanobody is a homodimer comprising two copies of one amino acid sequence, wherein the one amino acid sequence comprises a sequence selected from the group consisting of SEQ ID NO: 1058 through SEQ ID NO: 2114 and SEQ ID NO: 2117 through SEQ ID NO: 2119.

12. The nanobody of claim 11, wherein the nanobody is a homodimer comprising two copies of one amino acid sequence, wherein the one amino acid sequence comprises a sequence selected from the group consisting of SEQ ID NO: 1058-1072, 1074, 1075, 1077, 1078, 1079, 1080, 1082, 1083, 1084, 1085, 1086, 1087, 1089, 1090, 1091, 1092, 1093, 1096, 1097, 1098, 1099, 1100, 1103, 1105, 1106, 1111, 1118, 1122, 1127, 1128, 1129, 1130, 1132, 1133, 1134, 1138, 1146, 1147, 1148, 1149, 1150, 1153, 1154, 1155, 1156, 1157, 1161, 1185, 1191, 1193, 1194, 1196, 1197, 1198, 1200, 1203, 1204, 1205, 1208, 1209, 1211, 1220, 1221, 1222, 1229, 1234, 1237, 1240, 1241, 1249, 1260, 1261, 1262, 1271, 1274, 1276, 1278, 1286, 1287, 1288, 1291, 1294, 2117, 2118, and 2119.

13. The nanobody of claim 1, wherein the nanobody is a heterodimer comprising two different amino acid sequences, wherein each different amino acid sequence comprises a sequence selected from the group consisting of SEQ ID NO: 1058 through SEQ ID NO: 2114 and SEQ ID NO: 2117 through SEQ ID NO: 2119.

14. The nanobody of claim 13, wherein the nanobody is a heterodimer comprising two different amino acid sequences, wherein each different amino acid sequence comprises a sequence selected from the group consisting of SEQ ID NO: 1058-1072, 1074, 1075, 1077, 1078, 1079, 1080, 1082, 1083, 1084, 1085, 1086, 1087, 1089, 1090, 1091, 1092, 1093, 1096, 1097, 1098, 1099, 1100, 1103, 1105, 1106, 1111, 1118, 1122, 1127, 1128, 1129, 1130, 1132, 1133, 1134, 1138, 1146, 1147, 1148, 1149, 1150, 1153, 1154, 1155, 1156, 1157, 1161, 1185, 1191, 1193, 1194, 1196, 1197, 1198, 1200, 1203, 1204, 1205, 1208, 1209, 1211, 1220, 1221, 1222, 1229, 1234, 1237, 1240, 1241, 1249, 1260, 1261, 1262, 1271, 1274, 1276, 1278, 1286, 1287, 1288, 1291, 1294, 2117, 2118, and 2119.

15. The nanobody of claim 13, wherein the heterodimer comprises SEQ ID NO: 1147 and SEQ ID NO: 1161.

16. The nanobody of claim 1, wherein the nanobody comprises a sequence of SEQ ID NO: 1075, SEQ ID NO: 1078, SEQ ID NO: 1084, SEQ ID NO: 1086, SEQ ID NO: 1122, SEQ ID NO: 1147, SEQ ID NO: 1161, SEQ ID NO: 1208, SEQ ID NO: 1211, SEQ ID NO: 1249, SEQ ID NO: 1276, SEQ ID NO: 2117, or SEQ ID NO: 2118.

17. The nanobody of claim 1, wherein the nanobody comprises an amino acid sequence having the same amino acids residues at positions 30, 31, 32, 33, 47, 48, 49, 50, 51, 52, 53, 54, 55, 56, 57, 58, 59, 60, 61, 62, 63, 105, 106, 107, 108, 109, 110 and 114 relative to SEQ ID NO: 1211, and wherein the nanobody specifically binds to amino acids at positions 453, 455, 456, 472, 473, 475, 476, 477, 483, 484, 485, 486, 487, 488, 489, 490, 491, 492 and 493 of SEQ ID NO: 2120.

18. The nanobody of claim 1, wherein the nanobody comprises an amino acid sequence having the same amino acids residues at positions 1, 2, 3, 25, 26, 27, 28, 29, 30, 31, 32, 33, 52, 53, 54, 74, 100, 101, 102, 103, 104, 105, 106, 111, 113 and 114 relative to SEQ ID NO: 1276, and wherein the nanobody specifically binds to amino acids at positions 364, 365, 366, 367, 369, 370, 371, 372, 374, 375, 376, 377, 378, 379, 380, 381, 382, 383, 384, 385, 386, 387, 388, 389, 407, 408, 409, 410, 411, 412, 413, 414, 429, 431, 432, 433, 435 and 526 of SEQ ID NO: 2120.

19. The nanobody of claim 1, wherein the nanobody comprises an amino acid sequence having the same amino acids residues at positions 1, 2, 28, 29, 30, 31, 32, 33, 57, 59, 98, 99, 100, 101, 102, 103, 104, 108, 109, 110, and 111 relative to SEQ ID NO: 2118, and wherein the nanobody specifically binds to amino acids at positions 368, 369, 370, 371, 374, 376, 377, 378, 379, 380, 381, 382, 383, 384, 385, 408, 410, 411, 412, 413, 414, 415, 426, 427, 428, 429 and 430 of SEQ ID NO: 2120.

20. The nanobody of claim 1, wherein the nanobody comprises an amino acid sequence having the same amino acids residues at positions 1, 2, 26, 30, 31, 32, 52, 56, 98, 99, 100, 101, 102, 103, 104, 107, 108, 109 and 110 relative to SEQ ID NO: 1078, and wherein the nanobody specifically binds to amino acids at positions 369, 372, 377, 378, 379, 380, 381, 382, 383, 384, 385, 386, 411, 412, 413, 414, 415, 427, 428, and 429 of SEQ ID NO: 2120.

21. The nanobody of claim 1, wherein the nanobody comprises an amino acid sequence having the same amino acids residues at positions 47, 52, 53, 54, 56, 57, 58, 59, 60, 61, 62, 65, 99, 100, 101, 102, 103, 104, 105, 106, 107, 108, 109, 110, 111, 112, 113 and 114 relative to SEQ ID NO: 1084, and wherein the nanobody specifically binds to amino acids at positions 368, 369, 370, 371, 372, 373, 374, 375, 376, 377, 378, 379, 380, 381, 382, 383, 384, 385, 386, 404, 405, 407, 408, 411, 412, 432, 433, 434, 435, 436, 437, 503, 504, 508 and 510 of SEQ ID NO: 2120.

22. The nanobody of claim 1, wherein the nanobody comprises an amino acid sequence having the same amino acids residues at positions 1, 2, 28, 31, 32, 99, 100, 101, 102, 103, 104, 106, 107, 110, 111, 112, 113, 114, 115, 116 and 117 relative to SEQ ID NO: 1075, and wherein the nanobody specifically binds to amino acids at positions 375, 376, 377, 378, 379, 380, 381, 382, 383, 384, 385, 386, 387, 408, 410, 411, 412, 413, 414, 415, 416, 426, 427, 428, 429, 430, 431 and 432 of SEQ ID NO: 2120.

23. The nanobody of claim 1, wherein the nanobody comprises an amino acid sequence having the same amino acids residues at positions 2, 26, 28, 30, 31, 32, 52, 53, 54, 99, 100, 101, 102, 103, 104, 107, 112, 113, 114, 115, 116 and 117 relative to SEQ ID NO: 1147, and wherein the nanobody specifically binds to amino acids at positions 369, 370, 374, 375, 376, 377, 378, 379, 380, 381, 382, 383, 384, 385, 390, 408, 410, 411, 412, 413, 414, 426, 427, 428, 429 and 430 of SEQ ID NO: 2120.

24. The nanobody of claim 1, wherein the nanobody comprises an amino acid sequence having the same amino acids residues at positions 31, 52, 56, 57, 100, 101, 102, 103, 104, 105, 106, 107, 108, 109, 110, 111, 112, 113, 114 and 115 relative to SEQ ID NO: 1122, and wherein the nanobody specifically binds to amino acids at positions 337, 340, 346, 348, 349, 351, 352, 353, 354, 355, 356, 357, 358, 359, 394, 396, 454, 464, 465, 466, 467, 468, 469 and 516 of SEQ ID NO: 2120.

25. The nanobody of claim 1, wherein the nanobody comprises an amino acid sequence having the same amino acids residues at positions 33, 38, 43, 44, 46, 47, 50, 56, 57, 58, 59, 60, 61, 62, 63, 64, 65, 101, 102, 103, 104, 105 and 106 relative to SEQ ID NO: 1249, and wherein the nanobody specifically binds to amino acids at positions 357, 380, 381, 382, 393, 394, 396, 426, 427, 428, 429, 430, 462, 463, 516, 517, 518, 519, 520, 521, 522 and 523 of SEQ ID NO: 2120.

26. The nanobody of claim 1, wherein the nanobody comprises an amino acid sequence having the same amino acids residues at positions 1, 2, 26, 27, 28, 30, 31, 32, 52, 53, 54, 97, 98, 99, 100, 101, 102, 103, 104, 105, 107, 108, 109, 110 and 111 relative to SEQ ID NO: 1161 and wherein the nanobody specifically binds to amino acids at positions 380, 381, 382, 386, 387, 389, 390, 391, 392, 393, 426, 428, 429, 430, 431, 462, 464, 513, 514, 515, 516, 517, 518, 519, 520, 521, 522, 52 and 525 of SEQ ID NO: 2120.

27. The nanobody of claim 1, wherein the nanobody comprises an amino acid sequence having the same amino acids residues at positions 27, 28, 29, 30, 31, 32, 33, 50, 52, 53, 54, 55, 56, 58, 60, 100, 101, 102, 103, 104, 105, 106, 107, 110 and 111 relative to SEQ ID NO: 2117 and wherein the nanobody specifically binds to amino acids at positions 355, 380, 381, 382, 383, 385, 386, 387, 388, 389, 390, 391, 392, 393, 394, 396, 426, 427, 428, 429, 430, 462, 463, 464, 465, 515, 516, 517, 518, 519, 520, 521 and 522 of SEQ ID NO: 2120.

28. The nanobody of claim 1, wherein the nanobody comprises an amino acid sequence having the same amino acids residues at positions 46, 48, 115, 116, 117, 118, 119, 120, 121, 122, 123, 124 and 125 relative to SEQ ID NO: 1208 and wherein the nanobody specifically binds to amino acids at positions 355, 396, 426, 428, 429, 430, 431, 463, 464, 514, 515, 516, 517, 518, 519 and 520 of SEQ ID NO: 2120.

29. The nanobody of claim 1, wherein the nanobody comprises an amino acid sequence having the same amino acids residues at positions 45, 47, 49, 57, 58, 59, 60, 61, 65, 99, 101, 103, 104, 105, 106, 107, 108, 109, 110, 111, 112, 113 and 115 relative to SEQ ID NO: 1086 and wherein the nanobody specifically binds to amino acids at positions 333, 334, 336, 359, 360, 361, 362, 363, 364, 365, 382, 384, 386, 387, 388, 389, 390, 391, 392, 393, 517, 518, 519, 520, 521, 522, 523, 524, 525 and 526 of SEQ ID NO: 2120.

30. (canceled)

31. (canceled)

32. (canceled)

33. (canceled)

34. (canceled)

35. A method of treating a coronavirus infection in a subject comprising administering to the subject a therapeutically effective amount of a nanobody of claim 1.

36. (canceled)

37. The method of claim 35, wherein the coronavirus is a sarbecovirus.

38. The method of claim 37, wherein the sarbecovirus is SARS-CoV-2 or SARS-CoV.

39. The method of claim 35, wherein the nanobody is conjugated or linked to a human serum albumin binding nanobody or nanobody fragment and the conjugate has an increased serum half-life or in vivo stability as compared to a control.

40. A method of preventing a coronavirus infection in a subject comprising administering to the subject a therapeutically effective amount of a nanobody of claim 1.

41. (canceled)

42. The method of claim 40, wherein the coronavirus is a sarbecovirus.

43. The method of claim 42, wherein the sarbecovirus is SARS-CoV-2 or SARS-CoV.

44. The method of claim 40, wherein the nanobody is conjugated or linked to a human serum albumin binding nanobody or nanobody fragment and the conjugate has an increased serum half-life or in vivo stability as compared to a control.
